(19) United States
(12) Patent Application Publication
(10) Pub. No.: US 2026/0183353 A1
(43) Pub. Date: Jul. 2, 2026

(54) METHOD OF REDUCING ADENOVIRAL VECTOR-ASSOCIATED TTS

(71) Applicants: Vector Sciences, Inc., Lynn, MA (US); Beth Israel Deaconess Medical Center, Inc., Boston, MA (US)

(72) Inventors: Peter Abbink, Lynn, MA (US); Dan H. Barouch, Newton, MA (US)

(73) Assignees: Vector Sciences, Inc., Lynn, MA (US); Beth Israel Deaconess Medical Center, Inc., Boston, MA (US)

(21) Appl. No.: 18/869,934

(22) PCT Filed: Jun. 2, 2023
(86) PCT No.: PCT/US2023/067871

§ 371 Date: Nov. 27, 2024

Related U.S. Application Data
(60) Provisional application No. 63/365,720, filed on Jun. 2, 2022.
(60) Provisional application No. 63/401,323, filed on Aug. 26, 2022.

(30) Foreign Application Priority Data
May 31, 2023 (WO) PCT/US2023/067709

(51) Int. Cl. A61K 35/761; A61K 39/00; A61K 39/235; A61P 7/02; C12N 15/86

(52) U.S. Cl. CPC A61K 35/761; A61K 39/235; A61P 7/02; C12N 15/86; A61K 2039/58; C12N 2710/10334; C12N 2710/10343

(57) ABSTRACT

The present disclosure relates to methods of reducing the risk of thrombosis with thrombocytopenia syndrome (TTS) associated with adenoviral vector therapy.

This application claims the priority benefit of U.S. provisional application No. 63/365,720, filed Jun. 2, 2022; U.S. provisional application No. 63/401,323, filed Aug. 26, 2022; and PCT patent application PCT/US23/67709, filed May 31, 2023, the contents of each of which are incorporated herein in their entireties by reference thereto.

### 1. SEQUENCE LISTING

The instant application contains a Sequence Listing which has been submitted electronically in XML format and is hereby incorporated by reference in its entirety. Said XML Sequence Listing, created on Jun. 29, 2023 is named VSI-001US_SL.xml and is 304,085 bytes in size.

### 2. BACKGROUND

Recombinant adenoviruses were originally developed for gene therapy, but the strong and sustained transgene-specific immune responses elicited by these gene delivery agents prompted their use as vaccine carriers. In addition to being highly immunogenic, adenoviruses offer many other advantages for clinical vaccine development. The adenoviral genome is relatively small (between 26 and 45 kbp), well characterized and easy to manipulate. The deletion of a single transcriptional unit, E1, renders the virus replication-incompetent which increases its predictability and reduces side effects in clinical applications. Recombinant adenoviruses can accommodate relatively large transgenes, in some cases up to 8 kb, allowing flexibility in subunit design, and have a relatively broad tropism facilitating transgene delivery to a wide variety of cells and tissues. Importantly for clinical applications, methods for scaled-up production and purification of recombinant adenoviruses to high titer are well established.

However, with large deployment of adenovirus vectored vaccines in the COVID-19 pandemic, a complication of adenovirus vectors emerged. In particular, some patients receiving adenovirus vectored COVID-19 vaccines developed thrombosis with thrombocytopenia syndrome (TTS). Symptoms of TTS include venous or arterial thrombosis (often cerebral or abdominal), thrombocytopenia (platelet count <150×10⁹/L), markedly elevated D-dimer (>4 times upper limit of normal), and the presence of anti-platelet factor 4 (“PF4”) antibodies similar to heparin-induced thrombocytopenia. For AZD1222, a ChAdOx1-vectored COVID-19 vaccine, TTS symptoms appeared within 4 and 42 days of administration. See, e.g., Pavord et al., 2021, N Engl J Med 2021; 385:1680-1689. The condition is often fatal.

There is a need in the art for methods of reducing the risk of developing TTS following administration of adenovirus vectored therapies.

### 3. SUMMARY

The present invention addresses the need in the art for reducing the risk of TTS associated with adenovirus vectored therapies. The need is addressed by administering to subjects at risk of TTS low negative charge adenoviral vector therapy.

Accordingly, the present disclosure provides methods of treating subjects at risk of thrombosis with thrombocytopenia syndrome (“TTS”) with adenoviral vector therapy, comprising administering to a subject at risk of TTS low negative charge adenoviral vector therapy. The present disclosure further provides methods of reducing the risk of thrombosis with thrombocytopenia syndrome (“TTS”) associated with adenoviral vector therapy, comprising administering to a subject at risk of TTS low negative charge adenoviral vector therapy.

In certain aspects, the risk of TTS is reduced as compared to administration of human or chimp adenoviral vector therapy, for example the corresponding therapy in a human Ad5 vector, a human Ad26 vector, and/or a chimp AdY25 (ChAdOx1) vector. In some embodiments, the reduction in risk of TTS is reflected as a reduction in TTS in a patient population when administering a transgene using the low negative charge adenoviral vectors of the disclosure as compared to administering the transgene in one or more chimp and/or human adenoviral vectors, e.g., a human Ad5 vector, a human Ad26 vector, and/or a chimp AdY25 (ChAdOx1) vector.

Examples of low negative charge adenoviral vectors for use in the methods of the disclosure are disclosed in Section 5.2 and numbered embodiments 7 to 61 and 91 to 92.

Examples of patient populations at risk of TTS for whom the methods of the disclosure are suitable are disclosed in Section 5.3 and numbered embodiments 65 to 90. The subjects can be selected, identified and/or classified prior to administration of the low negative charge adenoviral therapy in accordance with the methods of the disclosure, for example as disclosed in numbered embodiments, 62 to 64.

Examples of therapies the low negative charge adenoviral vectors are useful for, e.g., transgenes that the low negative charge adenoviral vectors may incorporate, together with disease indications suitable for treatment or prevention, are disclosed in Section 5.4 and numbered embodiments 116 to 182.

Examples of pharmaceutical compositions and methods and routes of administration suitable for the methods of the disclosure are disclosed in Section 5.5 and numbered embodiments 93 to 115.

### 4. BRIEF DESCRIPTION OF THE FIGURES

FIG. 1 shows the sequence alignments of the hypervariable regions (HVRs) of adenovirus hexon amino acid sequences for HuAd5; ChAdOx1; HuAd26; HuAd48; RhAd51; RhAd52; RhAd53; RhAd54; RhAd55; RhAd56; RhAd57; RhAd58; RhAd59; RhAd60; RhAd61; RhAd62; RhAd63; RhAd64; RhAd65; RhAd66; and RhAd67. FIG. 1A, HVR1 (SEQ ID NOS: 1-22); FIG. 1B, HVR2 (SEQ ID NOS: 23-44); FIG. 1C, HVR3 (SEQ ID NOS: 45-66) and HVR4 (SEQ ID NOS: 67-88); FIG. 1D, HVR5 (SEQ ID NOS: 89-110) and HVR6 (SEQ ID NOS: 111-132); FIG. 1E, HVR7 (SEQ ID NOS: 133-154).

FIG. 2 shows the ribbon structures of protein modeling for the adenovirus hexon amino acid sequences for ChAdOx1: YP006272963.1 (SEQ ID NO: 156); Ad5: BAG48782.1 (SEQ ID NO: 155); Ad26: ABO61316.1 (SEQ ID NO:157); and RhAd52: AIY35086.1 (SEQ ID NO: 160). The surface charges of the protein structures are emphasized. The top view is the surface that is exposed on the outside of a virus particle.

FIG. 3 shows the cartoons of protein modeling for the adenovirus hexon amino acid sequences for ChAdOx1: YP006272963.1 (SEQ ID NO: 156); Ad5: BAG48782.1 (SEQ ID NO: 155); Ad26: ABO61316.1 (SEQ ID NO: 157); and RhAd52: AIY35086.1 (SEQ ID NO: 160). The surface charges of the cartoons are emphasized. The top view is the surface that is exposed on the outside of a virus particle.

FIG. 4 shows the cartoons of protein modeling for the adenovirus hexon amino acid sequences for RhAd51; RhAd52; RhAd53; RhAd54; RhAd55; RhAd56; RhAd57; RhAd58; RhAd59; RhAd60; RhAd61; RhAd62; RhAd63; RhAd64; RhAd65; and RhAd66 (SEQ ID NOS: 159-175, respectively). The surface charges of the cartoons are emphasized.

FIG. 5 shows the full hexon charge at pH 7.4 for adenovirus hexon amino acid sequences for ChAdY25; Ad26; RhAd52; and RhAd56. The net electrostatic charge of the protein is indicated by z. The pH at which the protein would be neutrally charged is indicated by pl. FIG. 5A, ChAdY25 (ChAdOx1) (SEQ ID NO:156); FIG. 5B, RhAd52 (SEQ ID NO:160); FIG. 5C, HuAd26 (SEQ ID NO:157); FIG. 5D, RhAd56 (SEQ ID NO: 164).

FIG. 6 shows the hypervariable regions (HVRs) charge at pH 7.4 for the amino acid sequences of HVR1-HVR7 for adenovirus for ChAdY25 and RhAd56. The net electric charge of the protein is indicated by z. The pH at which the protein would be neutrally charged is indicated by pl. FIG. 6A, ChAdY25 (ChAdOx1) HVR1 (SEQ ID NO:3); FIG. 6B, ChAdY25 (ChAdOx1) HVR2 (SEQ ID NO:25); FIG. 6C, ChAdY25 (ChAdOx1) HVR3 (SEQ ID NO: 47); FIG. 6D, ChAdY25 (ChAdOx1) HVR4 (SEQ ID NO:69); FIG. 6E, ChAdY25 (ChAdOx1) HVR5 (SEQ ID NO:91); FIG. 6F, ChAdY25 (ChAdOx1) HVR6 (SEQ ID NO:113); FIG. 6G, ChAdY25 (ChAdOx1) HVR7 (SEQ ID NO: 135); FIG. 6H, RhAd56 HVR1 (SEQ ID NO: 11); FIG. 6I, RhAd56 HVR2 (SEQ ID NO:33); FIG. 6J, RhAd56 HVR3 (SEQ ID NO:55); FIG. 6K, RhAd56 HVR4 (SEQ ID NO:77); FIG. 6L, RhAd56 HVR5 (SEQ ID NO:99); FIG. 6M, RhAd56 HVR6 (SEQ ID NO:121); FIG. 6N, RhAd56 HVR7 (SEQ ID NO:143).

FIG. 7 shows sensorgram plots of surface plasmon resonance analysis of PF4 binding by RhAd52 as compared to HuAd26. FIG. 7A, HuAd26 (Ad26); FIG. 7B, RhAd52.

### 5. DETAILED DESCRIPTION

### 5.1. Definitions

As used herein, the following terms are intended to have the following meanings:

A/An: As used herein the specification, “a” or “an” means one or more unless the context dictates otherwise. As used herein in the claim(s), when used in conjunction with the word “comprising”, the words “a” or “an” mean one or more than one.

Adenovirus: The term “adenovirus” refers to a medium-sized (90-100 nm), nonenveloped icosahedral virus that includes a capsid and a double-stranded linear DNA genome. The adenovirus can be a naturally occurring adenovirus or a recombinant adenovirus. The term adenovirus encompasses replication-defective and replication-competent adenoviruses.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelised antibodies, chimeric antibodies, bispecific or multispecific antibodies, anti-idiotypic (anti-id) antibodies and antibody fragments with antigen-binding capability, such as single-chain Fvs (scFv), a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment, which consists of a VH domain; and an isolated complementarity determining region (CDR). The antibodies can be of any isotype/class (e.g., IgG, IgE, IgM, IgD, IgA and IgY) or subclass (e.g., IgG1, IgG2, IgG3, IgG4, IgA1 and IgA2).

Cancer: The term “cancer” refers to a disease characterized by the uncontrolled (and often rapid) growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the.

Comprise: Throughout this specification and claims, the word “comprise” and variations such as “comprises” or “comprising” will be understood to imply the inclusion of a stated integer or group of integers but not the exclusion of any other integer or group of integers.

Concurrently: The term “concurrently” is not limited to the administration of two or more agents (e.g., a low negative charge adenoviral vector and a second agent) at exactly the same time, but rather it is meant that the agents are administered to a subject in a sequence and within a time interval such that the agents can act together to provide an increased benefit than if they were administered otherwise.

Deletion: By “deletion” of an adenoviral genomic region is meant the partial or complete removal, the disruption (e.g., by an insertion mutation), or the functional inactivation (e.g., by a missense mutation) of a specified genomic region (e.g., the E1, E2, E3, and/or E4 region), or any specific open-reading frame within the specified region.

Hexon: The term “hexon” refers to a naturally occurring or engineered adenoviral hexon protein. A hexon protein for use in the method of the disclosure typically shares at least one function of a naturally occurring hexon protein, for example ability to enter a host cell. It can be easily determined if a recombinant adenovirus can enter a host cell. For example, in one embodiment, after contacting a host cell with an adenovirus comprising the hexon protein, the recombinant host cell can be washed and lysed and it can be determined whether an adenoviral RNA and/or DNA is found in the host cell using an appropriate primers and/or probes specific for adenoviral RNA and/or DNA. Alternatively or additionally, e.g., if the adenoviral genome encodes a marker (such as a fluorescent) protein, the host can be evaluated for expression of the marker protein following infection with the adenovirus.

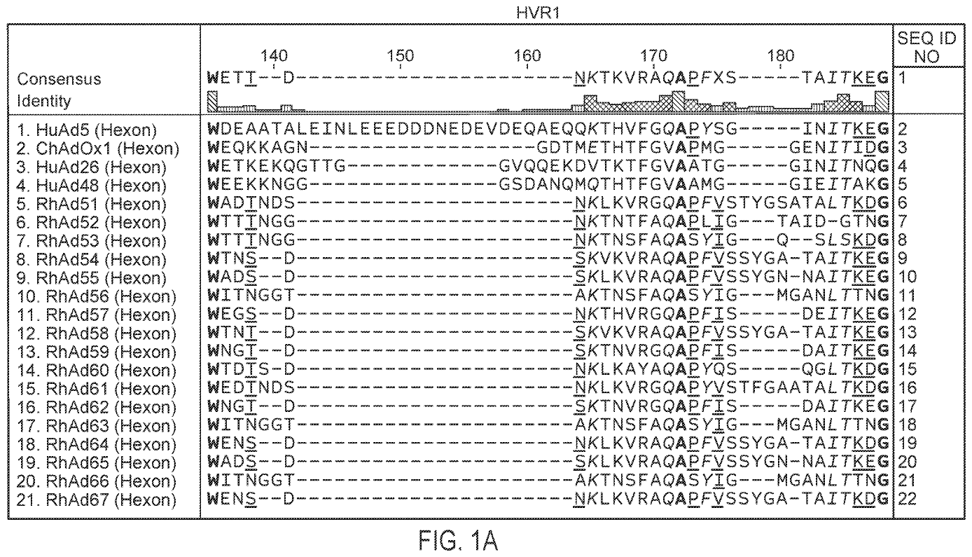

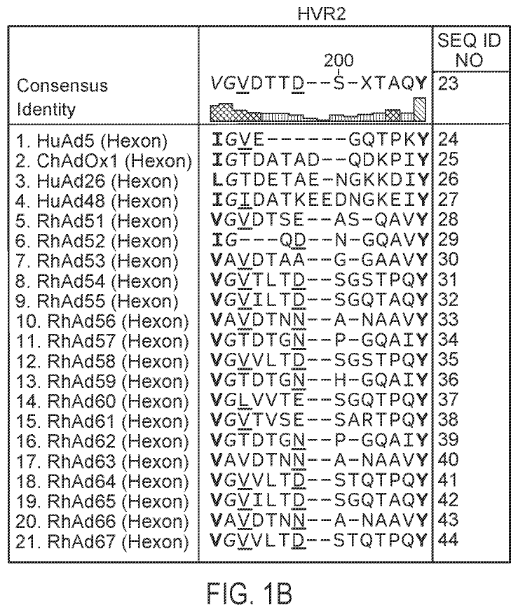

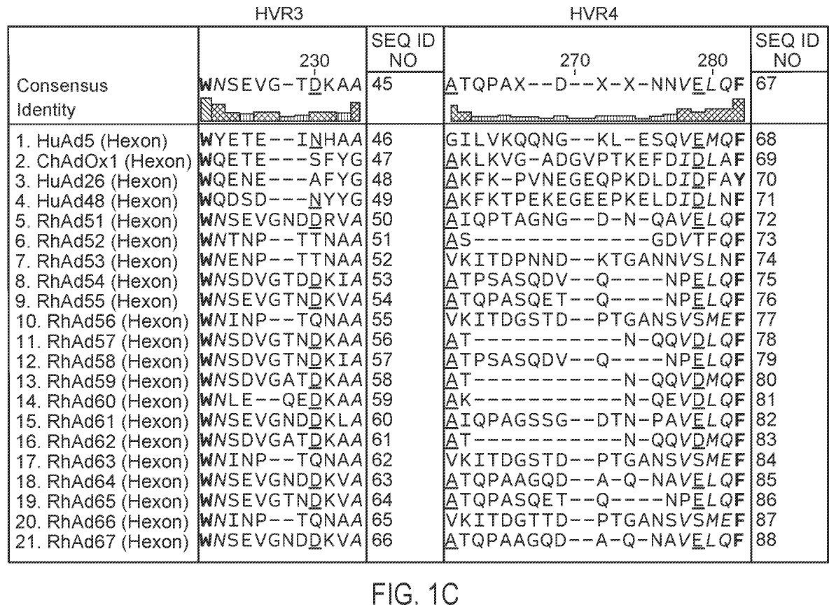

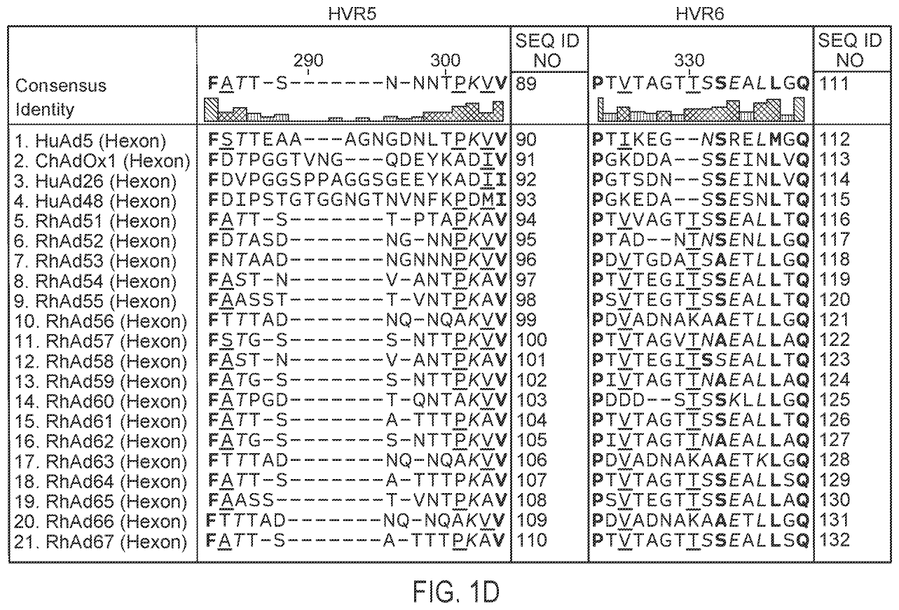

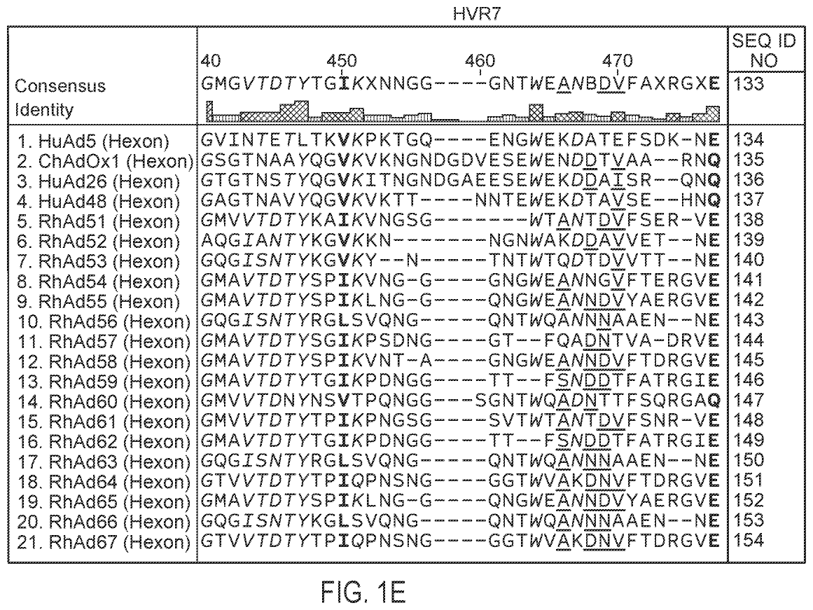

Hypervariable Region or HVR: The term “hypervariable region” or HVR refers to a domain with high sequence variation between adenoviral strains, located at the solvent-exposed surface of the hexon protein. Hexon protein HVRs occur in loops at the top of the molecule that lie on the exterior of the virion and cover nearly its entire surface. Thus, the HVRs are exposed at the outside of the viral capsid. With respect to the hexon proteins of any of Ad5, Ad48, ChAdOx1, and any of rhesus adenoviruses rhAd51 through rhAd67, the terms “hypervariable region” and “HVR” refer to the sequences shown in FIG. 1A-FIG. 1E and whose boundaries are set forth in Table 2. To ascertain the boundaries of the HVRs of another hexon protein, the hexon protein is aligned against the hexon protein of human Ad5 using the Clustal Omega algorithm as described in Example 1, and HVRs of such hexon protein for the purposes of the present invention shall be the amino acid positions corresponding to the amino acids of the HVRs of human Ad5.

Immune Response: The term “immune response” encompasses both the innate immune responses to a protein encoded by a transgene (e.g., a tumor-associated antigen), as well as memory responses that are a result of acquired immunity. The immune response elicited by the transgene-encoded protein may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing cytokines, e.g., interferon gamma (IFN gamma), tumor necrosis factor alpha (TNF alpha) and/or interleukin 2 (IL2). Alternatively, or additionally, the antigen specific T cell response comprises a CD8+ T cell response, such as a response involving CD8+ T cells expressing cytokines, e.g., IFN gamma, TNF alpha and/or IL2.

Monkeypox: The term “monkeypox” refers to a disease caused by the monkeypox virus (MPXV). A person with monkeypox disease can be contagious from the time their symptoms begin until clearance of all symptoms, which may last between two and four weeks (Alakunle, 2022, Nat rev Microbiol, 20 (9): 507-508). Interspecies transmission of MPXV to humans may involve bites or scratches inflicted by an infected animal, coming into close contact or the consumption of inappropriately cooked infected animals (Singhal, 2022, Indian J. Pediatr. doi: 10.1007/s12098-022-04348-0). The human-to-human spread of MPXV typically involves coming into prolonged contact with a contaminated surface, such as direct skin contact with monkeypox rash, or scabs; contact with bodily fluids such as mucus or large respiratory droplets of an infected individual or indirect contact with contaminated fomites, such as bedding, clothing, towels, or other items previously used by an infected individual (Lai, 2022, J Microbiol Immunol Infect. doi: 10.1016/j.jmii.2022.07.004; Singhal, 2022, Indian J. Pediatr. doi: 10.1007/s12098-022-04348-0). Moreover, transmission of MPXV from mother to fetus or newborn has also been reported (Singhal, 2022, Indian J. Pediatr. doi: 10.1007/s12098-022-04348-0).

Monkeypox Virus: MPXV was first characterized in nonhuman primates in 1953 and the monkeypox disease caused by this virus has been reported in humans since 1970s. This disease remained endemic in Africa for decades; however, the most recent monkeypox disease outbreak began in Europe in May 2022. By mid-August 2022, there have been more than 40,000 confirmed cases worldwide (CDC, 2022). MPXV belongs to the Poxviridae family, the Chordopoxvirinae subfamily, and the genus Orthopoxvirus. The Poxviridae family are brick-shaped, enveloped, double-stranded deoxyribonucleic acid viruses which infect a range of animals. Members of the genus Orthopoxvirus are large viruses that cause disease in humans and other animals. Some other members of the genus Orthopoxvirus that are related to the MPXV are the cowpox virus, camelpox virus, vaccinia virus, and the smallpox-causing variola virus. There are two main clades of MPXV: the Central African clade and the West African clade. The genomic analysis of samples isolated from cases in the current outbreak indicate that the spread involves the West African clade (Lai, 2022, J Microbiol Immunol Infect. doi: 10.1016/j.jmii.2022.07.004; Titanji, 2022, Open Forum Infect Dis 9 (7): ofac310; Benites-Zapata, 2022, Ann Clin Microbiol Antimicrob, 21:36). Many viral isolates of the 2022 MPXV have been sequenced, including MPVX isolates MPXV_USA_2022_IL001; collected May 2022 (GenBank accession no. ON959131.1); MPXV/human/USA/UW-WA-0042/2022, collected July 2022 (GenBank accession no.OP169336.1); MPXV_USA_2022_NY003, collected May 2022 (GenBank accession no.ON959134.1); MPXV_USA_2022_GA001, collected May 2022 (GenBank accession no. OP150925.1); MPXV_USA_2022_CA006, collected June 2022 (GenBank accession no. OP150924.1); MPXV-ROK-P1-2022, collected August 2022 in South Korea (GenBank accession no. OP204857.1); and MPXV/Germany/2022/RKI252, collected August 2022 (GenBank accession no.OP215269.1).

Nucleic Acid: The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

Or: The use of the term “or” in the claims or specification is intended to mean “and/or” unless explicitly indicated to refer to alternatives only or the alternatives are mutually exclusive.

Peptide, protein, and polypeptide: The terms “peptide,” “protein,” and “polypeptide” are used interchangeably to refer to a natural or synthetic molecule comprising two or more amino acids linked by the carboxyl group of one amino acid to the alpha amino group of another. The amino acids may be natural or synthetic, and can contain chemical modifications such as disulfide bridges, substitution of radioisotopes, phosphorylation, substrate chelation (e.g., chelation of iron or copper atoms), glycosylation, acetylation, formylation, amidation, biotinylation, and a wide range of other modifications. A polypeptide may be attached to other molecules, for instance molecules required for function. Examples of molecules which may be attached to a polypeptide include, without limitation, cofactors, polynucleotides, lipids, metal ions, phosphate, etc. Non-limiting examples of polypeptides include peptide fragments, denatured/unstructured polypeptides, polypeptides having quaternary or aggregated structures, etc. There is expressly no requirement that a polypeptide must contain an intended function; a polypeptide can be functional, non-functional, function for unexpected/unintended purposes, or have unknown function. There twenty, standard naturally occurring amino acids in naturally occurring proteins, although natural and synthetic amino acids which are not members of the standard twenty amino acids may also be used. The standard twenty amino acids are alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

Percent identity: As used herein, the “percent identity” between two nucleotide sequences or between two amino acid sequences is calculated by multiplying the number of matches between a pair of aligned sequences by 100, and dividing by the length of the aligned region. Identity scoring only counts perfect matches and does not consider the degree of similarity of amino acids to one another, nor does it consider substitutions or deletions as matches. Percentage identity may be calculated over contiguous sequences, where one sequence is aligned with the other sequence and each nucleotide or amino acid in one sequence is directly compared with the corresponding nucleotide or amino acid in the other sequence, one residue at a time. This is called an “ungapped” alignment. Typically, such ungapped alignments are performed only over a relatively short number of residues. Although this is a very simple and consistent method, it fails to take into consideration that, for example, in an otherwise highly similar or identical pair of sequences, one insertion or deletion in the nucleotide or amino acid sequence may cause the following nucleotides or amino acids to be put out of alignment, thus potentially resulting in a large reduction in percent homology when a global alignment is performed. Consequently, most sequence comparison methods are designed to produce optimal alignments that take into consideration possible insertions and deletions without penalizing unduly the overall sequence identity. This is achieved by inserting “gaps” in the sequence alignment to try to maximize local homology. These more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affinity gap costs” are typically used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension. Calculation of maximum percentage identity therefore firstly requires the production of an optimal alignment, taking into consideration gap penalties. A suitable computer program for carrying out such an alignment is Clustal Omega (Sievers & Higgins, 2018, Protein Science 27:135-145). Clustal Omega is included in the Geneious software package.

Provoke(s) or elicit(s) an immune response: By “provoke(s) an immune response” or “elicit(s) an immune response” is meant eliciting a humoral response (e.g., the production of antibodies) or a cellular response (e.g., the activation of T cells, macrophages, neutrophils, and natural killer cells) directed against, for example, one or more infective agents (e.g., a bacterium, virus, parasite, fungus, or combination thereof) or protein targets (e.g., a tumor associated antigen) in a subject to which a low negative charge adenoviral vector therapy (e.g., a vaccine) has been administered. Immune responses include both cell-mediated immune responses (i.e., responses mediated by antigen-specific and non-specific T-cells, such as CD8+ T-cells, Th1 cells, Th2 cells, and Th17 cells) as well as humoral immune responses (i.e., responses characterized by B-cell activation and the production of antigen-specific antibodies).

Recombinant: By “recombinant” with respect to a vector or virus, is meant a vector or virus that has been manipulated in vitro, such as a vector or virus that includes a heterologous nucleotide sequence (e.g., a sequence encoding an antigenic or therapeutic gene product) or a vector or virus bearing an alteration, disruption, or deletion in the vector or virus, such as an alteration, disruption, or deletion in a viral E1, E3, and/or E4 region, relative to a wild-type vector or virus.

Therapy: As used herein in the context of low negative charge adenoviral vector therapy, the term “therapy” is an approach for obtaining beneficial or desired results, such as clinical results. Beneficial or desired results can include, but are not limited to, eliciting an immune response in order to prevent or treat a disease, condition, or infection; alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable.

Transgene: A “transgene” is a nucleic acid sequence heterologous to the adenoviral vector sequences flanking it, which encodes an RNA or polypeptide of interest. The nucleic acid coding sequence can be operatively linked to regulatory components in a manner which permits transgene transcription, translation, and/or expression following administration to a subject. Reference to a “transgene” throughout the specification, unless the context dictates otherwise, includes coding sequences heterologous an adenoviral vector that are or are not operably linked to regulatory components. For ease of reference, the reference to a transgene sometimes refers to a protein encoded thereby (e.g., the specification may refer to an immune response against a transgene, which refers to an immune response against a protein encoded by the transgene).

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens (“TSAs”).

Vaccine: The term “vaccine” as used herein refers to material used to provoke or elicit an immune response and may confer immunity after administration of the vaccine to a subject.

Vector: By “vector” is meant a composition that includes one or more genes (non-structural or structural), or fragments thereof, from a viral species, such as an adenoviral species (e.g., RhAd51-RhAd67), that may be used to transmit one or more heterologous genes from a viral or non-viral source to a host or subject. The nucleic acid material of the viral vector may be encapsulated, e.g., in a lipid membrane or by structural proteins (e.g., capsid proteins), that may include one or more viral polypeptides (e.g., a glycoprotein). The viral vector can be used to infect cells of a subject, which, in turn, promotes the translation of the heterologous gene(s) of the viral vector into a protein product.

Virus: The term “virus” as used herein refers to an infectious agent that is unable to grow or reproduce outside a host cell and that infects mammals (e.g., humans) or birds.

Wild-type: The term “wild type” in reference to a genomic DNA or polypeptide sequence, refers to a genomic DNA sequence or polypeptide sequence that predominates in a species, e.g., Homo sapiens, or in a naturally occurring viral strain, e.g., any of RhAd51-67.

### 5.2. Low Negative Charge Adenoviral Vectors

The present disclosure relates to the use of low negative charge adenoviral vector therapy. The adenoviral capsid consists of seven structural proteins; three major capsid proteins hexon, fiber and penton; and four minor ‘cement’ proteins protein Illa (pllla), VI, VIII and protein IX (pIX). Hexon, as a major capsid component, is a target for host immune responses against adenovirus, resulting in anti-vector immunity which may hamper with adenoviral vector efficacy. As used herein, the phrase “low negative charge adenoviral vector” refers to an adenoviral vector whose hexon protein and/or its surface exposed regions have a net electrostatic charge (Z) that is greater (i.e., is more positive) than the net electrostatic charge of the hexon of protein of a commonly used human or chimp adenoviral vector and/or its surface exposed regions, e.g., HVRs, at pH 7.4. In some embodiments, the commonly used adenoviral vector is HuAd26. The GenBank accession no. of HuAd26 hexon protein is ABO61316.1 (SEQ ID NO: 157). In other embodiments, the commonly used adenoviral vector is ChAdY25, also known as ChAdOx1. The GenBank accession no. of ChAdY25 hexon protein is YP_006272963.1 (SEQ ID NO:156). The net electrostatic charge of a hexon protein or its individual surface exposed regions, e.g., HVRs, at pH 7.4 can be determined as described in Examples 1 and 2.

In certain aspects, the low negative charge adenoviral vector comprises a hexon protein having hypervariable regions having a Z (charge) average of −2.5 or greater at pH 7.4. In various embodiments, the low negative charge adenoviral vector comprises a hexon protein having hypervariable regions having a Z (charge) average of −2.3 or greater, −2 or greater, −1.5 or greater, or −1.3 or greater at pH 7.4.-2.5

In further aspects of any of the preceding embodiments, the low negative charge adenoviral vector comprises a hexon protein having hypervariable regions having a Z (charge) sum of −15 or greater at pH 7.4. In various embodiments, the low negative charge adenoviral vector comprises a hexon protein having hypervariable regions having a Z (charge) sum of −12.5 or greater or −10 or greater at pH 7.4.

In yet further aspects of any of the preceding embodiments, the low negative charge adenoviral therapy comprises a hexon protein that does not have any individual hypervariable region having a Z (charge) of less than-3.5 at pH 7.4. In various embodiments, the low negative charge adenoviral vector comprises a hexon protein that does not have any individual hypervariable region having a Z (charge) of less than-3.25, less than-3, or less than-2.75 at pH 7.4.

In yet further aspects of any of the preceding embodiments, the low negative charge adenoviral therapy comprises a hexon protein that has a Z (charge) of −17 or greater at pH 7.4. In various embodiments, the low negative charge adenoviral vector comprises a hexon protein having hypervariable regions having a Z (charge) of −16 or greater, −15 or greater, −14 or greater, −13 or greater, −12 or greater, −11 or greater, or −10 or greater at pH 7.4.

In yet further aspects of any of the preceding embodiments, the low negative charge adenoviral vector comprises a hexon protein having hypervariable regions (HVRs) having at least 90%, at least 95%, at least 97%, at least 98% sequence identity, at least 99% sequence identity or 100% sequence identity to the HVRs of a “reference” hexon protein. Examples of “reference” hexon proteins are the hexon proteins of RhAd51 (also known as sAd4287) (SEQ ID NO:159), RhAd52 (also known as sAd4310A) (SEQ ID NO: 160), RhAd53 (also known as sAd4312) (SEQ ID NO:161), RhAd54 (also known as RhAd4282) (SEQ ID NO: 162), RhAd55 (also known as RhAd4300) (SEQ ID NO: 163), RhAd56 (also known as RhAd4302) (SEQ ID NO: 164), RhAd57 (also known as RhAd4305) (SEQ ID NO:165), RhAd58 (also known as RhAd4308) (SEQ ID NO: 166), RhAd59 (also known as RhAd4309) (SEQ ID NO:167), RhAd60 (also known as RhAd4310B) (SEQ ID NO:168), RhAd61 (also known as RhAd6665) (SEQ ID NO: 169), RhAd62 (also known as RhAd6666) (SEQ ID NO: 170), RhAd63 (also known as RhAd6668A) (SEQ ID NO:171), RhAd64 (also known as RhAd6668B) (SEQ ID NO: 172), RhAd65 (also known as RhAd6669) (SEQ ID NO:173), RhAd66 (also known as RhAd6672) (SEQ ID NO:174), or RhAd67 (also known as RhAd6673) (SEQ ID NO: 175). In particular embodiments, the “reference” hexon protein is the hexon protein of RhAd52, RhAd56, RhAd59 or RhAd63 (SEQ ID NO: 160, SEQ ID NO: 164, SEQ ID NO: 167, or SEQ ID NO: 171, respectively). Thus, in some embodiments, the low negative charge adenoviral vector for use in the methods of the disclosure comprises a hexon protein having hypervariable regions (HVRs) having at least 90%, at least 95%, at least 97%, at least 98% sequence identity, at least 99% sequence identity or 100% sequence identity to the HVRs of the RhAd52 hexon protein (SEQ ID NO:160). In other embodiments, the low negative charge adenoviral vector for use in the methods of the disclosure comprises a hexon protein having hypervariable regions (HVRs) having at least 90%, at least 95%, at least 97%, at least 98% sequence identity, at least 99% sequence identity or 100% sequence identity to the HVRs of the RhAd56 hexon protein (SEQ ID NO: 164). In yet other embodiments, the low negative charge adenoviral vector for use in the methods of the disclosure comprises a hexon protein having hypervariable regions (HVRs) having at least 90%, at least 95%, at least 97%, at least 98% sequence identity, at least 99% sequence identity or 100% sequence identity to the HVRs of the RhAd59 hexon protein (SEQ ID NO:167). In yet further embodiments, the low negative charge adenoviral vector for use in the methods of the disclosure comprises a hexon protein having hypervariable regions (HVRs) having at least 90%, at least 95%, at least 97%, at least 98% sequence identity, at least 99% sequence identity or 100% sequence identity to the HVRs of the RhAd63 hexon protein (SEQ ID NO: 171).

In some embodiments, the calculation of % sequence identity against a reference set of HVRs (e.g., the HVRs of the hexon protein any one of RhAd51 through RhAd67) comprises splicing the query HVR sequences (e.g., HVR sequences of a candidate hexon protein) to generate a query HVR sequence and splicing the reference set of HVR sequences into a single reference HVR sequence, aligning the spliced query HVR sequence and the spliced reference HVR sequence, and determining percent sequence identity between the two sequences. Accordingly, in some embodiments, the low negative charge adenoviral vector for use in the methods of the disclosure comprises a hexon protein having hypervariable regions (HVRs) having collectively, across the entire length of the spliced query HVR sequence, at least 90%, at least 95%, at least 97%, at least 98% sequence identity, at least 99% sequence identity or 100% sequence identity to the spliced reference HVR sequence. In other embodiments, percent sequence identity is calculated on an HVR-by-HVR basis. Accordingly, in some embodiments, the low negative charge adenoviral vector for use in the methods of the disclosure comprises a hexon protein having hypervariable regions (HVRs) that individually each have at least 90%, at least 95%, at least 97%, at least 98% sequence identity, at least 99% sequence identity or 100% sequence identity to the corresponding reference HVR when the HVR sequence of the candidate hexon is aligned against the corresponding HVR sequence in the reference hexon.

In yet further aspects of any of the preceding embodiments, the low negative charge adenoviral vector comprises a hexon protein having at least 90%, at least 95%, at least 97%, at least 98% sequence identity, at least 99% sequence identity or 100% sequence identity to a “reference” hexon protein. Examples of “reference” hexon proteins are the hexon proteins of RhAd51, RhAd52, RhAd53, RhAd54, RhAd55, RhAd56, RhAd57, RhAd58), RhAd59, RhAd60, RhAd61, RhAd62, RhAd63, RhAd64, RhAd65, RhAd66, or RhAd67 (SEQ ID NO: 159-175, respectively). In particular embodiments, the “reference” hexon protein is the hexon protein of RhAd52, RhAd56, RhAd59 or RhAd63 (SEQ ID NO: 160, SEQ ID NO: 164, SEQ ID NO: 167, or SEQ ID NO: 171, respectively). Thus, in some embodiments, the low negative charge adenoviral vector for use in the methods of the disclosure comprises a hexon protein having at least 90%, at least 95%, at least 97%, at least 98% sequence identity, at least 99% sequence identity or 100% sequence identity to the RhAd52 hexon protein (SEQ ID NO: 160). In other embodiments, the low negative charge adenoviral vector for use in the methods of the disclosure comprises a hexon protein having at least 90%, at least 95%, at least 97%, at least 98% sequence identity, at least 99% sequence identity or 100% sequence identity to the RhAd56 hexon protein (SEQ ID NO: 164). In yet other embodiments, the low negative charge adenoviral vector for use in the methods of the disclosure comprises a hexon protein having at least 90%, at least 95%, at least 97%, at least 98% sequence identity, at least 99% sequence identity or 100% sequence identity to the RhAd59 hexon protein (SEQ ID NO: 167). In yet further embodiments, the low negative charge adenoviral vector for use in the methods of the disclosure comprises a hexon protein having at least 90%, at least 95%, at least 97%, at least 98% sequence identity, at least 99% sequence identity or 100% sequence identity to the RhAd63 hexon protein (SEQ ID NO:171).

Despite their efficiency as gene therapy vectors, the presence of pre-existing immunity in humans against adenoviruses been demonstrated in clinical trials to reduce efficacy. For example, in an Ad5 vaccine clinical trial, vaccine efficiency was significantly reduced in subjects with Ad5 neutralizing antibodies. See, e.g., Moore et al., 2008, Science 320 (5877): 753-5. Thus, it is preferable that the low negative charge adenoviral vector used in the methods of the disclosure has (or is based on an adenoviral vector having) low seroprevalence and thus low pre-existing immunity in humans. Seroprevalence can be evaluated using a luciferase assay as described by Sprangers et al., 2003, J. Clin. Microbiol 41:5046-5052. In some embodiments, a low seroprevalence adenovirus is a virus for which 20% or less of a relevant population has detectable levels of neutralizing antibodies as measured by the method of Sprangers et al. In further embodiments, a low seroprevalence adenovirus is a virus for which 10% of less of a relevant population has detectable levels of neutralizing antibodies as measured by the method of Sprangers et al. As used herein, the term “relevant population” refers to a population of intended recipients for low negative charge adenoviral vector therapy, for example a general population in the case of a low negative charge adenoviral vector SARS-COV2 vaccine or a population of cancer patients in the case of low negative charge oncolytic adenovirus. The seroprevalence of a “relevant population” can be generally extrapolated from a sample of at least 50, at least 100 or at least 200 individuals.

A number of rhesus (simian) adenoviruses (RhAd) have been described on which the low negative charge adenoviral vectors useful in the methods of the disclosure can be based or derived. RhAd51, RhAd52, RhAd53, RhAd54, RhAd55, RhAd56, RhAd57, RhAd58, RhAd59, RhAd60, RhAd61, RhAd62, RhAd63, RhAd64, RhAd65, RhAd66, and RhAd67 (i.e., RhAd51-RhAd67) have been identified and their entire genomes determined. Examples of RhAd51, RhAd52, RhAd53 adenoviral vectors are described in WO2014/078688 A2, the contents of which are incorporated by reference in their entireties herein. Examples of RhAd54, RhAd55, RhAd56, RhAd57, RhAd58, RhAd59, RhAd60, RhAd61, RhAd62, RhAd63, RhAd64, RhAd65, RhAd66, and RhAd67 adenoviral vectors are described in WO2019/118480 A1, the contents of which are incorporated by reference in their entireties herein. As shown in Example 1, these RhAds have a greater hexon electrostatic charge than the electrostatic charge of the hexon proteins of HuAd26 (SEQ ID NO: 157) and ChAdY25 (SEQ ID NO: 156) and advantageously display low seroprevalence and potent immunogenicity, e.g., when used to deliver an immunogenic agent, such as an antigenic polypeptide. Thus, adenoviruses having surface exposed regions (e.g., HVRs) of these RhAds (e.g., adenoviruses having the hexon proteins of these RhAds) are particularly useful as low negative charge adenoviral vectors for use in the methods of the disclosure.

Any of the foregoing low negative charge adenoviral vectors may be replication-deficient (RD) or replication-competent (RC). Vectors have certain regions of the adenoviral genome deleted to provide space for foreign DNA (e.g., transgenes). Replication-deficient adenoviral vectors are formed by deletion of the E1 region (which comprises the DA and E1B essential early genes) in the adenoviral genome, which ensures complete inhibition of viral replication in cells. Amplification of replication-deficient adenoviral vectors containing DNA of non-viral origin is feasible if essential viral components are provided in the helper cell in trans. This can be accomplished by generation of stable cell lines which complement for the lacking genes (e.g., as described in Kovesdi and Hedley, 2010, Viruses 2:1681-1673, which is incorporated by reference herein in its entirety).

Replication-competent adenoviral vectors usually lack the E3 region, as the E3 genes are not essential for Ad replication in cell culture or in vivo. Further details regarding RD and RC vectors may be found in Wold et al., 2013, Curr. Gene Ther. 13:421-433, which is incorporated by reference herein in its entirety.

Unless otherwise specified, features of any of the concepts, aspects and/or embodiments described above are applicable mutatis mutandis to any of the following aspects and/or embodiments.

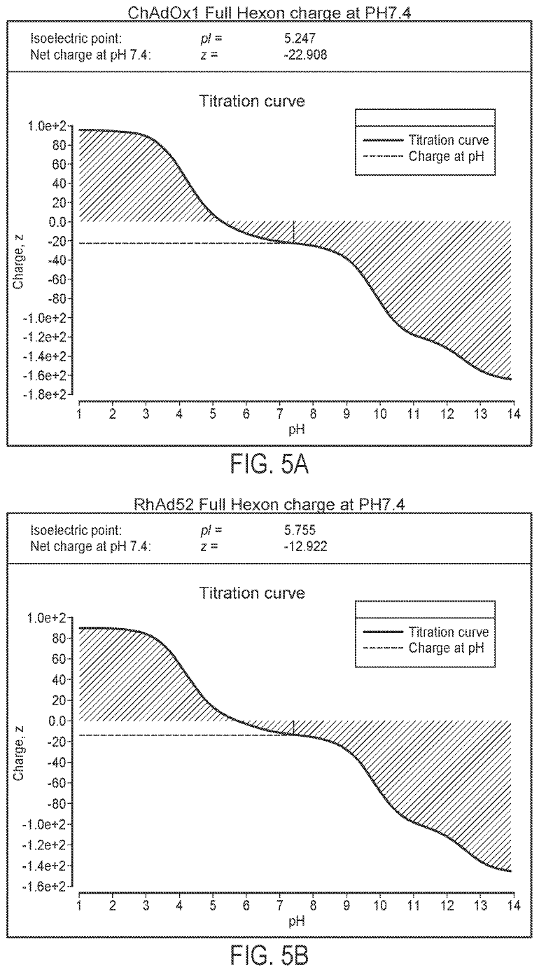

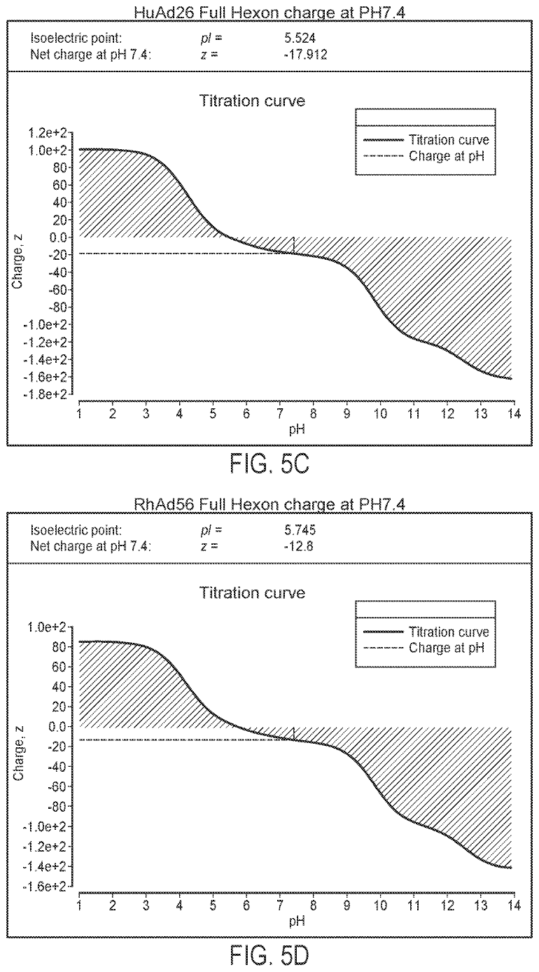

In certain aspects, a low negative charge adenoviral vector useful in the methods of the disclosure comprises a hexon protein comprising surface exposed regions, e.g., HVRs, having at least 80% sequence identity to the surface exposed regions of the hexon protein of any one of RhAd51-RhAd67. Preferably, the surface exposed regions have at least 80%, 85%, 90%, 95%, 96%, 97%, 98%, 99%, 99.5% or 99.9% sequency identity to the surface exposed regions of the hexon protein sequence of any one of RhAd51-RhAd67 (SEQ ID NO:159-175, respectively). The surface exposed regions of RhAd51-RhAd67 are illustrated in FIG. 5.

In further aspects, a low negative charge adenoviral vector useful in the methods of the disclosure comprises a hexon protein having at least 80% sequence identity to the hexon protein of any one of RhAd51-RhAd67 (SEQ ID NO: 159-175, respectively). Preferably, the sequence is at least 80%, 85%, 90%, 95%, 96%, 97%, 98%, 99%, 99.5% or 99.9% identical to the hexon protein sequence of any one of RhAd51-RhAd67 (SEQ ID NO: 159-175, respectively). The hexon protein sequence of RhAd51 is set forth as SEQ ID NO: 10 of WO2014/078688 A2 and SEQ ID NO:159 of the present disclosure. The hexon protein sequence of RhAd52 is set forth as SEQ ID NO:11 of WO2014/078688 A2 and SEQ ID NO: 160 of the present disclosure. The hexon protein sequence of RhAd53 is set forth as SEQ ID NO: 12 of WO2014/078688 A2 and SEQ ID NO: 161 of the present disclosure. The hexon protein sequence of RhAd54 is set forth as SEQ ID NO: 158 of WO2019/118480 A1 and SEQ ID NO: 162 of the present disclosure. The hexon protein sequence of RhAd55 is set forth as SEQ ID NO:159 of WO2019/118480 A1 and SEQ ID NO: 163 of the present disclosure. The hexon protein sequence of RhAd56 is set forth as SEQ ID NO: 160 of WO2019/118480 A1 and SEQ ID NO: 164 of the present disclosure. The hexon protein sequence of RhAd57 is set forth as SEQ ID NO: 161 of WO2019/118480 A1 and SEQ ID NO: 165 of the present disclosure. The hexon protein sequence of RhAd58 is set forth as SEQ ID NO: 162 of WO2019/118480 A1 and SEQ ID NO: 166 of the present disclosure. The hexon protein sequence of RhAd59 is set forth as SEQ ID NO: 163 of WO2019/118480 A1 and SEQ ID NO: 167 of the present disclosure. The hexon protein sequence of RhAd60 is set forth as SEQ ID NO: 164 of WO2019/118480 A1 and SEQ ID NO: 168 of the present disclosure. The hexon protein sequence of RhAd61 is set forth as SEQ ID NO: 165 of WO2019/118480 A1 and SEQ ID NO: 169 of the present disclosure. The hexon protein sequence of RhAd62 is set forth as SEQ ID NO: 166 of WO2019/118480 A1 and SEQ ID NO: 170 of the present disclosure. The hexon protein sequence of RhAd63 is set forth as SEQ ID NO: 167 of WO2019/118480 A1 and SEQ ID NO: 171 of the present disclosure. The hexon protein sequence of RhAd64 is set forth as SEQ ID NO: 168 of WO2019/118480 A1 and SEQ ID NO: 172 of the present disclosure. The hexon protein sequence of RhAd65 is set forth as SEQ ID NO: 169 of WO2019/118480 A1 and SEQ ID NO: 173 of the present disclosure. The hexon protein sequence of RhAd66 is set forth as SEQ ID NO: 170 of WO2019/118480 A1 and SEQ ID NO:174 of the present disclosure. The hexon protein sequence of RhAd67 is set forth as SEQ ID NO:171 of WO2019/118480 A1 and SEQ ID NO: 175 of the present disclosure. Each of these sequences is specifically incorporated by reference in its entirety herein.

In yet further aspects, a low negative charge adenoviral vector useful in the methods of the disclosure comprises a fiber protein (e.g., a fiber-1 and/or fiber-2 and/or long fiber protein) having at least 80% sequence identity to a corresponding fiber protein of any one of RhAd51-RhAd67 (SEQ ID NO: 176-219). Preferably, the sequence is at least 80%, 85%, 90%, 95%, 96%, 97%, 98%, 99%, 99.5% or 99.9% identical to a fiber protein sequence of any one of RhAd51-RhAd67 (SEQ ID NO: 176-219). The fiber-1 protein sequence of RhAd51 is encoded by the nucleotide sequence set forth as SEQ ID NO:4 of WO2014/078688 A2 (SEQ ID NO:176 of the present disclosure). The fiber-1 protein sequence of RhAd52 is encoded by the nucleotide sequence set forth as SEQ ID NO:5 of WO2014/078688 A2 (SEQ ID NO: 177 of the present disclosure). The fiber-1 protein sequence of RhAd53 is encoded by the nucleotide sequence set forth as SEQ ID NO:6 of WO2014/078688 A2 (SEQ ID NO: 178 of the present disclosure). The fiber-2 protein sequence of RhAd51 is encoded by the nucleotide sequence set forth as SEQ ID NO:7 of WO2014/078688 A2 (SEQ ID NO: 179 of the present disclosure). The fiber-2 protein sequence of RhAd52 is encoded by the nucleotide sequence set forth as SEQ ID NO:8 of WO2014/078688 A2 (SEQ ID NO: 180 of the present disclosure). The fiber-2 protein sequence of RhAd53 is encoded by the nucleotide sequence set forth as SEQ ID NO:9 of WO2014/078688 A2 (SEQ ID NO:181 of the present disclosure). The fiber-1 protein sequence of RhAd54 is set forth in SEQ ID NO: 120 of WO2019/118480 A1 (SEQ ID NO:182 of the present disclosure). The long fiber protein sequence of RhAd54 is encoded by the nucleotide sequence set forth in SEQ ID NO:44 of WO2019/118480 A1 (SEQ ID NO: 183 of the present disclosure). The fiber-1 protein sequence of RhAd55 is set forth in SEQ ID NO: 121 of WO2019/118480 A1 (SEQ ID NO: 184 of the present disclosure). The fiber-2 protein sequence of RhAd55 is set forth in SEQ ID NO: 122 of WO2019/118480 A1 (SEQ ID NO: 185 of the present disclosure). The long fiber protein sequence of RhAd55 is set forth in SEQ ID NO:145 of WO2019/118480 A1 (SEQ ID NO:186 of the present disclosure). The fiber-1 protein sequence of RhAd56 is set forth in SEQ ID NO:123 of WO2019/118480 A1 (SEQ ID NO:187 of the present disclosure). The long fiber protein sequence of RhAd56 is set forth in SEQ ID NO: 146 of WO2019/118480 A1 (SEQ ID NO: 188 of the present disclosure). The fiber-1 protein sequence of RhAd57 is set forth in SEQ ID NO:124 of WO2019/118480 A1 (SEQ ID NO: 189 of the present disclosure). The fiber-2 protein sequence of RhAd57 is set forth in SEQ ID NO:125 of WO2019/118480 A1 (SEQ ID NO:190 of the present disclosure). The long fiber protein sequence of RhAd57 is set forth in SEQ ID NO: 147 of WO2019/118480 A1 (SEQ ID NO:191 of the present disclosure). The fiber-1 protein sequence of RhAd58 is set forth in SEQ ID NO:126 of WO2019/118480 A1 (SEQ ID NO: 192 of the present disclosure). The long fiber protein sequence of RhAd58 is set forth in SEQ ID NO:148 of WO2019/118480 A1 (SEQ ID NO: 193 of the present disclosure). The fiber-1 protein sequence of RhAd59 is set forth in SEQ ID NO: 127 of WO2019/118480 A1 (SEQ ID NO:194 of the present disclosure). The fiber-2 protein sequence of RhAd59 is set forth in SEQ ID NO: 128 of WO2019/118480 A1 (SEQ ID NO: 195 of the present disclosure). The long fiber protein sequence of RhAd59 is set forth in SEQ ID NO:149 of WO2019/118480 A1 (SEQ ID NO:196 of the present disclosure). The fiber-1 protein sequence of RhAd60 is set forth in SEQ ID NO:129 of WO2019/118480 A1 (SEQ ID NO:197 of the present disclosure). The fiber-2 protein sequence of RhAd60 is set forth in SEQ ID NO: 130 of WO2019/118480 A1 (SEQ ID NO: 198 of the present disclosure). The long fiber protein sequence of RhAd60 is set forth in SEQ ID NO: 150 of WO2019/118480 A1 (SEQ ID NO: 199 of the present disclosure). The fiber-1 protein sequence of RhAd61 is set forth in SEQ ID NO:131 of WO2019/118480 A1 (SEQ ID NO:200 of the present disclosure). The fiber-2 protein sequence of RhAd61 is set forth in SEQ ID NO: 132 of WO2019/118480 A1 (SEQ ID NO:201 of the present disclosure). The long fiber protein sequence of RhAd61 is set forth in SEQ ID NO:151 of WO2019/118480 A1 (SEQ ID NO:202 of the present disclosure). The fiber-1 protein sequence of RhAd62 is set forth in SEQ ID NO: 133 of WO2019/118480 A1 (SEQ ID NO:203 of the present disclosure). The long fiber protein sequence of RhAd62 is set forth in SEQ ID NO:152 of WO2019/118480 A1 (SEQ ID NO:204 of the present disclosure). The fiber-1 protein sequence of RhAd63 is set forth in SEQ ID NO: 134 of WO2019/118480 A1 (SEQ ID NO:205 of the present disclosure). The fiber-2 protein sequence of RhAd63 is set forth in SEQ ID NO: 135 of WO2019/118480 A1 (SEQ ID NO: 206 of the present disclosure). The long fiber protein sequence of RhAd63 is set forth in SEQ ID NO:153 of WO2019/118480 A1 (SEQ ID NO:207 of the present disclosure). The fiber-1 protein sequence of RhAd64 is set forth in SEQ ID NO: 136 of WO2019/118480 A1 (SEQ ID NO:208 of the present disclosure). The fiber-2 protein sequence of RhAd64 is set forth in SEQ ID NO: 137 of WO2019/118480 A1 (SEQ ID NO:209 of the present disclosure). The long fiber protein sequence of RhAd64 is set forth in SEQ ID NO:154 of WO2019/118480 A1 (SEQ ID NO:210 of the present disclosure). The fiber-1 protein sequence of RhAd65 is set forth in SEQ ID NO: 138 of WO2019/118480 A1 (SEQ ID NO:211 of the present disclosure). The fiber-2 protein sequence of RhAd65 is set forth in SEQ ID NO: 139 of WO2019/118480 A1 (SEQ ID NO:212 of the present disclosure). The long fiber protein sequence of RhAd65 is set forth in SEQ ID NO: 155 of WO2019/118480 A1 (SEQ ID NO: 213 of the present disclosure). The fiber-1 protein sequence of RhAd66 is set forth in SEQ ID NO:140 of WO2019/118480 A1 (SEQ ID NO:214 of the present disclosure). The fiber-2 protein sequence of RhAd66 is set forth in SEQ ID NO: 141 of WO2019/118480 A1 (SEQ ID NO:215 of the present disclosure). The long fiber protein sequence of RhAd66 is set forth in SEQ ID NO: 156 of WO2019/118480 A1 (SEQ ID NO:216 of the present disclosure). The fiber-1 protein sequence of RhAd67 is set forth in SEQ ID NO: 142 of WO2019/118480 A1 (SEQ ID NO:217 of the present disclosure). The fiber-2 protein sequence of RhAd67 is set forth in SEQ ID NO: 143 of WO2019/118480 A1 (SEQ ID NO:218 of the present disclosure). The long fiber protein sequence of RhAd67 is set forth in SEQ ID NO: 157 of WO2019/118480 A1 (SEQ ID NO:219 of the present disclosure). Each of these sequences is specifically incorporated by reference in its entirety herein.

In additional aspects, a low negative charge adenoviral vector useful in the methods of the disclosure comprises a penton protein having at least 80% sequence identity to the penton protein of any one of RhAd51-RhAd67 (SEQ ID NO:220-236). Preferably, the sequence is at least 80%, 85%, 90%, 95%, 96%, 97%, 98%, 99%, 99.5% or 99.9% identical to the penton protein sequence of any one of RhAd51-RhAd67 (SEQ ID NO:220-236). The penton protein sequence of RhAd51 is provided in GenBank accession number AIY35070.1 (SEQ ID NO:220 of the present disclosure). The penton protein sequence of RhAd52 is provided in GenBank accession number AIY35085.1 (SEQ ID NO:221 of the present disclosure). The penton protein sequence of RhAd53 is provided in GenBank accession number AIY35100.1 (SEQ ID NO:222 of the present disclosure). The penton protein sequence of RhAd54 is set forth as SEQ ID NO:210 of WO2019/118480 A1 (SEQ ID NO: 223 of the present disclosure). The penton protein sequence of RhAd55 is set forth as SEQ ID NO:211 of WO2019/118480 A1 (SEQ ID NO:224 of the present disclosure). The penton protein sequence of RhAd56 is set forth as SEQ ID NO:212 of WO2019/118480 A1 (SEQ ID NO:225 of the present disclosure). The penton protein sequence of RhAd57 is set forth as SEQ ID NO:213 of WO2019/118480 A1 (SEQ ID NO:226 of the present disclosure). The penton protein sequence of RhAd58 is set forth as SEQ ID NO:214 of WO2019/118480 A1 (SEQ ID NO:227 of the present disclosure). The penton protein sequence of RhAd59 is set forth as SEQ ID NO:215 of WO2019/118480 A1 (SEQ ID NO:228 of the present disclosure). The penton protein sequence of RhAd60 is set forth as SEQ ID NO:216 of WO2019/118480 A1 (SEQ ID NO:229 of the present disclosure). The penton protein sequence of RhAd61 is set forth as SEQ ID NO:217 of WO2019/118480 A1 (SEQ ID NO: 230 of the present disclosure). The penton protein sequence of RhAd62 is set forth as SEQ ID NO:218 of WO2019/118480 A1 (SEQ ID NO:231 of the present disclosure). The penton protein sequence of RhAd63 is set forth as SEQ ID NO:219 of WO2019/118480 A1 (SEQ ID NO:232 of the present disclosure). The penton protein sequence of RhAd64 is set forth as SEQ ID NO:220 of WO2019/118480 A1 (SEQ ID NO:233 of the present disclosure). The penton protein sequence of RhAd65 is set forth as SEQ ID NO:221 of WO2019/118480 A1 (SEQ ID NO:234 of the present disclosure). The penton protein sequence of RhAd66 is set forth as SEQ ID NO:222 of WO2019/118480 A1 (SEQ ID NO:235 of the present disclosure). The penton protein sequence of RhAd67 is set forth as SEQ ID NO:223 of WO2019/118480 A1 (SEQ ID NO:236 of the present disclosure). Each of these sequences is specifically incorporated by reference in its entirety herein.

In some embodiments, the fiber, penton and hexon proteins are derived from (e.g., have at least 80%, at least 90%, at least 95%, at least 97%, at least 98% or at least 99% sequence identity to the corresponding fiber, penton and hexon protein from) the same adenoviral serotype.

Percent sequence identity between two sequences can be calculated following alignment. For short sequences, alignment can be performed manually/visually. For longer sequences, a suitable algorithm can be used, for example using Clustal Omega software as described in Example 1.

### 5.3. Patient Population

Certain subsets of the general population may have an increased risk of developing TTS following administration of adenovirus vectored therapies. The low negative charge adenoviral vector therapies can advantageously be administered to such individuals at risk of TTS. In some embodiments, the methods of the disclosure comprise screening for and/or identifying individuals at risk of TTS prior to administering a low negative charge adenoviral vector therapy according to the methods of the disclosure.

Unless otherwise specified, features of any of the concepts, aspects and/or embodiments described below are applicable mutatis mutandis to all other concepts, aspects and/or embodiments. For example, the subject to whom a low negative charge adenoviral vector therapy is administered in accordance with the methods of the disclosure may have one or any combination of two, three or more of the risk factors identified below.

The risk factors associated with thrombosis such as obesity, hypertension, diabetes, iron deficiency anemia, hypothyroidism, asthma, gastroesophageal reflux disease, obstructive sleep apnea, hyperlipidemia, seizure disorder, systemic estrogen therapy, cirrhosis, cancer, fertility, the placement of a venous catheter, trauma, immobility, genetic predisposition, autoimmune disease, and pregnancy may also be considered as risk factors for developing TTS. In particular embodiments, the low negative charge adenoviral vector therapy is administered to a subject having one or more of the foregoing conditions.

One of the risk factors for developing TTS is gender. In general, TTS is more common in females than in males but both genders are susceptible to developing TTS. Females on estrogen therapy (e.g., estrogen contraceptives or estrogen hormone replacement therapy) are at particular risk of TTS. In particular embodiments, the low negative charge adenoviral vector therapy is administered to a female, optionally a female who is receiving (or has received) estrogen therapy.

The onset of TTS following administration of adenovirus vectored therapies has a correlation with age of the subject. For example, subjects under the age of 65 have an increased likelihood of developing TTS. In particular the age group of 49 years old and younger, for example 18-49 year olds and 30-49 year olds have a higher likelihood of developing TTS. In particular embodiments, the low negative charge adenoviral vector therapy is administered to an 18-29 year old individual, a 30-39 year old individual, or a 40-49 year old individual.

The combination of the age of the subject and gender has a strong correlation of subjects developing TTS. The group of females ages 49 and younger appear to be particularly at risk. Thus, in particular embodiments, the subject to whom the low negative charge adenoviral vector therapy is administered according to the methods of the disclosure is a female aged 49 or younger, for example an adult female aged 18 to 49. Optionally, the female is receiving or has received estrogen therapy.

Race and/or ethnicity may also be associated with the risk of developing TTS following administration of adenovirus vectored therapies. White non-Hispanic subjects may have a higher risk of developing TTS compared to subjects of a different racial and/or ethnic group. Thus, in particular embodiments, the subject to whom the low negative charge adenoviral vector therapy is administered according to the methods of the disclosure is white, non-Hispanic subject.

An additional risk factor for developing TTS is the existence of anti-PF4 antibodies. Thus, in particular embodiments, the subject to whom the low negative charge adenoviral vector therapy is administered according to the methods of the disclosure has detectable levels of anti-PF4 antibodies. A suitable assay for anti-PF4 antibodies is described by Juhl et al., 2006, Eur J Haematol. 76 (5): 420-426. In some embodiments, the subject has low levels of anti-PF4 antibodies, indicated by a weak reaction in the Juhl et al. assay. In some embodiments, the subject has high levels of anti-PF4 antibodies, indicated by a strong reaction in the Juhl et al. assay. Strong and weak reactions are defined by Thiele et al., 2021, Blood 138 (4): 299-303.

In some embodiments, the low negative charge adenoviral therapy is administered to a subject who has at least one risk factor for TTS. In other embodiments, the low negative charge adenoviral therapy is administered to a subject who has at least two risk factors for TTS. In yet further embodiments, the low negative charge adenoviral therapy is administered to a subject who has three or more risk factors for TTS.

The subject to whom the low negative charge adenoviral therapy is administered may also have one or more of the risk factors or conditions which the transgene included in the vector is designed to treat. Various examples of risk factors or conditions and transgenes suitable therefor are described in Section 5.4 below.

### 5.4. Transgenes for Low Negative Charge Adenoviral Vector Therapy

The low negative charge adenoviral vector for use in the methods of the disclosure may comprise one or more exogeneous nucleotide sequences, for example 1, 2 or 3 or more exogeneous nucleotide sequences. Preferably, each exogeneous nucleotide sequence embodies a transgene.

In some embodiments, the transgene may be used as a vaccine and/or inducing an immune response, for example against a pathogen. In other embodiments, the transgene may be used to correct genetic deficiencies by correcting or replacing a defective or missing gene. In yet other embodiments, the transgene may be used as a cancer therapeutic.

Preferably, the transgene sequence is inserted into the site of a partially or fully deleted adenoviral gene, for example into the site of an E1 deletion or an E3 deletion. The transgene may be inserted into an existing adenoviral gene region to disrupt the function of that region. Alternatively, the transgene sequence may be inserted into a region of the adenoviral genome with no alteration to the function or sequence of the surrounding genes.

The transgene is preferably operably linked to regulatory sequences necessary to drive translation, transcription and/or expression of the exogeneous nucleotide sequence/transgene in a host cell, for example a mammalian cell. Such regulatory sequences include appropriate expression control sequences such as transcription initiation, termination, enhancer and promoter sequences, efficient RNA processing signals, such as splicing and polyadenylation signals, sequences that enhance translation efficiency and protein stability and sequences promote protein secretion. Additionally they may contain sequences for repression of transgene expression, for example during production in cell lines expression a transactivating receptor. Promoters and other regulatory sequences which control expression of a nucleic acid have been identified and are known in the art. Examples of suitable promoters include human CMV promoters, simian CMV promoters, murine CMV promoters, ubiquitin promoters, EF1 promoters, actin promoters and other mammalian promoters. In some embodiments, the promoters is a CMV promoter, such as a human CMV major immediate early promoter.

The transgene(s) may be introduced into the viral vector as part of a cassette. As used herein, the term “cassette” refers to a nucleic acid molecule comprising at least one nucleotide sequence to be expressed, along with its transcriptional and translational control sequences to allow the expression of the nucleotide sequence(s) in a host cell, and optionally restriction sites at the 5′ and 3′ ends of the cassette. Because of the restriction endonuclease sites, the cassettes can easily be inserted, removed or replaced with another cassette. Alternatively, any method known to one of skill in the art could be used to construct, modify or derive said cassette, for example PCR mutagenesis, In-Fusion®, recombineering, Gateway® cloning, site-specific recombination or topoisomerase cloning.

The expression control sequences preferably include the adenovirus elements necessary for replication and virion encapsidation. Preferably, the elements flank the exogeneous nucleotide sequence. Preferably, the low negative charge adenoviral vector comprises the 5′ inverted terminal repeat (ITR) sequences of a suitable adenovirus, e.g., a RhAd as described in WO2014/078688 A2 or WO2019/118480 A1, which function as origins of replication, and 3′ ITR sequences.

The packaging signal sequence functions to direct the assembly of the viral vector.

As one of skill in the art will appreciate, there are minimum and maximum constraints upon the length of the nucleic acid molecule that can be encapsidated in the viral vector. Therefore, if required, the nucleic acid molecule may also comprise “stuffing”, i.e., extra nucleotide sequence to bring the final vector genome up to the required size. Preferably, the nucleic acid molecule comprises sufficient “stuffing” to ensure that the nucleic acid molecule is about 80% to about 108% of the length of the wild-type nucleic acid molecule.

Examples of suitable transgenes are described below.

### 5.4.1. Transgenes Useful for Vaccine Applications

The low negative charge adenoviral vectors and pharmaceutical compositions may be used eliciting an immune response, e.g., when the low negative charge adenoviral vector comprises a transgene that encodes an antigenic protein, e.g., an antigenic protein of an infective agent in order to elicit an immune response against the infective agent, or a tumor-associated antigen to elicit an immune response against a cancer cell.

In various embodiments, the infective agent is a bacterium, a virus, a parasite, or a fungus. The low negative charge adenoviral vector may thus comprise a transgene that encodes a bacterial protein or fragment thereof, a viral protein or fragment thereof, a parasitic protein or fragment thereof, or a fungal protein or fragment thereof.

The low negative charge adenoviral vector may comprise a transgene that encodes a bacterial protein or fragment thereof from Mycobacterium tuberculosis, Mycobacterium bovis, Mycobacterium africanum, Mycobacterium microti, Mycobacterium leprae, Pseudomonas aeruginosa, Salmonella typhimurium, Escherichia coli, Klebsiella pneumoniae, Streptococcus pneumoniae, Staphylococcus aureus, Francisella tularensis, Brucella, Burkholderia mallei, Yersinia pestis, Corynebacterium diphtheria, Neisseria meningitidis, Bordetella pertussis, Clostridium tetani, or Bacillus anthracis.

The low negative charge adenoviral vector may comprise a transgene that encodes a viral protein or fragment from a viral family selected from the group consisting of Retroviridae, Flaviviridae, Arenaviridae, Bunyaviridae, Filoviridae, Togaviridae, Poxviridae, Herpesviridae, Orthomyxoviridae, Coronaviridae, Rhabdoviridae, Paramyxoviridae, Picornaviridae, Hepadnaviridae, Papillomaviridae, Parvoviridae, Astroviridae, Polyomaviridae, Calciviridae, and Reoviridae.

In some embodiments, the viral protein or fragment thereof, is from human immunodeficiency virus (HIV), human papillomavirus (HPV), hepatitis A virus (Hep A), hepatitis B virus (HBV), hepatitis C virus (HCV), Variola major, Variola minor, monkeypox virus, measles virus, rubella virus, mumps virus, varicella zoster virus (VZV), poliovirus, rabies virus, Japanese encephalitis virus, respiratory syncytial virus, influenza virus, herpes simplex virus (HSV), cytomegalovirus (CMV), rotavirus, influenza, Ebola virus, yellow fever virus, Zika virus, Marburg virus, or SARS-CoV2. In certain specific embodiments, the viral protein is a monkeypox virus protein, optionally wherein the monkeypox virus is a Central African clade monkeypox virus or a West African clade monkeypox virus, preferably wherein the monkeypox virus is a West African clade monkeypox virus.

In certain aspects, the infective agent is a pathogenic virus, e.g., a respiratory virus such as a coronavirus, an influenza virus or respiratory syncytial virus.

In some embodiments, the pathogenic virus is a coronavirus such as SARS-CoV-2. In some embodiments, the low negative charge adenoviral vector comprises a transgene that encodes a coronavirus spike protein or a fragment thereof.

In some embodiments, the low negative charge adenoviral vector comprises a transgene that encodes a coronavirus membrane polypeptide or a fragment thereof.

In some embodiments, the low negative charge adenoviral vector comprises a transgene that encodes a coronavirus nucleocapsid polypeptide or a fragment thereof.

In some embodiments, the low negative charge adenoviral vector comprises two or three transgenes, encoding two or more of a coronavirus spike protein or a fragment thereof, a coronavirus membrane polypeptide or a fragment thereof, and a coronavirus nucleocapsid polypeptide or a fragment thereof.

In some embodiments, the low negative charge adenoviral vector comprises a transgene that encodes a SARS-COV-2 polypeptide or fragment thereof as disclosed in WO2022/197720. Though not to be bound by theory, polypeptides and fragments thereof disclosed in WO2022/197720 can be used to produce a vaccine composition for producing neutralizing antibodies against currently existing SARS-COV-2 and its lineage variants, as well as newly arising variants. Each polypeptide and nucleotide sequence of WO2022/197720 is specifically incorporated by reference in its entirety herein.

In particular embodiments, the low negative charge adenoviral vector comprises a transgene that encodes an amino acid sequence having at least 85% identity, such as at least 90% identity, at least 95% identity, at least 96% identity, at least 97% identity, at least 98% identity, at least 99% identity, or 100% identity to any one of SEQ ID NOS: 237-266 (optionally any one of SEQ ID NOS: 237-240 and SEQ ID NOS: 249-266) or a fragment thereof capable of eliciting an immune response in a subject.

In some embodiments, the amino acid sequence or fragment thereof comprises one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, or more amino acids of any one of SEQ ID NOS: 237-266, such as any one of SEQ ID NOS: 237-240 and SEQ ID NOS: 249-266.

In some embodiments, the amino acid sequence has at least 85% identity, such as at least 90% identity, at least 95% identity, at least 96% identity, at least 97% identity, at least 98% identity, or at least 99% identity to SEQ ID NO:237, SEQ ID NO:238; SEQ ID NO: 239, or SEQ ID NO: 240, or a fragment thereof, in which the amino acid sequence comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, or thirty-two of the following mutations: S13I, L18F, T20N, P26S, Δ69-70, D80A, D80Y, L141F, Δ144, W152C, M153T, M153I, F157L, Δ242-244, D253G, S255F, A262S, V367F, K417N, K417T, N439K, L452R, Y453F, S477N, S477R, E484K, S494P, N501T, N501Y, Q613H, D614G, and P681R relative to the amino acid sequence of SEQ ID NO: 237 or SEQ ID NO:250.

In some embodiments, the amino acid sequence has (a) one, two, three, four, five, six, seven, eight, nine, ten, eleven, or twelve of the mutations: L18F, T20N, P26S, D80A, M153T, M153I, A242-244, K417N, Y453F, E484K, N501Y, and D614G relative to the amino acid sequence of SEQ ID NO:237 or SEQ ID NO:250; (b) one, two, three, four, five, six, seven, eight, or nine of the mutations: S13I, A69-70, 144, W152C, D253G, A262S, L452R, S477N, and D614G relative to the amino acid sequence of SEQ ID NO:237 or SEQ ID NO: 250; or (c) one, two, three, four, five, six, seven, eight, nine, ten, eleven, or twelve of the mutations: D80Y, L141F, F157L, S255F, V367F, K417T, N439K, S477R, S494P, N501T, Q613H, and P681R relative to the amino acid sequence of SEQ ID NO:237 or SEQ ID NO: 250.

In some embodiments, the amino acid sequence may include one or more additional modifications, such as deletion of a signal sequence (e.g., lacks a signal sequence), inclusion of a signal sequence (e.g., SEQ ID NO: 248), inclusion of one or more stabilizing mutations (e.g., proline substitutions corresponding to amino acids K969 and V970 of, e.g., SEQ ID NO: 249), an inactivation of a furin cleavage site (e.g., SEQ ID NO: 246), inclusion of a trimerization domain (e.g., a foldon trimerization domain, e.g., SEQ ID NO: 243, or other trimerization domain known in the art), and/or inclusion of a linker or spacer sequence(s) (e.g., SEQ ID NOs: 244 and 245).

In some embodiments, the amino acid sequence has at least 85% identity, such as at least 90% identity, at least 95% identity, at least 96% identity, at least 97% identity, at least 98% identity, or at least 99% identity to any one of SEQ ID NOs: 250, 253-256, and 257-260, or a fragment thereof, in which the amino acid sequence comprises has one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, or 333 of the following mutations: V3G, L5F, P9L, S13I, L18F, T19R, T20N, P26S, A27S, T33I, V36F, V36I, S45F, H49Y, Q52R, L54F, W64R, A67V, Δ69-70, G75V, T76I, D80A, D80G, P85S, S94F, T95I, S98F, 1105V, D111N, S112L, L118F, V120L, V126A, V127F, E132Q, D138Y, G142D, Δ144, N148T, W152R, Δ156-157, F157S, R158G, S162I, T167S, S172A, L176F, D178H, G181V, L189F, R190S, V193L, D198Y, 1203V, Y204H, 1210T, D215G, L216F, A222V, V227A, D228H, 1233V, R237K, Δ242-244, H245Y, Δ246-252, D253G, D253N, W258L, A262S, A263P, V267L, P272L, R273S, E281Q, T284I, V289L, A292S, T299I, K300M, T307I, V308L, E309Q, F318S, V320F, T323I, V227I, P330S, L335F, P337S, G339S, R346S, R346K, A348S, K356R, R357K, V362F, V367F, V367L, S371T, T376I, V382L, P384L, T385N, N394S, N394H, V395I, R403K, E406Q, R408I, Q414K, K417T, K417N, D427N, D427Y, T430I, N439K, N440S, N440K, L452M, L452R, L452Q, L461F, K462T, 1468V, T470I, E471Q, T478K, E484K, F490S, S494P, N501T, N501Y, Y505H, V510L, L513F, A520S, A522S, A522V, T531S, V534I, V534F, N540S, F543L, T547I, T549I, E554D, K558N, P561S, F565L, A570D, T572I, A575S, E583Q, E583D, L585F, G594S, V595I, I598V, T604A, T604I, Q607K, Q613H, D614G, N616S, V622F, A623S, D627G, P631S, T632S, T638I, S640F, N641S, A647S, A653V, H655Y, Y660F, 1670V, A672V, Q677H, P681H, P681R, A688V, S691P, A694V, M697I, A701V, S704L, A706V, T716I, T716V, T719I, M731I, T732I, T732A, M740V, T747I, T747N, N751D, S758G, T761I, T761R, G769V, A771S, V772I, Q779K, E780D, V785I, T791I, K795R, D796H, S803A, P809S, P812S, P812L, L822F, V826L, T827I, A831V, K835R, D843N, A845S, A845V, K854N, T859N, T859I, M869I, I870V, A871V, A879S, A879V, T883I, F888L, A890V, A892V, A899S, M902I, A903S, 1909V, V911I, V915I, L922F, A924S, S929I, D936Y, S939F, S940F, G946R, D950N, D950H, Q957R, T961M, V963A, S967N, V976F, S982A, K986N, K986R, R995G, 1997V, T998I, Q1005H, T1006I, T1009I, R1014K, A1016V, A1020S, A1025G, T1027I, V1033A, K1038Q, V1040F, K1045N, L1049I, M1050I, A1056V, H1058Y, L1063F, T1066N, Q1071H, K1073N, A1078S, D1084Y, D1084E, A1086S, V1094F, H1101D, H1101Y, V1104L, E1111K, D1118H, D1118Y, G1124V, D1127G, 11130V, V1133F, D1139H, L1141W, D1146Y, E1150D, D1153Y, P1162L, P1162S, D1163Y, N1173S, A1174V, V1176F, E1182D, R1185H, K1191N, N1192S, E1195Q, E1202Q, K1205N, Q1208H, 11216T, G1219V, G1219C, V1228L, M1229I, V1230L, M1237I, T1238I, C1243F, C1247F, S1252F, S1252P, D1260N, P1263L, V1264L, H1271Y, T1273I, and T1273A relative to the amino acid sequence of SEQ ID NO:237 or 250.

In some embodiments, the amino acid sequence has: (a) one, two, three, four, five, six, seven, eight, or nine of the mutations: 469-70, A144, N501Y, A570D, D614G, P681H, T716I, S982A, and D1118H relative to the amino acid sequence of SEQ ID NO:237 or 250; (b) one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, or 106 of the mutations: L5F, L18F, T20N, P26S, V36F, Q52R, D80G, T95I, 1105V, L118F, V127F, D138Y, A156-157, R158G, T167S, D178H, R190S, I203V, D215G, A222V, 1233V, A242-244, D253G, A262S, P272L, T284I, T299I, V308L, F318S, V227I, P337S, R346S, K356R, V367L, P384L, N394S, R408I, K417T, D427N, N439K, L452R, 1468V, T478K, E484K, L513F, A522S, T531S, N540S, T549I, K558N, E583D, G594S, T604A, Q613H, D614G, V622F, P631S, S640F, H655Y, 1670V, S691P, A701V, T732A, T747I, S758G, G769V, Q779K, D796H, P809S, L822F, A831V, A845S, T859N, 1870V, A879S, F888L, A899S, 1909V, L922F, S939F, D950N, T961M, 1997V, T1006I, A1016V, T1027I, V1040F, L1049I, H1058Y, Q1071H, D1084Y, V1094F, V1104L, 11130V, D1139H, D1153Y, P1162S, V1176F, K1191N, Q1208H, G1219V, V1228L, M1237I, S1252F, V1264L, and T1273I relative to the amino acid sequence of SEQ ID NO: 237 or 250; (c) one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, or 111 of the mutations: P9L, T19R, T33I, H49Y, A67V, A69-70, D80A, S98F, S112L, V126A, G142D, W152R, S162I, L176F, L189F, D198Y, 1210T, A222V, D228H, H245Y, W258L, V267L, E281Q, A292S, T307I, T323I, L335F, R346K, R357K, V367F, T376I, T385N, V395I, E406Q, K417N, D427Y, N440K, L452Q, K462T, E471Q, E484K, F490S, N501T, V510L, A520S, V534I, T547I, P561S, A570D, T572I, E583Q, V595I, T604I, A623S, T632S, N641S, A653V, A672V, P681R, A694V, S704L, T716V, M731I, M740V, N751D, T761I, A771S, E780D, T791I, S803A, P812S, T827I, D843N, K854N, A871V, T883I, A892V, M902I, V911I, A924S, D936Y, G946R, Q957R, S967N, V976F, K986N, T998I, T1009I, A1020S, V1033A, K1045N, A1056V, T1066N, A1078S, A1086S, H1101D, E1111K, G1124V, V1133F, D1146Y, P1162L, A1174V, R1185H, E1195Q, K1205N, G1219C, M1229I, T1238I, C1247F, D1260N, and H1271Y relative to the amino acid sequence of SEQ ID NO:237 or 250; or (d) one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 of the mutations: V3G, S13I, L18F, A27S, V36I, S45F, L54F, W64R, G75V, T76I, P85S, S94F, D111N, V120L, E132Q, N148T, F157S, S172A, G181V, V193L, Y204H, L216F, V227A, R237K, A246-252, D253N, A263P, R273S, V289L, K300M, E309Q, V320F, P330S, G339S, A348S, V362F, S371T, V382L, N394H, R403K, Q414K, T430I, N440S, L452M, L461F, T470I, T478K, S494P, N501Y, Y505H, A522V, V534F, F543L, E554D, F565L, A570D, A575S, L585F, 1598V, Q607K, D614G, N616S, D627G, T638I, A647S, Y660F, Q677H, A688V, M697I, A706V, T716I, T719I, T732I, T747N, T761R, V772I, V785I, K795R, P812L, V826L, K835R, A845V, T859I, M869I, A879V, A890V, A903S, V915I, S929I, S940F, D950H, V963A, V976F, S982A, K986R, R995G, Q1005H, R1014K, A1025G, K1038Q, M1050I, L1063F, K1073N, D1084E, H1101Y, D1118Y, D1127G, L1141W, E1150D, D1163Y, N1173S, E1182D, N1192S, E1202Q, 11216T, V1230L, C1243F, S1252P, P1263L, and T1273A relative to the amino acid sequence of SEQ ID NO:237 or 250.

In some embodiments, the adenovirus-based vaccine comprises two or three transgenes, optionally selected from (1) a transgene encoding an amino acid sequence having at least 85%, at least 90%, at least 95%, at least 96%, at least 97%, at least 98%, at least 99% or 100% sequence identity to any one of SEQ ID NOS: 237-240 and SEQ ID NOS: 249-260 or a fragment thereof; (2) a transgene encoding an amino acid sequence having at least 85%, at least 90%, at least 95%, at least 96%, at least 97%, at least 98%, at least 99% or 100% sequence identity to any one of SEQ ID NO:261-263 or a fragment thereof; (3) a transgene encoding an amino acid sequence having at least 85%, at least 90%, at least 95%, at least 96%, at least 97%, at least 98%, at least 99% or 100% sequence identity to any one of SEQ ID NOS: 264-266 or a fragment thereof; and (4) any combination of two or more of the foregoing.

In some embodiments, the adenovirus-based vaccine comprises two transgenes, one encoding an amino acid sequence having at least 85%, at least 90%, at least 95%, at least 96%, at least 97%, at least 98%, at least 99% or 100% sequence identity to any one of SEQ ID NOS: 237-240 and SEQ ID NO: 249-260 or a fragment thereof, and the other encoding an amino acid sequence having at least 85%, at least 90%, at least 95%, at least 96%, at least 97%, at least 98%, at least 99% or 100% sequence identity to any one of SEQ ID NO:264-266 or a fragment thereof.

Though not to be bound by theory, the inclusion of two or three transgenes may improve induction of both humoral and cellular immunity.

The low negative charge adenoviral vector may comprise a transgene that encodes a parasitic protein or fragment thereof from Toxoplasma gondii, Plasmodium falciparum, Plasmodium vivax, Plasmodium ovale, Plasmodium malariae, Trypanosoma spp., or Legionella spp.

The low negative charge adenoviral vector may comprise a transgene that encodes fungal protein or fragment thereof from Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus.

### 5.4.2. Transgenes Useful for Cancer Applications

The low negative charge adenoviral vector therapy for use in accordance with the methods of the disclosure may be a cancer therapeutic.

In some embodiments, the low negative charge adenoviral vector is an oncolytic virus. The term “oncolytic virus” generally refers to a non-pathogenic viral strain that selectively kills malignant cells, while sparing their non-malignant counterparts. See Pol et al., 2016, Oncoimmunology 5: e1117740. Such an oncotoxic activity (which can be natural or the result of precise genetic manipulations) generally reflect an elevated degree of oncotropism (i.e., the ability of some viruses to preferentially enter neoplastic cells over normal cells of the same type) and/or the pronounced susceptibility of some cancer cells to viral replication as such or to the expression of (endogenous or exogenous) cytotoxic gene products. Pol et al., 2016, Oncoimmunology 5: e1117740.

The therapeutic activity of oncolytic viruses may be ascribed in part to oncolysis. Because most cancer cells' own mechanisms of virus clearance are damaged (e.g., protein kinase R (PKR), a key factor for virus clearance in normal cells is missing in cancer cells), viruses are more likely to replicate and spread in cancer cells.

In other, more preferred embodiments, the oncolytic virus comprises a transgene that elicits an adaptive, tumor-targeting immune response or is capable of eliciting cancer cell death by other means (e.g., a suicide gene).

In various embodiments, oncolytic viral particles are engineered to express one or more transgenes that confers one or more of the following characteristics: (1) a refined oncotropism, based on the targeting of tumor-associated antigens (TAAs) exposed on the surface of malignant cells; (2) an optimized selectivity of replication, based on various systems that allow for the expression of essential viral proteins only in cells of a predetermined tissue, transformed cells, cells exhibiting specific molecular defects, or cells exposed to precise microenvironmental conditions (naturally or artificially); (3) an exacerbated cytotoxicity, based on the expression of potentially lethal enzymes or other tumor-targeting molecules; (4) an enhanced capacity to boost tumor-targeting immune responses, based on the expression of TAAs (in the context of so-called “oncolytic vaccination”, co-stimulatory molecules, immunostimulatory cytokines, or chemokines; and (5) a limited standalone immunogenicity, based on coating/encapsulation strategies or changes of the viral surface that reduce the recognition of circulating viruses by the immune system and reticular phagocytes. See Pol et al., 2016, Oncoimmunology 5: e1117740 and references cited therein; Baker et al., 2018, Cancers 10:201 and references cited therein; Goradel et al., 2018, J Cell Physiol 1-11 DOI 10.1002/jcp.27850 and references cited therein; Peter and Kuhnel, 2020, Cancers 12:3354 and references cited therein; and Bulcha et al., 2021, Signal Transduction and Targeted Therapy 6:53 and references cited therein.

Thus, in some embodiments, the low negative charge adenoviral vector comprises a transgene that encodes a TAA or a fragment thereof, e.g., prostate-specific antigen, MAGE-A3, human papilloma virus (HPV) E6/E7, carcinoembryonic antigen (CEA), or a fragment of any of the foregoing.

In other embodiments, the low negative charge adenoviral vector comprises a transgene that encodes an anti-TAA antibody, e.g., an anti-EGFR or anti-HER2 antibody.

In yet other embodiments, the low negative charge adenoviral vector comprises a transgene that encodes a cytokine capable of promoting an anti-tumor immune response, for example GM-CSF, IFN-alpha, CD40 ligand (CD40L), interleukin-12 (IL12), or interleukin-18 (IL18).

In yet further embodiments, the low negative charge adenoviral vector comprises a transgene that encodes a cancer immunotherapy agent, e.g., an anti-CTLA antibody, an anti-PD-L1 antibody, or an anti-TAA/anti-CD3 bispecific antibody, or CD40 ligand.

In yet further embodiments, the low negative charge adenoviral vector comprises a transgene that encodes a suicide gene to cause cell cycle arrest or apoptosis, e.g., p53.

The low negative charge adenoviral vector may be modified to alter its tropism, so that has preferential selectivity for cancer cells. See, e.g., references 106-113 of Bulcha et al., 2021, Signal Transduction and Targeted Therapy 6:53.

### 5.4.3. Transgenes Useful for Other Gene Therapy Applications

The low negative charge adenoviral vectors and pharmaceutical composition may be used for gene therapy for delivering a transgene into a subject's cells or tissues.

In certain aspects, the transgene is designed to replace a deleterious mutant or non-functional allele(s) of a gene with a wild-type or functional allele(s), e.g., where the non-functional or mutant version is associated with a disease or condition. In some embodiments, a functional allele is inserted into a non-specific location within the genome to replace the non-functional allele. Alternatively, the non-functional allele may be swapped for the functional allele through homologous recombination. Subsequent expression of the functional allele within the target cell restores the target cell to a normal state and thus provides a treatment for the disease. The wild-type or functional allele(s) may be inserted into the genome of a subject at risk of TTS using a low negative charge adenoviral vector as described herein.

Accordingly, in some embodiments the low negative charge adenoviral vector for use in the methods of the present disclosure comprises a transgene encoding a functional or normal protein and/or encodes a protein that is missing or mutant in the subject. In further embodiments, the low negative charge adenoviral vector for use in the methods of the present disclosure comprises a transgene encoding a protein that inhibits an aberrant or overexpressed gene product in the subject. The aberrant or overexpressed gene product may be an endogenous gene product (e.g., VEGF) or an exogenous gene product (e.g., a retroviral gene product).

In some embodiments, the subject has AADC deficiency and the transgene encodes AADC. AADC refers to aromatic I-amino acid decarboxylase.

In some embodiments, the subject has Batten Disease and the transgene encodes CLN2 or CLN6. CLN2 refers to neuronal ceroid lipofuscinosis type 2 and CLN6 refers to neuronal ceroid lipofuscinosis type 6.

In some embodiments, the subject has MPS-IIIB and the transgene encodes NAGLU. MPS refers to mucopolysaccharidosis and NAGLU refers to N-α-acetylglucosaminidase.

In some embodiments, the subject has Parkinson's Disease and the transgene encodes AADC, GDNF, or Neurturin. AADC refers to aromatic I-amino acid decarboxylase and GFNF refers to glial cell line-derived neurotrophic factor.

In some embodiments, the subject SMA and the transgene encodes SMN. SMA refers to spinal muscular atrophy and SMN refers to survival of motor neuron.

In some embodiments, the subject has GAN deficiency or the related condition giant axonal neuropath and the transgene encodes GAN. GAN refers to gigaxonin.

In some embodiments, the subject has achromatopsia and the transgene encodes CNGB3. CNGB3 refers to cyclic nucleotide-gated channel-β3.

In some embodiments, the subject has choroideremia and the transgene encodes REP1. REP1 refers to RAB escort protein 1.

In some embodiments, the subject has LCA and the transgene encodes RPE65. LCA refers to Leber congenital amaurosis and RPE65 refers to retinal pigment epithelium-specific 65 kDa protein.

In some embodiments, the subject has LHON and the transgene encodes ND4. LHON refers to Leber hereditary optic neuropathy and ND4 refers to NADH-ubiquinone oxidoreductase chain 4.

In some embodiments, the subject has RP (RLBP1) and the transgene encodes RLBP1. RP refers to retinitis pigmentosa and RLBP1 refers to retinaldehyde-binding protein 1.

In some embodiments, the subject has wet AMD and the transgene encodes an anti-VEGF protein, optionally wherein the anti-VEGF protein is an antibody. VEGF refers to vascular endothelial growth factor and AMD refers to age-related macular degeneration.

In some embodiments, the subject has X-linked RP and the transgene encodes RPGR. RPGR refers to retinitis pigmentosa GTPase regulator.

In some embodiments, the subject has X-linked retinoschisis and the transgene encodes RS1. RS1 refers to retinoschisin 1.

In some embodiments, the subject has Crigler-Najjar syndrome and the transgene encodes UGT1A1. UGT1A1 refers to UDP glucuronosyltransferase family 1 member A1.

In some embodiments, the subject homozygous FH and the transgene encodes LDLR. FH refers to familial hypercholesterolemia and LDLR refers to low-density lipoprotein receptor.

In some embodiments, the subject has GSD1a and the transgene encodes G6PC. GSD1a refers to glycogen storage disease type 1a and G6PC refers to glucose-6-phosphatase catalytic subunit.

In some embodiments, the subject has hemophilia A and the transgene encodes FVIII. FVIII refers to factor VIII.

In some embodiments, the subject has hemophilia A and the transgene encodes FVIX. FVIII refers to factor IX.

In some embodiments, the subject has MPS-I and the transgene encodes ZFN1, ZFN2, IDUA donor. MPS refers to mucopolysaccharidosis, ZFN refers to zinc finger nuclease and IDUA refers to α-L-iduronidase.

In some embodiments, the subject has MPS-II and the transgene encodes ZFN1, ZFN2, IDS donor. MPS refers to mucopolysaccharidosis, ZFN refers to zinc finger nuclease and IDS refers to iduronate-2-sulfatase.

In some embodiments, the subject has MPS-IIIA and the transgene encodes SGSH. MPS refers to mucopolysaccharidosis and SGSH refers to N-sulfoglucosamine sulfohydrolase.

In some embodiments, the subject has MPS-VI and the transgene encodes ARSB. MPS refers to mucopolysaccharidosis and ARSB refers to arylsulfatase B.

In some embodiments, the subject has OTC deficiency and the transgene encodes OTC. OTC refers to OTC ornithine transcarbamylase.

In some embodiments, the subject has A1AT deficiency and the transgene encodes A1AT. A1AT refers to α1 antitrypsin.

In some embodiments, the subject has CMT1A and the transgene encodes NTF3. CMT1A refers to Charcot-Marie-Tooth disease type 1A and NTF3 refers to neurotrophin 3.

In some embodiments, the subject has DMD and the transgene encodes a microdystrophin or a mini-dystrophin. DMD refers to Duchenne muscular dystrophy.

In some embodiments, the subject has LGMD type 2E and the transgene encodes LGMD2E. LGMD refers to limb girdle muscular dystrophy.

In some embodiments, the subject has dysferlinopathy and the transgene encodes DYSF. DYSF refers to dysferlin.

In some embodiments, the subject has an HIV infection and the transgene encodes a PG9 antibody or a VRC07 antibody.

In some embodiments, the subject has Pompe Disease and the transgene encodes GAA. GAA refers to α-glucosidase.

In some embodiments, the subject has X-linked MTM and the transgene encodes MTM1. MTM refers to myotubular myopathy and MTM1 refers to myotubularin 1.

Examples of specific transgenes suitable for the foregoing gene therapy applications can be found in Table 2 of Bulcha et al., 2021, Signal Transduction and Targeted Therapy 6:53 and references cited therein, which are incorporated by reference herein.

### 5.5. Pharmaceutical Compositions and Methods of Administration

The low negative charge adenoviral vectors for use in the methods of the disclosure can be administered in the form of a pharmaceutical composition comprising the adenoviral vector in combination with one or more additional active ingredients, a pharmaceutically acceptable carrier, diluent, excipient or adjuvant.

Suitable carriers and/or diluents are well known in the art and include pharmaceutical grade starch, mannitol, lactose, magnesium stearate, sodium saccharin, talcum, cellulose, glucose, sucrose, (or other sugar), magnesium carbonate, gelatin, oil, alcohol, detergents, emulsifiers or water (preferably sterile).

The pharmaceutical composition may be administered by any suitable method, for example by oral (including by inhalation), intramuscular, parenteral, mucosal (e.g., buccal, sublingual, nasal), and, for cancer applications, intravesical, intraperitoneal or intratumoral administration. The pharmaceutical compositions can be adapted for the route of administration.

For another example, the pharmaceutical composition may be administered subcutaneously.

In some embodiments, the pharmaceutical composition is administered by a microneedle or a microneedle patch, e.g., a microneedle or a microneedle patch comprising microneedles coated with the pharmaceutical composition or the adenoviral vector thereof. Exemplary microneedle patches include those reported by Moon, et al., 2022, npj Vaccines, 7:26 and vander Straeten, et al., 2023, Nat Biotechnol, https://doi.org/10.1038/s41587-023-01774-z.

In some embodiments, the pharmaceutical composition is administered by a microneedle (or a microneedle patch comprising microneedles) coated with the pharmaceutical composition or the adenoviral vector thereof.

For oral administration, the pharmaceutical composition can be formulated as liquids or solids, for example solutions, syrups, suspensions or emulsions, tablets, capsules and lozenges.

A liquid formulation will generally consist of a suspension or solution of the compound or physiologically acceptable salt in a suitable aqueous or non-aqueous liquid carrier(s) for example water, ethanol, glycerin, polyethylene glycol or oil. The formulation may also contain a suspending agent, preservative, flavoring or coloring agent.

Pharmaceutical compositions for oral administration may be designed to protect the active ingredient against degradation as it passes through the alimentary tract, for example by an outer coating of the formulation on a tablet or capsule.

Typical parenteral compositions consist of a solution or suspension of the low negative charge adenoviral vector in a sterile aqueous or non-aqueous carrier or parenterally acceptable oil, for example polyethylene glycol, polyvinyl pyrrolidone, lecithin, arachis oil or sesame oil.

Pharmaceutical compositions for nasal or oral administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve, which is intended for disposal once the contents of the container have been exhausted. Where the dosage form comprises an aerosol dispenser, it will contain a pharmaceutically acceptable propellant. The aerosol dosage forms can also take the form of a pump-atomizer.

The pharmaceutical composition is preferably sterile. It is preferably pyrogen-free. It is preferably buffered, e.g., at between pH 6 and pH 8, generally around pH 7.

Preferably, the composition is substantially isotonic with humans.

For buccal administration, the pharmaceutical composition can be administered with a buccal swab or buccal spray.

For intranasal administration, the pharmaceutical composition can be administered by way of nasal spray.

In certain embodiments, the pharmaceutical compositions of the present disclosure are capable of delivering an effective amount of the low negative charge adenoviral vector to a subject at risk of TTS. Suitable patient populations are described in Section 5.3, supra.

As used herein an “effective amount” means that the administration of that amount to an individual, either as a single dose or as a series of doses, is effective for prevention or treatment of a disease or condition. Typically, a single dose comprises 1×10 to 1×10¹² viral particles. In some embodiments a single dose comprises 1×10² to 1×10⁵ viral particles, 1×10⁵ to 1×10⁸ viral particles, 10⁸ to 1×10¹⁰ viral particles, 1×10¹⁰ to 1×10¹² viral particles, or any range bounded by any two of the foregoing values (e.g., 10⁸ to 1×10¹² viral particles).

In some embodiments, the low negative charge adenoviral vectors is administered in a multidose regimen. The multiple doses can be administered via the same route or via different routes. For example, one dose can be given intramuscularly and another dose can be given mucosally. In some embodiments, the intramuscular administration is given first and the mucosal administration is given second. The formulations and doses for the multidose regimens can be the same or different.

In some embodiments, wherein the low negative charge adenoviral vector is an adenovirus-based vaccine that comprises a transgene that encodes an amino acid sequence capable of eliciting an immune response in a subject against a coronavirus such as SARS-COV-2, the adenovirus-based vaccine is administered intranasally. Alternatively or in addition, the adenovirus-based vaccine is administered in one dose or in two, three, four, or more doses one, two, three, four, five, six, seven, eight, nine, ten, eleven, twelve, or thirteen weeks apart, such as two doses one, two, three, four, five, six, seven, eight, nine, ten, eleven, twelve, or thirteen weeks apart, e.g. two doses administered four, five, six, seven, or eight weeks apart.

In some embodiments, wherein the low negative charge adenoviral vector is an adenovirus-based vaccine that comprises a transgene that encodes an amino acid sequence capable of eliciting an immune response in a subject against a coronavirus such as SARS-COV-2, a booster of the adenovirus-based vaccine can be administered annually.

Any period of time referred to herein as a week, a month, a year/annually, etc. encompasses reasonable variations, e.g. for patient schedules, practitioner schedules, inconsistency in vaccine supplies, etc. For example, an annual booster can be administered from nine to fifteen months after a prior dose of an adenovirus-based vaccine. For another example, a week can encompass 7±3 days. In some specific embodiments, a week encompasses 7±2 days. In other specific embodiments, a week encompasses 7±1 day

In certain aspects, the pharmaceutical composition is an immunogenic and/or antigenic composition. The immunogenic and/or antigenic compositions may be prophylactic (to prevent infection), post-exposure (to treat after infection but before disease) or therapeutic (to treat disease). Preferably, the immunogenic and/or antigenic composition is a vaccine. In such aspects, an effective amount means that a sufficient amount of the adenoviral vector is delivered to the subject over a suitable timeframe such that a sufficient amount of the transgene is produced by the subject's cells to stimulate an immune response which is effective for prevention or treatment of a disease or condition. This amount varies depending on the health and physical condition of the individual to be treated, age, the capacity of the individual's immune system, the degree of protection desired, the formulation of the vaccine, the doctor's assessment of the medical situation and other relevant factors.

Immunogenic and/or antigenic compositions can be formulated with one or more adjuvants. Suitable adjuvants are well known in the art and include incomplete Freund's adjuvant, complete Freund's adjuvant, Freund's adjuvant with MDP (muramyldipeptide), alum (aluminum hydroxide), alum plus Bordatella pertussis and immune stimulatory complexes (ISCOMs, typically a matrix of Quil A containing adenoviral proteins).

Preferably, transduction with the low negative charge adenoviral vector results in the stable delivery of the transgene into cells in the subject at risk of TTS.

Where the transgene encodes an antigen, expression of the transgene in a subject will result in the elicitation of a primary immune response to that antigen, leading to the development of an immunological memory which will provide an enhanced response in the event of a secondary encounter, for example upon infection by the pathogen from which the antigen was derived.

In some embodiments, the subject is a naive subject, e.g., a subject who has not previously been exposed to the pathogen or antigens in question. In other embodiments, the low negative charge adenoviral vector can be used to boost the immune response of a subject previously exposed to the antigen or pathogen.

In further embodiments, the subject has been previously exposed to the antigen in question, or “primed”. For example, the subject may have previously been inoculated or vaccinated with a composition comprising the antigen, or may have previously been infected with the pathogen from which the antigen was derived. The subject may be latently infected with the pathogen from which the antigen was derived.

Thus, the low negative charge adenoviral vectors are used to elicit, induce or boost an antigen-specific immune response in a subject at risk of TTS, for example against an antigen as described in Section 5.4.1.

The low negative charge adenoviral vector may be administered to a subject at risk of TTS either as a single immunization or multiple immunizations. Preferably, the adenoviral vector or pharmaceutical composition thereof are administered as part of a single, double or triple vaccination strategy. They may also be administered as part of a homologous or heterologous prime-boost immunization regimen.

The vaccination strategy or immunization regime may include second or subsequent administrations of the low negative charge adenoviral vector or pharmaceutical composition. The second administration can be administered over a short time period or over a long time period. The doses may be administered over a period of hours, days, weeks, months or years, for example up to or at least 1, 2, 3, 4, 5, 6, 7, 8, 9, or 10 or more weeks or 0.25, 0.5, 0.75, 1, 5, 10, 15, 20, 25, 30, 35 or 40 or more years after the first administration. Preferably, the second administration occurs at least one month or at least 2 months after the first administration. Preferably, the second administration occurs up to 10 years after the first administration. These time intervals preferably apply mutatis mutandis to the period between any subsequent doses.

To further reduce the risk of TTS, in some embodiments the methods of the disclosure comprise administering the low negative charge adenoviral vectors in combination with one or more blood thinners, e.g., one or more anticoagulants and/or antiplatelet medications. Examples of suitable anticoagulants include, but are not limited to, apixaban (marketed as Eliquis™), dabigatran (marketed as Pradaxa™), dalteparin (marketed as Fragmin™), edoxaban (marketed as Savaysa™), enoxaparin (marketed as Lovenox™), fondaparinux (marketed as Arixtra™), heparin (marketed as Innohep™), rivaroxaban (marketed as Xarelto™), and warfarin (marketed as Coumadin™ and Jantoven™). Examples of suitable antiplatelet medications include, but are not limited to, aspirin, cilostazol, clopidogrel (marketed as Plavix™), dipyridamole (marketed as Persantine™), eptifibatide (marketed as Integrilin™), prasugrel (marketed as Effient™), ticagrelor (marketed as Brilinta™), tirofiban (marketed as Aggrastat™), and vorapaxar (marketed as Zontivity™). The blood thinner(s) and the low negative charge adenoviral vector therapy can be administered simultaneously, sequentially or separately.

Alternatively or in addition, subcutaneous administration of the adenoviral vector, e.g., by microneedle or microneedle patch, may further modulate the risk of TTS.

For cancer therapy, the low negative charge adenoviral vectors and pharmaceutical compositions can be administered in combination with one or more chemotherapeutic agents. The administration can be concurrent, successive or sequential.

### 6. FURTHER EMBODIMENTS

Set out below are certain further numbered embodiments of the invention according to the disclosures elsewhere herein. Features from embodiments of the invention set out above described as relating to the invention disclosed herein also relate to each and every one of these further numbered embodiments unless the context dictates otherwise. It is further intended that unless otherwise specified, features of any of the concepts, aspects and/or embodiments described in the preceding detailed description are applicable mutatis mutandis to the embodiments set forth below.

1. A method of treating a subject at risk of thrombosis with thrombocytopenia syndrome (“TTS”) with adenoviral vector therapy, comprising administering to a subject at risk of TTS low negative charge adenoviral vector therapy.

2. A method of reducing the risk of thrombosis with thrombocytopenia syndrome (“TTS”) associated with adenoviral vector therapy, comprising administering to a subject at risk of TTS low negative charge adenoviral vector therapy.

3. The method of embodiment 2, wherein the risk of TTS is reduced as compared to administration of human or chimp adenoviral vector therapy.

4. The method of embodiment 3, wherein the human or chimp adenoviral vector is a human Ad5 vector.

5. The method of embodiment 3, wherein the human or chimp adenoviral vector is a human Ad26 vector.

6. The method of embodiment 3, wherein the human or chimp adenoviral vector is a chimp AdY25 (ChAdOx1) vector.

7. The method of any one of embodiments 1 to 6, wherein the low negative charge adenoviral vector comprises a hexon protein having hypervariable regions having a Z (charge) average of −2.5 or greater at pH 7.4, optionally wherein the hexon protein has hypervariable regions having a Z (charge) average of −2.3 or greater at pH 7.4.

8. The method of embodiment 7, wherein the low negative charge adenoviral vector comprises a hexon protein having hypervariable regions having a Z (charge) average of −2 or greater at pH 7.4.

9. The method of embodiment 9, wherein the low negative charge adenoviral vector comprises a hexon protein having hypervariable regions having a Z (charge) average of −1.5 or greater at pH 7.4.

10. The method of embodiment 9, wherein the low negative charge adenoviral vector comprises a hexon protein having hypervariable regions having a Z (charge) average of −1.3 or greater at pH 7.4.

11. The method of any one of embodiments 1 to 10, wherein the low negative charge adenoviral vector comprises a hexon protein having hypervariable regions having a Z (charge) sum of −15 or greater at pH 7.4.

12. The method of embodiment 11, wherein the low negative charge adenoviral vector comprises a hexon protein having hypervariable regions having a Z (charge) sum of −12.5 or greater at pH 7.4.

13. The method of embodiment 11, wherein the low negative charge adenoviral vector comprises a hexon protein having hypervariable regions having a Z (charge) sum of −10 or greater at pH 7.4.

14. The method of any one of embodiments 1 to 13, wherein the low negative charge adenoviral therapy comprises a hexon protein that does not have any individual hypervariable region having a Z (charge) of less than-3.5 at pH 7.4, optionally wherein the hexon protein does not have any individual hypervariable region having a Z (charge) of less than-3.25 at pH 7.4.

15. The method of embodiment 14, wherein the low negative charge adenoviral vector comprises a hexon protein that does not have any individual hypervariable region having a Z (charge) of less than-3 at pH 7.4.

16. The method of embodiment 15, wherein the low negative charge adenoviral vector comprises a hexon protein that does not have any individual hypervariable region having a Z (charge) of less than-2.75 at pH 7.4.

17. The method of any one of embodiments 1 to 16, wherein the low negative charge adenoviral vector comprises a hexon protein that has a Z (charge) of −17 or greater at pH 7.4.

18. The method of embodiment 17, wherein the low negative charge adenoviral vector comprises a hexon protein that has a Z (charge) of −16 or greater at pH 7.4.

19. The method of embodiment 18, wherein the low negative charge adenoviral vector comprises a hexon protein that has a Z (charge) of −15 or greater at pH 7.4.

20. The method of embodiment 19, wherein the low negative charge adenoviral vector comprises a hexon protein that has a Z (charge) of −14 or greater at pH 7.4, optionally wherein:
(a) the hexon protein has a Z (charge) of −13 or greater at pH 7.4;
(b) the hexon protein has a Z (charge) of −12 or greater at pH 7.4;
(c) the hexon protein has a Z (charge) of −11 or greater at pH 7.4; or
(d) the hexon protein has a Z (charge) of −10 or greater at pH 7.4.

21. The method of any one of embodiments 1 to 20, wherein the low negative charge adenoviral vector comprises a hexon protein having hypervariable regions (HVRs) having at least 90% sequence identity to the HVRs of the hexon protein of RhAd51 (whose hexon protein is SEQ ID NO: 159 and whose HVRs 1-7 are SEQ ID NO:6, SEQ ID NO:28, SEQ ID NO:50, SEQ ID NO:72, SEQ ID NO:94, SEQ ID NO: 116, and SEQ ID NO: 138, respectively), RhAd52 (whose hexon protein is SEQ ID NO: 160 and whose HVRs 1-7 are SEQ ID NO:7, SEQ ID NO:29, SEQ ID NO:51, SEQ ID NO:73, SEQ ID NO:95, SEQ ID NO: 117, and SEQ ID NO: 139, respectively), RhAd53 (whose hexon protein is SEQ ID NO: 161 and whose HVRs 1-7 are SEQ ID NO:8, SEQ ID NO:30, SEQ ID NO:52, SEQ ID NO: 74, SEQ ID NO: 96, SEQ ID NO: 118, and SEQ ID NO: 140, respectively), RhAd54 (whose hexon protein is SEQ ID NO: 162 and whose HVRs 1-7 are SEQ ID NO:9, SEQ ID NO: 31, SEQ ID NO:53, SEQ ID NO: 75, SEQ ID NO:97, SEQ ID NO: 119, and SEQ ID NO: 141, respectively), RhAd55 (whose hexon protein is SEQ ID NO: 163 and whose HVRs 1-7 are SEQ ID NO: 10, SEQ ID NO:32, SEQ ID NO:54, SEQ ID NO:76, SEQ ID NO:98, SEQ ID NO: 120, and SEQ ID NO: 142, respectively), RhAd56 (whose hexon protein is SEQ ID NO: 164 and whose HVRs 1-7 are SEQ ID NO: 11, SEQ ID NO:33, SEQ ID NO:55, SEQ ID NO: 77, SEQ ID NO: 99, SEQ ID NO: 121, and SEQ ID NO: 143, respectively), RhAd57 (whose hexon protein is SEQ ID NO: 165 and whose HVRs 1-7 are SEQ ID NO:12, SEQ ID NO: 34, SEQ ID NO:56, SEQ ID NO: 78, SEQ ID NO: 100, SEQ ID NO: 122, and SEQ ID NO: 144, respectively), RhAd58 (whose hexon protein is SEQ ID NO: 166 and whose HVRs 1-7 are SEQ ID NO: 13, SEQ ID NO:35, SEQ ID NO:57, SEQ ID NO:79, SEQ ID NO: 101, SEQ ID NO: 123, and SEQ ID NO:145, respectively), RhAd59 (whose hexon protein is SEQ ID NO: 167 and whose HVRs 1-7 are SEQ ID NO: 14, SEQ ID NO:36, SEQ ID NO:58, SEQ ID NO: 80, SEQ ID NO: 102, SEQ ID NO: 124, and SEQ ID NO: 146, respectively), RhAd60 (whose hexon protein is SEQ ID NO: 168 and whose HVRs 1-7 are SEQ ID NO:15, SEQ ID NO: 37, SEQ ID NO: 59, SEQ ID NO: 81, SEQ ID NO: 103, SEQ ID NO: 125, and SEQ ID NO: 147, respectively), RhAd61 (whose hexon protein is SEQ ID NO:169 and whose HVRs 1-7 are SEQ ID NO: 16, SEQ ID NO:38, SEQ ID NO:60, SEQ ID NO:82, SEQ ID NO: 104, SEQ ID NO: 126, and SEQ ID NO: 148, respectively), RhAd62 (whose hexon protein is SEQ ID NO: 170 and whose HVRs 1-7 are SEQ ID NO: 17, SEQ ID NO:39, SEQ ID NO:61, SEQ ID NO: 83, SEQ ID NO: 105, SEQ ID NO: 127, and SEQ ID NO: 149, respectively), RhAd63 (whose hexon protein is SEQ ID NO:171 and whose HVRs 1-7 are SEQ ID NO:18, SEQ ID NO: 40, SEQ ID NO: 62, SEQ ID NO:84, SEQ ID NO: 106, SEQ ID NO: 128, and SEQ ID NO: 150, respectively), RhAd64 (whose hexon protein is SEQ ID NO: 172 and whose HVRs 1-7 are SEQ ID NO: 19, SEQ ID NO:41, SEQ ID NO: 63, SEQ ID NO: 85, SEQ ID NO: 107, SEQ ID NO: 129, and SEQ ID NO: 151, respectively), RhAd65 (whose hexon protein is SEQ ID NO: 173 and whose HVRs 1-7 are SEQ ID NO:20, SEQ ID NO:42, SEQ ID NO:64, SEQ ID NO: 86, SEQ ID NO: 108, SEQ ID NO: 130, and SEQ ID NO: 152, respectively), RhAd66 (whose hexon protein is SEQ ID NO:174 and whose HVRs 1-7 are SEQ ID NO:21, SEQ ID NO: 43, SEQ ID NO: 65, SEQ ID NO: 87, SEQ ID NO: 109, SEQ ID NO: 131, and SEQ ID NO: 153, respectively), or RhAd67 (whose hexon protein is SEQ ID NO: 175 and whose HVRs 1-7 are SEQ ID NO:22, SEQ ID NO:44, SEQ ID NO:66, SEQ ID NO:88, SEQ ID NO: 110, SEQ ID NO: 132, and SEQ ID NO: 154, respectively), and in specific embodiments the hexon protein of:
(a) RhAd52 (whose hexon protein is SEQ ID NO:160 and whose HVRs 1-7 are SEQ ID NO:7, SEQ ID NO:29, SEQ ID NO:51, SEQ ID NO:73, SEQ ID NO:95, SEQ ID NO: 117, and SEQ ID NO: 139, respectively);
(b) RhAd56 (whose hexon protein is SEQ ID NO: 164 and whose HVRs 1-7 are SEQ ID NO: 11, SEQ ID NO:33, SEQ ID NO:55, SEQ ID NO:77, SEQ ID NO:99, SEQ ID NO: 121, and SEQ ID NO: 143, respectively);
(c) RhAd59 (whose hexon protein is SEQ ID NO: 167 and whose HVRs 1-7 are SEQ ID NO: 14, SEQ ID NO:36, SEQ ID NO:58, SEQ ID NO:80, SEQ ID NO: 102, SEQ ID NO: 124, and SEQ ID NO: 146, respectively); or
(d) RhAd63 (whose hexon protein is SEQ ID NO: 171 and whose HVRs 1-7 are SEQ ID NO: 18, SEQ ID NO:40, SEQ ID NO:62, SEQ ID NO:84, SEQ ID NO: 106, SEQ ID NO: 128, and SEQ ID NO: 150, respectively).

22. The method of embodiment 21, wherein the low negative charge adenoviral vector comprises a hexon protein having hypervariable regions (HVRs) having at least 95% sequence identity to the HVRs of the hexon protein of RhAd51 (whose hexon protein is SEQ ID NO: 159 and whose HVRs 1-7 are SEQ ID NO:6, SEQ ID NO:28, SEQ ID NO:50, SEQ ID NO:72, SEQ ID NO:94, SEQ ID NO: 116, and SEQ ID NO: 138, respectively), RhAd52 (whose hexon protein is SEQ ID NO: 160 and whose HVRs 1-7 are SEQ ID NO:7, SEQ ID NO:29, SEQ ID NO:51, SEQ ID NO:73, SEQ ID NO:95, SEQ ID NO: 117, and SEQ ID NO: 139, respectively), RhAd53 (whose hexon protein is SEQ ID NO: 161 and whose HVRs 1-7 are SEQ ID NO:8, SEQ ID NO:30, SEQ ID NO:52, SEQ ID NO:74, SEQ ID NO: 96, SEQ ID NO: 118, and SEQ ID NO: 140, respectively), RhAd54 (whose hexon protein is SEQ ID NO: 162 and whose HVRs 1-7 are SEQ ID NO:9, SEQ ID NO:31, SEQ ID NO:53, SEQ ID NO:75, SEQ ID NO:97, SEQ ID NO: 119, and SEQ ID NO: 141, respectively), RhAd55 (whose hexon protein is SEQ ID NO: 163 and whose HVRs 1-7 are SEQ ID NO: 10, SEQ ID NO:32, SEQ ID NO:54, SEQ ID NO:76, SEQ ID NO:98, SEQ ID NO: 120, and SEQ ID NO: 142, respectively), RhAd56 (whose hexon protein is SEQ ID NO: 164 and whose HVRs 1-7 are SEQ ID NO:11, SEQ ID NO:33, SEQ ID NO:55, SEQ ID NO:77, SEQ ID NO: 99, SEQ ID NO: 121, and SEQ ID NO: 143, respectively), RhAd57 (whose hexon protein is SEQ ID NO: 165 and whose HVRs 1-7 are SEQ ID NO: 12, SEQ ID NO:34, SEQ ID NO: 56, SEQ ID NO: 78, SEQ ID NO: 100, SEQ ID NO: 122, and SEQ ID NO: 144, respectively), RhAd58 (whose hexon protein is SEQ ID NO: 166 and whose HVRs 1-7 are SEQ ID NO: 13, SEQ ID NO:35, SEQ ID NO:57, SEQ ID NO:79, SEQ ID NO: 101, SEQ ID NO: 123, and SEQ ID NO: 145, respectively), RhAd59 (whose hexon protein is SEQ ID NO: 167 and whose HVRs 1-7 are SEQ ID NO: 14, SEQ ID NO:36, SEQ ID NO:58, SEQ ID NO: 80, SEQ ID NO: 102, SEQ ID NO: 124, and SEQ ID NO: 146, respectively), RhAd60 (whose hexon protein is SEQ ID NO: 168 and whose HVRs 1-7 are SEQ ID NO: 15, SEQ ID NO: 37, SEQ ID NO: 59, SEQ ID NO: 81, SEQ ID NO: 103, SEQ ID NO: 125, and SEQ ID NO: 147, respectively), RhAd61 (whose hexon protein is SEQ ID NO:169 and whose HVRs 1-7 are SEQ ID NO: 16, SEQ ID NO:38, SEQ ID NO:60, SEQ ID NO:82, SEQ ID NO: 104, SEQ ID NO: 126, and SEQ ID NO: 148, respectively), RhAd62 (whose hexon protein is SEQ ID NO: 170 and whose HVRs 1-7 are SEQ ID NO: 17, SEQ ID NO:39, SEQ ID NO:61, SEQ ID NO: 83, SEQ ID NO: 105, SEQ ID NO: 127, and SEQ ID NO: 149, respectively), RhAd63 (whose hexon protein is SEQ ID NO: 171 and whose HVRs 1-7 are SEQ ID NO:18, SEQ ID NO: 40, SEQ ID NO: 62, SEQ ID NO: 84, SEQ ID NO: 106, SEQ ID NO: 128, and SEQ ID NO: 150, respectively), RhAd64 (whose hexon protein is SEQ ID NO: 172 and whose HVRs 1-7 are SEQ ID NO: 19, SEQ ID NO:41, SEQ ID NO:63, SEQ ID NO:85, SEQ ID NO: 107, SEQ ID NO: 129, and SEQ ID NO: 151, respectively), RhAd65 (whose hexon protein is SEQ ID NO: 173 and whose HVRs 1-7 are SEQ ID NO:20, SEQ ID NO:42, SEQ ID NO:64, SEQ ID NO: 86, SEQ ID NO: 108, SEQ ID NO: 130, and SEQ ID NO: 152, respectively), RhAd66 (whose hexon protein is SEQ ID NO:174 and whose HVRs 1-7 are SEQ ID NO:21, SEQ ID NO: 43, SEQ ID NO: 65, SEQ ID NO: 87, SEQ ID NO: 109, SEQ ID NO: 131, and SEQ ID NO: 153, respectively), or RhAd67 (whose hexon protein is SEQ ID NO: 175 and whose HVRs 1-7 are SEQ ID NO:22, SEQ ID NO:44, SEQ ID NO:66, SEQ ID NO:88, SEQ ID NO: 110, SEQ ID NO: 132, and SEQ ID NO: 154, respectively), and in specific embodiments the hexon protein of:
(a) RhAd52 (whose hexon protein is SEQ ID NO: 160 and whose HVRs 1-7 are SEQ ID NO:7, SEQ ID NO:29, SEQ ID NO:51, SEQ ID NO:73, SEQ ID NO:95, SEQ ID NO: 117, and SEQ ID NO: 139, respectively);
(b) RhAd56 (whose hexon protein is SEQ ID NO:164 and whose HVRs 1-7 are SEQ ID NO: 11, SEQ ID NO:33, SEQ ID NO:55, SEQ ID NO:77, SEQ ID NO:99, SEQ ID NO: 121, and SEQ ID NO: 143, respectively);
(c) RhAd59 (whose hexon protein is SEQ ID NO: 167 and whose HVRs 1-7 are SEQ ID NO: 14, SEQ ID NO:36, SEQ ID NO:58, SEQ ID NO:80, SEQ ID NO: 102, SEQ ID NO: 124, and SEQ ID NO: 146, respectively); or
(d) RhAd63 (whose hexon protein is SEQ ID NO:171 and whose HVRs 1-7 are SEQ ID NO: 18, SEQ ID NO:40, SEQ ID NO: 62, SEQ ID NO:84, SEQ ID NO: 106, SEQ ID NO: 128, and SEQ ID NO: 150, respectively).

23. The method of embodiment 22, wherein the low negative charge adenoviral vector comprises a hexon protein having hypervariable regions (HVRs) having at least 97% sequence identity to the HVRs of the hexon protein of RhAd51 (whose hexon protein is SEQ ID NO: 159 and whose HVRs 1-7 are SEQ ID NO:6, SEQ ID NO:28, SEQ ID NO:50, SEQ ID NO:72, SEQ ID NO:94, SEQ ID NO: 116, and SEQ ID NO: 138, respectively), RhAd52 (whose hexon protein is SEQ ID NO: 160 and whose HVRs 1-7 are SEQ ID NO:7, SEQ ID NO:29, SEQ ID NO:51, SEQ ID NO:73, SEQ ID NO:95, SEQ ID NO:117, and SEQ ID NO: 139, respectively), RhAd53 (whose hexon protein is SEQ ID NO:161 and whose HVRs 1-7 are SEQ ID NO:8, SEQ ID NO:30, SEQ ID NO:52, SEQ ID NO:74, SEQ ID NO: 96, SEQ ID NO: 118, and SEQ ID NO: 140, respectively), RhAd54 (whose hexon protein is SEQ ID NO: 162 and whose HVRs 1-7 are SEQ ID NO:9, SEQ ID NO:31, SEQ ID NO:53, SEQ ID NO:75, SEQ ID NO:97, SEQ ID NO: 119, and SEQ ID NO: 141, respectively), RhAd55 (whose hexon protein is SEQ ID NO: 163 and whose HVRs 1-7 are SEQ ID NO: 10, SEQ ID NO:32, SEQ ID NO:54, SEQ ID NO: 76, SEQ ID NO:98, SEQ ID NO: 120, and SEQ ID NO: 142, respectively), RhAd56 (whose hexon protein is SEQ ID NO: 164 and whose HVRs 1-7 are SEQ ID NO:11, SEQ ID NO:33, SEQ ID NO:55, SEQ ID NO:77, SEQ ID NO: 99, SEQ ID NO: 121, and SEQ ID NO: 143, respectively), RhAd57 (whose hexon protein is SEQ ID NO: 165 and whose HVRs 1-7 are SEQ ID NO: 12, SEQ ID NO:34, SEQ ID NO: 56, SEQ ID NO:78, SEQ ID NO: 100, SEQ ID NO: 122, and SEQ ID NO: 144, respectively), RhAd58 (whose hexon protein is SEQ ID NO: 166 and whose HVRs 1-7 are SEQ ID NO: 13, SEQ ID NO:35, SEQ ID NO:57, SEQ ID NO: 79, SEQ ID NO: 101, SEQ ID NO: 123, and SEQ ID NO: 145, respectively), RhAd59 (whose hexon protein is SEQ ID NO: 167 and whose HVRs 1-7 are SEQ ID NO: 14, SEQ ID NO:36, SEQ ID NO:58, SEQ ID NO: 80, SEQ ID NO: 102, SEQ ID NO: 124, and SEQ ID NO: 146, respectively), RhAd60 (whose hexon protein is SEQ ID NO: 168 and whose HVRs 1-7 are SEQ ID NO:15, SEQ ID NO: 37, SEQ ID NO:59, SEQ ID NO:81, SEQ ID NO: 103, SEQ ID NO: 125, and SEQ ID NO: 147, respectively), RhAd61 (whose hexon protein is SEQ ID NO: 169 and whose HVRs 1-7 are SEQ ID NO: 16, SEQ ID NO:38, SEQ ID NO:60, SEQ ID NO:82, SEQ ID NO: 104, SEQ ID NO: 126, and SEQ ID NO:148, respectively), RhAd62 (whose hexon protein is SEQ ID NO: 170 and whose HVRs 1-7 are SEQ ID NO: 17, SEQ ID NO:39, SEQ ID NO:61, SEQ ID NO: 83, SEQ ID NO: 105, SEQ ID NO: 127, and SEQ ID NO: 149, respectively), RhAd63 (whose hexon protein is SEQ ID NO:171 and whose HVRs 1-7 are SEQ ID NO:18, SEQ ID NO: 40, SEQ ID NO:62, SEQ ID NO: 84, SEQ ID NO: 106, SEQ ID NO: 128, and SEQ ID NO: 150, respectively), RhAd64 (whose hexon protein is SEQ ID NO: 172 and whose HVRs 1-7 are SEQ ID NO: 19, SEQ ID NO:41, SEQ ID NO:63, SEQ ID NO:85, SEQ ID NO: 107, SEQ ID NO: 129, and SEQ ID NO:151, respectively), RhAd65 (whose hexon protein is SEQ ID NO: 173 and whose HVRs 1-7 are SEQ ID NO:20, SEQ ID NO:42, SEQ ID NO:64, SEQ ID NO: 86, SEQ ID NO: 108, SEQ ID NO: 130, and SEQ ID NO: 152, respectively), RhAd66 (whose hexon protein is SEQ ID NO: 174 and whose HVRs 1-7 are SEQ ID NO:21, SEQ ID NO: 43, SEQ ID NO: 65, SEQ ID NO: 87, SEQ ID NO: 109, SEQ ID NO: 131, and SEQ ID NO: 153, respectively), or RhAd67 (whose hexon protein is SEQ ID NO: 175 and whose HVRs 1-7 are SEQ ID NO:22, SEQ ID NO:44, SEQ ID NO: 66, SEQ ID NO:88, SEQ ID NO: 110, SEQ ID NO: 132, and SEQ ID NO: 154, respectively), and in specific embodiments the hexon protein of:
(a) RhAd52 (whose hexon protein is SEQ ID NO: 160 and whose HVRs 1-7 are SEQ ID NO:7, SEQ ID NO:29, SEQ ID NO:51, SEQ ID NO:73, SEQ ID NO:95, SEQ ID NO: 117, and SEQ ID NO: 139, respectively);
(b) RhAd56 (whose hexon protein is SEQ ID NO: 164 and whose HVRs 1-7 are SEQ ID NO: 11, SEQ ID NO:33, SEQ ID NO:55, SEQ ID NO:77, SEQ ID NO:99, SEQ ID NO: 121, and SEQ ID NO: 143, respectively);
(c) RhAd59 (whose hexon protein is SEQ ID NO: 167 and whose HVRs 1-7 are SEQ ID NO: 14, SEQ ID NO:36, SEQ ID NO:58, SEQ ID NO:80, SEQ ID NO: 102, SEQ ID NO: 124, and SEQ ID NO: 146, respectively); or
(d) RhAd63 (whose hexon protein is SEQ ID NO:171 and whose HVRs 1-7 are SEQ ID NO: 18, SEQ ID NO:40, SEQ ID NO: 62, SEQ ID NO:84, SEQ ID NO: 106, SEQ ID NO: 128, and SEQ ID NO: 150, respectively).

24. The method of any one of embodiments 1 to 23, wherein the low negative charge adenoviral vector comprises a hexon protein comprising HVRs having at least 90% sequence identity to the HVRs of the hexon protein of RhAd51 (whose hexon protein is SEQ ID NO: 159 and whose HVRs 1-7 are SEQ ID NO:6, SEQ ID NO:28, SEQ ID NO:50, SEQ ID NO: 72, SEQ ID NO:94, SEQ ID NO: 116, and SEQ ID NO: 138, respectively), RhAd52 (whose hexon protein is SEQ ID NO: 160 and whose HVRs 1-7 are SEQ ID NO:7, SEQ ID NO: 29, SEQ ID NO:51, SEQ ID NO:73, SEQ ID NO: 95, SEQ ID NO: 117, and SEQ ID NO: 139, respectively), RhAd53 (whose hexon protein is SEQ ID NO: 161 and whose HVRs 1-7 are SEQ ID NO:8, SEQ ID NO:30, SEQ ID NO:52, SEQ ID NO: 74, SEQ ID NO: 96, SEQ ID NO: 118, and SEQ ID NO: 140, respectively), RhAd54 (whose hexon protein is SEQ ID NO: 162 and whose HVRs 1-7 are SEQ ID NO:9, SEQ ID NO:31, SEQ ID NO:53, SEQ ID NO:75, SEQ ID NO:97, SEQ ID NO: 119, and SEQ ID NO:141, respectively), RhAd55 (whose hexon protein is SEQ ID NO:163 and whose HVRs 1-7 are SEQ ID NO:10, SEQ ID NO:32, SEQ ID NO:54, SEQ ID NO: 76, SEQ ID NO:98, SEQ ID NO: 120, and SEQ ID NO: 142, respectively), RhAd56 (whose hexon protein is SEQ ID NO: 164 and whose HVRs 1-7 are SEQ ID NO: 11, SEQ ID NO:33, SEQ ID NO:55, SEQ ID NO:77, SEQ ID NO: 99, SEQ ID NO: 121, and SEQ ID NO: 143, respectively), RhAd57 (whose hexon protein is SEQ ID NO: 165 and whose HVRs 1-7 are SEQ ID NO: 12, SEQ ID NO:34, SEQ ID NO: 56, SEQ ID NO:78, SEQ ID NO: 100, SEQ ID NO: 122, and SEQ ID NO: 144, respectively), RhAd58 (whose hexon protein is SEQ ID NO: 166 and whose HVRs 1-7 are SEQ ID NO: 13, SEQ ID NO:35, SEQ ID NO:57, SEQ ID NO:79, SEQ ID NO: 101, SEQ ID NO: 123, and SEQ ID NO: 145, respectively), RhAd59 (whose hexon protein is SEQ ID NO: 167 and whose HVRs 1-7 are SEQ ID NO: 14, SEQ ID NO:36, SEQ ID NO:58, SEQ ID NO: 80, SEQ ID NO: 102, SEQ ID NO: 124, and SEQ ID NO: 146, respectively), RhAd60 (whose hexon protein is SEQ ID NO: 168 and whose HVRs 1-7 are SEQ ID NO: 15, SEQ ID NO: 37, SEQ ID NO:59, SEQ ID NO:81, SEQ ID NO: 103, SEQ ID NO: 125, and SEQ ID NO: 147, respectively), RhAd61 (whose hexon protein is SEQ ID NO: 169 and whose HVRs 1-7 are SEQ ID NO: 16, SEQ ID NO:38, SEQ ID NO:60, SEQ ID NO:82, SEQ ID NO: 104, SEQ ID NO: 126, and SEQ ID NO: 148, respectively), RhAd62 (whose hexon protein is SEQ ID NO: 170 and whose HVRs 1-7 are SEQ ID NO: 17, SEQ ID NO:39, SEQ ID NO:61, SEQ ID NO: 83, SEQ ID NO: 105, SEQ ID NO: 127, and SEQ ID NO: 149, respectively), RhAd63 (whose hexon protein is SEQ ID NO:171 and whose HVRs 1-7 are SEQ ID NO:18, SEQ ID NO: 40, SEQ ID NO: 62, SEQ ID NO: 84, SEQ ID NO: 106, SEQ ID NO: 128, and SEQ ID NO: 150, respectively), RhAd64 (whose hexon protein is SEQ ID NO: 172 and whose HVRs 1-7 are SEQ ID NO: 19, SEQ ID NO:41, SEQ ID NO:63, SEQ ID NO:85, SEQ ID NO: 107, SEQ ID NO: 129, and SEQ ID NO: 151, respectively), RhAd65 (whose hexon protein is SEQ ID NO: 173 and whose HVRs 1-7 are SEQ ID NO:20, SEQ ID NO:42, SEQ ID NO:64, SEQ ID NO: 86, SEQ ID NO: 108, SEQ ID NO: 130, and SEQ ID NO: 152, respectively), RhAd66 (whose hexon protein is SEQ ID NO: 174 and whose HVRs 1-7 are SEQ ID NO:21, SEQ ID NO: 43, SEQ ID NO: 65, SEQ ID NO: 87, SEQ ID NO: 109, SEQ ID NO: 131, and SEQ ID NO: 153, respectively), or RhAd67 (whose hexon protein is SEQ ID NO:175 and whose HVRs 1-7 are SEQ ID NO:22, SEQ ID NO:44, SEQ ID NO:66, SEQ ID NO:88, SEQ ID NO: 110, SEQ ID NO: 132, and SEQ ID NO: 154, respectively), and in specific embodiments the hexon protein of:
(a) RhAd52 (whose hexon protein is SEQ ID NO: 160 and whose HVRs 1-7 are SEQ ID NO:7, SEQ ID NO:29, SEQ ID NO:51, SEQ ID NO:73, SEQ ID NO:95, SEQ ID NO: 117, and SEQ ID NO: 139, respectively);
(b) RhAd56 (whose hexon protein is SEQ ID NO: 164 and whose HVRs 1-7 are SEQ ID NO: 11, SEQ ID NO:33, SEQ ID NO:55, SEQ ID NO:77, SEQ ID NO:99, SEQ ID NO: 121, and SEQ ID NO: 143, respectively);
(c) RhAd59 (whose hexon protein is SEQ ID NO:167 and whose HVRs 1-7 are SEQ ID NO: 14, SEQ ID NO:36, SEQ ID NO:58, SEQ ID NO:80, SEQ ID NO: 102, SEQ ID NO: 124, and SEQ ID NO: 146, respectively); or
(d) RhAd63 (whose hexon protein is SEQ ID NO:171 and whose HVRs 1-7 are SEQ ID NO: 18, SEQ ID NO:40, SEQ ID NO:62, SEQ ID NO:84, SEQ ID NO: 106, SEQ ID NO: 128, and SEQ ID NO: 150, respectively).

25. The method of embodiment 24, wherein the low negative charge adenoviral vector comprises a hexon protein comprising HVRs having at least 95% sequence identity to the HVRs of the hexon protein of RhAd51 (whose hexon protein is SEQ ID NO:159 and whose HVRs 1-7 are SEQ ID NO:6, SEQ ID NO:28, SEQ ID NO:50, SEQ ID NO:72, SEQ ID NO: 94, SEQ ID NO: 116, and SEQ ID NO: 138, respectively), RhAd52 (whose hexon protein is SEQ ID NO: 160 and whose HVRs 1-7 are SEQ ID NO:7, SEQ ID NO:29, SEQ ID NO: 51, SEQ ID NO:73, SEQ ID NO:95, SEQ ID NO: 117, and SEQ ID NO: 139, respectively), RhAd53 (whose hexon protein is SEQ ID NO: 161 and whose HVRs 1-7 are SEQ ID NO:8, SEQ ID NO:30, SEQ ID NO:52, SEQ ID NO:74, SEQ ID NO:96, SEQ ID NO: 118, and SEQ ID NO: 140, respectively), RhAd54 (whose hexon protein is SEQ ID NO: 162 and whose HVRs 1-7 are SEQ ID NO:9, SEQ ID NO:31, SEQ ID NO:53, SEQ ID NO: 75, SEQ ID NO:97, SEQ ID NO: 119, and SEQ ID NO: 141, respectively), RhAd55 (whose hexon protein is SEQ ID NO: 163 and whose HVRs 1-7 are SEQ ID NO:10, SEQ ID NO: 32, SEQ ID NO: 54, SEQ ID NO: 76, SEQ ID NO: 98, SEQ ID NO: 120, and SEQ ID NO: 142, respectively), RhAd56 (whose hexon protein is SEQ ID NO: 164 and whose HVRs 1-7 are SEQ ID NO:11, SEQ ID NO:33, SEQ ID NO:55, SEQ ID NO:77, SEQ ID NO:99, SEQ ID NO: 121, and SEQ ID NO: 143, respectively), RhAd57 (whose hexon protein is SEQ ID NO: 165 and whose HVRs 1-7 are SEQ ID NO: 12, SEQ ID NO:34, SEQ ID NO:56, SEQ ID NO: 78, SEQ ID NO: 100, SEQ ID NO: 122, and SEQ ID NO: 144, respectively), RhAd58 (whose hexon protein is SEQ ID NO: 166 and whose HVRs 1-7 are SEQ ID NO: 13, SEQ ID NO: 35, SEQ ID NO:57, SEQ ID NO:79, SEQ ID NO: 101, SEQ ID NO: 123, and SEQ ID NO: 145, respectively), RhAd59 (whose hexon protein is SEQ ID NO: 167 and whose HVRs 1-7 are SEQ ID NO: 14, SEQ ID NO:36, SEQ ID NO:58, SEQ ID NO:80, SEQ ID NO: 102, SEQ ID NO: 124, and SEQ ID NO: 146, respectively), RhAd60 (whose hexon protein is SEQ ID NO: 168 and whose HVRs 1-7 are SEQ ID NO: 15, SEQ ID NO:37, SEQ ID NO:59, SEQ ID NO: 81, SEQ ID NO: 103, SEQ ID NO: 125, and SEQ ID NO: 147, respectively), RhAd61 (whose hexon protein is SEQ ID NO: 169 and whose HVRs 1-7 are SEQ ID NO: 16, SEQ ID NO: 38, SEQ ID NO:60, SEQ ID NO:82, SEQ ID NO: 104, SEQ ID NO: 126, and SEQ ID NO: 148, respectively), RhAd62 (whose hexon protein is SEQ ID NO: 170 and whose HVRs 1-7 are SEQ ID NO: 17, SEQ ID NO:39, SEQ ID NO:61, SEQ ID NO:83, SEQ ID NO: 105, SEQ ID NO: 127, and SEQ ID NO: 149, respectively), RhAd63 (whose hexon protein is SEQ ID NO: 171 and whose HVRs 1-7 are SEQ ID NO: 18, SEQ ID NO:40, SEQ ID NO:62, SEQ ID NO: 84, SEQ ID NO: 106, SEQ ID NO: 128, and SEQ ID NO: 150, respectively), RhAd64 (whose hexon protein is SEQ ID NO: 172 and whose HVRs 1-7 are SEQ ID NO:19, SEQ ID NO: 41, SEQ ID NO: 63, SEQ ID NO: 85, SEQ ID NO: 107, SEQ ID NO: 129, and SEQ ID NO: 151, respectively), RhAd65 (whose hexon protein is SEQ ID NO:173 and whose HVRs 1-7 are SEQ ID NO:20, SEQ ID NO:42, SEQ ID NO:64, SEQ ID NO:86, SEQ ID NO: 108, SEQ ID NO: 130, and SEQ ID NO: 152, respectively), RhAd66 (whose hexon protein is SEQ ID NO: 174 and whose HVRs 1-7 are SEQ ID NO:21, SEQ ID NO:43, SEQ ID NO:65, SEQ ID NO: 87, SEQ ID NO: 109, SEQ ID NO: 131, and SEQ ID NO: 153, respectively), or RhAd67 (whose hexon protein is SEQ ID NO:175 and whose HVRs 1-7 are SEQ ID NO:22, SEQ ID NO: 44, SEQ ID NO: 66, SEQ ID NO: 88, SEQ ID NO: 110, SEQ ID NO: 132, and SEQ ID NO: 154, respectively), and in specific embodiments the hexon protein of:
(a) RhAd52 (whose hexon protein is SEQ ID NO: 160 and whose HVRs 1-7 are SEQ ID NO:7, SEQ ID NO:29, SEQ ID NO:51, SEQ ID NO:73, SEQ ID NO:95, SEQ ID NO: 117, and SEQ ID NO: 139, respectively);
(b) RhAd56 (whose hexon protein is SEQ ID NO:164 and whose HVRs 1-7 are SEQ ID NO: 11, SEQ ID NO:33, SEQ ID NO:55, SEQ ID NO:77, SEQ ID NO:99, SEQ ID NO: 121, and SEQ ID NO: 143, respectively);
(c) RhAd59 (whose hexon protein is SEQ ID NO: 167 and whose HVRs 1-7 are SEQ ID NO: 14, SEQ ID NO:36, SEQ ID NO:58, SEQ ID NO:80, SEQ ID NO: 102, SEQ ID NO: 124, and SEQ ID NO: 146, respectively); or
(d) RhAd63 (whose hexon protein is SEQ ID NO: 171 and whose HVRs 1-7 are SEQ ID NO: 18, SEQ ID NO:40, SEQ ID NO:62, SEQ ID NO:84, SEQ ID NO: 106, SEQ ID NO: 128, and SEQ ID NO: 150, respectively).

26. The method of embodiment 25, wherein the low negative charge adenoviral vector comprises a hexon protein comprising HVRs having at least 97% sequence identity to the HVRs of the hexon protein of RhAd51 (whose hexon protein is SEQ ID NO:159 and whose HVRs 1-7 are SEQ ID NO:6, SEQ ID NO:28, SEQ ID NO:50, SEQ ID NO:72, SEQ ID NO: 94, SEQ ID NO: 116, and SEQ ID NO: 138, respectively), RhAd52 (whose hexon protein is SEQ ID NO: 160 and whose HVRs 1-7 are SEQ ID NO:7, SEQ ID NO:29, SEQ ID NO: 51, SEQ ID NO: 73, SEQ ID NO:95, SEQ ID NO: 117, and SEQ ID NO: 139, respectively), RhAd53 (whose hexon protein is SEQ ID NO: 161 and whose HVRs 1-7 are SEQ ID NO:8, SEQ ID NO:30, SEQ ID NO:52, SEQ ID NO: 74, SEQ ID NO:96, SEQ ID NO: 118, and SEQ ID NO: 140, respectively), RhAd54 (whose hexon protein is SEQ ID NO: 162 and whose HVRs 1-7 are SEQ ID NO:9, SEQ ID NO:31, SEQ ID NO:53, SEQ ID NO: 75, SEQ ID NO: 97, SEQ ID NO: 119, and SEQ ID NO: 141, respectively), RhAd55 (whose hexon protein is SEQ ID NO:163 and whose HVRs 1-7 are SEQ ID NO:10, SEQ ID NO: 32, SEQ ID NO:54, SEQ ID NO:76, SEQ ID NO:98, SEQ ID NO: 120, and SEQ ID NO: 142, respectively), RhAd56 (whose hexon protein is SEQ ID NO: 164 and whose HVRs 1-7 are SEQ ID NO:11, SEQ ID NO:33, SEQ ID NO:55, SEQ ID NO:77, SEQ ID NO:99, SEQ ID NO: 121, and SEQ ID NO:143, respectively), RhAd57 (whose hexon protein is SEQ ID NO: 165 and whose HVRs 1-7 are SEQ ID NO: 12, SEQ ID NO:34, SEQ ID NO:56, SEQ ID NO: 78, SEQ ID NO: 100, SEQ ID NO: 122, and SEQ ID NO: 144, respectively), RhAd58 (whose hexon protein is SEQ ID NO:166 and whose HVRs 1-7 are SEQ ID NO:13, SEQ ID NO: 35, SEQ ID NO:57, SEQ ID NO: 79, SEQ ID NO: 101, SEQ ID NO: 123, and SEQ ID NO: 145, respectively), RhAd59 (whose hexon protein is SEQ ID NO: 167 and whose HVRs 1-7 are SEQ ID NO: 14, SEQ ID NO:36, SEQ ID NO:58, SEQ ID NO:80, SEQ ID NO: 102, SEQ ID NO: 124, and SEQ ID NO: 146, respectively), RhAd60 (whose hexon protein is SEQ ID NO: 168 and whose HVRs 1-7 are SEQ ID NO: 15, SEQ ID NO:37, SEQ ID NO:59, SEQ ID NO: 81, SEQ ID NO: 103, SEQ ID NO: 125, and SEQ ID NO: 147, respectively), RhAd61 (whose hexon protein is SEQ ID NO: 169 and whose HVRs 1-7 are SEQ ID NO: 16, SEQ ID NO: 38, SEQ ID NO: 60, SEQ ID NO:82, SEQ ID NO: 104, SEQ ID NO: 126, and SEQ ID NO: 148, respectively), RhAd62 (whose hexon protein is SEQ ID NO: 170 and whose HVRs 1-7 are SEQ ID NO:17, SEQ ID NO:39, SEQ ID NO:61, SEQ ID NO:83, SEQ ID NO: 105, SEQ ID NO: 127, and SEQ ID NO: 149, respectively), RhAd63 (whose hexon protein is SEQ ID NO: 171 and whose HVRs 1-7 are SEQ ID NO:18, SEQ ID NO:40, SEQ ID NO:62, SEQ ID NO: 84, SEQ ID NO: 106, SEQ ID NO: 128, and SEQ ID NO: 150, respectively), RhAd64 (whose hexon protein is SEQ ID NO: 172 and whose HVRs 1-7 are SEQ ID NO:19, SEQ ID NO: 41, SEQ ID NO:63, SEQ ID NO: 85, SEQ ID NO: 107, SEQ ID NO: 129, and SEQ ID NO: 151, respectively), RhAd65 (whose hexon protein is SEQ ID NO: 173 and whose HVRs 1-7 are SEQ ID NO:20, SEQ ID NO:42, SEQ ID NO:64, SEQ ID NO:86, SEQ ID NO: 108, SEQ ID NO: 130, and SEQ ID NO:152, respectively), RhAd66 (whose hexon protein is SEQ ID NO: 174 and whose HVRs 1-7 are SEQ ID NO:21, SEQ ID NO:43, SEQ ID NO:65, SEQ ID NO: 87, SEQ ID NO: 109, SEQ ID NO: 131, and SEQ ID NO: 153, respectively), or RhAd67 (whose hexon protein is SEQ ID NO: 175 and whose HVRs 1-7 are SEQ ID NO:22, SEQ ID NO: 44, SEQ ID NO: 66, SEQ ID NO: 88, SEQ ID NO: 110, SEQ ID NO: 132, and SEQ ID NO: 154, respectively), and in specific embodiments the hexon protein of:
(a) RhAd52 (whose hexon protein is SEQ ID NO: 160 and whose HVRs 1-7 are SEQ ID NO:7, SEQ ID NO:29, SEQ ID NO:51, SEQ ID NO:73, SEQ ID NO:95, SEQ ID NO: 117, and SEQ ID NO: 139, respectively);
(b) RhAd56 (whose hexon protein is SEQ ID NO:164 and whose HVRs 1-7 are SEQ ID NO: 11, SEQ ID NO:33, SEQ ID NO:55, SEQ ID NO:77, SEQ ID NO:99, SEQ ID NO: 121, and SEQ ID NO: 143, respectively);
(c) RhAd59 (whose hexon protein is SEQ ID NO: 167 and whose HVRs 1-7 are SEQ ID NO: 14, SEQ ID NO:36, SEQ ID NO:58, SEQ ID NO:80, SEQ ID NO: 102, SEQ ID NO: 124, and SEQ ID NO: 146, respectively); or
(d) RhAd63 (whose hexon protein is SEQ ID NO:171 and whose HVRs 1-7 are SEQ ID NO: 18, SEQ ID NO:40, SEQ ID NO:62, SEQ ID NO:84, SEQ ID NO: 106, SEQ ID NO: 128, and SEQ ID NO:150, respectively).

27. The method of any one of embodiments 1 to 26, wherein the low negative charge adenoviral vector comprises a hexon protein comprising HVRs having at least 90% sequence identity to the HVRs of hexon protein of RhAd52 (whose hexon protein is SEQ ID NO: 160 and whose HVRs 1-7 are SEQ ID NO:7, SEQ ID NO:29, SEQ ID NO:51, SEQ ID NO: 73, SEQ ID NO:95, SEQ ID NO: 117, and SEQ ID NO: 139, respectively).

28. The method of embodiment 27, wherein the low negative charge adenoviral vector comprises a hexon protein comprising HVRs having at least 95% sequence identity to the HVRs of hexon protein of RhAd52 (whose hexon protein is SEQ ID NO:160 and whose HVRs 1-7 are SEQ ID NO:7, SEQ ID NO:29, SEQ ID NO:51, SEQ ID NO:73, SEQ ID NO: 95, SEQ ID NO: 117, and SEQ ID NO: 139, respectively).

29. The method of embodiment 28, wherein the low negative charge adenoviral vector comprises a hexon protein comprising HVRs having at least 97% sequence identity to the HVRs of hexon protein of RhAd52 (whose hexon protein is SEQ ID NO: 160 and whose HVRs 1-7 are SEQ ID NO:7, SEQ ID NO:29, SEQ ID NO:51, SEQ ID NO:73, SEQ ID NO: 95, SEQ ID NO: 117, and SEQ ID NO: 139, respectively).

30. The method of embodiment 29, wherein the low negative charge adenoviral vector comprises a hexon protein comprising the HVRs of hexon protein of RhAd52 (whose hexon protein is SEQ ID NO: 160 and whose HVRs 1-7 are SEQ ID NO:7, SEQ ID NO:29, SEQ ID NO:51, SEQ ID NO:73, SEQ ID NO:95, SEQ ID NO: 117, and SEQ ID NO: 139, respectively).

31. The method of any one of embodiments 1 to 26, wherein the low negative charge adenoviral vector comprises a hexon protein comprising HVRs having at least 90% sequence identity to the HVRs of hexon protein of RhAd56 (whose hexon protein is SEQ ID NO: 164 and whose HVRs 1-7 are SEQ ID NO:11, SEQ ID NO:33, SEQ ID NO:55, SEQ ID NO: 77, SEQ ID NO:99, SEQ ID NO: 121, and SEQ ID NO: 143, respectively).

32. The method of embodiment 31, wherein the low negative charge adenoviral vector comprises a hexon protein comprising HVRs having at least 95% sequence identity to the HVRs of hexon protein of RhAd56 (whose hexon protein is SEQ ID NO: 164 and whose HVRs 1-7 are SEQ ID NO: 11, SEQ ID NO:33, SEQ ID NO:55, SEQ ID NO:77, SEQ ID NO: 99, SEQ ID NO: 121, and SEQ ID NO: 143, respectively).

33. The method of embodiment 32, wherein the low negative charge adenoviral vector comprises a hexon protein comprising HVRs having at least 97% sequence identity to the HVRs of hexon protein of RhAd56 (whose hexon protein is SEQ ID NO: 164 and whose HVRs 1-7 are SEQ ID NO:11, SEQ ID NO:33, SEQ ID NO:55, SEQ ID NO:77, SEQ ID NO: 99, SEQ ID NO: 121, and SEQ ID NO: 143, respectively).

34. The method of embodiment 33, wherein the low negative charge adenoviral vector comprises a hexon protein comprising the HVRs of hexon protein of RhAd56 (whose hexon protein is SEQ ID NO: 164 and whose HVRs 1-7 are SEQ ID NO: 11, SEQ ID NO:33, SEQ ID NO: 55, SEQ ID NO:77, SEQ ID NO:99, SEQ ID NO: 121, and SEQ ID NO: 143, respectively).

35. The method of any one of embodiments 1 to 25, wherein the low negative charge adenoviral vector comprises a hexon protein comprising HVRs having at least 90% sequence identity to the HVRs of hexon protein of RhAd59 (whose hexon protein is SEQ ID NO: 167 and whose HVRs 1-7 are SEQ ID NO: 14, SEQ ID NO:36, SEQ ID NO:58, SEQ ID NO: 80, SEQ ID NO: 102, SEQ ID NO: 124, and SEQ ID NO: 146, respectively) or RhAd63 (whose hexon protein is SEQ ID NO:171 and whose HVRs 1-7 are SEQ ID NO: 18, SEQ ID NO: 40, SEQ ID NO: 62, SEQ ID NO:84, SEQ ID NO: 106, SEQ ID NO: 128, and SEQ ID NO: 150, respectively).

36. The method of embodiment 35, wherein the low negative charge adenoviral vector comprises a hexon protein comprising HVRs having at least 95% sequence identity to the HVRs of hexon protein of RhAd59 (whose hexon protein is SEQ ID NO:167 and whose HVRs 1-7 are SEQ ID NO: 14, SEQ ID NO:36, SEQ ID NO:58, SEQ ID NO:80, SEQ ID NO: 102, SEQ ID NO: 124, and SEQ ID NO: 146, respectively) or RhAd63 (whose hexon protein is SEQ ID NO: 171 and whose HVRs 1-7 are SEQ ID NO: 18, SEQ ID NO:40, SEQ ID NO: 62, SEQ ID NO:84, SEQ ID NO: 106, SEQ ID NO: 128, and SEQ ID NO: 150, respectively).

37. The method of embodiment 36, wherein the low negative charge adenoviral vector comprises a hexon protein comprising HVRs having at least 97% sequence identity to the HVRs of hexon protein of RhAd59 (whose hexon protein is SEQ ID NO: 167 and whose HVRs 1-7 are SEQ ID NO: 14, SEQ ID NO: 36, SEQ ID NO:58, SEQ ID NO:80, SEQ ID NO: 102, SEQ ID NO: 124, and SEQ ID NO: 146, respectively) or RhAd63 (whose hexon protein is SEQ ID NO: 171 and whose HVRs 1-7 are SEQ ID NO: 18, SEQ ID NO:40, SEQ ID NO: 62, SEQ ID NO:84, SEQ ID NO: 106, SEQ ID NO: 128, and SEQ ID NO: 150, respectively).

38. The method of embodiment 37, wherein the low negative charge adenoviral vector comprises a hexon protein comprising the HVRs of hexon protein of RhAd59 (whose hexon protein is SEQ ID NO: 167 and whose HVRs 1-7 are SEQ ID NO: 14, SEQ ID NO:36, SEQ ID NO:58, SEQ ID NO: 80, SEQ ID NO: 102, SEQ ID NO: 124, and SEQ ID NO: 146, respectively) or RhAd63 (whose hexon protein is SEQ ID NO:171 and whose HVRs 1-7 are SEQ ID NO: 18, SEQ ID NO:40, SEQ ID NO: 62, SEQ ID NO:84, SEQ ID NO: 106, SEQ ID NO: 128, and SEQ ID NO: 150, respectively).

39. The method of any one of embodiments 1 to 38, wherein the low negative charge adenoviral vector comprises a hexon protein having at least 90% sequence identity to the hexon protein of RhAd51 (SEQ ID NO: 159), RhAd52 (SEQ ID NO: 160), RhAd53 (SEQ ID NO: 161), RhAd54 (SEQ ID NO: 162), RhAd55 (SEQ ID NO: 163), RhAd56 (SEQ ID NO: 164), RhAd57 (SEQ ID NO: 165), RhAd58 (SEQ ID NO: 166), RhAd59 (SEQ ID NO: 167), RhAd60 (SEQ ID NO: 168), RhAd61 (SEQ ID NO: 169), RhAd62 (SEQ ID NO: 170), RhAd63 (SEQ ID NO: 171), RhAd64 (SEQ ID NO: 172), RhAd65 (SEQ ID NO: 173), RhAd66 (SEQ ID NO:174), or RhAd67 (SEQ ID NO: 175), and in specific embodiments the hexon protein of:
(a) RhAd52 (SEQ ID NO:160);
(b) RhAd56 (SEQ ID NO: 164);
(c) RhAd59 (SEQ ID NO:167); or
(d) RhAd63 (SEQ ID NO:171).

40. The method of embodiment 39, wherein the low negative charge adenoviral vector comprises a hexon protein having at least 95% sequence identity to the hexon protein of RhAd51 (SEQ ID NO:159), RhAd52 (SEQ ID NO: 160), RhAd53 (SEQ ID NO: 161), RhAd54 (SEQ ID NO: 162), RhAd55 (SEQ ID NO: 163), RhAd56 (SEQ ID NO: 164), RhAd57 (SEQ ID NO: 165), RhAd58 (SEQ ID NO: 166), RhAd59 (SEQ ID NO: 167), RhAd60 (SEQ ID NO: 168), RhAd61 (SEQ ID NO: 169), RhAd62 (SEQ ID NO: 170), RhAd63 (SEQ ID NO: 171), RhAd64 (SEQ ID NO: 172), RhAd65 (SEQ ID NO: 173), RhAd66 (SEQ ID NO: 174), or RhAd67 (SEQ ID NO: 175), and in specific embodiments the hexon protein of:
(a) RhAd52 (SEQ ID NO:160);
(b) RhAd56 (SEQ ID NO:164);
(c) RhAd59 (SEQ ID NO:167); or
(d) RhAd63 (SEQ ID NO:171).

41. The method of embodiment 40, wherein low negative charge adenoviral vector comprises a hexon protein having at least 97% sequence identity to the hexon protein of RhAd51 (SEQ ID NO:159), RhAd52 (SEQ ID NO: 160), RhAd53 (SEQ ID NO: 161), RhAd54 (SEQ ID NO: 162), RhAd55 (SEQ ID NO: 163), RhAd56 (SEQ ID NO: 164), RhAd57 (SEQ ID NO: 165), RhAd58 (SEQ ID NO: 166), RhAd59 (SEQ ID NO: 167), RhAd60 (SEQ ID NO: 168), RhAd61 (SEQ ID NO: 169), RhAd62 (SEQ ID NO: 170), RhAd63 (SEQ ID NO: 171), RhAd64 (SEQ ID NO: 172), RhAd65 (SEQ ID NO: 173), RhAd66 (SEQ ID NO: 174), or RhAd67 (SEQ ID NO: 175), and in specific embodiments the hexon protein of:
(a) RhAd52 (SEQ ID NO: 160);
(b) RhAd56 (SEQ ID NO:164);
(c) RhAd59 (SEQ ID NO:167); or
(d) RhAd63 (SEQ ID NO:171).

42. The method of any one of embodiments 1 to 41, wherein the low negative charge adenoviral vector comprises a hexon protein having at least 90% sequence identity to the hexon protein of RhAd52 (SEQ ID NO: 160).

43. The method of embodiment 42, wherein the low negative charge adenoviral vector comprises a hexon protein having at least 95% sequence identity to the hexon protein of RhAd52 (SEQ ID NO:160).

44. The method of embodiment 43, wherein the low negative charge adenoviral vector comprises a hexon protein having at least 97% sequence identity to the hexon protein of RhAd52 (SEQ ID NO:160).

45. The method of embodiment 44, wherein the low negative charge adenoviral vector comprises a hexon protein having at least 98% sequence identity to the hexon protein of RhAd52 (SEQ ID NO:160).

46. The method of embodiment 45, wherein the low negative charge adenoviral vector comprises the hexon protein of RhAd52 (SEQ ID NO: 160).

47. The method of any one of embodiments 1 to 41, wherein the low negative charge adenoviral vector comprises a hexon protein having at least 90% sequence identity to the hexon protein of RhAd56 (SEQ ID NO:164).

48. The method of embodiment 47, wherein the low negative charge adenoviral vector comprises a hexon protein having at least 95% sequence identity to the hexon protein of RhAd56 (SEQ ID NO:164).

49. The method of embodiment 48, wherein the low negative charge adenoviral vector comprises a hexon protein having at least 97% sequence identity to the hexon protein of RhAd56 (SEQ ID NO:164).

50. The method of embodiment 49, wherein the low negative charge adenoviral vector comprises a hexon protein having at least 98% sequence identity to the hexon protein of RhAd56 (SEQ ID NO:164).

51. The method of embodiment 50, wherein the low negative charge adenoviral vector comprises the hexon protein of RhAd56 (SEQ ID NO:164).

52. The method of any one of embodiments 1 to 41, wherein the low negative charge adenoviral vector comprises a hexon protein having at least 90% sequence identity to the hexon protein of RhAd59 (SEQ ID NO: 167) or RhAd63 (SEQ ID NO:171).

53. The method of embodiment 52, wherein the low negative charge adenoviral vector comprises a hexon protein having at least 95% sequence identity to the hexon protein of RhAd59 (SEQ ID NO:167) or RhAd63 (SEQ ID NO:171).

54. The method of embodiment 53, wherein the low negative charge adenoviral vector comprises a hexon protein having at least 97% sequence identity to the hexon protein of RhAd59 (SEQ ID NO: 167) or RhAd63 (SEQ ID NO:171).

55. The method of embodiment 54, wherein the low negative charge adenoviral vector comprises a hexon protein having at least 98% sequence identity to the hexon protein of RhAd59 (SEQ ID NO:167) or RhAd63 (SEQ ID NO:171).

56. The method of embodiment 55, wherein the low negative charge adenoviral vector comprises the hexon protein of RhAd59 (SEQ ID NO: 167) or RhAd63 (SEQ ID NO: 171).

57. The method of any one of embodiments 1 to 56, wherein the low negative charge adenoviral vector is a RhAd51, RhAd52, RhAd53, RhAd54, RhAd55, RhAd56, RhAd57, RhAd58, RhAd59, RhAd60, RhAd61, RhAd62, RhAd63, RhAd64, RhAd65, RhAd66, or RhAd67 vector, and in specific embodiments the vector is:
(a) a RhAd52 vector;
(b) a RhAd56 vector;
(c) a RhAd59 vector; or
(d) a RhAd63 vector.

58. The method of embodiment 57, wherein the low negative charge adenoviral vector is a RhAd52 vector.

59. The method of embodiment 57, wherein the low negative charge adenoviral vector is a RhAd56 vector.

60. The method of embodiment 57, wherein the low negative charge adenoviral vector is a RhAd59 or RhAd63 vector.

61. The method of any one of embodiments 1 to 60, wherein the low negative charge adenoviral vector is a low seroprevalence vector.

62. The method of any one of embodiments 1 to 61, which further comprises classifying a subject's risk of TTS.

63. The method of any one of embodiments 1 to 62, which further comprises identifying the subject at risk of TTS.

64. The method of any one of embodiments 1 to 63, which further comprises selecting the subject at risk of TTS for rhesus adenoviral vector therapy.

65. The method of any one of embodiments 1 to 64, wherein the subject is a human subject.

66. The method of any one of embodiments 1 to 65, wherein the subject is positive for anti-PF4 antibodies.

67. The method of embodiment 66, which further comprises testing the subject for anti-PF4 antibodies prior to said administering step.

68. The method of any one of embodiments 1 to 67, wherein the subject is a female.

69. The method of any one of embodiments 1 to 68, wherein the subject is between the ages of 18 and 70.

70. The method of embodiment 69, wherein the subject is 50-64 years old.

71. The method of embodiment 69, wherein the subject is less than 50 years old.

72. The method of embodiment 71, wherein the subject is 40-49 years old.

73. The method of embodiment 71, wherein the subject is 30-39 years old.

74. The method of embodiment 71, wherein the subject is 20-29 years old.

75. The method of embodiment 71, wherein the subject is 18-29 years old.

76. The method of any one of embodiments 1 to 75, wherein the subject is white.

77. The method of embodiment 76, wherein the subject is non-Hispanic.

78. The method of any one of embodiments 1 to 77, wherein the subject is obese.

79. The method of any one of embodiments 1 to 78, wherein the subject has hypertension.

80. The method of any one of embodiments 1 to 79, wherein the subject is diabetic.

81. The method of any one of embodiments 1 to 80, wherein the subject is on estrogen therapy.

82. The method of embodiment 81, wherein the estrogen therapy is an estrogen-based contraceptive.

83. The method of embodiment 81, wherein the estrogen therapy is an estrogen-based hormone replacement therapy.

84. The method of any one of embodiments 1 to 83, wherein the subject has a venous thrombosis risk factor.

85. The method of embodiment 84, wherein the venous thrombosis risk factor is cirrhosis.

86. The method of embodiment 84, wherein the venous thrombosis risk factor is malignancy.

87. The method of embodiment 84, wherein the venous thrombosis risk factor is fertility treatment.

88. The method of embodiment 84, wherein the venous thrombosis risk factor is a venous catheter.

89. The method of any one of embodiments 1 to 88, wherein the subject has iron deficiency anemia.

90. The method of any one of embodiments 1 to 89, wherein the subject has hypothyroidism.

91. The method of any one of embodiments 1 to 90, wherein the low negative charge adenoviral vector is replication competent.

92. The method of any one of embodiments 1 to 90, wherein the low negative charge adenoviral vector is replication defective.

93. The method of any one of embodiments 1 to 92, wherein the low negative charge adenoviral vector therapy is administered intramuscularly.

94. The method of embodiment 93, wherein the low negative charge adenoviral vector therapy is administered to a deltoid.

95. The method of any one of embodiments 1 to 92, wherein the low negative charge adenoviral vector therapy is administered mucosally.

96. The method of embodiment 95, wherein the mucosal administration comprises buccal administration.

97. The method of embodiment 96, wherein the buccal administration is by way of buccal swab.

98. The method of embodiment 96, wherein the buccal administration is by way of buccal spray.

99. The method of embodiment 95, wherein the mucosal administration comprises intranasal administration.

100. The method of embodiment 99, wherein the nasal administration is by way of nasal spray.

101. The method of any one of embodiments 1 to 92, wherein the low negative charge adenoviral vector therapy is administered orally.

102. The method of any one of embodiments 1 to 92, wherein the low negative charge adenoviral vector therapy is administered subcutaneously.

103. The method of any one of embodiments 1 to 92 and 102, wherein the low negative charge adenoviral vector therapy is administered via microneedle administration.

104. The method of embodiment 103, wherein the microneedle administration is by way of one or more microneedle patches.

105. The method of any one of embodiments 1 to 101, which further comprises administering an additional dose of the low negative charge adenoviral vector therapy to the subject.

106. The method of embodiment 103, wherein the additional dose of the low negative charge adenoviral vector therapy is administered via the same route as the preceding dose.

107. The method of embodiment 103, wherein the additional dose of the low negative charge adenoviral vector therapy is administered via a different route as the preceding dose.

108. The method of embodiment 107, wherein the initial administration is intramuscular and the additional dose is administered mucosally.

109. The method of any one of embodiments 103 to 108, wherein the low negative charge adenoviral vector therapy is a vaccine and the additional dose is a booster.

110. The method of any one of embodiments 1 to 109, which further comprises administering one or more additional agents to the subject.

111. The method of embodiment 110, wherein the one or more additional agents comprise a blood thinner.

112. The method of embodiment 111, wherein the one or more blood thinners comprise an anticoagulant.

113. The method of embodiment 112, wherein the anticoagulant is apixaban, dabigatran, dalteparin, edoxaban, enoxaparin, fondaparinux, heparin, rivaroxaban, or warfarin.

114. The method of any one of embodiments 111 to 113, wherein the one or more blood thinners comprise an antiplatelet medication.

115. The method of embodiment 114, wherein the antiplatelet medication is aspirin, cilostazol, clopidogrel, dipyridamole, eptifibatide, prasugrel, ticagrelor, tirofiban, or vorapaxar.

116. The method of any one of embodiments 1 to 115, wherein the low negative charge adenoviral vector therapy is an adenovirus-based vaccine.

117. The method of embodiment 116, wherein the adenovirus-based vaccine is a vaccine that elicits an immune response against a pathogenic virus.

118. The method of embodiment 117, wherein the pathogenic virus is a respiratory virus.

119. The method of embodiment 118, wherein the pathogenic virus is a coronavirus.

120. The method of embodiment 119, wherein the coronavirus is SARS-COV-2.

121. The method of embodiment 119 or embodiment 120, wherein the adenovirus-based vaccine comprises a transgene that encodes a coronavirus spike protein or a fragment thereof.

122. The method of embodiment 118, wherein the pathogenic virus is an influenza virus.

123. The method of embodiment 118, wherein the pathogenic virus is respiratory syncytial virus.

124. The method of any one of embodiments 1 to 111, wherein the low negative charge adenoviral vector therapy is vaccine therapy.

125. The method of any one of embodiments 1 to 116, wherein the low negative charge adenoviral vector therapy comprises a transgene that encodes a protein that promotes an immune response in the subject against an infective agent.

126. The method of embodiment 125, wherein the infective agent is a bacterium, a virus, a parasite, or a fungus.

127. The method of embodiment 125 or embodiment 126, wherein the transgene encodes a bacterial protein or fragment thereof, a viral protein or fragment thereof, a parasitic protein or fragment thereof, or a fungal protein or fragment thereof.

128. The method of embodiment 127, wherein the bacterial protein or fragment thereof is from Mycobacterium tuberculosis, Mycobacterium bovis, Mycobacterium africanum, Mycobacterium microti, Mycobacterium leprae, Pseudomonas aeruginosa, Salmonella typhimurium, Escherichia coli, Klebsiella pneumoniae, Streptococcus pneumoniae, Staphylococcus aureus, Francisella tularensis, Brucella, Burkholderia mallei, Yersinia pestis, Corynebacterium diphtheria, Neisseria meningitidis, Bordetella pertussis, Clostridium tetani, or Bacillus anthracis.

129. The method of embodiment 127, wherein the viral protein or fragment thereof, is from a viral family selected from the group consisting of Retroviridae, Flaviviridae, Arenaviridae, Bunyaviridae, Filoviridae, Togaviridae, Poxviridae, Herpesviridae, Orthomyxoviridae, Coronaviridae, Rhabdoviridae, Paramyxoviridae, Picornaviridae, Hepadnaviridae, Papillomaviridae, Parvoviridae, Astroviridae, Polyomaviridae, Calciviridae, and Reoviridae.

130. The method of embodiment 127, wherein the viral protein or fragment thereof, is from human immunodeficiency virus (HIV), human papillomavirus (HPV), hepatitis A virus (Hep A), hepatitis B virus (HBV), hepatitis C virus (HCV), Variola major, Variola minor, monkeypox virus, measles virus, rubella virus, mumps virus, varicella zoster virus (VZV), poliovirus, rabies virus, Japanese encephalitis virus, herpes simplex virus (HSV), cytomegalovirus (CMV), rotavirus, influenza, Ebola virus, yellow fever virus, Zika virus, or Marburg virus.

131. The method of embodiment 127, wherein the viral protein or fragment thereof, is from Epstein-Barr virus (EBV).

132. The method of embodiment 127, wherein the parasitic protein or fragment thereof, is from Toxoplasma gondii, Plasmodium falciparum, Plasmodium vivax, Plasmodium ovale, Plasmodium malariae, Trypanosoma spp., or Legionella spp.

133. The method of embodiment 127, wherein the fungal protein or fragment thereof, is from Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus.

134. The method of any one of embodiments 1 to 115, wherein the low negative charge adenoviral vector therapy is a cancer therapeutic.

135. The method of embodiment 134, wherein the low negative charge adenoviral vector is an oncolytic virus.

136. The method of embodiment 134 or embodiment 135, wherein the low negative charge adenoviral vector comprises a transgene that encodes a tumor-associated antigen (TAA) or a fragment thereof.

137. The method of embodiment 136, wherein the TAA is prostate-specific antigen, MAGE-A3, human papilloma virus (HPV) E6/E7, carcinoembryonic antigen (CEA).

138. The method of embodiment 134 or embodiment 135, wherein the low negative charge adenoviral vector comprises a transgene that encodes an anti-TAA antibody.

139. The method of embodiment 138, wherein the anti-TAA antibody is an anti-EGFR or anti-HER2 antibody.

140. The method of embodiment 134 or embodiment 135, wherein the low negative charge adenoviral vector comprises a transgene that encodes a cytokine capable of promoting an anti-tumor immune response.

141. The method of embodiment 140, wherein the cytokine is GM-CSF, IFN-alpha, CD40 ligand (CD40L), interleukin-12 (IL12), or interleukin-18 (IL18).

142. The method of embodiment 134 or embodiment 135, wherein the low negative charge adenoviral vector comprises a transgene that encodes a cancer immunotherapy agent.

143. The method of embodiment 142, wherein the cancer immunotherapy agent is an anti-CTLA antibody, an anti-PD-L1 antibody, or an anti-TAA/anti-CD3 bispecific antibody, or CD40 ligand.

144. The method of embodiment 134 or embodiment 135, wherein the low negative charge adenoviral vector comprises a transgene that encodes a suicide protein.

145. The method of embodiment 144, wherein the suicide protein is p53.

146. The method of any one of embodiments 134 to 145, wherein the low negative charge adenoviral vector is modified to alter its tropism to have preferential selectivity for cancer cells.

147. The method of any one of embodiments 1 to 115, wherein the low negative charge adenoviral vector therapy is a gene therapy.

148. The method of embodiment 147, wherein the low negative charge adenoviral vector therapy comprises a transgene that encodes a protein that is missing or mutant in the subject.

149. The method of embodiment 147 or embodiment 148, wherein the low negative charge adenoviral vector therapy comprises a transgene that encodes a protein that inhibits an aberrant or overexpressed gene product in the subject.

150. The method of any one of embodiments 147 to 149, wherein the subject has AADC deficiency and the transgene encodes AADC.

151. The method of any one of embodiments 147 to 149, wherein the subject has Batten Disease and the transgene encodes CLN2.

152. The method of any one of embodiments 147 to 149, wherein the subject has Batten Disease and the transgene encodes CLN6.

153. The method of any one of embodiments 147 to 149, wherein the subject has MPS-IIIB and the transgene encodes NAGLU.

154. The method of any one of embodiments 147 to 149, wherein the subject has Parkinson's Disease and the transgene encodes AADC, GDNF, or Neurturin.

155. The method of any one of embodiments 147 to 149, wherein the subject SMA and the transgene encodes SMN.

156. The method of any one of embodiments 147 to 149, wherein the subject has GAN and the transgene encodes GAN.

157. The method of any one of embodiments 147 to 149, wherein the subject has achromatopsia and the transgene encodes CNGB3.

158. The method of any one of embodiments 147 to 149, wherein the subject has choroideremia and the transgene encodes REP1.

159. The method of any one of embodiments 147 to 149, wherein the subject has LCA and the transgene encodes RPE65.

160. The method of any one of embodiments 147 to 149, wherein the subject has LHON and the transgene encodes ND4.

161. The method of any one of embodiments 147 to 149, wherein the subject RP (RLBP1) and the transgene encodes RLBP1.

162. The method of any one of embodiments 147 to 149, wherein the subject has wet AMD and the transgene encodes an anti-VEGF protein, optionally wherein the anti-VEGF protein is an antibody.

163. The method of any one of embodiments 147 to 149, wherein the subject has X-linked RP and the transgene encodes RPGR.

164. The method of any one of embodiments 147 to 149, wherein the subject has X-linked retinoschisis and the transgene encodes RS1.

165. The method of any one of embodiments 147 to 149, wherein the subject has Crigler-Najjar syndrome and the transgene encodes UGT1A1.

166. The method of any one of embodiments 147 to 149, wherein the subject homozygous FH and the transgene encodes LDLR.

167. The method of any one of embodiments 147 to 149, wherein the subject has GSD1a and the transgene encodes G6PC.

168. The method of any one of embodiments 147 to 149, wherein the subject has hemophilia A and the transgene encodes FVIII.

169. The method of any one of embodiments 147 to 149, wherein the subject has hemophilia A and the transgene encodes FVIX.

170. The method of any one of embodiments 147 to 149, wherein the subject has MPS-I and the transgene encodes ZFN1, ZFN2, IDUA donor.

171. The method of any one of embodiments 147 to 149, wherein the subject has MPS-II and the transgene encodes ZFN1, ZFN2, IDS donor.

172. The method of any one of embodiments 147 to 149, wherein the subject has MPS-IIIA and the transgene encodes SGSH.

173. The method of any one of embodiments 147 to 149, wherein the subject has MPS-VI and the transgene encodes ARSB.

174. The method of any one of embodiments 147 to 149, wherein the subject has OTC deficiency and the transgene encodes OTC.

175. The method of any one of embodiments 147 to 149, wherein the subject has A1AT deficiency and the transgene encodes A1AT.

176. The method of any one of embodiments 147 to 149, wherein the subject has CMT1A and the transgene encodes NTF3.

177. The method of any one of embodiments 147 to 149, wherein the subject has DMD and the transgene encodes a microdystrophin or a mini-dystrophin.

178. The method of any one of embodiments 147 to 149, wherein the subject has LGMD type 2E and the transgene encodes LGMD2E.

179. The method of any one of embodiments 147 to 149, wherein the subject has dysferlinopathy and the transgene encodes DYSF.

180. The method of any one of embodiments 147 to 149, wherein the subject has an HIV infection and the transgene encodes a PG9 antibody or a VRC07 antibody.

181. The method of any one of embodiments 147 to 149, wherein the subject has Pompe Disease and the transgene encodes GAA.

182. The method of any one of embodiments 147 to 149, wherein the subject has X-linked MTM and the transgene encodes MTM1 183. The method of any one of embodiments 119 to 121, wherein the adenovirus-based vaccine comprises a transgene that encodes an amino acid sequence having at least 85% identity to any one of SEQ ID NOS: 237-266 (optionally any one of SEQ ID NOS: 237-240 and SEQ ID NOS: 249-266) or a fragment thereof capable of eliciting an immune response in a subject.

184. The method of embodiment 183, wherein the amino acid sequence has at least 90% identity to any one of SEQ ID NOS: 237-266 (optionally any one of SEQ ID NOS: 237-240 and SEQ ID NOS: 249-266) or a fragment thereof capable of eliciting an immune response in a subject.

185. The method of embodiment 183 or embodiment 184, wherein the amino acid sequence has at least 95% identity to any one of SEQ ID NOS: 237-266 (optionally any one of SEQ ID NOS: 237-240 and SEQ ID NOS: 249-266) or a fragment thereof capable of eliciting an immune response in a subject.

186. The method of any one of embodiments 183 to 185, wherein the amino acid sequence has at least 96% identity to any one of SEQ ID NOS: 237-266 (optionally any one of SEQ ID NOS: 237-240 and SEQ ID NOS: 249-266) or a fragment thereof capable of eliciting an immune response in a subject.

187. The method of any one of embodiments 183 to 186, wherein the amino acid sequence has at least 97% identity to any one of SEQ ID NOS: 237-266 (optionally any one of SEQ ID NOS: 237-240 and SEQ ID NOS: 249-266) or a fragment thereof capable of eliciting an immune response in a subject.

188. The method of any one of embodiments 183 to 187, wherein the amino acid sequence has at least 98% identity to any one of SEQ ID NOS: 237-266 (optionally any one of SEQ ID NOS: 237-240 and SEQ ID NOS: 249-266) or a fragment thereof capable of eliciting an immune response in a subject.

189. The method of any one of embodiments 183 to 188, wherein the amino acid sequence has at least 99% identity to any one of SEQ ID NOS: 237-266 (optionally any one of SEQ ID NOS: 237-240 and SEQ ID NOS: 249-266) or a fragment thereof capable of eliciting an immune response in a subject.

190. The method of any one of embodiments 183 to 189, wherein the amino acid sequence has 100% identity to any one of SEQ ID NOS: 237-266 (optionally any one of SEQ ID NOS: 237-240 and SEQ ID NOS: 249-266) or a fragment thereof capable of eliciting an immune response in a subject.

191. The method of any one of embodiments 183 to 190, wherein the amino acid sequence has at least 85% identity to SEQ ID NO:237, SEQ ID NO:238; SEQ ID NO:239, or SEQ ID NO:240, or a fragment thereof, in which the amino acid sequence comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, or thirty-two of the following mutations: S13I, L18F, T20N, P26S, Δ69-70, D80A, D80Y, L141F, Δ144, W152C, M153T, M153I, F157L, Δ242-244, D253G, S255F, A262S, V367F, K417N, K417T, N439K, L452R, Y453F, S477N, S477R, E484K, S494P, N501T, N501Y, Q613H, D614G, and P681R relative to the amino acid sequence of SEQ ID NO:237 or SEQ ID NO:250.

192. The method of embodiment 191, in which the amino acid sequence comprises at least three of the mutations.

193. The method of embodiment 191, in which the amino acid sequence comprises at least five of the mutations.

194. The method of embodiment 191, in which the amino acid sequence comprises at least ten of the mutations.

195. The method of any one of embodiments 183 to 194, wherein the amino acid sequence has at least 90% identity to SEQ ID NO:237, SEQ ID NO:238; SEQ ID NO:239, or SEQ ID NO:240, or a fragment thereof, in which the amino acid sequence comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, or thirty-two of the following mutations: S13I, L18F, T20N, P26S, Δ69-70, D80A, D80Y, L141F, Δ144, W152C, M153T, M153I, F157L, Δ242-244, D253G, S255F, A262S, V367F, K417N, K417T, N439K, L452R, Y453F, S477N, S477R, E484K, S494P, N501T, N501Y, Q613H, D614G, and P681R relative to the amino acid sequence of SEQ ID NO:237 or SEQ ID NO:250.

196. The method of embodiment 195, in which the amino acid sequence comprises at least three of the mutations.

197. The method of embodiment 195, in which the amino acid sequence comprises at least five of the mutations.

198. The method of embodiment 195, in which the amino acid sequence comprises at least ten of the mutations.

199. The method of any one of embodiments 183 to 198, wherein the amino acid sequence has at least 95% identity to SEQ ID NO:237, SEQ ID NO:238; SEQ ID NO:239, or SEQ ID NO:240, or a fragment thereof, in which the amino acid sequence comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, or thirty-two of the following mutations: S13I, L18F, T20N, P26S, Δ69-70, D80A, D80Y, L141F, Δ144, W152C, M153T, M153I, F157L, Δ242-244, D253G, S255F, A262S, V367F, K417N, K417T, N439K, L452R, Y453F, S477N, S477R, E484K, S494P, N501T, N501Y, Q613H, D614G, and P681R relative to the amino acid sequence of SEQ ID NO:237 or SEQ ID NO:250.

200. The method of embodiment 199, in which the amino acid sequence comprises at least three of the mutations.

201. The method of embodiment 199, in which the amino acid sequence comprises at least five of the mutations.

202. The method of embodiment 199, in which the amino acid sequence comprises at least ten of the mutations.

203. The method of any one of embodiments 183 to 202, wherein the amino acid sequence has at least 96% identity to SEQ ID NO:237, SEQ ID NO:238; SEQ ID NO:239, or SEQ ID NO:240, or a fragment thereof, in which the amino acid sequence comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, or thirty-two of the following mutations: S13I, L18F, T20N, P26S, Δ69-70, D80A, D80Y, L141F, Δ144, W152C, M153T, M153I, F157L, Δ242-244, D253G, S255F, A262S, V367F, K417N, K417T, N439K, L452R, Y453F, S477N, S477R, E484K, S494P, N501T, N501Y, Q613H, D614G, and P681R relative to the amino acid sequence of SEQ ID NO:237 or SEQ ID NO:250.

204. The method of embodiment 203, in which the amino acid sequence comprises at least three of the mutations.

205. The method of embodiment 203, in which the amino acid sequence comprises at least five of the mutations.

206. The method of embodiment 203, in which the amino acid sequence comprises at least ten of the mutations.

207. The method of any one of embodiments 183 to 206, wherein the amino acid sequence has at least 97% identity to SEQ ID NO:237, SEQ ID NO:238; SEQ ID NO:239, or SEQ ID NO:240, or a fragment thereof, in which the amino acid sequence comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, or thirty-two of the following mutations: S13I, L18F, T20N, P26S, Δ69-70, D80A, D80Y, L141F, Δ144, W152C, M153T, M153I, F157L, Δ242-244, D253G, S255F, A262S, V367F, K417N, K417T, N439K, L452R, Y453F, S477N, S477R, E484K, S494P, N501T, N501Y, Q613H, D614G, and P681R relative to the amino acid sequence of SEQ ID NO:237 or SEQ ID NO:250.

208. The method of embodiment 207, in which the amino acid sequence comprises at least three of the mutations.

209. The method of embodiment 207, in which the amino acid sequence comprises at least five of the mutations.

210. The method of embodiment 207, in which the amino acid sequence comprises at least ten of the mutations.

211. The method of any one of embodiments 183 to 210, wherein the amino acid sequence has at least 98% identity to SEQ ID NO:237, SEQ ID NO:238; SEQ ID NO:239, or SEQ ID NO:240, or a fragment thereof, in which the amino acid sequence comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, or thirty-two of the following mutations: S13I, L18F, T20N, P26S, Δ69-70, D80A, D80Y, L141F, Δ144, W152C, M153T, M153I, F157L, Δ242-244, D253G, S255F, A262S, V367F, K417N, K417T, N439K, L452R, Y453F, S477N, S477R, E484K, S494P, N501T, N501Y, Q613H, D614G, and P681R relative to the amino acid sequence of SEQ ID NO:237 or SEQ ID NO:250.

212. The method of embodiment 211, in which the amino acid sequence comprises at least three of the mutations.

213. The method of embodiment 211, in which the amino acid sequence comprises at least five of the mutations.

214. The method of embodiment 211, in which the amino acid sequence comprises at least ten of the mutations.

215. The method of any one of embodiments 183 to 214, wherein the amino acid sequence has at least 99% identity to SEQ ID NO:237, SEQ ID NO:238; SEQ ID NO:239, or SEQ ID NO:240, or a fragment thereof, in which the amino acid sequence comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, or thirty-two of the following mutations: S13I, L18F, T20N, P26S, Δ69-70, D80A, D80Y, L141F, Δ144, W152C, M153T, M153I, F157L, Δ242-244, D253G, S255F, A262S, V367F, K417N, K417T, N439K, L452R, Y453F, S477N, S477R, E484K, S494P, N501T, N501Y, Q613H, D614G, and P681R relative to the amino acid sequence of SEQ ID NO:237 or SEQ ID NO:250.

216. The method of embodiment 215, in which the amino acid sequence comprises at least three of the mutations.

217. The method of embodiment 215, in which the amino acid sequence comprises at least five of the mutations.

218. The method of embodiment 215, in which the amino acid sequence comprises at least ten of the mutations.

219. The method of any one of embodiments 183 to 218, wherein the amino acid sequence has one, two, three, four, five, six, seven, eight, nine, ten, eleven, or twelve of the mutations: L18F, T20N, P26S, D80A, M153T, M153I, A242-244, K417N, Y453F, E484K, N501Y, and D614G relative to the amino acid sequence of SEQ ID NO:237 or SEQ ID NO: 250.

220. The method of embodiment 219, in which the amino acid sequence comprises at least three of the mutations.

221. The method of embodiment 219, in which the amino acid sequence comprises at least five of the mutations.

222. The method of embodiment 219, in which the amino acid sequence comprises at least ten of the mutations.

223. The method of any one of embodiments 183 to 222, wherein the amino acid sequence has one, two, three, four, five, six, seven, eight, or nine of the mutations: S13I, Δ69-70, Δ144, W152C, D253G, A262S, L452R, S477N, and D614G relative to the amino acid sequence of SEQ ID NO:237 or SEQ ID NO:250.

224. The method of embodiment 223, in which the amino acid sequence comprises at least three of the mutations.

225. The method of embodiment 223, in which the amino acid sequence comprises at least five of the mutations.

226. The method of any one of embodiments 183 to 225, wherein the amino acid sequence has one, two, three, four, five, six, seven, eight, nine, ten, eleven, or twelve of the mutations: D80Y, L141F, F157L, S255F, V367F, K417T, N439K, S477R, S494P, N501T, Q613H, and P681R relative to the amino acid sequence of SEQ ID NO:237 or SEQ ID NO: 250.

227. The method of embodiment 226, in which the amino acid sequence comprises at least three of the mutations.

228. The method of embodiment 226, in which the amino acid sequence comprises at least five of the mutations.

229. The method of embodiment 226, in which the amino acid sequence comprises at least ten of the mutations.

230. The method of any one of embodiments 183 to 229, wherein the amino acid sequence has at least 85% identity to any one of SEQ ID NOs: 250, 253-256, and 257-260, or a fragment thereof, in which the amino acid sequence comprises has one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, or 333 of the following mutations: V3G, L5F, P9L, S13I, L18F, T19R, T20N, P26S, A27S, T33I, V36F, V36I, S45F, H49Y, Q52R, L54F, W64R, A67V, A69-70, G75V, T76I, D80A, D80G, P85S, S94F, T95I, S98F, 1105V, D111N, S112L, L118F, V120L, V126A, V127F, E132Q, D138Y, G142D, Δ144, N148T, W152R, Δ156-157, F157S, R158G, S162I, T167S, S172A, L176F, D178H, G181V, L189F, R190S, V193L, D198Y, 1203V, Y204H, 1210T, D215G, L216F, A222V, V227A, D228H, 1233V, R237K, Δ242-244, H245Y, A246-252, D253G, D253N, W258L, A262S, A263P, V267L, P272L, R273S, E281Q, T284I, V289L, A292S, T299I, K300M, T307I, V308L, E309Q, F318S, V320F, T323I, V227I, P330S, L335F, P337S, G339S, R346S, R346K, A348S, K356R, R357K, V362F, V367F, V367L, S371T, T376I, V382L, P384L, T385N, N394S, N394H, V395I, R403K, E406Q, R408I, Q414K, K417T, K417N, D427N, D427Y, T430I, N439K, N440S, N440K, L452M, L452R, L452Q, L461F, K462T, 1468V, T470I, E471Q, T478K, E484K, F490S, S494P, N501T, N501Y, Y505H, V510L, L513F, A520S, A522S, A522V, T531S, V534I, V534F, N540S, F543L, T547I, T549I, E554D, K558N, P561S, F565L, A570D, T572I, A575S, E583Q, E583D, L585F, G594S, V595I, I598V, T604A, T604I, Q607K, Q613H, D614G, N616S, V622F, A623S, D627G, P631S, T632S, T638I, S640F, N641S, A647S, A653V, H655Y, Y660F, 1670V, A672V, Q677H, P681H, P681R, A688V, S691P, A694V, M697I, A701V, S704L, A706V, T716I, T716V, T719I, M731I, T732I, T732A, M740V, T747I, T747N, N751D, S758G, T761I, T761R, G769V, A771S, V772I, Q779K, E780D, V785I, T791I, K795R, D796H, S803A, P809S, P812S, P812L, L822F, V826L, T827I, A831V, K835R, D843N, A845S, A845V, K854N, T859N, T859I, M869I, I870V, A871V, A879S, A879V, T883I, F888L, A890V, A892V, A899S, M902I, A903S, 1909V, V911I, V915I, L922F, A924S, S929I, D936Y, S939F, S940F, G946R, D950N, D950H, Q957R, T961M, V963A, S967N, V976F, S982A, K986N, K986R, R995G, 1997V, T998I, Q1005H, T1006I, T1009I, R1014K, A1016V, A1020S, A1025G, T1027I, V1033A, K1038Q, V1040F, K1045N, L1049I, M1050I, A1056V, H1058Y, L1063F, T1066N, Q1071H, K1073N, A1078S, D1084Y, D1084E, A1086S, V1094F, H1101D, H1101Y, V1104L, E1111K, D1118H, D1118Y, G1124V, D1127G, 11130V, V1133F, D1139H, L1141W, D1146Y, E1150D, D1153Y, P1162L, P1162S, D1163Y, N1173S, A1174V, V1176F, E1182D, R1185H, K1191N, N1192S, E1195Q, E1202Q, K1205N, Q1208H, 11216T, G1219V, G1219C, V1228L, M1229I, V1230L, M1237I, T1238I, C1243F, C1247F, S1252F, S1252P, D1260N, P1263L, V1264L, H1271Y, T1273I, and T1273A relative to the amino acid sequence of SEQ ID NO:237 or 250.

231. The method of embodiment 230, in which the amino acid sequence comprises at least three of the mutations.

232. The method of embodiment 230, in which the amino acid sequence comprises at least five of the mutations.

233. The method of embodiment 230, in which the amino acid sequence comprises at least ten of the mutations.

234. The method of any one of embodiments 183 to 233, wherein the amino acid sequence has at least 90% identity to any one of SEQ ID NOs: 250, 253-256, and 257-260, or a fragment thereof, in which the amino acid sequence comprises has one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, or 333 of the following mutations: V3G, L5F, P9L, S13I, L18F, T19R, T20N, P26S, A27S, T33I, V36F, V36I, S45F, H49Y, Q52R, L54F, W64R, A67V, A69-70, G75V, T76I, D80A, D80G, P85S, S94F, T95I, S98F, 1105V, D111N, S112L, L118F, V120L, V126A, V127F, E132Q, D138Y, G142D, Δ144, 148, W152R, Δ156-157, F157S, R158G, S162I, T167S, S172A, L176F, D178H, G181V, L189F, R190S, V193L, D198Y, 1203V, Y204H, 1210T, D215G, L216F, A222V, V227A, D228H, 1233V, R237K, Δ242-244, H245Y, A246-252, D253G, D253N, W258L, A262S, A263P, V267L, P272L, R273S, E281Q, T284I, V289L, A292S, T299I, K300M, T307I, V308L, E309Q, F318S, V320F, T323I, V227I, P330S, L335F, P337S, G339S, R346S, R346K, A348S, K356R, R357K, V362F, V367F, V367L, S371T, T376I, V382L, P384L, T385N, N394S, N394H, V395I, R403K, E406Q, R408I, Q414K, K417T, K417N, D427N, D427Y, T430I, N439K, N440S, N440K, L452M, L452R, L452Q, L461F, K462T, 1468V, T470I, E471Q, T478K, E484K, F490S, S494P, N501T, N501Y, Y505H, V510L, L513F, A520S, A522S, A522V, T531S, V534I, V534F, N540S, F543L, T547I, T549I, E554D, K558N, P561S, F565L, A570D, T572I, A575S, E583Q, E583D, L585F, G594S, V595I, I598V, T604A, T604I, Q607K, Q613H, D614G, N616S, V622F, A623S, D627G, P631S, T632S, T638I, S640F, N641S, A647S, A653V, H655Y, Y660F, 1670V, A672V, Q677H, P681H, P681R, A688V, S691P, A694V, M697I, A701V, S704L, A706V, T716I, T716V, T719I, M731I, T732I, T732A, M740V, T747I, T747N, N751D, S758G, T761I, T761R, G769V, A771S, V772I, Q779K, E780D, V785I, T791I, K795R, D796H, S803A, P809S, P812S, P812L, L822F, V826L, T827I, A831V, K835R, D843N, A845S, A845V, K854N, T859N, T859I, M869I, I870V, A871V, A879S, A879V, T883I, F888L, A890V, A892V, A899S, M902I, A903S, 1909V, V911I, V915I, L922F, A924S, S929I, D936Y, S939F, S940F, G946R, D950N, D950H, Q957R, T961M, V963A, S967N, V976F, S982A, K986N, K986R, R995G, 1997V, T998I, Q1005H, T1006I, T1009I, R1014K, A1016V, A1020S, A1025G, T1027I, V1033A, K1038Q, V1040F, K1045N, L1049I, M1050I, A1056V, H1058Y, L1063F, T1066N, Q1071H, K1073N, A1078S, D1084Y, D1084E, A1086S, V1094F, H1101D, H1101Y, V1104L, E1111K, D1118H, D1118Y, G1124V, D1127G, 11130V, V1133F, D1139H, L1141W, D1146Y, E1150D, D1153Y, P1162L, P1162S, D1163Y, N1173S, A1174V, V1176F, E1182D, R1185H, K1191N, N1192S, E1195Q, E1202Q, K1205N, Q1208H, 11216T, G1219V, G1219C, V1228L, M1229I, V1230L, M1237I, T1238I, C1243F, C1247F, S1252F, S1252P, D1260N, P1263L, V1264L, H1271Y, T1273I, and T1273A relative to the amino acid sequence of SEQ ID NO:237 or 250.

235. The method of embodiment 234, in which the amino acid sequence comprises at least three of the mutations.

236. The method of embodiment 234, in which the amino acid sequence comprises at least five of the mutations.

237. The method of embodiment 234, in which the amino acid sequence comprises at least ten of the mutations.

238. The method of any one of embodiments 183 to 237, wherein the amino acid sequence has at least 95% identity to any one of SEQ ID NOs: 250, 253-256, and 257-260, or a fragment thereof, in which the amino acid sequence comprises has one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, or 333 of the following mutations: V3G, L5F, P9L, S13I, L18F, T19R, T20N, P26S, A27S, T33I, V36F, V36I, S45F, H49Y, Q52R, L54F, W64R, A67V, A69-70, G75V, T76I, D80A, D80G, P85S, S94F, T95I, S98F, 1105V, D111N, S112L, L118F, V120L, V126A, V127F, E132Q, D138Y, G142D, A144, N148T, W152R, A156-157, F157S, R158G, S162I, T167S, S172A, L176F, D178H, G181V, L189F, R190S, V193L, D198Y, 1203V, Y204H, 1210T, D215G, L216F, A222V, V227A, D228H, 1233V, R237K, A242-244, H245Y, A246-252, D253G, D253N, W258L, A262S, A263P, V267L, P272L, R273S, E281Q, T284I, V289L, A292S, T299I, K300M, T307I, V308L, E309Q, F318S, V320F, T323I, V227I, P330S, L335F, P337S, G339S, R346S, R346K, A348S, K356R, R357K, V362F, V367F, V367L, S371T, T376I, V382L, P384L, T385N, N394S, N394H, V395I, R403K, E406Q, R408I, Q414K, K417T, K417N, D427N, D427Y, T430I, N439K, N440S, N440K, L452M, L452R, L452Q, L461F, K462T, 1468V, T470I, E471Q, T478K, E484K, F490S, S494P, N501T, N501Y, Y505H, V510L, L513F, A520S, A522S, A522V, T531S, V534I, V534F, N540S, F543L, T547I, T549I, E554D, K558N, P561S, F565L, A570D, T572I, A575S, E583Q, E583D, L585F, G594S, V595I, I598V, T604A, T604I, Q607K, Q613H, D614G, N616S, V622F, A623S, D627G, P631S, T632S, T638I, S640F, N641S, A647S, A653V, H655Y, Y660F, 1670V, A672V, Q677H, P681H, P681R, A688V, S691P, A694V, M697I, A701V, S704L, A706V, T716I, T716V, T719I, M731I, T732I, T732A, M740V, T747I, T747N, N751D, S758G, T761I, T761R, G769V, A771S, V772I, Q779K, E780D, V785I, T791I, K795R, D796H, S803A, P809S, P812S, P812L, L822F, V826L, T827I, A831V, K835R, D843N, A845S, A845V, K854N, T859N, T859I, M869I, I870V, A871V, A879S, A879V, T883I, F888L, A890V, A892V, A899S, M902I, A903S, 1909V, V911I, V915I, L922F, A924S, S929I, D936Y, S939F, S940F, G946R, D950N, D950H, Q957R, T961M, V963A, S967N, V976F, S982A, K986N, K986R, R995G, 1997V, T998I, Q1005H, T1006I, T1009I, R1014K, A1016V, A1020S, A1025G, T1027I, V1033A, K1038Q, V1040F, K1045N, L1049I, M1050I, A1056V, H1058Y, L1063F, T1066N, Q1071H, K1073N, A1078S, D1084Y, D1084E, A1086S, V1094F, H1101D, H1101Y, V1104L, E1111K, D1118H, D1118Y, G1124V, D1127G, 11130V, V1133F, D1139H, L1141W, D1146Y, E1150D, D1153Y, P1162L, P1162S, D1163Y, N1173S, A1174V, V1176F, E1182D, R1185H, K1191N, N1192S, E1195Q, E1202Q, K1205N, Q1208H, 11216T, G1219V, G1219C, V1228L, M1229I, V1230L, M1237I, T1238I, C1243F, C1247F, S1252F, S1252P, D1260N, P1263L, V1264L, H1271Y, T1273I, and T1273A relative to the amino acid sequence of SEQ ID NO:237 or 250.

239. The method of embodiment 238, in which the amino acid sequence comprises at least three of the mutations.

240. The method of embodiment 238, in which the amino acid sequence comprises at least five of the mutations.

241. The method of embodiment 238, in which the amino acid sequence comprises at least ten of the mutations.

242. The method of any one of embodiments 183 to 241, wherein the amino acid sequence has at least 96% identity to any one of SEQ ID NOs: 250, 253-256, and 257-260, or a fragment thereof, in which the amino acid sequence comprises has one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, or 333 of the following mutations: V3G, L5F, P9L, S13I, L18F, T19R, T20N, P26S, A27S, T33I, V36F, V36I, S45F, H49Y, Q52R, L54F, W64R, A67V, A69-70, G75V, T76I, D80A, D80G, P85S, S94F, T95I, S98F, 1105V, D111N, S112L, L118F, V120L, V126A, V127F, E132Q, D138Y, G142D, A144, N148T, W152R, A156-157, F157S, R158G, S162I, T167S, S172A, L176F, D178H, G181V, L189F, R190S, V193L, D198Y, 1203V, Y204H, 1210T, D215G, L216F, A222V, V227A, D228H, 1233V, R237K, A242-244, H245Y, A246-252, D253G, D253N, W258L, A262S, A263P, V267L, P272L, R273S, E281Q, T284I, V289L, A292S, T299I, K300M, T307I, V308L, E309Q, F318S, V320F, T323I, V227I, P330S, L335F, P337S, G339S, R346S, R346K, A348S, K356R, R357K, V362F, V367F, V367L, S371T, T376I, V382L, P384L, T385N, N394S, N394H, V395I, R403K, E406Q, R408I, Q414K, K417T, K417N, D427N, D427Y, T430I, N439K, N440S, N440K, L452M, L452R, L452Q, L461F, K462T, 1468V, T470I, E471Q, T478K, E484K, F490S, S494P, N501T, N501Y, Y505H, V510L, L513F, A520S, A522S, A522V, T531S, V534I, V534F, N540S, F543L, T547I, T549I, E554D, K558N, P561S, F565L, A570D, T572I, A575S, E583Q, E583D, L585F, G594S, V595I, I598V, T604A, T604I, Q607K, Q613H, D614G, N616S, V622F, A623S, D627G, P631S, T632S, T638I, S640F, N641S, A647S, A653V, H655Y, Y660F, 1670V, A672V, Q677H, P681H, P681R, A688V, S691P, A694V, M697I, A701V, S704L, A706V, T716I, T716V, T719I, M731I, T732I, T732A, M740V, T747I, T747N, N751D, S758G, T761I, T761R, G769V, A771S, V772I, Q779K, E780D, V785I, T791I, K795R, D796H, S803A, P809S, P812S, P812L, L822F, V826L, T827I, A831V, K835R, D843N, A845S, A845V, K854N, T859N, T859I, M869I, I870V, A871V, A879S, A879V, T883I, F888L, A890V, A892V, A899S, M902I, A903S, 1909V, V911I, V915I, L922F, A924S, S929I, D936Y, S939F, S940F, G946R, D950N, D950H, Q957R, T961M, V963A, S967N, V976F, S982A, K986N, K986R, R995G, 1997V, T998I, Q1005H, T1006I, T1009I, R1014K, A1016V, A1020S, A1025G, T1027I, V1033A, K1038Q, V1040F, K1045N, L1049I, M1050I, A1056V, H1058Y, L1063F, T1066N, Q1071H, K1073N, A1078S, D1084Y, D1084E, A1086S, V1094F, H1101D, H1101Y, V1104L, E1111K, D1118H, D1118Y, G1124V, D1127G, 11130V, V1133F, D1139H, L1141W, D1146Y, E1150D, D1153Y, P1162L, P1162S, D1163Y, N1173S, A1174V, V1176F, E1182D, R1185H, K1191N, N1192S, E1195Q, E1202Q, K1205N, Q1208H, 11216T, G1219V, G1219C, V1228L, M1229I, V1230L, M1237I, T1238I, C1243F, C1247F, S1252F, S1252P, D1260N, P1263L, V1264L, H1271Y, T1273I, and T1273A relative to the amino acid sequence of SEQ ID NO:237 or 250.

243. The method of embodiment 242, in which the amino acid sequence comprises at least three of the mutations.

244. The method of embodiment 242, in which the amino acid sequence comprises at least five of the mutations.

245. The method of embodiment 242, in which the amino acid sequence comprises at least ten of the mutations.

246. The method of any one of embodiments 183 to 245, wherein the amino acid sequence has at least 97% identity to any one of SEQ ID NOs: 250, 253-256, and 257-260, or a fragment thereof, in which the amino acid sequence comprises has one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, or 333 of the following mutations: V3G, L5F, P9L, S13I, L18F, T19R, T20N, P26S, A27S, T33I, V36F, V36I, S45F, H49Y, Q52R, L54F, W64R, A67V, A69-70, G75V, T76I, D80A, D80G, P85S, S94F, T95I, S98F, 1105V, D111N, S112L, L118F, V120L, V126A, V127F, E132Q, D138Y, G142D, A144, N148T, W152R, A156-157, F157S, R158G, S162I, T167S, S172A, L176F, D178H, G181V, L189F, R190S, V193L, D198Y, 1203V, Y204H, 1210T, D215G, L216F, A222V, V227A, D228H, 1233V, R237K, A242-244, H245Y, A246-252, D253G, D253N, W258L, A262S, A263P, V267L, P272L, R273S, E281Q, T284I, V289L, A292S, T299I, K300M, T307I, V308L, E309Q, F318S, V320F, T323I, V227I, P330S, L335F, P337S, G339S, R346S, R346K, A348S, K356R, R357K, V362F, V367F, V367L, S371T, T376I, V382L, P384L, T385N, N394S, N394H, V395I, R403K, E406Q, R408I, Q414K, K417T, K417N, D427N, D427Y, T430I, N439K, N440S, N440K, L452M, L452R, L452Q, L461F, K462T, |468V, T470I, E471Q, T478K, E484K, F490S, S494P, N501T, N501Y, Y505H, V510L, L513F, A520S, A522S, A522V, T531S, V534I, V534F, N540S, F543L, T547I, T549I, E554D, K558N, P561S, F565L, A570D, T572I, A575S, E583Q, E583D, L585F, G594S, V595I, I598V, T604A, T604I, Q607K, Q613H, D614G, N616S, V622F, A623S, D627G, P631S, T632S, T638I, S640F, N641S, A647S, A653V, H655Y, Y660F, 1670V, A672V, Q677H, P681H, P681R, A688V, S691P, A694V, M697I, A701V, S704L, A706V, T716I, T716V, T719I, M731I, T732I, T732A, M740V, T747I, T747N, N751D, S758G, T761I, T761R, G769V, A771S, V772I, Q779K, E780D, V785I, T791I, K795R, D796H, S803A, P809S, P812S, P812L, L822F, V826L, T827I, A831V, K835R, D843N, A845S, A845V, K854N, T859N, T859I, M869I, I870V, A871V, A879S, A879V, T883I, F888L, A890V, A892V, A899S, M902I, A903S, 1909V, V911I, V915I, L922F, A924S, S929I, D936Y, S939F, S940F, G946R, D950N, D950H, Q957R, T961M, V963A, S967N, V976F, S982A, K986N, K986R, R995G, 1997V, T998I, Q1005H, T1006I, T1009I, R1014K, A1016V, A1020S, A1025G, T1027I, V1033A, K1038Q, V1040F, K1045N, L1049I, M1050I, A1056V, H1058Y, L1063F, T1066N, Q1071H, K1073N, A1078S, D1084Y, D1084E, A1086S, V1094F, H1101D, H1101Y, V1104L, E1111K, D1118H, D1118Y, G1124V, D1127G, 11130V, V1133F, D1139H, L1141W, D1146Y, E1150D, D1153Y, P1162L, P1162S, D1163Y, N1173S, A1174V, V1176F, E1182D, R1185H, K1191N, N1192S, E1195Q, E1202Q, K1205N, Q1208H, 11216T, G1219V, G1219C, V1228L, M1229I, V1230L, M1237I, T1238I, C1243F, C1247F, S1252F, S1252P, D1260N, P1263L, V1264L, H1271Y, T1273I, and T1273A relative to the amino acid sequence of SEQ ID NO:237 or 250.

247. The method of embodiment 246, in which the amino acid sequence comprises at least three of the mutations.

248. The method of embodiment 246, in which the amino acid sequence comprises at least five of the mutations.

249. The method of embodiment 246, in which the amino acid sequence comprises at least ten of the mutations.

250. The method of any one of embodiments 183 to 249, wherein the amino acid sequence has at least 98% identity to any one of SEQ ID NOs: 250, 253-256, and 257-260, or a fragment thereof, in which the amino acid sequence comprises has one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, or 333 of the following mutations: V3G, L5F, P9L, S13I, L18F, T19R, T20N, P26S, A27S, T33I, V36F, V36I, S45F, H49Y, Q52R, L54F, W64R, A67V, A69-70, G75V, T76I, D80A, D80G, P85S, S94F, T95I, S98F, 1105V, D111N, S112L, L118F, V120L, V126A, V127F, E132Q, D138Y, G142D, Δ144, N148T, W152R, Δ156-157, F157S, R158G, S162I, T167S, S172A, L176F, D178H, G181V, L189F, R190S, V193L, D198Y, 1203V, Y204H, 1210T, D215G, L216F, A222V, V227A, D228H, 1233V, R237K, Δ242-244, H245Y, Δ246-252, D253G, D253N, W258L, A262S, A263P, V267L, P272L, R273S, E281Q, T284I, V289L, A292S, T299I, K300M, T307I, V308L, E309Q, F318S, V320F, T323I, V227I, P330S, L335F, P337S, G339S, R346S, R346K, A348S, K356R, R357K, V362F, V367F, V367L, S371T, T376I, V382L, P384L, T385N, N394S, N394H, V395I, R403K, E406Q, R408I, Q414K, K417T, K417N, D427N, D427Y, T430I, N439K, N440S, N440K, L452M, L452R, L452Q, L461F, K462T, 1468V, T470I, E471Q, T478K, E484K, F490S, S494P, N501T, N501Y, Y505H, V510L, L513F, A520S, A522S, A522V, T531S, V534I, V534F, N540S, F543L, T547I, T549I, E554D, K558N, P561S, F565L, A570D, T572I, A575S, E583Q, E583D, L585F, G594S, V595I, I598V, T604A, T604I, Q607K, Q613H, D614G, N616S, V622F, A623S, D627G, P631S, T632S, T638I, S640F, N641S, A647S, A653V, H655Y, Y660F, 1670V, A672V, Q677H, P681H, P681R, A688V, S691P, A694V, M697I, A701V, S704L, A706V, T716I, T716V, T719I, M731I, T732I, T732A, M740V, T747I, T747N, N751D, S758G, T761I, T761R, G769V, A771S, V772I, Q779K, E780D, V785I, T791I, K795R, D796H, S803A, P809S, P812S, P812L, L822F, V826L, T827I, A831V, K835R, D843N, A845S, A845V, K854N, T859N, T859I, M869I, I870V, A871V, A879S, A879V, T883I, F888L, A890V, A892V, A899S, M902I, A903S, 1909V, V911I, V915I, L922F, A924S, S929I, D936Y, S939F, S940F, G946R, D950N, D950H, Q957R, T961M, V963A, S967N, V976F, S982A, K986N, K986R, R995G, 1997V, T998I, Q1005H, T1006I, T1009I, R1014K, A1016V, A1020S, A1025G, T1027I, V1033A, K1038Q, V1040F, K1045N, L1049I, M1050I, A1056V, H1058Y, L1063F, T1066N, Q1071H, K1073N, A1078S, D1084Y, D1084E, A1086S, V1094F, H1101D, H1101Y, V1104L, E1111K, D1118H, D1118Y, G1124V, D1127G, 11130V, V1133F, D1139H, L1141W, D1146Y, E1150D, D1153Y, P1162L, P1162S, D1163Y, N1173S, A1174V, V1176F, E1182D, R1185H, K1191N, N1192S, E1195Q, E1202Q, K1205N, Q1208H, 11216T, G1219V, G1219C, V1228L, M1229I, V1230L, M1237I, T1238I, C1243F, C1247F, S1252F, S1252P, D1260N, P1263L, V1264L, H1271Y, T1273I, and T1273A relative to the amino acid sequence of SEQ ID NO:237 or 250.

251. The method of embodiment 250, in which the amino acid sequence comprises at least three of the mutations.

252. The method of embodiment 250, in which the amino acid sequence comprises at least five of the mutations.

253. The method of embodiment 250, in which the amino acid sequence comprises at least ten of the mutations.

254. The method of any one of embodiments 183 to 253, wherein the amino acid sequence has at least 99% identity to any one of SEQ ID NOs: 250, 253-256, and 257-260, or a fragment thereof, in which the amino acid sequence comprises has one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, or 333 of the following mutations: V3G, L5F, P9L, S13I, L18F, T19R, T20N, P26S, A27S, T33I, V36F, V36I, S45F, H49Y, Q52R, L54F, W64R, A67V, A69-70, G75V, T76I, D80A, D80G, P85S, S94F, T95I, S98F, 1105V, D111N, S112L, L118F, V120L, V126A, V127F, E132Q, D138Y, G142D, A144, N148T, W152R, A156-157, F157S, R158G, S162I, T167S, S172A, L176F, D178H, G181V, L189F, R190S, V193L, D198Y, 1203V, Y204H, 1210T, D215G, L216F, A222V, V227A, D228H, 1233V, R237K, A242-244, H245Y, A246-252, D253G, D253N, W258L, A262S, A263P, V267L, P272L, R273S, E281Q, T284I, V289L, A292S, T299I, K300M, T307I, V308L, E309Q, F318S, V320F, T323I, V227I, P330S, L335F, P337S, G339S, R346S, R346K, A348S, K356R, R357K, V362F, V367F, V367L, S371T, T376I, V382L, P384L, T385N, N394S, N394H, V395I, R403K, E406Q, R408I, Q414K, K417T, K417N, D427N, D427Y, T430I, N439K, N440S, N440K, L452M, L452R, L452Q, L461F, K462T, 1468V, T470I, E471Q, T478K, E484K, F490S, S494P, N501T, N501Y, Y505H, V510L, L513F, A520S, A522S, A522V, T531S, V534I, V534F, N540S, F543L, T547I, T549I, E554D, K558N, P561S, F565L, A570D, T572I, A575S, E583Q, E583D, L585F, G594S, V595I, I598V, T604A, T604I, Q607K, Q613H, D614G, N616S, V622F, A623S, D627G, P631S, T632S, T638I, S640F, N641S, A647S, A653V, H655Y, Y660F, 1670V, A672V, Q677H, P681H, P681R, A688V, S691P, A694V, M697I, A701V, S704L, A706V, T716I, T716V, T719I, M731I, T732I, T732A, M740V, T747I, T747N, N751D, S758G, T761I, T761R, G769V, A771S, V772I, Q779K, E780D, V785I, T791I, K795R, D796H, S803A, P809S, P812S, P812L, L822F, V826L, T827I, A831V, K835R, D843N, A845S, A845V, K854N, T859N, T859I, M869I, I870V, A871V, A879S, A879V, T883I, F888L, A890V, A892V, A899S, M902I, A903S, 1909V, V911I, V915I, L922F, A924S, S929I, D936Y, S939F, S940F, G946R, D950N, D950H, Q957R, T961M, V963A, S967N, V976F, S982A, K986N, K986R, R995G, 1997V, T998I, Q1005H, T1006I, T1009I, R1014K, A1016V, A1020S, A1025G, T1027I, V1033A, K1038Q, V1040F, K1045N, L1049I, M1050I, A1056V, H1058Y, L1063F, T1066N, Q1071H, K1073N, A1078S, D1084Y, D1084E, A1086S, V1094F, H1101D, H1101Y, V1104L, E1111K, D1118H, D1118Y, G1124V, D1127G, 11130V, V1133F, D1139H, L1141W, D1146Y, E1150D, D1153Y, P1162L, P1162S, D1163Y, N1173S, A1174V, V1176F, E1182D, R1185H, K1191N, N1192S, E1195Q, E1202Q, K1205N, Q1208H, 11216T, G1219V, G1219C, V1228L, M1229I, V1230L, M1237I, T1238I, C1243F, C1247F, S1252F, S1252P, D1260N, P1263L, V1264L, H1271Y, T1273I, and T1273A relative to the amino acid sequence of SEQ ID NO:237 or 250.

255. The method of embodiment 254, in which the amino acid sequence comprises at least three of the mutations.

256. The method of embodiment 254, in which the amino acid sequence comprises at least five of the mutations.

257. The method of embodiment 254, in which the amino acid sequence comprises at least ten of the mutations.

258. The method of any one of embodiments 183 to 257, wherein the amino acid sequence has one, two, three, four, five, six, seven, eight, or nine of the mutations: A69-70, A144, N501Y, A570D, D614G, P681H, T716I, S982A, and D1118H relative to the amino acid sequence of SEQ ID NO:237 or 250.

259. The method of embodiment 258, in which the amino acid sequence comprises at least three of the mutations.

260. The method of embodiment 258, in which the amino acid sequence comprises at least five of the mutations.

261. The method of any one of embodiments 183 to 260, wherein the amino acid sequence has one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, or 106 of the mutations: L5F, L18F, T20N, P26S, V36F, Q52R, D80G, T95I, 1105V, L118F, V127F, D138Y, A156-157, R158G, T167S, D178H, R190S, 1203V, D215G, A222V, 1233V, A242-244, D253G, A262S, P272L, T284I, T299I, V308L, F318S, V227I, P337S, R346S, K356R, V367L, P384L, N394S, R408I, K417T, D427N, N439K, L452R, 1468V, T478K, E484K, L513F, A522S, T531S, N540S, T549I, K558N, E583D, G594S, T604A, Q613H, D614G, V622F, P631S, S640F, H655Y, 1670V, S691P, A701V, T732A, T747I, S758G, G769V, Q779K, D796H, P809S, L822F, A831V, A845S, T859N, 1870V, A879S, F888L, A899S, 1909V, L922F, S939F, D950N, T961M, 1997V, T1006I, A1016V, T1027I, V1040F, L1049I, H1058Y, Q1071H, D1084Y, V1094F, V1104L, 11130V, D1139H, D1153Y, P1162S, V1176F, K1191N, Q1208H, G1219V, V1228L, M1237I, S1252F, V1264L, and T1273I relative to the amino acid sequence of SEQ ID NO:237 or 250.

262. The method of embodiment 261, in which the amino acid sequence comprises at least three of the mutations.

263. The method of embodiment 261, in which the amino acid sequence comprises at least five of the mutations.

264. The method of embodiment 261, in which the amino acid sequence comprises at least ten of the mutations.

265. The method of any one of embodiments 183 to 264, wherein the amino acid sequence has one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, or 111 of the mutations: P9L, T19R, T33I, H49Y, A67V, A69-70, D80A, S98F, S112L, V126A, G142D, W152R, S162I, L176F, L189F, D198Y, 1210T, A222V, D228H, H245Y, W258L, V267L, E281Q, A292S, T307I, T323I, L335F, R346K, R357K, V367F, T376I, T385N, V395I, E406Q, K417N, D427Y, N440K, L452Q, K462T, E471Q, E484K, F490S, N501T, V510L, A520S, V534I, T547I, P561S, A570D, T572I, E583Q, V595I, T604I, A623S, T632S, N641S, A653V, A672V, P681R, A694V, S704L, T716V, M731I, M740V, N751D, T761I, A771S, E780D, T791I, S803A, P812S, T827I, D843N, K854N, A871V, T883I, A892V, M902I, V911I, A924S, D936Y, G946R, Q957R, S967N, V976F, K986N, T998I, T1009I, A1020S, V1033A, K1045N, A1056V, T1066N, A1078S, A1086S, H1101D, E1111K, G1124V, V1133F, D1146Y, P1162L, A1174V, R1185H, E1195Q, K1205N, G1219C, M1229I, T1238I, C1247F, D1260N, and H1271Y relative to the amino acid sequence of SEQ ID NO:237 or 250.

266. The method of embodiment 265, in which the amino acid sequence comprises at least three of the mutations.

267. The method of embodiment 265, in which the amino acid sequence comprises at least five of the mutations.

268. The method of embodiment 265, in which the amino acid sequence comprises at least ten of the mutations.

269. The method of any one of embodiments 183 to 268, wherein the amino acid sequence has one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 of the mutations: V3G, S13I, L18F, A27S, V36I, S45F, L54F, W64R, G75V, T76I, P85S, S94F, D111N, V120L, E132Q, N148T, F157S, S172A, G181V, V193L, Y204H, L216F, V227A, R237K, A246-252, D253N, A263P, R273S, V289L, K300M, E309Q, V320F, P330S, G339S, A348S, V362F, S371T, V382L, N394H, R403K, Q414K, T430I, N440S, L452M, L461F, T470I, T478K, S494P, N501Y, Y505H, A522V, V534F, F543L, E554D, F565L, A570D, A575S, L585F, 1598V, Q607K, D614G, N616S, D627G, T638I, A647S, Y660F, Q677H, A688V, M697I, A706V, T716I, T719I, T732I, T747N, T761R, V772I, V785I, K795R, P812L, V826L, K835R, A845V, T859I, M869I, A879V, A890V, A903S, V915I, S929I, S940F, D950H, V963A, V976F, S982A, K986R, R995G, Q1005H, R1014K, A1025G, K1038Q, M1050I, L1063F, K1073N, D1084E, H1101Y, D1118Y, D1127G, L1141W, E1150D, D1163Y, N1173S, E1182D, N1192S, E1202Q, 11216T, V1230L, C1243F, S1252P, P1263L, and T1273A relative to the amino acid sequence of SEQ ID NO:237 or 250.

270. The method of embodiment 269, in which the amino acid sequence comprises at least three of the mutations.

271. The method of embodiment 269, in which the amino acid sequence comprises at least five of the mutations.

272. The method of embodiment 269, in which the amino acid sequence comprises at least ten of the mutations.

273. The method of any one of embodiments 183 to 272, wherein the amino acid sequence comprises deletion of a signal sequence (e.g., lacks a signal sequence) or inclusion of a signal sequence (e.g., SEQ ID NO: 248).

274. The method of any one of embodiments 183 to 270, wherein the amino acid sequence comprises inclusion of one or more stabilizing mutations (e.g., proline substitutions corresponding to amino acids K969 and V970 of, e.g., SEQ ID NO: 249).

275. The method of any one of embodiments 183 to 274, wherein the amino acid sequence comprises an inactivation of a furin cleavage site (e.g., SEQ ID NO: 246).

276. The method of any one of embodiments 183 to 275, wherein the amino acid sequence comprises inclusion of a trimerization domain (e.g., a foldon trimerization domain, e.g., SEQ ID NO: 243.

277. The method of any one of embodiments 183 to 276, wherein the amino acid sequence comprises inclusion of a linker or spacer sequence(s) (e.g., SEQ ID NOs: 244 and 245).

278. The method of any one of embodiments 183 to 277, wherein the fragment comprises one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, or more amino acids of any one of SEQ ID NOS: 237-266, such as any one of SEQ ID NOS: 237-240 and SEQ ID NOS: 249-266.

279. The method of any one of embodiments 183 to 278, wherein the adenovirus-based vaccine is administered intranasally.

280. The method of any one of embodiments 183 to 279, wherein the adenovirus-based vaccine is administered in two doses 1-13 weeks apart.

281. The method of embodiment 280, wherein the two doses are administered 4-8 weeks apart.

282. The method of embodiment 280 or embodiment 281, wherein the two doses are administered one, two, three, four, five, six, seven, eight, nine, ten, eleven, twelve, or thirteen weeks apart.

283. The method of embodiment 280 or embodiment 281, wherein a booster of the adenovirus-based vaccine is administered annually.

284. The method of any one of embodiments 183 to 282, wherein the adenovirus-based vaccine comprises two or three transgenes, optionally selected from (1) a transgene encoding an amino acid sequence having at least 85%, at least 90%, at least 95%, at least 96%, at least 97%, at least 98%, at least 99% or 100% sequence identity to any one of SEQ ID NOS: 237-240 and SEQ ID NOS: 249-260 or a fragment thereof; (2) a transgene encoding an amino acid sequence having at least 85%, at least 90%, at least 95%, at least 96%, at least 97%, at least 98%, at least 99% or 100% sequence identity to any one of SEQ ID NO:261-263 or a fragment thereof; (3) a transgene encoding an amino acid sequence having at least 85%, at least 90%, at least 95%, at least 96%, at least 97%, at least 98%, at least 99% or 100% sequence identity to any one of SEQ ID NOS: 264-266 or a fragment thereof; and (4) any combination of two or more of the foregoing.

285. The method of embodiment 284, wherein the adenovirus-based vaccine comprises two transgenes, one encoding an amino acid sequence having at least 85%, at least 90%, at least 95%, at least 96%, at least 97%, at least 98%, at least 99% or 100% sequence identity to any one of SEQ ID NOS: 237-240 and SEQ ID NO:249-260 or a fragment thereof, and the other encoding an amino acid sequence having at least 85%, at least 90%, at least 95%, at least 96%, at least 97%, at least 98%, at least 99% or 100% sequence identity to any one of SEQ ID NO:264-266 or a fragment thereof.

This disclosure is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### 7. EXAMPLES

### 7.1. Example 1: HVR analysis of rhesus adenovirus hexon amino acid sequences

Adenovirus hexon amino acid sequences were obtained from GenBank and a multisequence alignment carried out using Geneious v2022.0.2, Build 2022 Jan. 26 14:24, Java Version 11.0.12±7 (64 bit) and Clustal Omega v1.2.2 alignment with following preset parameters:

| Alignment order: | Group sequences by similarity |
|---|---|
| Number of refinement iterations: | 0 |
| Initial guide tree: | Use fast clustering (mBed algorithm) |
| Refinement iteration guide tree: | Use fast clustering (mBed algorithm) |
| Cluster size for mBed guide trees: | 100 |

Following the alignment, the HVRs of human Ad5, which are well characterized by X-ray crystallographic analysis, were used to define the HVRs of RhAds 51-67. An excerpt from the alignment, showing the HVRs of RhAds 51-67 together with those of ChAdOx1, HuAd5, HuAd26 and HuAd48, is shown in FIG. 1.

### 7.2. Example 2: Protein Modeling of Adenovirus Hexon Sequences

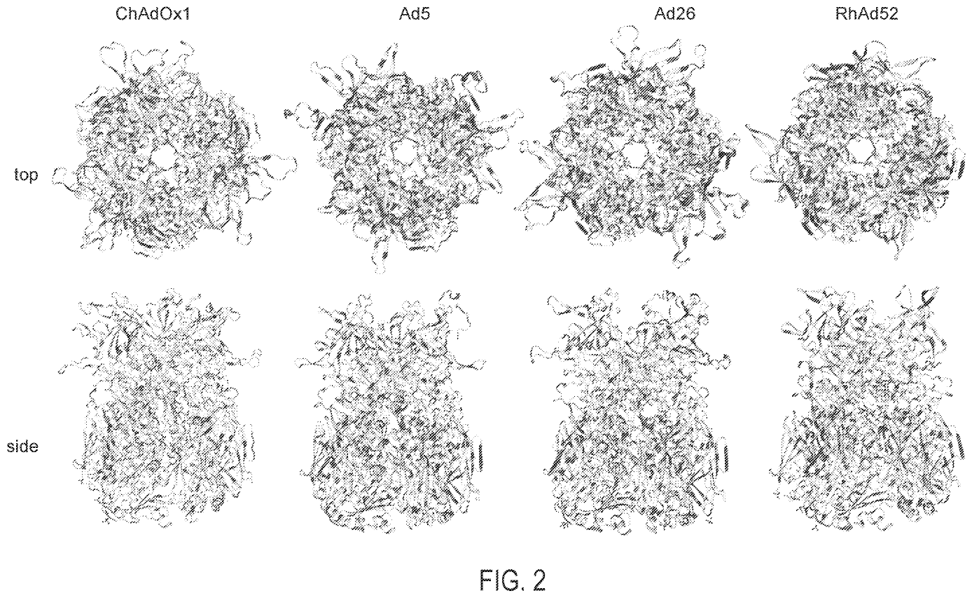

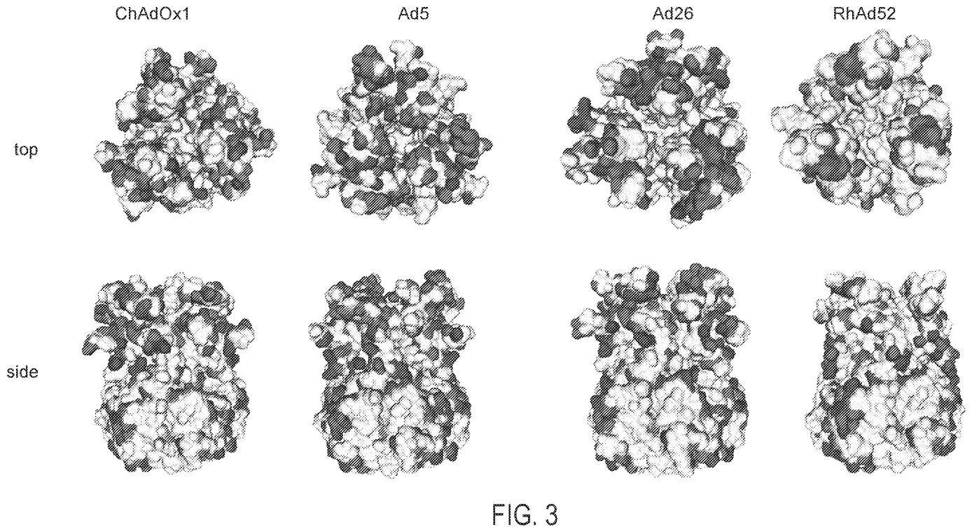

Protein modeling of the hexon sequences for ChAdOx1, Ad5, Ad26 and RhAd52 was performed using the online tool found at the swissmodel.expasy.org web site. Modeling was performed using the standard parameters and using automated alignment with close targets of known hexon protein models. As can be seen from the ribbon structures of FIG. 2 and the cartoons of FIG. 3, the exposed surfaces of RhAd52 show a reduced charge as compared to the surfaces of ChAdOx1, Ad5 and Ad26.

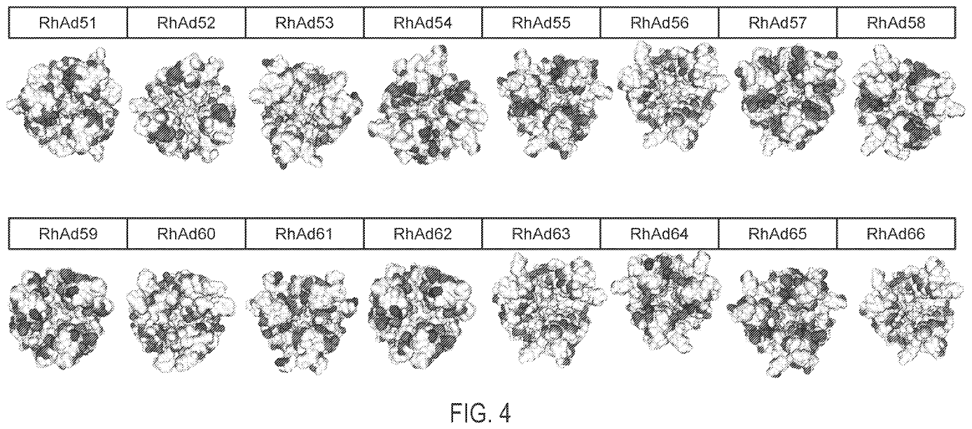

Modeling was then carried out for rhesus adenovirus hexon amino acid sequences for RhAd51; RhAd52; RhAd53; RhAd54; RhAd55; RhAd56; RhAd57; RhAd58; RhAd59; RhAd60; RhAd61; RhAd62; RhAd63; RhAd64; RhAd65; and RhAd66 using the online tool found at the swissmodel.expasy.org website. Modeling was performed using the standard parameters and using automated alignment with close targets of known hexon protein models. As can be seen from the cartons in FIG. 4, all of the RhAds analyzed show reduced surface charges as compared to the surface charges of human and chimp hexon proteins.

### 7.3. Example 3: Determination of the Electrostatic Charge of Adenoviral Hexon Proteins and their HVRs

The overall charges of adenoviral hexon proteins were assessed using the online tool available at the website www.protpi.ch/Calculator/ProteinTool Version: Release 2.2.29.150 Published Jan. 25, 2022. GenBank sequences were inserted into the online tool and the calculation of the charge of the protein was performed at pH 7.4. ProMOST was used as the data source of pKa values for calculation of isoelectric point. The net charge calculator does not take into account the charge on the exposed surface of the protein, but rather of the full amino acid sequence. Referring to FIG. 5, the net electrostatic charge of the protein is indicated by z and the pH at which the protein would be neutrally charged is indicated by pl.

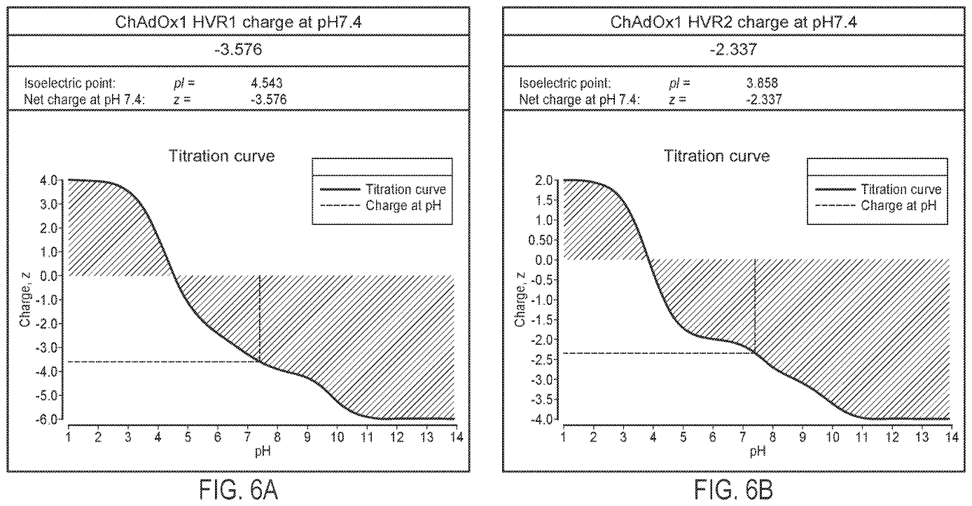

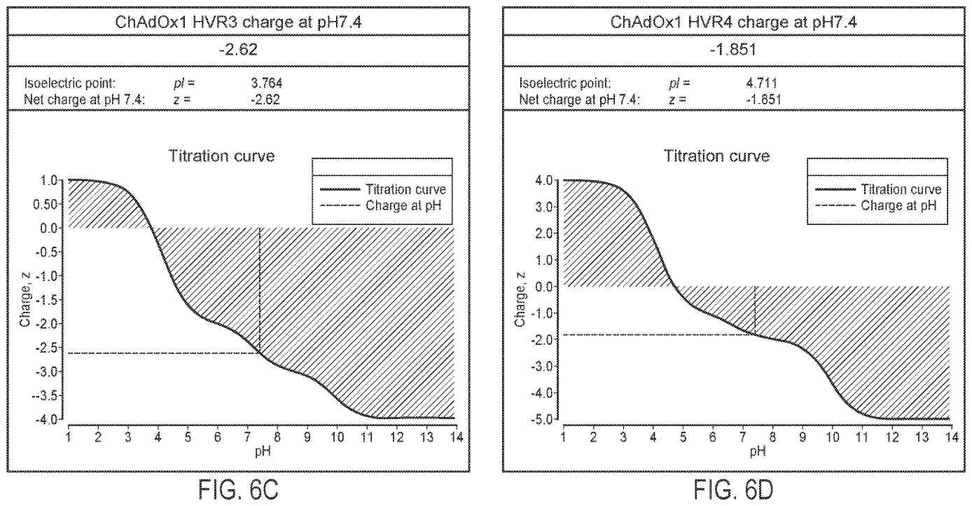

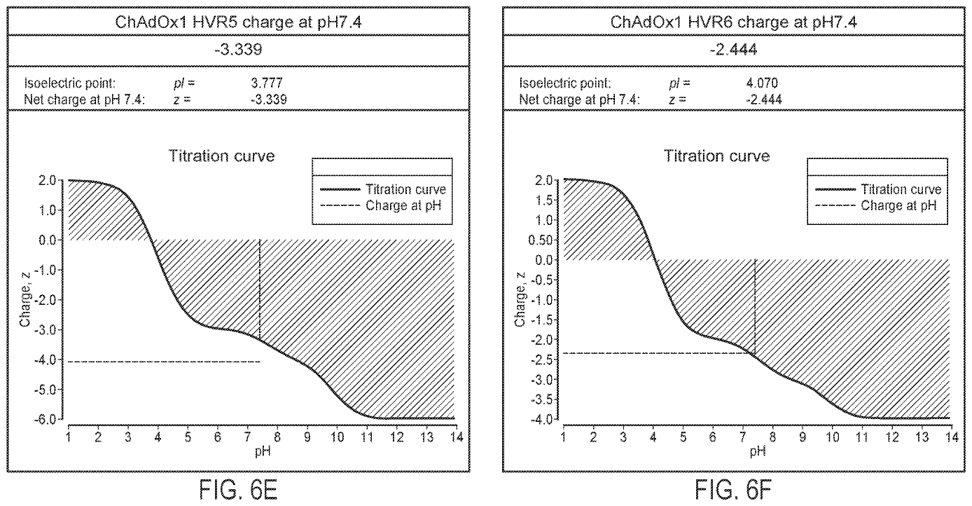

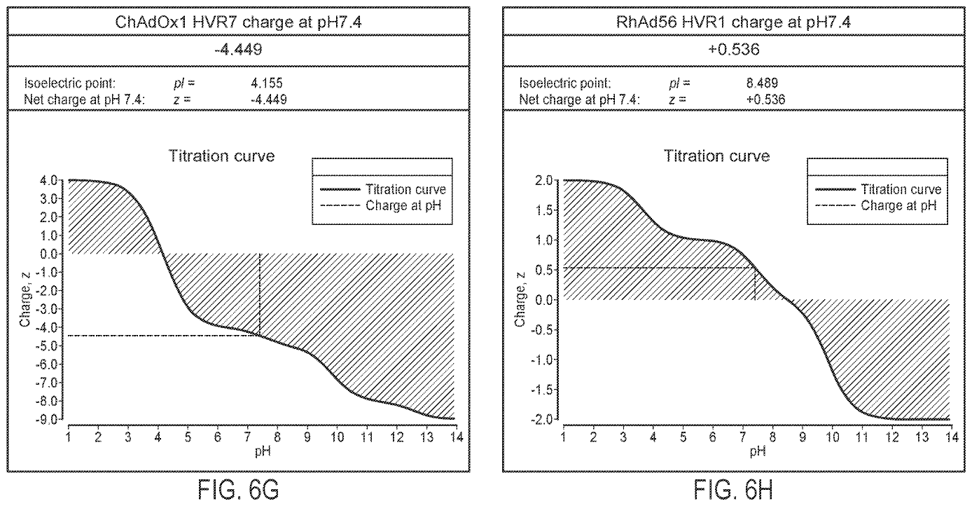

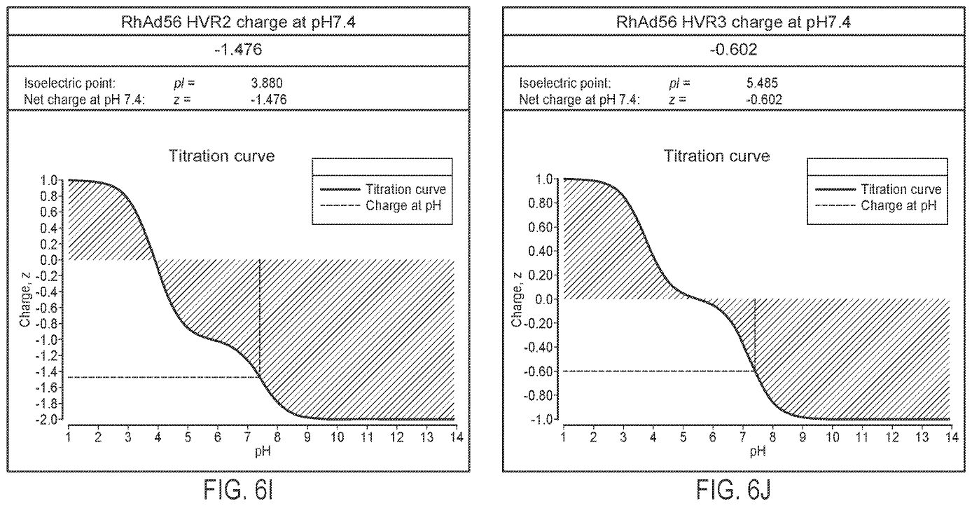

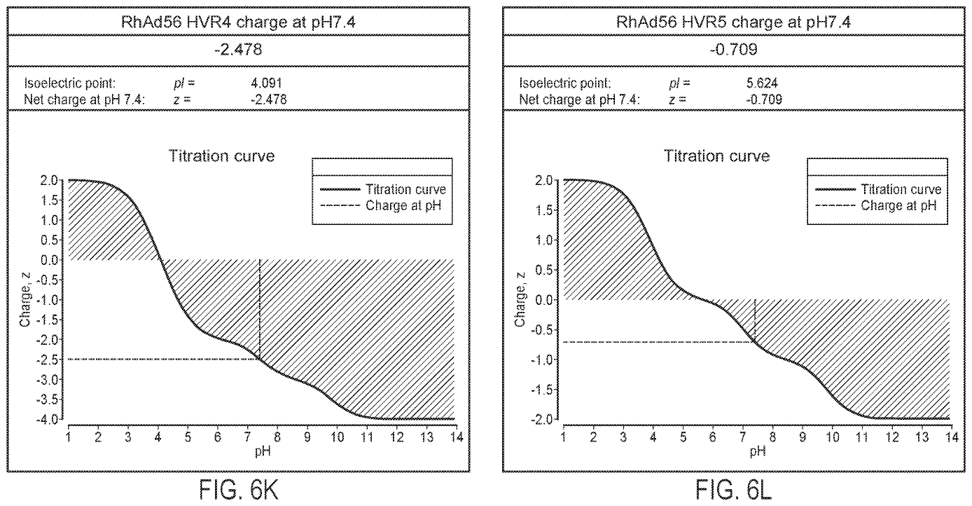

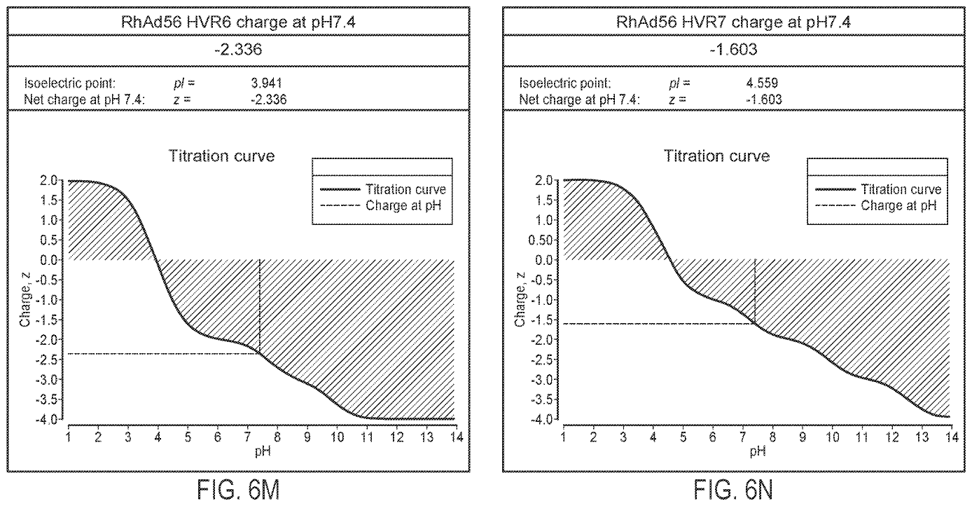

The overall charges of the HVRs for ChAdY25 (also referred to as ChAdOx1), HuAd26, HuAd48 and RhAd51 through RhAd67 were calculated using the tool available at the website www.protpi.ch/Calculator/ProteinTool Version: Release 2.2.29.150 Published on Jan. 25, 2022. The HVR sequences indicated in the sequence alignments of FIG. 1 were individually inserted into the online tool and the calculation of the charge of the protein was performed at pH 7.4. ProMOST was used as the data source of pKa values for calculation of isoelectric point. Referring to FIG. 6, the net electrostatic charge of the protein is indicated by z and the pH at which the protein would be neutrally charged is indicated by pl for HVRs 1-7 for ChAdY25 and RhAd56. Table 1 below shows the electrostatic charge for each of the HVRs of ChAdY25, HuAd26, HuAd48 and RhAd51 through RhAd67, together with the sum of the electrostatic charges for all HVRs of each hexon protein and the average electrostatic charge for the HVRs of each hexon protein.

TABLE 1
Electrostatic virus of hexon protein HVRs

| | | | | | | | | | | Full |
|---|---|---|---|---|---|---|---|---|---|---|
| Ad | HVR1 | HVR2 | HVR3 | HVR4 | HVR5 | HVR6 | HVR7 | Sum | Average | hexon |
| ChAdOx1 | −3.576 | −2.337 | −2.62 | −1.851 | −3.339 | −2.444 | −4.449 | −20.616 | −2.9451 | −22.908 |
| HuAd26 | −0.631 | −2.71 | −2.825 | −2.406 | −3.338 | −2.441 | −3.725 | −18.076 | −2.5823 | −17.912 |
| HuAd48 | −1.579 | −3.458 | −2.83 | −3.407 | −1.337 | −2.444 | −1.417 | −16.472 | −2.3531 | −17.821 |
| RhAd51 | 0.589 | −2.475 | −2.6 | −2.396 | 0.598 | −1.705 | −1.451 | −9.44 | −1.3486 | −16.807 |
| RhAd52 | −0.709 | −1.823 | −0.602 | −1.397 | −1.337 | −2.704 | −0.618 | −9.19 | −1.3129 | −12.922 |
| RhAd53 | 0.284 | −1.476 | −1.601 | −0.484 | −0.605 | −3.332 | −1.615 | −8.829 | −1.2613 | −12.814 |
| RhAd54 | 1.282 | −1.476 | −2.604 | −2.396 | 0.598 | −2.703 | −2.446 | −9.745 | −1.3921 | −14.806 |
| RhAd55 | 0.59 | −1.476 | −1.604 | −2.395 | 0.598 | −2.774 | −3.449 | −10.51 | −1.5014 | −14.813 |
| RhAd56 | 0.539 | −1.476 | −0.602 | −2.478 | −0.709 | −2.336 | −1.603 | −8.665 | −1.2379 | −12.8 |
| RhAd57 | −2.576 | −1.705 | −1.605 | −1.397 | 0.22 | −1.704 | −3.447 | −12.214 | −1.7449 | −15.761 |
| RhAd58 | 1.282 | −1.476 | −1.605 | −2.396 | 0.598 | −2.703 | −3.446 | −9.746 | −1.3923 | −14.807 |
| RhAd59 | −0.607 | −1.659 | −1.605 | −1.397 | 0.598 | −1.453 | −3.447 | −9.57 | −1.3671 | −15.765 |
| RhAd60 | −0.722 | −1.464 | −2.603 | −1.4 | −0.401 | −2.337 | −1.445 | −10.372 | −1.4817 | −16.814 |
| RhAd61 | −0.631 | −0.476 | −2.604 | −2.396 | 0.598 | −1.705 | −1.448 | −8.662 | −1.2374 | −14.809 |
| RhAd62 | −0.607 | −1.705 | −1.605 | −1.397 | 0.598 | −1.453 | −3.447 | −9.616 | −1.3737 | −14.812 |
| RhAd63 | 0.539 | −1.476 | −0.602 | −2.478 | −0.709 | −2.336 | −1.603 | −8.665 | −1.2379 | −13.8 |
| RhAd64 | 0.369 | −1.476 | −2.604 | −2.396 | 0.598 | −1.704 | −2.448 | −9.661 | −1.3801 | −14.819 |
| RhAd65 | 0.59 | −1.476 | −1.604 | −2.395 | 0.598 | −2.774 | −3.449 | −10.51 | −1.5014 | −14.813 |
| RhAd66 | 0.539 | −1.476 | −0.602 | −2.478 | −0.709 | −2.336 | −1.607 | −8.669 | −1.2384 | −13.804 |
| RhAd67 | 0.369 | −1.476 | −2.604 | −2.396 | 0.598 | −1.704 | −2.448 | −9.661 | −1.3801 | −14.819 |

Table 2 below indicates the amino acid boundaries of the individual HVRs within each hexon protein as used in the electrostatic charge calculations of Example 3.

TABLE 2
Amino acid boundaries of HVRs

| Ad | HVR1 | HVR2 | HVR3 | HVR4 | HVR5 | HVR6 | HVR7 |
|---|---|---|---|---|---|---|---|
| ChAdOx1 | 136-164 | 170-180 | 198-205 | 233-252 | 255-273 | 292-303 | 405-444 |
| HuAd26 | 136-170 | 176-187 | 205-212 | 240-259 | 262-283 | 302-313 | 415-454 |
| HuAd48 | 136-167 | 173-185 | 203-210 | 238-258 | 261-282 | 301-312 | 414-449 |
| RhAd51 | 136-165 | 171-180 | 198-208 | 236-252 | 255-267 | 286-299 | 401-434 |
| RhAd52 | 136-161 | 167-173 | 191-199 | 227-234 | 237-250 | 269-280 | 382-414 |
| RhAd53 | 136-160 | 166-175 | 193-201 | 229-247 | 250-264 | 283-296 | 398-430 |
| RhAd54 | 136-162 | 168-178 | 196-206 | 234-248 | 251-263 | 282-295 | 397-433 |
| RhAd55 | 136-162 | 168-178 | 196-206 | 234-248 | 251-264 | 283-196 | 398-434 |
| RhAd56 | 136-162 | 168-172 | 195-203 | 231-249 | 252-265 | 284-297 | 399-433 |
| RhAd57 | 136-158 | 164-173 | 191-201 | 229-237 | 240-252 | 271-284 | 386-420 |
| RhAd58 | 136-162 | 168-178 | 196-206 | 234-248 | 251-263 | 282-295 | 397-433 |
| RhAd59 | 136-158 | 164-173 | 191-201 | 229-237 | 240-252 | 271-284 | 386-421 |
| RhAd60 | 136-159 | 165-175 | 193-201 | 229-237 | 240-253 | 272-283 | 385-423 |
| RhAd61 | 136-165 | 171-181 | 199-209 | 237-254 | 257-269 | 288-301 | 403-439 |
| RhAd62 | 135-158 | 164-173 | 191-201 | 229-237 | 240-252 | 271-284 | 386-421 |
| RhAd63 | 136-162 | 168-172 | 195-203 | 231-249 | 252-265 | 284-297 | 399-433 |
| RhAd64 | 136-162 | 168-178 | 196-206 | 234-250 | 253-265 | 284-297 | 399-436 |
| RhAd65 | 136-162 | 168-178 | 196-206 | 234-248 | 251-263 | 282-295 | 397-433 |
| RhAd66 | 136-162 | 168-177 | 195-203 | 231-249 | 252-165 | 284-297 | 399-433 |
| RhAd67 | 136-162 | 168-178 | 196-206 | 234-250 | 253-165 | 284-297 | 399-436 |

### 7.4. Example 4: Binding of Adenoviruses to Platelet Factor 4

### 7.4.1. Materials & Methods

Surface plasmon assays were performed to compare the binding of human adenovirus 26 (HuAd26) and rhesus adenovirus 52 (RhAd52) platelet factor 4.

The assays were performed using a BIAcore 3000, Cytiva (formerly GE Healthcare). The assay immobilization buffer was HBS-EP [10 mM HEPES, 150 mM NaCl, 3 mM EDTA, and 0.005% (v/v) Surfactant P20]. Virus at ˜1×10¹¹ VP/ml was diluted 1:5 in acetate 4.5 buffer and immobilized to a C-1 sensor chip using a standard amine coupling protocol involving several 5-10 min sample injection cycles. Typically, 400 to 500 RU of virus was immobilized. A reference sensor surface was created using the same amine coupling protocol but without the virus. Samples were injected with an association time of 120 s and a dissociation time of 120 seconds at a flow rate of 50 μL/min. The surface was regenerated with a 30-s injection of 25 mM NaOH at a flow rate of 50 μL/min. All sensorgram plots were subtracted from the reference flow cell and a buffer cycle to remove the nonspecific responses, bulk refractive index changes, and systematic instrument noise.

### 7.4.2. Results

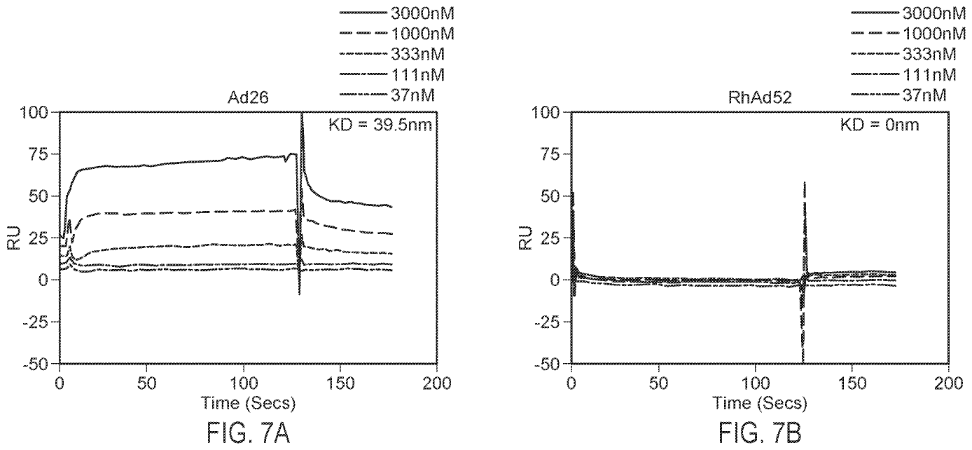

The results of the study are shown in FIG. 7. FIG. 7 demonstrates that RhAd52 had no detectable binding to PF4, while the negatively charged HuAd26 binds to PF4 with a low nanomolar dissociation constant (kD). The binding of PF4 to polyanions results in conformational changes that expose binding site(s) for anti-PF4/polyanion complex antibodies. The binding of negatively charged non-rhesus adenoviral proteins to PF4 is believed to stimulate memory B-cells, leading to the production of such anti-PF4 antibodies. Complexes of PF4 and the antibodies can lead to platelet activation and consequent thrombocytopenia, as occurs the prothrombotic adverse drug reaction heparin-induced thrombocytopenia (HIT) and adenoviral vaccine-induced TTS. See Nguyen et al., 2017, Nat. Commun. 8:14945 and Baker et al., 2021, Sci. Adv. 7: eabl8213.

In contrast, as shown herein, rhesus adenovirus proteins have low surface negative charges and do not detectably bind to PF4. Accordingly, the inventors believe that the use of rhesus adenoviral vectors does not initiate TTS or similar prothrombotic events, making rhesus adenoviral vectors particularly well-suited for individuals at risk of TTS.

### 8. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

### 9. SEQUENCES

The following table provides sequences referenced in the disclosure, with “NO” referencing SEQ ID NO:, “Source” referencing virus of origin (or consensus), and “Desc.” describing viral protein or protein region.

| NO | Source | Desc. | Sequence |
|---|---|---|---|
| 1 | Consensus | HVR1 | WETTDNKTKVRAQAPFXSTAITKEG |
| 2 | HuAd5 | HVR1 | WDEAATALEINLEEEDDDNEDEVDEQAEQQKTHVFGQAPYS |
| | | | GINITKEG |
| 3 | ChAdOx1 | HVR1 | WEQKKAGNGDTMETHTFGVAPMGGENITIDG |
| 4 | HuAd26 | HVR1 | WETKEKQGTTGGVQQEKDVTKTFGVAATGGINITNQG |
| 5 | HuAd48 | HVR1 | WEEKKNGGGSDANQMQTHTFGVAAMGGIEITAKG |
| 6 | RhAd51 | HVR1 | WADTNDSNKLKVRGQAPFVSTYGSATALTKDG |
| 7 | RhAd52 | HVR1 | WTTTNGGNKTNTFAQAPLIGTAIDGTNG |
| 8 | RhAd53 | HVR1 | WTTTNGGNKTNSFAQASYIGQSLSKDG |
| 9 | RhAd54 | HVR1 | WTNSDSKVKVRAQAPFVSSYGATAITKEG |
| 10 | RhAd55 | HVR1 | WADSDSKLKVRAQAPFVSSYGNNAITKEG |
| 11 | RhAd56 | HVR1 | WITNGGTAKTNSFAQASYIGMGANLTTNG |
| 12 | RhAd57 | HVR1 | WEGSDNKTHVRGQAPFISDEITKEG |
| 13 | RhAd58 | HVR1 | WTNTDSKVKVRAQAPFVSSYGATAITKEG |
| 14 | RhAd59 | HVR1 | WNGTDSKTNVRGQAPFISDAITKEG |
| 15 | RhAd60 | HVR1 | WTDTSDNKLKAYAQAPYQSQGLTKDG |
| 16 | RhAd61 | HVR1 | WEDTNDSNKLKVRGQAPYVSTFGAATALTKDG |
| 17 | RhAd62 | HVR1 | WNGTDSKTNVRGQAPFISDAITKEG |
| 18 | RhAd63 | HVR1 | WITNGGTAKTNSFAQASYIGMGANLTTNG |
| 19 | RhAd64 | HVR1 | WENSDNKLKVRAQAPFVSSYGATAITKDG |
| 20 | RhAd65 | HVR1 | WADSDSKLKVRAQAPFVSSYGNNAITKEG |
| 21 | RhAd66 | HVR1 | WITNGGTAKTNSFAQASYIGMGANLTTNG |
| 22 | RhAd67 | HVR1 | WENSDNKLKVRAQAPFVSSYGATAITKDG |
| 23 | Consensus | HVR2 | VGVDTTDSXTAQY |
| 24 | HuAd5 | HVR2 | IGVEGQTPKY |
| 25 | ChAdOx1 | HVR2 | IGTDATADQDKPIY |
| 26 | HuAd26 | HVR2 | LGTDETAENGKKDIY |
| 27 | HuAd48 | HVR2 | IGIDATKEEDNGKEIY |
| 28 | RhAd51 | HVR2 | VGVDTSEASQAVY |
| 29 | RhAd52 | HVR2 | IGQDNGQAVY |
| 30 | RhAd53 | HVR2 | VAVDTAAGGAAVY |
| 31 | RhAd54 | HVR2 | VGVTLTDSGSTPQY |
| 32 | RhAd55 | HVR2 | VGVILTDSGQTAQY |
| 33 | RhAd56 | HVR2 | VAVDTNNANAAVY |
| 34 | RhAd57 | HVR2 | VGTDTGNPGQAIY |
| 35 | RhAd58 | HVR2 | VGVVLTDSGSTPQY |
| 36 | RhAd59 | HVR2 | VGTDTGNHGQAIY |
| 37 | RhAd60 | HVR2 | VGLWVTESGQTPQY |
| 38 | RhAd61 | HVR2 | VGVTVSESARTPQY |
| 39 | RhAd62 | HVR2 | VGTDTGNPGQAIY |
| 40 | RhAd63 | HVR2 | VAVDTNNANAAVY |
| 41 | RhAd64 | HVR2 | VGWLTDSTQTPQY |
| 42 | RhAd65 | HVR2 | VGVILTDSGQTAQY |
| 43 | RhAd66 | HVR2 | VAVDTNNANAAVY |
| 44 | RhAd67 | HVR2 | VGWVLTDSTQTPQY |
| 45 | Consensus | HVR3 | WNSEVGTDKAA |
| 46 | HuAd5 | HVR3 | WYETEINHAA |
| 47 | ChAdOx1 | HVR3 | WQETESFYG |
| 48 | HuAd26 | HVR3 | WQENEAFYG |
| 49 | HuAd48 | HVR3 | WQDSDNYYG |
| 50 | RhAd51 | HVR3 | WNSEVGNDDRVA |
| 51 | RhAd52 | HVR3 | WNTNPTTNAA |
| 52 | RhAd53 | HVR3 | WNENPTTNAA |
| 53 | RhAd54 | HVR3 | WNSDVGTDDKIA |
| 54 | RhAd55 | HVR3 | WNSEVGTNDKVA |
| 55 | RhAd56 | HVR3 | WNINPTQNAA |
| 56 | RhAd57 | HVR3 | WNSDVGTNDKAA |
| 57 | RhAd58 | HVR3 | WNSDVGTNDKIA |
| 58 | RhAd59 | HVR3 | WNSDVGATDKAA |
| 59 | RhAd60 | HVR3 | WNLEQEDKAA |
| 60 | RhAd61 | HVR3 | WNSEVGNDDKLA |
| 61 | RhAd62 | HVR3 | WNSDVGATDKAA |
| 62 | RhAd63 | HVR3 | WNINPTQNAA |
| 63 | RhAd64 | HVR3 | WNSEVGNDDKVA |
| 64 | RhAd65 | HVR3 | WNSEVGTNDKVA |
| 65 | RhAd66 | HVR3 | WNINPTQNAA |
| 66 | RhAd67 | HVR3 | WNSEVGNDDKVA |
| 67 | Consensus | HVR4 | ATQPAXDXXNNVELQF |
| 68 | HuAd5 | HVR4 | GILVKQQNGKLESQVEMQF |
| 69 | ChAdOx1 | HVR4 | AKLKVGADGVPTKEFDIDLAF |
| 70 | HuAd26 | HVR4 | AKFKPVNEGEQPKDLDIDFAY |
| 71 | HuAd48 | HVR4 | AKFKTPEKEGEEPKELDIDLNF |
| 72 | RhAd51 | HVR4 | AIQPTAGNGDNQAVELQF |
| 73 | RhAd52 | HVR4 | ASGDVTFQF |
| 74 | RhAd53 | HVR4 | VKITDPNNDKTGANNVSLNF |
| 75 | RhAd54 | HVR4 | ATPSASQDVQNPELQF |
| 76 | RhAd55 | HVR4 | ATQPASQETQNPELQF |
| 77 | RhAd56 | HVR4 | VKITDGSTDPTGANSVSMEF |
| 78 | RhAd57 | HVR4 | ATNQQVDLQF |
| 79 | RhAd58 | HVR4 | ATPSASQDVQNPELQF |
| 80 | RhAd59 | HVR4 | ATNQQVDMQF |
| 81 | RhAd60 | HVR4 | AKNQEVDLQF |
| 82 | RhAd61 | HVR4 | AIQPAGSSGDTNPAVELQF |
| 83 | RhAd62 | HVR4 | ATNQQVDMQF |
| 84 | RhAd63 | HVR4 | VKITDGSTDPTGANSVSMEF |
| 85 | RhAd64 | HVR4 | ATQPAAGQDAQNAVELQF |
| 86 | RhAd65 | HVR4 | ATQPASQETQNPELQF |
| 87 | RhAd66 | HVR4 | VKITDGTTDPTGANSVSMEF |
| 88 | RhAd67 | HVR4 | ATQPAAGQDAQNAVELQF |
| 89 | Consensus | HVR5 | FATTSNNNTPKVV |
| 90 | HuAd5 | HVR5 | FSTTEAAAGNGDNLTPKVV |
| 91 | ChAdOx1 | HVR5 | FDTPGGTVNGQDEYKADIV |
| 92 | HuAd26 | HVR5 | FDVPGGSPPAGGSGEEYKADII |
| 93 | HuAd48 | HVR5 | FDIPSTGTGGNGTNVNFKPDMI |
| 94 | RhAd51 | HVR5 | FATTSTPTAPKAV |
| 95 | RhAd52 | HVR5 | FDTASDNGNNPKVV |
| 96 | RhAd53 | HVR5 | FNTAADNGNNNPKWV |
| 97 | RhAd54 | HVR5 | FASTNVANTPKAV |
| 98 | RhAd55 | HVR5 | FAASSTTVNTPKAV |
| 99 | RhAd56 | HVR5 | FTTTADNQNQAKVV |
| 100 | RhAd57 | HVR5 | FSTGSSNTTPKWV |
| 101 | RhAd58 | HVR5 | FASTNVANTPKAV |
| 102 | RhAd59 | HVR5 | FATGSSNTTPKWV |
| 103 | RhAd60 | HVR5 | FATPGDTQNTAKVV |
| 104 | RhAd61 | HVR5 | FATTSATTTPKAV |
| 105 | RhAd62 | HVR5 | FATGSSNTTPKWV |
| 106 | RhAd63 | HVR5 | FTTTADNQNQAKVV |
| 107 | RhAd64 | HVR5 | FATTSATTTPKAV |
| 108 | RhAd65 | HVR5 | FAASSTVNTPKAV |
| 109 | RhAd66 | HVR5 | FTTTADNQNQAKVV |
| 110 | RhAd67 | HVR5 | FATTSATTTPKAV |
| 111 | Consensus | HVR6 | PTVTAGTTSSEALLGQ |
| 112 | HuAd5 | HVR6 | PTIKEGNSRELMGQ |
| 113 | ChAdOx1 | HVR6 | PGKDDASSEINLVQ |
| 114 | HuAd26 | HVR6 | PGTSDNSSEINLVQ |
| 115 | HuAd48 | HVR6 | PGKEDASSESNLTQ |
| 116 | RhAd51 | HVR6 | PTWVAGTTSSEALLTQ |
| 117 | RhAd52 | HVR6 | PTADNTNSENLLGQ |
| 118 | RhAd53 | HVR6 | PDVTGDATSAETLLGQ |
| 119 | RhAd54 | HVR6 | PTVTEGITSSEALLTQ |
| 120 | RhAd55 | HVR6 | PSVTEGTTSSEALLTQ |
| 121 | RhAd56 | HVR6 | PDVADNAKAAETLLGQ |
| 122 | RhAd57 | HVR6 | PTVTAGVTNAEALLAQ |
| 123 | RhAd58 | HVR6 | PTVTEGITSSEALLTQ |
| 124 | RhAd59 | HVR6 | PIVTAGTTNAEALLAQ |
| 125 | RhAd60 | HVR6 | PDDDSTSSKLLLGQ |
| 126 | RhAd61 | HVR6 | PTVTAGTTSSEALLTQ |
| 127 | RhAd62 | HVR6 | PIVTAGTTNAEALLAQ |
| 128 | RhAd63 | HVR6 | PDVADNAKAAETLLGQ |
| 129 | RhAd64 | HVR6 | PTVTAGTTSSEALLSQ |
| 130 | RhAd65 | HVR6 | PSVTEGTTSSEALLAQ |
| 131 | RhAd66 | HVR6 | PDVADNAKAAETLLGQ |
| 132 | RhAd67 | HVR6 | PTVTAGTTSSEALLSQ |
| 133 | Consensus | HVR7 | GMGVTDTYTGIKXNNGGGNTWEANBDVFAXRGXE |
| 134 | HuAd5 | HVR7 | GVINTETLTKVKPKTGQENGWEKDATEFSDKNE |
| 135 | ChAdOx1 | HVR7 | GSGTNAAYQGVKVKNGNDGDVESEWENDDTVAARNQ |
| 136 | HuAd26 | HVR7 | GTGTNSTYQGVKITNGNDGAEESEWEKDDAISRQNQ |
| 137 | HuAd48 | HVR7 | GAGTNAVYQGVKVKTTNNTEWEKDTAVSEHNQ |
| 138 | RhAd51 | HVR7 | GMVVTDTYKAIKVNGSGWTANTDVFSERVE |
| 139 | RhAd52 | HVR7 | AQGIANTYKGVKKNNGNWAKDDAVVETNE |
| 140 | RhAd53 | HVR7 | GQGISNTYKGVKYNTNTWTQDTDVVTTNE |
| 141 | RhAd54 | HVR7 | GMAVTDTYSPIKVNGGGNGWEANNGVFTERGVE |
| 142 | RhAd55 | HVR7 | GMAVTDTYSPIKLNGGQNGWEANNDVYAERGVE |
| 143 | RhAd56 | HVR7 | GQGISNTYRGLSVQNGQNTWQANNNAAENNE |
| 144 | RhAd57 | HVR7 | GMAVTDTYSGIKPSDNGGTFQADNTVADRVE |
| 145 | RhAd58 | HVR7 | GMAVTDTYSPIKVNTAGNGWEANNDVFTDRGVE |
| 146 | RhAd59 | HVR7 | GMAVTDTYTGIKPDNGGTTFSNDDTFATRGIE |
| 147 | RhAd60 | HVR7 | GMVVTDNYNSVTPQNGGSGNTWQADNTTFSQRGAQ |
| 148 | RhAd61 | HVR7 | GMVVTDTYTPIKPNGSGSVTWTANTDVFSNRVE |
| 149 | RhAd62 | HVR7 | GMAVTDTYTGIKPDNGGTTFSNDDTFATRGIE |
| 150 | RhAd63 | HVR7 | GQGISNTYRGLSVQNGQNTWQANNNAAENNE |
| 151 | RhAd64 | HVR7 | GTVTDTYTPIQPNSNGGGTWVAKDNVFTDRGVE |
| 152 | RhAd65 | HVR7 | GMAVTDTYSPIKLNGGQNGWEANNDVYAERGVE |
| 153 | RhAd66 | HVR7 | GQGISNTYKGLSVQNGQNTWQANNNAAENNE |
| 154 | RhAd67 | HVR7 | GTVVTDTYTPIQPNSNGGGTWVAKDNVFTDRGVE |
| 155 | HuAd5 | Hexon | MATPSMMPQWSYMHISGQDASEYLSPGLVQFARATETYFS |
| | | protein | LNNKFRNPTVAPTHDVTTDRSQRLTLRFIPVDREDTAYSY |
| | | | KARFTLAVGDNRVLDMASTYFDIRGVLDRGPTFKPYSGTA |
| | | | YNALAPKGAPNPCEWDEAATALEINLEEEDDDNEDEVDEQ |
| | | | AEQQKTHVFGQAPYSGINITKEGIQIGVEGQTPKYADKTF |
| | | | QPEPQIGESQWYETEINHAAGRVLKKTTPMKPCYGSYAKP |
| | | | TNENGGQGILVKQQNGKLESQVEMQFFSTTEATAGNGDNL |
| | | | TPKWLYSEDVDIETPDTHISYMPTIKEGNSRELMGQQSMP |
| | | | NRPNYIAFRDNFIGLMYYNSTGNMGVLAGQASQLNAVVDL |
| | | | QDRNTELSYQLLLDSIGDRTRYFSMWNQAVDSYDPDVRII |
| | | | ENHGTEDELPNYCFPLGGVINTETLTKVKPKTGQENGWEK |
| | | | DATEFSDKNEIRVGNNFAMEINLNANLWRNFLYSNIALYL |
| | | | PDKLKYSPSNVKISDNPNTYDYMNKRWVAPGLVDCYINLG |
| | | | ARWSLDYMDNVNPFNHHRNAGLRYRSMLLGNGRYVPFHIQ |
| | | | VPQKFFAIKNLLLLPGSYTYEWNFRKDVNMVLQSSLGNDL |
| | | | RVDGASIKFDSICLYATFFPMAHNTASTLEAMLRNDTNDQ |
| | | | SFNDYLSAANMLYPIPANATNVPISIPSRNWAAFRGWAFT |
| | | | RLKTKETPSLGSGYDPYYTYSGSIPYLDGTFYLNHTFKKV |
| | | | AITFDSSVSWPGNDRLLTPNEFEIKRSVDGEGYNVAQCNM |
| | | | TKDWFLVQMLANYNIGYQGFYIPESYKDRMYSFFRNFQPM |
| | | | SRQVVDDTKYKDYQQVGILHQHNNSGFVGYLAPTMREGQA |
| | | | YPANFPYPLIGKTAVDSITQKKFLCDRTLWRIPFSSNFMS |
| | | | MGALTDLGQNLLYANSAHALDMTFEVDPMDEPTLLYVLFE |
| | | | VFDWRVHRPHRGVIETVYLRTPFSAGNATT |
| 156 | ChAdOx1 | Hexon | MATPSMLPQWAYMHIAGQDASEYLSPGLVQFARATDTYFS |
| | | protein | LGNKFRNPTVAPTHDVTTDRSQRLTLRFVPVDREDNTYSY |
| | | | KVRYTLAVGDNRVLDMASTYFDIRGVLDRGPSFKPYSGTA |
| | | | YNSLAPKGAPNSSQWEQKKAGNGDTMETHTFGVAPMGGEN |
| | | | ITIDGLQIGTDATADQDKPIYADKTFQPEPQVGEENWQET |
| | | | ESFYGGRALKKDTSMKPCYGSYARPTNVKGGQAKLKVGAD |
| | | | GVPTKEFDIDLAFFDTPGGTVNGQDEYKADIVMYTENTYL |
| | | | ETPDTHVVYKPGKDDASSEINLVQQSMPNRPNYIGFRDNF |
| | | | IGLMYYNSTGNMGVLAGQASQLNAVVDLQDRNTELSYQLL |
| | | | LDSLGDRTRYFSMWNQAVDSYDPDVRIIENHGVEDELPNY |
| | | | CFPLDGSGTNAAYQGVKVKNGNDGDVESEWENDDTVAARN |
| | | | QLCKGNIFAMEINLQANLWRSFLYSNVALYLPDSYKYTPA |
| | | | NITLPTNTNTYDYMNGRWPPSLVDAYINIGARWSLDPMDN |
| | | | VNPFNHHRNAGLRYRSMLLGNGRYVPFHIQVPQKFFAIKS |
| | | | LLLLPGSYTYEWNFRKDVNMILQSSLGNDLRTDGASISFT |
| | | | SINLYATFFPMAHNTASTLEAMLRNDTNDQSFNDYLSAAN |
| | | | MLYPIPANATNVPISIPSRNWAAFRGWSFTRLKTKETPSL |
| | | | GSGFDPYFVYSGSIPYLDGTFYLNHTFKKVSITFDSSVSW |
| | | | PGNDRLLTPNEFEIKRTVDGEGYNVAQCNMTKDWFLVQML |
| | | | AHYNIGYQGFYVPEGYKDRMYSFFRNFQPMSRQVVDEVNY |
| | | | KDYQAVTLAYQHNNSGFVGYLAPTMRQGQPYPANYPYPLI |
| | | | GKSAVTSVTQKKFLCDRVMWRIPFSSNFMSMGALTDLGQN |
| | | | MLYANSAHALDMNFEVDPMDESTLLYVVFEVFDWRVHQPH |
| | | | RGVIEAVYLRTPFSAGNATT |
| 157 | HuAd26 | Hexon | MCLTARERAKMATPSMMPQWAYMHIAGQDASEYLSPGLVQ |
| | | protein | FARATDTYFSLGNKFRNPTVAPTHDVTTDRSQRLTLRFVP |
| | | | VDREDTTYSYKARFTLAVGDNRVLDMASTYFDIRGVLDRG |
| | | | PSFKPYSGTAYNSLAPKGAPNPSQWETKEKQGTTGGVQQE |
| | | | KDVTKTFGVAATGGINITNQGLLLGTDETAENGKKDIYAD |
| | | | KTFQPEPQVGEENWQENEAFYGGRALKKDTKMKPCYGSFA |
| | | | RPTNEKGGQAKFKPVNEGEQPKDLDIDFAYFDVPGGSPPA |
| | | | GGSGEEYKADIILYTENVNLETPDTHVVYKPGTSDNSSEI |
| | | | NLVQQSMPNRPNYIGFRDNFVGLMYYNSTGNMGVLAGQAS |
| | | | QLNAVVDLQDRNTELSYQLLLDSLGDRTRYFSMWNSAVDS |
| | | | YDPDVRIIENHGVEDELPNYCFPLNGTGTNSTYQGVKITN |
| | | | GNDGAEESEWEKDDAISRQNQICKGNVYAMEINLQANLWK |
| | | | SFLYSNVALYLPDSYKYTPANVKLPANTNTYEYMNGRWVA |
| | | | PSLVDAYINIGARWSLDPMDNVNPFNHHRNAGLRYRSMLL |
| | | | GNGRYVPFHIQVPQKFFAIKNLLLLPGSYTYEWNFRKDVN |
| | | | MILQSSLGNDLRVDGASVRFDSVNLYATFFPMAHNTASTL |
| | | | EAMLRNDTNDQSFNDYLSAANMLYPIPAKATNVPISIPSR |
| | | | NWAAFRGWSFTRLKTKETPSLGSGFDPYFVYSGSIPYLDG |
| | | | TFYLNHTFKKVSIMFDSSVSWPGNDRLLTPNEFEIKRSVD |
| | | | GEGYNVAQCNMTKDWFLVQMLSHYNIGYQGFHVPEGYKDR |
| | | | MYSFFRNFQPMSRQWVDEINYKDYKAVTLPFQHNNSGFTG |
| | | | YLAPTMRQGQPYPANFPYPLIGQTAVPSVTQKKFLCDRVM |
| | | | WRIPFSSNFMSMGALTDLGQNMLYANSAHALDMTFEVDPM |
| | | | DEPTLLYLLFEVFDWVRVHQPHRGVIEAVYLRTPFSAGNA |
| | | | TT |
| 158 | HuAd48 | Hexon | MATPSMMPQWAYMHIAGQDASEYLSPGLVQFARATDTYFS |
| | | protein | LGNKFRNPTVAPTHDVTTDRSQRLTLRFVPVDREDTTYSY |
| | | | KARFTLAVGDNRVLDMASTYFDIRGVLDRGPSFKPYSGTA |
| | | | YNSLAPKGAPNPSQWEEKKNGGGSDANQMQTHTFGVAAMG |
| | | | GIEITAKGLQIGIDATKEEDNGKEIYADKTFQPEPQIGEE |
| | | | NWQDSDNYYGGRAIKKETKMKPCYGSFARPTNEKGGQAKF |
| | | | KTPEKEGEEPKELDIDLNFFDIPSTGTGGNGTNVNFKPDM |
| | | | IMYAENVNLETPDTHIVYKPGKEDASSESNLTQQSMPNRP |
| | | | NYIGFRDNFVGLMYYNSTGNMGVLAGQASQLNAVVDLQDR |
| | | | NTELSYQLLLDSLGDRTRYFSMWNSAVDSYDPDVRIIENH |
| | | | GVEDELPNYCFPLDGAGTNAVYQGVKVKTTNNTEWEKDTA |
| | | | VSEHNQICKGNVYAMEINLQANLWKSFLYSNVALYLPDSY |
| | | | KYTPANVTLPTNTNTYEYMNGRWAPSLVDAYINIGARWSL |
| | | | DPMDNVNPFNHHRNAGLRYRSMLLGNGRYVPFHIQVPQKF |
| | | | FAIKNLLLLPGSYTYEWNFRKDVNMILQSSLGNDLRVDGA |
| | | | SVRFDSVNLYATFFPMAHNTASTLEAMLRNDTNDQSFNDY |
| | | | LSAANMLYPIPAKATNVPISIPSRNWAAFRGWSFTRLKTK |
| | | | ETPSLGSGFDPYFVYSGSIPYLDGTFYLNHTFKKVSIMFD |
| | | | SSVSWPGNDRLLTPNEFEIKRSVDGEGYNVAQCNMTKDWF |
| | | | LVQMLSHYNIGYQGFHVPEGYKDRMYSFFRNFQPMSRQWV |
| | | | DEINYKDYKAVTLPFQHNNSGFTGYLAPTMRQGQPYPANF |
| | | | PYPLIGQTAVPSVTQKKFLCDRVMWRIPFSSNFMSMGALT |
| | | | DLGQNMLYANSAHALDMTFEVDPMDEPTLLYLLFEVFDWV |
| | | | RVHQPHRGVIEAVYLRTPFSAGNATT |
| 159 | RhAd51 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPAEWADTNDSNKLKVRGQAPFVSTYGSAT |
| | | | ALTKDGIQVGVDTSEASQAVYADRSYQPEPQIGETEWNSE |
| | | | VGNDDRVAGRVLKKTTPMFPCYGSYAKPTNEKGGQAIQPT |
| | | | AGNGDNQAVELQFFATTSTPTAPKAVLYAEDVAIEAPDTH |
| | | | LVFKPTWVAGTTSSEALLTQQAAPNRPNYIAFRDNFIGLM |
| | | | YYNSTGNMGVLAGQASQLNAVVDLQDRNTELSYQLMLDAL |
| | | | GDRSRYFSMWNQAVDSYDPDVRIVENHGVEDELPNYCFPL |
| | | | GGMVVTDTYKAIKVNGSGWTANTDVFSERVEIGSGNLFAM |
| | | | EINLQANLWRSFLYSNIGLYLPDSLKLTPDNITLPENKNT |
| | | | YQYMNGRVTPPGLVDTYVNVGARWSPDVMDSINPFNHHRN |
| | | | AGLRYRSMLLGNGRYVPFHIQVPQKFFAIKNLLLLPGSYT |
| | | | YEWNFRKDVNMILQSSLGNDLRVDGASIRFDSINLYANFF |
| | | | PMAHNTASTLEAMLRNDTNDQSFNDYLCAANMLYPIPANA |
| | | | TSVPISIPSRNWAAFRGWSFTRLKTKETPSLGSGFDPYFV |
| | | | YSGSIPYLDGTFYLNHTFKKVSIMFDSSVSWPGNDRLLTP |
| | | | NEFEIKRSVDGEGYNVAQSNMTKDWFLVQMLSHYNIGYQG |
| | | | FYVPENYKDRMYSFFRNFQPMSRQVVDSVAYRDYYQDVKL |
| | | | PYQHNNSGFVGYMGPTMREGQAYPANYPYPLIGETAVPSL |
| | | | TQKKFLCDRVMWRIPFSSNFMSMGSLTDLGQNMLYANSAH |
| | | | ALDMTFEVDPMDEPTLLYVLFEVFDWVRIHQPHRGVIEAV |
| | | | YLRTPFSAGNATT |
| 160 | RhAd52 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYTGTA |
| | | | YNALAPKGAPNACQWTTTNGGNKTNTFAQAPLIGTAIDGT |
| | | | NGLQIGQDNGQAVYADKTFQPEPQVGESQWNTNPTTNAAG |
| | | | RVLKTTTRMLPCYGSFARPTNEKGGQASGDVTFQFFDTAS |
| | | | DNGNNPKVVLYGEDVNIESPDTHLIYKPTADNTNSENLLG |
| | | | QQAAPNRANYIAFRDNFIGLMYYNSTGNMGVLAGQASQLN |
| | | | AVVDLQDRNTELSYQLMLDAIGDRSRYFSMWNQAVDSYDP |
| | | | DVRIIENHGVEDELPNYCFPLNAQGIANTYKGVKKNNGNW |
| | | | AKDDAWETNEIGIGNVFAMEINLTANLWRNFLYSNIALYL |
| | | | PDSYKYSPGNITLPENKNSYNYINGRVTAPGLVDTFVNIG |
| | | | ARWSPDPMDNVNPFNHHRNAGLRYRSMLLGNGRYVPFHIQ |
| | | | VPQKFFAIKNLLLLPGSYTYEWNFRKDVNMILQSTLGNDL |
| | | | RVDGASVRFDSINLYANFFPMAHNTASTLEAMLRNDTNDQ |
| | | | SFNDYLCAANMLYPIPANATSVPISIPSRNWAAFRGWSFT |
| | | | RLKTKETPSLGSGFDPYFVYSGSIPYLDGTFYLNHTFKKV |
| | | | SIMFDSSVSWPGNDRLLTPNEFEIKRSVDGEGYNVAQSNM |
| | | | TKDWFLIQMLSHYNIGYQGFYVPEAYKDRMYSFFRNFQPM |
| | | | SRQVVDADRYEQYKKVTVEYQHNNSGFVGYMGPTMREGQA |
| | | | YPANYPYPLIGDTAVPSLTQKKFLCDRTMWRIPFSSNFMS |
| | | | MGALTDLGQNMLYANSAHALDMTFEVDPMDEPTLLYVLFE |
| | | | VFDWVRIHQPHRGVIEAVYLRTPFSAGNATT |
| 161 | RhAd53 | Hexon | MATPSMMPQWSYMHIAGQDASEYLSPGLVQFARATESYFS |
| | | protein | LGNKFRNPTVAPTHDVTTDRSQRLTLRFVPVDREDTAYSY |
| | | | KVRFTLAVGDNRVLDMASTYFDIRGMLDRGPSFKPYSGTA |
| | | | YNSLAPKGAPNPSQWTTTNGGNKTNSFAQASYIGQSLSKD |
| | | | GVQVAVDTAAGGAAVYADKTFQPEPQVGISQWNENPTTNA |
| | | | AGRILKPTTAMRPCYGSYAYPTNEKGGQVKITDPNNDKTG |
| | | | ANNVSLNFFNTAADNGNNNPKWVLYSEDVNLEGPDTHLVF |
| | | | KPDVTGDATSAETLLGQQAAPNRPNYIGFRDNFIGLMYYN |
| | | | STGNMGVLAGQASQLNAVVDLQDRNTELSYQLMLDALGDR |
| | | | SRYFSMWNQAVDSYDPDVRIIENHGVEDELPNYCFPLNGQ |
| | | | GISNTYKGVKYNTNTWTQDTDWTTNEISIGNIFAMEINLA |
| | | | ANLWRSFLYSNVALYLPDSYKYTPDNIELPTNKNSYGYIN |
| | | | GRVTAPTAIDTYVNIGARWSPDPMDNVNPFNHHRNAGLRY |
| | | | RSMLLGNGRYVPFHIQVPQKFFAIKNLLLLPGSYTYEWNF |
| | | | RKDVNMILQSTLGNDLRVDGASVRFDSINLYANFFPMAHN |
| | | | TASTLEAMLRNDTNDQSFNDYLCAANMLYPIPANATSVPI |
| | | | SIPSRNWAAFRGWSFTRLKTKETPSLGSGFDPYFVYSGSI |
| | | | PYLDGTFYLNHTFKKVSIMFDSSVSWPGNDRLLTPNEFEI |
| | | | KRSVDGEGYNVAQSNMTKDWFLIQMLSHYNIGYQGFYVPE |
| | | | AYKDRMYSFFRNFQPMSRQWVDTVNYANYKEVTMPFQHNN |
| | | | SGFVGYMGPTMREGQAYPANYPYPLIGATAVPSLTQKKFL |
| | | | CDRTMWRIPFSSNFMSMGALTDLGQNMLYANSAHALDMTF |
| | | | EVDPMDEPTLLYVLFEVFDVVRIHQPHRGVIEAVYLRTPF |
| | | | SAGNATT |
| 162 | RhAd54 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPAEWTNSDSKVKVRAQAPFVSSYGATAIT |
| | | | KEGIQVGVTLTDSGSTPQYADKTYQPEPQIGELQWNSDVG |
| | | | TDDKIAGRVLKKTTPMFPCYGSYARPTNEKGGQATPSASQ |
| | | | DVQNPELQFFASTNVANTPKAVLYAEDVSIEAPDTHLVFK |
| | | | PTVTEGITSSEALLTQQAAPNRPNYIAFRDNFIGLMYYNS |
| | | | TGNMGVLAGQASQLNAVVDLQDRNTELSYQLMLDALGDRS |
| | | | RYFSMWNQAVDSYDPDVRIIENHGVEDELPNYCFPLGGMA |
| | | | VTDTYSPIKVNGGGNGWEANNGVFTERGVEIGSGNMFAME |
| | | | INLQANLWRSFLYSNIGLYLPDSLKITPDNITLPENKTTY |
| | | | QYMNGRVTPPGLVDTYVNVGARWSPDVMDSINPFNHHRNA |
| | | | GLRYRSMLLGNGRYVPFHIQVPQKFFAIKNLLLLPGSYTY |
| | | | EWNFRKDVNMILQSSLGNDLRVDGASIRFDSINLYANFFP |
| | | | MAHNTASTLEAMLRNDTNDQSFNDYLCAANMLYPIPANAT |
| | | | SVPISIPSRNWAAFRGWSFTRLKTKETPSLGSGFDPYFVY |
| | | | SGSIPYLDGTFYLNHTFKKVSIMFDSSVSWPGNDRLLTPN |
| | | | EFEIKRSVDGEGYNVAQSNMTKDWFLVQMLSHYNIGYQGF |
| | | | YVPENYKDRMYSFFRNFQPMSRQIVDSTAYTNYQDVKLPY |
| | | | QHNNSGFVGYMGPTMREGQAYPANYPYPLIGATAVPSLTQ |
| | | | KKFLCDRVMWRIPFSSNFMSMGSLTDLGQNMLYANSAHAL |
| | | | DMTFEVDPMDEPTLLYVLFEVFDWVRIHQPHRGVIEAVYL |
| | | | RTPFSAGNATT |
| 163 | RhAd55 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPAEWADSDSKLKVRAQAPFVSSYGNNAIT |
| | | | KEGIQVGVILTDSGQTAQYADKTYQPEPQIGELQWNSEVG |
| | | | TNDKVAGRVLKKTTPMFPCYGSYAKPTNAKGGQATQPASQ |
| | | | ETQNPELQFFAASSTTVNTPKAVLYAEDVSIEAPDTHLVF |
| | | | KPSVTEGTTSSEALLTQQAAPNRPNYIAFRDNFIGLMYYN |
| | | | STGNMGVLAGQASQLNAVVDLQDRNTELSYQLMLDALGDR |
| | | | SRYFSMWNQAVDSYDPDVRIVENHGVEDELPNYCFPLGGM |
| | | | AVTDTYSPIKLNGGQNGWEANNDVYAERGVEIGSGNLFAM |
| | | | EINLQANLWRSFLYSNIGLYLPDSLKITPDNITLPENKNT |
| | | | YQYMNGRVTPPGLIDTYVNVGARWSPDVMDSINPFNHHRN |
| | | | AGLRYRSMLLGNGRYVPFHIQVPQKFFAIKNLLLLPGSYT |
| | | | YEWNFRKDVNMILQSSLGNDLRVDGASIRFDSINLYANFF |
| | | | PMAHNTASTLEAMLRNDTNDQSFNDYLCAANMLYPIPANA |
| | | | TSVPISIPSRNWAAFRGWSFTRLKTKETPSLGSGFDPYFV |
| | | | YSGSIPYLDGTFYLNHTFKKVSIMFDSSVSWPGNDRLLTP |
| | | | NEFEIKRSVDGEGYNVAQSNMTKDWFLIQMLSHYNIGYQG |
| | | | FYVPENYKDRMYSFFRNFQPMSRQIVDSTAYTNYQDVKLP |
| | | | YQHNNSGFVGYMGPTMREGQAYPANYPYPLIGATAVPSLT |
| | | | QKKFLCDRVMWRIPFSSNFMSMGSLTDLGQNMLYANSAHA |
| | | | LDMTFEVDPMDEPTLLYVLFEVFDWVRIHQPHRGVIEAVY |
| | | | LRTPFSAGNATT |
| 164 | RhAd56 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRFTLAVGDNRVLDMASTYFDIRGMLDRGPSFKPYSGTA |
| | | | YNSLAPKGAPNPSQWITNGGTAKTNSFAQASYIGMGANLT |
| | | | TNGIQVAVDTNNANAAVYANKIYQPEPQVGTSRWNINPTQ |
| | | | NAAGRILKPTTPMQPCYGSYAYPTNENGGQVKITDGSTDP |
| | | | TGANSVSMEFFTTTADNQNQAKVVLYSEDVNLEAPDTHLV |
| | | | FKPDVADNAKAAETLLGQQSAPNRPNYIGFRDNFIGLMYY |
| | | | NSTGNMGVLAGQASQLNAVVDLQDRNTELSYQLMLDALGD |
| | | | RSRYFSMWNQAVDSYDPDVRIIENHGVEDELPNYCFPLNG |
| | | | QGISNTYRGLSVQNGQNTWQANNNAAENNEISIGNIFAME |
| | | | INLAANLWRSFLYSNVALYLPDSYKYTPDNIELPTNQNSY |
| | | | GYINGRVTAPNAIDTYVNIGARWSPDPMDNVNPFNHHRNA |
| | | | GLRYRSMLLGNGRYVPFHIQVPQKFFAIKNLLLLPGSYTY |
| | | | EWNFRKDVNMILQSTLGNDLRVDGASVRFDSINLYANFFP |
| | | | MAHNTASTLEAMLRNDTNDQSFNDYLCAANMLYPIPANAT |
| | | | SVPISIPSRNWAAFRGWSFTRLKTKETPSLGSGFDPYFVY |
| | | | SGSIPYLDGTFYLNHTFKKVSIMFDSSVSWPGNDRLLTPN |
| | | | EFEIKRSVDGEGYNVAQSNMTKDWFLIQMLSHYNIGYQGF |
| | | | YVPENYKDRMYSFFRNFQPMSRQVVDSVNYANYKEVKMPF |
| | | | QHNNSGFVGYMGPTMREGQAYPANYPYPLIGATAVPSLTQ |
| | | | KKFLCDRVMWRIPFSSNFMSMGSLTDLGQNMLYANSAHAL |
| | | | DMTFEVDPMDEPTLLYVLFEVFDWRIHQPHRGVIEAVYLR |
| | | | TPFSAGNATT |
| 165 | RhAd57 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPSEWEGSDNKTHVRGQAPFISDEITKEGI |
| | | | KVGTDTGNPGQAIYAKKAYQPEPQVGETQWNSDVGTNDKA |
| | | | AGRILKKTTPMVPCYGSYAKPTNEEGGQATNQQVDLQFFS |
| | | | TGSSNTTPKWVLYSEDVSLETPDTHLVFKPTVTAGVTNAE |
| | | | ALLAQQAAPNRPNYIAFRDNFIGLMYYNSTGNMGVLAGQA |
| | | | SQLNAVVDLQDRNTELSYQLMLDALGDRSRYFSMWNQAVD |
| | | | SYDPDVRIVENHGVEDELPNYCFPLGGMAVTDTYSGIKPS |
| | | | DNGGTFQADNTVADRVEIGSGNMFAMEINLQANLWRGFLY |
| | | | SNIGLYLPDTLKLTPDNITLPENKNTYQYMNGRVTPPGLV |
| | | | DTYVNVGARWSPDVMDSINPFNHHRNAGLRYRSMLLGNGR |
| | | | YVPFHIQVPQKFFAIKNLLLLPGSYTYEWNFRKDVNMILQ |
| | | | SSLGNDLRVDGASIRFDSINLYANFFPMAHNTASTLEAML |
| | | | RNDTNDQSFNDYLCAANMLYPIPANATSVPISIPSRNWAA |
| | | | FRGWSFTRLKTKETPSLGSGFDPYFVYSGSIPYLDGTFYL |
| | | | NHTFKKVSIMFDSSVSWPGNDRLLTPNEFEIKRSVDGEGY |
| | | | NVAQSNMTKDWFLIQMLSHYNIGYQGFYVPENYKDRMYSF |
| | | | FRNFQPMSRQIVDSTAYTNYQDVKLPYQHNNSGFVGYMGP |
| | | | TMREGQAYPANYPYPLIGATAVPSLTQKKFLCDRVMWRIP |
| | | | FSSNFMSMGSLTDLGQNMLYANSAHALDMTFEVDPMDEPT |
| | | | LLYVLFEVFDWVRIHQPHRGVIEAVYLRTPFSAGNATT |
| 166 | RhAd58 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPAEWTNTDSKVKVRAQAPFVSSYGATAIT |
| | | | KEGVQVGWLTDSGSTPQYADKTYQPEPQIGEQQWNSDVGT |
| | | | NDKIAGRVLKKTTPMFPCYGSYARPTNEKGGQATPSASQD |
| | | | VQNPELQFFASTNVANTPKAVLYAEDVAIEAPDTHLVFKP |
| | | | TVTEGITSSEALLTQQAAPNRPNYIAFRDNFIGLMYYNST |
| | | | GNMGVLAGQASQLNAVVDLQDRNTELSYQLMLDALGDRSR |
| | | | YFSMWNQAVDSYDPDVRIIENHGVEDELPNYCFPLGGMAV |
| | | | TDTYSPIKVNTAGNGWEANNDVFTDRGVEIGSGNMFAMEI |
| | | | NLQANLWRSFLYSNIGLYLPDSLKITPDNITLPENKNTYQ |
| | | | YMNGRVTPPGLVDTYVNVGARWSPDVMDSINPFNHHRNAG |
| | | | LRYRSMLLGNGRYVPFHIQVPQKFFAIKNLLLLPGSYTYE |
| | | | WNFRKDVNMILQSSLGNDLRVDGASIRFDSINLYANFFPM |
| | | | AHNTASTLEAMLRNDTNDQSFNDYLCAANMLYPIPANATS |
| | | | VPISIPSRNWAAFRGWSFTRLKTKETPSLGSGFDPYFVYS |
| | | | GSIPYLDGTFYLNHTFKKVSIMFDSSVSWPGNDRLLTPNE |
| | | | FEIKRSVDGEGYNVAQSNMTKDWFLIQMLSHYNIGYQGFY |
| | | | VPENYKDRMYSFFRNFQPMSRQIVDSTSYTNYQDVKLPYQ |
| | | | HNNSGFVGYMGPTMREGQAYPANYPYPLIGATAVPSLTQK |
| | | | KFLCDRVMWRIPFSSNFMSMGALTDLGQNMLYANSAHALD |
| | | | MTFELDPMDEPTLLYVLFEVFDWVRIHQPHRGVIEAVYLR |
| | | | TPFSAGNATT |
| 167 | RhAd59 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPSEWNGTDSKTNVRGQAPFISDAITKEGI |
| | | | KVGTDTGNHGQAIYAKKEYQPEPQVGETQWNSDVGATDKA |
| | | | AGRILKKTTPMVPCYGSYAKPTNEEGGQATNQQVDMQFFA |
| | | | TGSSNTTPKWLYSEDVTIETPDTHLVFKPIVTAGTTNAEA |
| | | | LLAQQAAPNRPNYIGFRDNFIGLMYYNSTGNMGVLAGQAS |
| | | | QLNAVVDLQDRNTELSYQLMLDALGDRSRYFSMWNQAVDS |
| | | | YDPDVRIVENHGVEDELPNYCFPLGGMAVTDTYTGIKPDN |
| | | | GGTTFSNDDTFATRGIEIGSGNMFAMEINLQANLWRSFLY |
| | | | SNIGLYLPDTLKITPDNITLPDNKNTYQYMNGRVTPPGLV |
| | | | DTYVNVGARWSPDVMDGINPFNHHRNAGLRYRSMLLGNGR |
| | | | YVPFHIQVPQKFFAIKNLLLLPGSYTYEWNFRKDVNMILQ |
| | | | SSLGNDLRVDGASIRFDSINLYANFFPMAHNTASTLEAML |
| | | | RNDTNDQSFNDYLCAANMLYPIPANATSVPISIPSRNWAA |
| | | | FRGWSFTRLKTKETPSLGSGFDPYFVYSGSIPYLDGTFYL |
| | | | NHTFKKVSIMFDSSVSWPGNDRLLTPNEFEIKRSVDGEGY |
| | | | NVAQSNMTKDWFLIQMLSHYNIGYQGFYVPENYKDRMYSF |
| | | | FRNFQPMSRQWDTVTYTDYKDVKLPYQHNNSGFVGYMGPT |
| | | | MREGQAYPANYPYPLIGETAVPSLTQKKFLCDRVMWRIPF |
| | | | SSNFMSMGSLTDLGQNMLYANSAHALDMTFEVDPMDEPTL |
| | | | LYVLFEVFDWVRIHQPHRGVIEAVYLRTPFSAGNATT |
| 168 | RhAd60 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPSEWTDTSDNKLKAYAQAPYQSQGLTKDG |
| | | | IQVGLVVTESGQTPQYANKVYQPEPQIGENQWNLEQEDKA |
| | | | AGRVLKKDTPMFPCYGSYARPTNEQGGQAKNQEVDLQFFA |
| | | | TPGDTQNTAKVVLYAENVNLETPDTHLVFKPDDDSTSSKL |
| | | | LLGQQAAPNRPNYIGFRDNFIGLMYYNSTGNMGVLAGQAS |
| | | | QLNAVVDLQDRNTELSYQLMLDALGDRSRYFSMWNQAVDS |
| | | | YDPDVRIIENHGVEDELPNYCFPLGGMVVTDNYNSVTPQN |
| | | | GGSGNTWQADNTTFSQRGAQIGSGNMFALEINLQANLWRG |
| | | | FLYSNIGLYLPDSLKITPDNITLPENKNTYQYMNGRVTPP |
| | | | GLIDTYVNVGARWSPDVMDSINPFNHHRNAGLRYRSMLLG |
| | | | NGRYVPFHIQVPQKFFAIKNLLLLPGSYTYEWNFRKDVNM |
| | | | ILQSSLGNDLRVDGASIRFDSINLYANFFPMAHNTASTLE |
| | | | AMLRNDTNDQSFNDYLCAANMLYPIPANATSVPISIPSRN |
| | | | WAAFRGWSFTRLKTKETPSLGSGFDPYFVYSGSIPYLDGT |
| | | | FYLNHTFKKVSIMFDSSVSWPGNDRLLTPNEFEIKRSVDG |
| | | | EGYNVAQSNMTKDWFLIQMLSHYNIGYQGFYVPENYKDRM |
| | | | YSFFRNFQPMSRQVVDTVTYTDYKDVKLPYQHNNSGFVGY |
| | | | MGPTMREGQAYPANYPYPLIGETAVPSLTQKKFLCDRVMW |
| | | | RIPFSSNFMSMGSLTDLGQNMLYANSAHALDMTFEVDPMD |
| | | | EPTLLYVLFEVFDWRIHQPHRGVIEAVYLRTPFSAGNATT |
| 169 | RhAd61 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPAEWEDTNDSNKLKVRGQAPYVSTFGAAT |
| | | | ALTKDGIQVGVTVSESARTPQYADKSYQPEPQIGETEWNS |
| | | | EVGNDDKLAGRVLKKSTPMFPCYGSYAKPTNEKGGQAIQP |
| | | | AGSSGDTNPAVELQFFATTSATTTPKAVLYAEDVAIEAPD |
| | | | THLVFKPTVTAGTTSSEALLTQQAAPNRPNYIAFRDNFIG |
| | | | LMYYNSTGNMGVLAGQASQLNAVVDLQDRNTELSYQLMLD |
| | | | ALGDRSRYFSMWNQAVDSYDPDVRIVENHGVEDELPNYCF |
| | | | PLGGMWTDTYTPIKPNGSGSVTWTANTDVFSNRVEIGSGN |
| | | | LFAMEINLQANLWRSFLYSNIGLYLPDSLKITPDNITLPE |
| | | | NKNTYQYMNGRVTPPGLIDTYVNVGARWSPDVMDSINPFN |
| | | | HHRNAGLRYRSMLLGNGRYVPFHIQVPQKFFAIKNLLLLP |
| | | | GSYTYEWNFRKDVNMILQSSLGNDLRVDGASIRFDSINLY |
| | | | ANFFPMAHNTASTLEAMLRNDTNDQSFNDYLCAANMLYPI |
| | | | PANATSVPISIPSRNWAAFRGWSFTRLKTKETPSLGSGFD |
| | | | PYFVYSGSIPYLDGTFYLNHTFKKVSIMFDSSVSWPGNDR |
| | | | LLTPNEFEIKRSVDGEGYNVAQSNMTKDWFLIQMLSHYNI |
| | | | GYQGFYVPENYKDRMYSFFRNFQPMSRQIVDSTAYTNYQD |
| | | | VKLPYQHNNSGFVGYMGPTMREGQAYPANYPYPLIGATAV |
| | | | PSLTQKKFLCDRVMWRIPFSSNFMSMGSLTDLGQNMLYAN |
| | | | SAHALDMTFEVDPMDEPTLLYVLFEVFDWRIHQPHRGVIE |
| | | | AVYLRTPFSAGNATT |
| 170 | RhAd62 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPSEWNGTDSKTNVRGQAPFISDAITKEGI |
| | | | KVGTDTGNPGQAIYAKKEYQPEPQVGETQWNSDVGATDKA |
| | | | AGRILKKTTPMVPCYGSYAKPTNEEGGQATNQQVDMQFFA |
| | | | TGSSNTTPKWVLYSEDVTIETPDTHLVFKPIVTAGTTNAE |
| | | | ALLAQQAAPNRPNYIGFRDNFIGLMYYNSTGNMGVLAGQA |
| | | | SQLNAVVDLQDRNTELSYQLMLDALGDRSRYFSMWNQAVD |
| | | | SYDPDVRIVENHGVEDELPNYCFPLGGMAVTDTYTGIKPD |
| | | | NGGTTFSNDDTFATRGIEIGSGNMFAMEINLQANLWRSFL |
| | | | YSNIGLYLPDTLKITPDNITLPDNKNTYQYMNGRVTPPGL |
| | | | VDTYVNVGARWSPDVMDGINPFNHHRNAGLRYRSMLLGNG |
| | | | RYVPFHIQVPQKFFAIKNLLLLPGSYTYEWNFRKDVNMIL |
| | | | QSSLGNDLRVDGASIRFDSINLYANFFPMAHNTASTLEAM |
| | | | LRNDTNDQSFNDYLCAANMLYPIPANATSVPISIPSRNWA |
| | | | AFRGWSFTRLKTKETPSLGSGFDPYFVYSGSIPYLDGTFY |
| | | | LNHTFKKVSIMFDSSVSWPGNDRLLTPNEFEIKRSVDGEG |
| | | | YNVAQSNMTKDWFLIQMLSHYNIGYQGFYVPENYKDRMYS |
| | | | FFRNFQPMSRQWDTVTYTDYKDVKLPYQHNNSGFVGYMGP |
| | | | TMREGQAYPANYPYPLIGATAVPSLTQKKFLCDRVMWRIP |
| | | | FSSNFMSMGSLTDLGQNMLYANSAHALDMTFEVDPMDEPT |
| | | | LLYVLFEVFDWVRIHQPHRGVIEAVYLRTPFSAGNATT |
| 171 | RhAd63 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRFTLAVGDNRVLDMASTYFDIRGMLDRGPSFKPYSGTA |
| | | | YNSLAPKGAPNPSQWITNGGTAKTNSFAQASYIGMGANLT |
| | | | TNGIQVAVDTNNANAAVYANKIYQPEPQVGTSQWNINPTQ |
| | | | NAAGRILKPTTPMQPCYGSYAYPTNENGGQVKITDGSTDP |
| | | | TGANSVSMEFFTTTADNQNQAKVVLYSEDVNLEAPDTHLV |
| | | | FKPDVADNAKAAETLLGQQSAPNRPNYIGFRDNFIGLMYY |
| | | | NSTGNMGVLAGQASQLNAVVDLQDRNTELSYQLMLDALGD |
| | | | RSRYFSMWNQAVDSYDPDVRIIENHGVEDELPNYCFPLNG |
| | | | QGISNTYRGLSVQNGQNTWQANNNAAENNEISIGNIFAME |
| | | | INLAANLWRSFLYSNVALYLPDSYKYTPDNIELPTNQNSY |
| | | | GYINGRVTAPNAIDTYVNIGARWSPDPMDNVNPFNHHRNA |
| | | | GLRYRSMLLGNGRYVPFHIQVPQKFFAIKNLLLLPGSYTY |
| | | | EWNFRKDVNMILQSTLGNDLRVDGASVRFDSINLYANFFP |
| | | | MAHNTASTLEAMLRNDTNDQSFNDYLCAANMLYPIPANAT |
| | | | SVPISIPSRNWAAFRGWSFTRLKTKETPSLGSGFDPYFVY |
| | | | SGSIPYLDGTFYLNHTFKKVSIMFDSSVSWPGNDRLLTPN |
| | | | EFEIKRSVDGEGYNVAQSNMTKDWFLIQMLSHYNIGYQGF |
| | | | YVPENYKDRMYSFFRNFQPMSRQVVDSVNYANYKEVKMPF |
| | | | QHNNSGFVGYMGPTMREGQAYPANYPYPLIGATAVPSLTQ |
| | | | KKFLCDRVMWRIPFSSNFMSMGSLTDLGQNMLYANSAHAL |
| | | | DMTFEVDPMDEPTLLYVLFEVFDWRIHQPHRGVIEAVYLR |
| | | | TPFSAGNATT |
| 172 | RhAd64 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPAEWENSDNKLKVRAQAPFVSSYGATAIT |
| | | | KDGIQVGWVLTDSTQTPQYADKTYQPEPQIGELQWNSEVG |
| | | | NDDKVAGRVLKKTTPMFPCYGSYAKPTNDKGGQATQPAAG |
| | | | QDAQNAVELQFFATTSATTTPKAVLYAEDVSIETPDTHLV |
| | | | FKPTVTAGTTSSEALLSQQAAPNRPNYIAFRDNFIGLMYY |
| | | | NSTGNMGVLAGQASQLNAVVDLQDRNTELSYQLMLDALGD |
| | | | RSRYFSMWNQAVDSYDPDVRIVENHGVEDELPNYCFPLGG |
| | | | TVVTDTYTPIQPNSNGGGTWVAKDNVFTDRGVEISSGNMF |
| | | | AMEINLQANLWRSFLYSNIGLYLPDTLKITPDNITLPENK |
| | | | NTYQYMNGRVTPPGLVDTYVNVGARWSPDVMDSINPFNHH |
| | | | RNAGLRYRSMLLGNGRYVPFHIQVPQKFFAIKNLLLLPGS |
| | | | YTYEWNFRKDVNMILQSSLGNDLRVDGASIRFDSINLYAN |
| | | | FFPMAHNTASTLEAMLRNDTNDQSFNDYLCAANMLYPIPA |
| | | | NATSVPISIPSRNWAAFRGWSFTRLKTKETPSLGSGFDPY |
| | | | FVYSGSIPYLDGTFYLNHTFKKVSIMFDSSVSWPGNDRLL |
| | | | TPNEFEIKRSVDGEGYNVAQSNMTKDWFLIQMLSHYNIGY |
| | | | QGFYVPENYKDRMYSFFRNFQPMSRQVVDTVAYKDYYQDV |
| | | | KLPYQHNNSGFVGYMGPTMREGQAYPANYPYPLIGATAVP |
| | | | SLTQKKFLCDRVMWRIPFSSNFMSMGSLTDLGQNMLYANS |
| | | | AHALDMTFEVDPMDEPTLLYVLFEVFDWVRIHQPHRGVIE |
| | | | AVYLRTPFSAGNATT |
| 173 | RhAd65 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPAEWADSDSKLKVRAQAPFVSSYGNNAIT |
| | | | KEGIQVGVILTDSGQTAQYADKTYQPEPQIGELQWNSEVG |
| | | | TNDKVAGRVLKKTTPMFPCYGSYAKPTNAKGGQATQPASQ |
| | | | ETQNPELQFFAASSTVNTPKAVLYAEDVSIEAPDTHLVFK |
| | | | PSVTEGTTSSEALLAQQAAPNRPNYIAFRDNFIGLMYYNS |
| | | | TGNMGVLAGQASQLNAVVDLQDRNTELSYQLMLDALGDRS |
| | | | RYFSMWNQAVDSYDPDVRIVENHGVEDELPNYCFPLGGMA |
| | | | VTDTYSPIKLNGGQNGWEANNDVYAERGVEIGSGNLFAME |
| | | | INLQANLWRSFLYSNIGLYLPDTLKITPDNITLPENKNTY |
| | | | QYMNGRVTPPGLVDTYVNVGARWSPDVMDSINPFNHHRNA |
| | | | GLRYRSMLLGNGRYVPFHIQVPQKFFAIKNLLLLPGSYTY |
| | | | EWNFRKDVNMILQSSLGNDLRVDGASIRFDSINLYANFFP |
| | | | MAHNTASTLEAMLRNDTNDQSFNDYLCAANMLYPIPANAT |
| | | | SVPISIPSRNWAAFRGWSFTRLKTKETPSLGSGFDPYFVY |
| | | | SGSIPYLDGTFYLNHTFKKVSIMFDSSVSWPGNDRLLTPN |
| | | | EFEIKRSVDGEGYNVAQSNMTKDWFLIQMLSHYNIGYQGF |
| | | | YVPENYKDRMYSFFRNFQPMSRQIVDSTAYTNYQDVKLPY |
| | | | QHNNSGFVGYMGPTMREGQAYPANYPYPLIGATAVPSLTQ |
| | | | KKFLCDRVMWRIPFSSNFMSMGSLTDLGQNMLYANSAHAL |
| | | | DMTFEVDPMDEPTLLYVLFEVFDWRIHQPHRGVIEAVYLR |
| | | | TPFSAGNATT |
| 174 | RhAd66 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRFTLAVGDNRVLDMASTYFDIRGMLDRGPSFKPYSGTA |
| | | | YNSLAPKGAPNPSQWITNGGTAKTNSFAQASYIGMGANLT |
| | | | TNGIQVAVDTNNANAAVYANKIYQPEPQVGTSQWNINPTQ |
| | | | NAAGRILKPTTPMQPCYGSYAYPTNENGGQVKITDGTTDP |
| | | | TGANSVSMEFFTTTADNQNQAKVVLYSEDVNLEAPDTHLV |
| | | | FKPDVADNAKAAETLLGQQSAPNRPNYIGFRDNFIGLMYY |
| | | | NSTGNMGVLAGQASQLNAVVDLQDRNTELSYQLMLDALGD |
| | | | RSRYFSMWNQAVDSYDPDVRIIENHGVEDELPNYCFPLNG |
| | | | QGISNTYKGLSVQNGQNTWQANNNAAENNEISIGNIFAME |
| | | | INLAANLWRSFLYSNVALYLPDSYKYTPDNIELPTNQNSY |
| | | | GYINGRVTAPNAIDTYVNIGARWSPDPMDNVNPFNHHRNA |
| | | | GLRYRSMLLGNGRYVPFHIQVPQKFFAIKNLLLLPGSYTY |
| | | | EWNFRKDVNMILQSTLGNDLRVDGASVRFDSINLYANFFP |
| | | | MAHNTASTLEAMLRNDTNDQSFNDYLCAANMLYPIPANAT |
| | | | SVPISIPSRNWAAFRGWSFTRLKTKETPSLGSGFDPYFVY |
| | | | SGSIPYLDGTFYLNHTFKKVSIMFDSSVSWPGNDRLLTPN |
| | | | EFEIKRSVDGEGYNVAQSNMTKDWFLIQMLSHYNIGYQGF |
| | | | YVPENYKDRMYSFFRNFQPMSRQVVDSVNYANYKEVKMPF |
| | | | QHNNSGFVGYMGPTMREGQAYPANYPYPLIGATAVPSLTQ |
| | | | KKFLCDRVMWRIPFSSNFMSMGSLTDLGQNMLYANSAHAL |
| | | | DMTFEVDPMDEPTLLYVLFEVFDVVRIHQPHRGVIEAVYL |
| | | | RTPFSAGNATT |
| 175 | RhAd67 | Hexon | MATPSMMPQWSYMHIAGQDASEYLSPGLVQFARATDTYFS |
| | | protein | LGNKFRNPTVAPTHDVTTDRSQRLTLRFVPVDREDTAYSY |
| | | | KVRYTLAVGDNRVLDMASTYFDIRGVLDRGPSFKPYSGTA |
| | | | YNSLAPKGAPNPAEWENSDNKLKVRAQAPFVSSYGATAIT |
| | | | KDGIQVGWVLTDSTQTPQYADKTYQPEPQIGELQWNSEVG |
| | | | NDDKVAGRVLKKTTPMFPCYGSYAKPTNDKGGQATQPAAG |
| | | | QDAQNAVELQFFATTSATTTPKAVLYAEDVSIETPDTHLV |
| | | | FKPTVTAGTTSSEALLSQQAAPNRPNYIAFRDNFIGLMYY |
| | | | NSTGNMGVLAGQASQLNAVVDLQDRNTELSYQLMLDALGD |
| | | | RSRYFSMWNQAVDSYDPDVRIVENHGVEDELPNYCFPLGG |
| | | | TWVTDTYTPIQPNSNGGGTWVAKDNVFTDRGVEISSGNMF |
| | | | AMEINLQANLWRSFLYSNIGLYLPDTLKITPDNITLPENK |
| | | | NTYQYMNGRVTPPGLVDTYVNVGARWSPDVMDSINPFNHH |
| | | | RNAGLRYRSMLLGNGRYVPFHIQVPQKFFAIKNLLLLPGS |
| | | | YTYEWNFRKDVNMILQSSLGNDLRVDGASIRFDSINLYAN |
| | | | FFPMAHNTASTLEAMLRNDTNDQSFNDYLCAANMLYPIPA |
| | | | NATSVPISIPSRNWAAFRGWSFTRLKTKETPSLGSGFDPY |
| | | | FVYSGSIPYLDGTFYLNHTFKKVSIMFDSSVSWPGNDRLL |
| | | | TPNEFEIKRSVDGEGYNVAQSNMTKDWFLIQMLSHYNIGY |
| | | | QGFYVPENYKDRMYSFFRNFQPMSRQWDTVAYKDYYQDVK |
| | | | LPYQHNNSGFVGYMGPTMREGQAYPANYPYPLIGATAVPS |
| | | | LTQKKFLCDRVMWRIPFSSNFMSMGSLTDLGQNMLYANSA |
| | | | HALDMTFEVDPMDEPTLLYVLFEVFDWVRIHQPHRGVIEA |
| | | | VYLRTPFSAGNATT |
| 176 | RhAd51 | fiber-1 | MKRTRVDEDFNPVYPYDSTSTPAVPFISPPFVNSDGLQEN |
| | | protein | PPGVLSLRIAKPLYFDMERKLALSLGRGLAITSTGQLEST |
| | | | QSVQTTPPLVVNNSNTLVLRYSSPLGLSGDNLILNCSDPL |
| | | | RVVNNSLTFSYLSPLRFEGGSLTFNYTSPLKLLNSSLAIG |
| | | | INSNKGLGNDSDELSVKLTSDLKENNDGKIAFGIQSLCTT |
| | | | PTAASNCTVFTNGDSLLCLCLTKCGAHVLGSVSLTGMQGT |
| | | | ITAMTQNYISIQFLFDNNGALTSSPLLNNNTWGIRQNDTS |
| | | | SANPAYNALAFMPNSTVYVRGQSGEPRNNYYTQTYLRGNV |
| | | | KKPIILTVTYNSAASGYSLTFKWDAVVTEKFATPTSSFCY |
| | | | ITEQ |
| 177 | RhAd52 | fiber-1 | MKRTRVDEDFNPVYPYDTTTTPAVPFISPPFVNSDGLQEN |
| | | protein | PPGVLSLRIAKPLYFDMERKLALSLGRGLTITAAGQLEST |
| | | | QSVQTNPPLIITNNNTLTLRHSPPLNLTDNSLVLGYSSPL |
| | | | RVTDNKLTFNFTSPLRYENENLTFNYTEPLKLINNSLAID |
| | | | INSSKGLSSVGGSLAVNLSSDLKFDSNGSIAFGIQTLWTA |
| | | | PTSTGNCTVYSEGDSLLSLCLTKCGAHVLGSVSLTGLTGT |
| | | | ITQMTDISVTIQFTFDNNGKLLSSPLINNAFSIRQNDSTA |
| | | | SNPTYNALAFMPNSTIYARGGGGEPRNNYYVQTYLRGNVQ |
| | | | KPIILTVTYNSAATGYSLSFKWTALAREKFATPTTSFCYI |
| | | | TEQ |
| 178 | RhAd53 | fiber-1 | MKRTRVDEDFNPVYPYDTTTTPAVPFISPPFVNSDGLQEN |
| | | protein | PPGVLSLRIAKPLYFDMERKLALSLGRGLTITANGQLEST |
| | | | QSVQTNPPLTVTNNNTLILRHSSPLILTDNNLTVGFSSPL |
| | | | RVIDNKLTFTFTSPLRYENETLTFNYTEPLTLMNSNLALN |
| | | | VNSSKGLRVDGGSLGTNLSPDLRFNSSGAIAFGIQTLWTP |
| | | | PTSNPNCTVYTESDSLLSLCLTKCGAHVLGSVSLTGVAGT |
| | | | MINMAETSLAIEFTFDDTGKLLHSPLVNTTFSIRQGDSPA |
| | | | SNPTYNALAFMPNSTLYARGGSGEPRNNYYVQTYLRGNVQ |
| | | | RPITLTVTFNSAATGYSLSFKWTAVAREKFAAPATSFCYI |
| 179 | RhAd51 | fiber-2 | MKRARVDEDFNPVYPYDPPYAPVMPFITPPFTSSDGLQEK |
| | | protein | PLGVLSLNYKDPITTQNGSLTLKIGNGLTLDNQGQLTSTA |
| | | | GEVEPPLTNANNKLALAYSEPLAVKSNRLTLSHTAPLVVA |
| | | | NNSLALQVSEPIFINDDDKLALQTAAPLVTNAGTLRLQSA |
| | | | APLGLVENTLRLLFSKPLYLQNDFLALGIERPLAIAAAGT |
| | | | LALQLTPPLKTNDDGLTLSTVEPLTVKNGNLGLQISRPLV |
| | | | VQNSSLSLAITPPLRLFNSDPVLGLGFTFPLAVTDNLLSL |
| | | | NMGDGVKLTYNKLTANLGRDLQFENGAIAVTLTAESPLQY |
| | | | TNKLQLNIGAGLRYNGASRKLDVNINQNKGLTWDNDAVIP |
| | | | KLGSGLQFDPNGNIAVIPETVKPQTLWTTADPSPNCSVYQ |
| | | | DLDARLWLALVKSGDMVHGSIALKALKGTLLNPTASYISI |
| | | | VIYFYSNGVRRTNYPTFDNEGTLANSATWGYREGQSANTN |
| | | | VTNATEFMPSSTRYPVNKGDNIQNQSFSYTCIKGDFAMPV |
| | | | PFRVTYNHALEGYSLKFTWRVVANQAFDIPCCSFSYITE |
| 180 | RhAd52 | fiber-2 | MKRARVDEDFNPVYPYDPPHAPVMPFITPPFTSSDGLQEK |
| | | protein | PLGVLSLNYRDPITTQNGSLTVKLGNGLTLDNQGQLTSTA |
| | | | GEVEPPLTNANNKLALVYSDPLAVKRNSLTLSHTAPLVIA |
| | | | DNSLALQVSEPIFINDKDKLALQTAAPLVTNAGTLRLQSA |
| | | | APLGIADQTLKLLFTNPLYLQNNFLTLAIERPLAITNSGK |
| | | | LALQLSPPLQTADTGLTLQTNVPLTVSNGTLGLAIKRPLI |
| | | | VQDNNLFLDFRAPLRLFNSDPVLGLNFYTPLAVRDEALTV |
| | | | NTGRGLTVSYDGLILNLGKDLRFDNNTVSVALSAALPLQY |
| | | | TDQLRLNVGAGLRYNPVSKKLDVNPNQNKGLTWENDYLIV |
| | | | KLGNGLGFDGNGNIAVSPQVTSPDTLWTTADPSPNCSIYT |
| | | | DLDAKMWLSLVKQGGVVHGSVALKALKGTLLSPTESAIVI |
| | | | ILHFDNYGVRILNYPTLGTQGTLGNNATWGYRQGESADTN |
| | | | VLNALAFMPSSKRYPRGRGSEVQNQTVGYTCIQGDLSMPV |
| | | | PYQIQYNYGPTGYSFKFIWRTVSRQPFDIPCCFFSYITEE |
| 181 | RhAd53 | fiber-2 | MKRARVDEDFNPVYPYDPPYAPIMPFITPPFTSSDGLQEK |
| | | protein | PLGVLSLKYKDPITTQNGSLTLKLGNGLNINNQGQLTSSA |
| | | | GEVEPPLTNADNKLALAYSDPLTLKNSRLTLSHNAPLAIN |
| | | | NNSLSLEVSEPIFINNDNKLSLKADAPLTTSAGTLRLQSA |
| | | | APLGLAEQTLKLLFSNPLYLRGDFLTLAIERPLAVTADGL |
| | | | LSLALNPPLTTTNTGLALSTAVPLTVTNGNLSLNVKRPFI |
| | | | IQDGSLYMDFRPPLYLFNSEPQLGVNFNAPLTVRDNGLAI |
| | | | NTGDGLTVTYNKLTLNLGRDLQYENGAAAVKLSTAPPLQY |
| | | | TTQLQLNLGAGLRLGPTRNLDVAINHNKGLAWENNEVVTK |
| | | | LGQGLYFDSSGSIALSPTNPRPDTLWTTADPSPNCTVYES |
| | | | LDSRLWLALVKCGGMVHGSIALQAEKGQLLRPTASFISIV |
| | | | IYFYSDGVRRTNYPTIGNDEGTLANSATWGYRQGQSADTN |
| | | | VTNAVEFMPSLHRYPINQGDNIKNQMITYTCIQGNVNMPV |
| | | | PLKITFNHALEGYSLKFTWRWANEKFDIPCCSFSYITEQ |
| 182 | RhAd54 | fiber-1 | MKRTRVDEDFNPVYPYDTTTTPAVPFISPPFVNSDGLQEN |
| | | protein | PPGVLSLRIAKPLYFDMERKLALSLGRGLTITAAGQLEST |
| | | | QSVQTNPPLIITNNNTLTLRHSPPLNLTDNSLVLGYSSPL |
| | | | RVTDNKLTFNFTSPLRYENENLTFKYTEPLKLINNSLAID |
| | | | INSSKGLSSVGGSLAVNLSSDLKFDSNGSIAFGIQTLWTA |
| | | | PTSTGNCTVYSEGDSLLSLCLTKCGAHVLGSVSLTGLTGT |
| | | | ITQMTDISVTIQFTFDNNGKLLSSPLINNAFSIRQNDSTA |
| | | | SNPTYNALAFMPNSTIYARGGGGEPRNNYYVQTYLRGNVQ |
| | | | KPIILTVTYNSAATGYSLSFKWTALAREKFATPTTSFCYI |
| | | | TEQ |
| 183 | RhAd54 | long fiber | MKRVRIDEDFNPVYPYDPPYAPVMPFITPPFTSSDGLQEK |
| | | protein | PLGVLSLNYKDPITTQNGSLTLKIGNGLTLDNQGQLTSTA |
| | | | GEVEPPLTNANNKLALAYSEPLAVKSNRITLSHTAPLVVA |
| | | | DDSLALQVSEPIFIDDDDKLALQTAAPLVTNAGTLRLQSA |
| | | | APLGLVENTLRLLFSKPLYLQNDFLALAIERPLAIAAAGT |
| | | | LALQLTPPLKTNDDGLTLSTVEPLTVKNGNLGLQISRPLW |
| | | | VQNSSLSLAITPPLRLFNSDPVLGLGFTFPLAVTNNLLSL |
| | | | NMGDGVKLTYNKLTANLGRDLQFENGAIAVTLTAELPLQY |
| | | | TNKLQLNIGAGLRYNGASRKLDVNINQNKGLTWDNDAVIP |
| | | | KLGSGLQFDPNGNIAVIPETVKPQTLWTTADPSPNCSVYQ |
| | | | NLDARLWLALVKSGDMVHGSIALKALKGTLLNPTASYISI |
| | | | VIYFYSNGVRRTNYPTFDNEGTLANSATWGYREGQSANTN |
| | | | VTNATEFMPSSTRYPVNKGDNIQNQSFSYTCIKGDFAMPV |
| | | | PFRVTYNHALEGYSLKFTWRWVANQAFDIPCCSFSYITE |
| 184 | RhAd55 | fiber-1 | MYLLITTHDFHLSQMKRTRVDEDFNPVYPYDAEQSSSVPF |
| | | protein | VTPPFTSSNGLTESPSGVLTLNYSQPLTTINGKLTLNVDG |
| | | | GLNINNDKKLQVRTNENEGVTLQNNAVSVKIGNDLRFDSR |
| | | | GQITLNPVSLWTTPSESANCTLYNSSANFFLCLTRNDAHV |
| | | | VGTVNLTGINGKFSSMTENSVIVQLAFDENGTLQSSPLAQ |
| | | | NVWGIRQTPNSPNAVNLLTFMPSSLVYPREGGGKPKNNLY |
| | | | FNTFLRGNLSRPITLTLTLNSLPTGYTLTFKWGTYLNEPF |
| | | | SVPTTCFCYVAEQ |
| 185 | RhAd55 | fiber-2 | MKRARVDESFNPVYPYDSTSTPAIPFVTPPFVSNDGLQEN |
| | | protein | PPGILSLQIAKPLFFNTQRKIALSLGRGLAITSAGELENT |
| | | | QRIQSTPPLLLNSNTLSMNYSSPLTVLDNKLALNYLSPLR |
| | | | VVNDSLTFNFTSPLHVADGNLTFSYTSPLQVTNNSLALNY |
| | | | SAPLRLLNNSLTFTYANPFKIANSSLSLKLDYPSALRLNN |
| | | | NGSLQVIAGWGLGIAGNNDLYVKRQAPLAVYESGSNAGTL |
| | | | YLRLGPNSALRTVNDNLTLNTGQGLQITNNTLSVKLGNGL |
| | | | RFNNTGAIELAQ |
| 186 | RhAd55 | long fiber | MKRARVDEDFNPVYPYDPPYAPIMPFITPPFTSSDGLQEK |
| | | protein | PFGVLSINYKDPITTQNGSLTLKVGNGLSINNQGQLTSTQ |
| | | | TTVTPPLTNESDTLGLAYSDPLTVKNNSLTLSHSAPFAVS |
| | | | NHSLSLKTTTPFFINQQNELALSTDAPLQVTAGSLRLQSA |
| | | | APLGIAEQTLRLLFSNPLFLQDNFLTLAIERPLAITSSGN |
| | | | LTVQLNPPFQSTSTGLALSLSNPLTISNGTLAMSIKRPFI |
| | | | IDDNKLYMDFRPPLRLFNSEPQLGVHFNPPITVQQGGLAL |
| | | | NTGDGLQVYLNKLTLNIGRDLQYENGSLVVKLTPAPPLQY |
| | | | TTQLQLNMGAGLHLGATKQLTLDVDTNRGLTWNNNKLAVN |
| | | | LGDGLQFNSYGQIIPTVKKDTLWTTADPSPNCSIYKDLDA |
| | | | KLWLSLVKSGGMVHGSIALQALKDTLLSPTDSFITIILYF |
| | | | DENGVRAPYPTFDNEGTLAKDATWGYRQGPSADTNVTNAL |
| | | | EFMPSSARYPRGKHEQAQNQTFGYTCLQGNFSWPIPFRVQ |
| | | | YNYVRTGFSFKFTWQWVNRQKFDIPCCSFSYITEQ |
| 187 | RhAd56 | fiber-1 | MKRTRVDEDFNPVYPYDTTTTPAVPFISPPFVNSDGLQEN |
| | | protein | PPGVLSLRIAKPLYFDMERKLALSLGRGLTITAAGQLEST |
| | | | QSVQTNPPLIITNNNTLTLRHSPPLNLTDNSLVLGYSSPL |
| | | | RVTDNKLTFNFTSPLRYENENLTFNYTEPLKLINNSLAID |
| | | | INSSKGLSSVGGSLAVNLSSDLKFDSNGSIAFGIQTLWTA |
| | | | PTSTGNCTVYSEGDSLLSLCLTKCGAHVLGSVSLTGLTGT |
| | | | ITQMTDISVTIQFTFDNNGKLLSSPLINNAFSIRQNDSTA |
| | | | SNPTYNALAFMPNSTIYARGGGGEPRNNYYVQTYLRGNVQ |
| | | | KPIILTVTYNSAATGYSLSFKWTALAREKFATPTTSFCYI |
| | | | TEQ |
| 188 | RhAd56 | long fiber | MKRARVDEDFNPVYPYDPPHAPVMPFITPPFTSSDGLQEK |
| | | protein | PLGVLSLNYRDPITTQNGSLTVKLGNGLTLDNQGQLTSTA |
| | | | GEVEPPLTNANNKLALVYSDPLAVKRNSLTLSHTAPLVIA |
| | | | DNSLALQVSEPIFINDKDKLALQTAAPLVTNAGTLRLQSA |
| | | | APLGIADQTLKLLFTNPLYLQNNFLTLAIERPLAITNSGK |
| | | | LALQLSPPLQTADTGLTLQTNVPLTVSNGTLGLAIKRPLI |
| | | | VQDNNLFLDFRAPLRLFNSDPVLGLNFYTPLAVRDEALTV |
| | | | NTGRGLTVSYDGLILNLGKDLRFDNNTVSVALSAALPLQY |
| | | | TDQLRLNVGAGLRYNPVSKKLDVNPNQNKGLTWENDYLIV |
| | | | KLGNGLGFDGNGNIAVSPQVTSPDTLWTTADPSPNCSIYT |
| | | | DLDAKMWLSLVKQGGVVHGSVALKALKGTLLSPTESAIVI |
| | | | ILHFDNYGVRILNYPTLGTQGTLGNNATWGYRQGESADTN |
| | | | VLNALAFMPSSKRYPRGRGSEVQNQTVGYTCIQGDLSMPV |
| | | | PYQIQYNYGPTGYSFKFIWRTVSRQPFDIPCCFFSYITEE |
| 189 | RhAd57 | fiber-1 | MYLLITTHDFHLSQMKRTRVDEDFNPVYPYDAEQSSSVPF |
| | | protein | VTPPFTSSNGLTESPSGVLTLNYSQPLTTINGKLTLNVDG |
| | | | GLNINNDKKLQVRTNENEGVTLQNNAVSVKIGNDLRFDSR |
| | | | GQITLNPVSLWTTPSESANCTLYNSSANFFLCLTRNDAHV |
| | | | VGTVNLTGINGKFSSMTENSVIVQLAFDENGTLQSSPLAQ |
| | | | NVWGIRQTPNSPNAVNLLTFMPSSLVYPREGGGKPKNNLY |
| | | | FNTFLRGNLSRPITLTLTLNSLPTGYTLTFKWGTYLNEPF |
| | | | SVPTTCFCYVAEQ |
| 190 | RhAd57 | fiber-2 | MKRARVDESFNPVYPYDSTSTPAIPFVTPPFVSNDGLQEN |
| | | protein | PPGILSLQIAKPLFFNTQRKIALSLGRGLAITSAGELENT |
| | | | QRIQSTPPLLLNSNTLSMNYSSPLTVLDNKLALNYLSPLR |
| | | | WNDSLTFNFTSPLHVADGNLTFSYTSPLQVTNNSLALNYS |
| | | | APLRLLNNSLTFTYANPFKIANSSLSLKLDYPSALRLNNN |
| | | | GSLQVIAGWGLGIAGNNDLYVKRQAPLAVYESGSNAGTLY |
| | | | LRLGPNSALRTVNDNLTLNTGQGLQITNNTLSVKLGNGLR |
| | | | FNNTGAIELAQ |
| 191 | RhAd57 | long fiber | MKRARVDEDFNPVYPYDPPYAPIMPFITPPFTSSDGLQEK |
| | | protein | PFGVLSINYKDPITTQNGSLTLKVGNGLSINNQGQLTSTQ |
| | | | TTVTPPLTNESDTLGLAYSDPLTVKNNSLTLSHSAPFAVS |
| | | | NHSLSLKTTTPFFINQQNELALSTDAPLQVTAGSLRLQSA |
| | | | APLGIAEQTLRLLFSNPLFLQDNFLTLAIERPLAITSSGN |
| | | | LTVQLNPPFQSTSTGLALSLSNPLTISNGTLAMSIKRPFI |
| | | | IDDNKLYMDFRPPLRLFDSEPQLGVHFNPPITVQQGGLAL |
| | | | NTGDGLQVYLNKLTLNIGRDLQYENGSLVVKLTPAPPLQY |
| | | | TTQLQLNMGAGLHLGATKQLTLDVDTNRGLTWNNNKLAVN |
| | | | LGDGLQFNSYGQIIPTVKKDTLWTTADPSPNCSIYKDLDA |
| | | | KLWLSLVKSGGMVHGSIALQALKDTLLSPTDSFITIILYF |
| | | | DENGVRAPYPTFDNEGTLAKDATWGYRQGPSADTNVTNAL |
| | | | EFMPSSARYPRGKHEQAQNQTFGYTCLQGNFSWPIPFRVQ |
| | | | YNYVRTGFSFKFTWQWVNRQKFDIPCCSFSYITEQ |
| 192 | RhAd58 | fiber-1 | MKRTRVDEDFNPVYPYDTTSTPAVPFISPPFVNSDGLQEN |
| | | protein | PPGILSLRIDRPLYFNTQRQLALSLGNGLAITAGGELEST |
| | | | LSVRPNPPLVINGNSLSLRYSDPLRVVNDSLTFNFTSPLR |
| | | | FESGSLTFHYASPLKLMNDSLALDVEPSKGLSTDGNKLAV |
| | | | KLSSDLKFDNNGAIAFGIQSLWTAPTTTPNCMTYAENDSL |
| | | | LSLCLTKCGAHVLGSVSLTGFTGDLLNMKQTTVSIEFRFD |
| | | | NNGILTTSPLVNSSSWGIRQNNGIYPNPVYNALAFMPNST |
| | | | VYSRGGGGEPRNNYYTQTYLRGNTQIPITLTVTYNSANTG |
| | | | YSLTFKWTALTNQKFAAPTASFCYISEQ |
| 193 | RhAd58 | long fiber | MKRVRIDEDFNPVYPYDPPYAPVMPFITPPFTSSDGLQEK |
| | | protein | PLGVLSLNYKDPITTQNGSLTLKIGNGLTLDNQGQLTSTA |
| | | | GEVEPPLTNANNKLALAYSEPLAVKSNRITLSHTAPLWVA |
| | | | DDSLALQVSEPIFIDDDDKLALQTAAPLVTNAGTLRLQSA |
| | | | APLGLVENTLRLLFSKPLYLQNDFLALAIERPLAIAAAGT |
| | | | LALQLTPPLKTNDDGLTLSTVEPLTVKNGNLGLQISRPLW |
| | | | VQNSSLSLAITPPLRLFNSDPVLGLGFTFPLAVTNNLLSL |
| | | | NMGDGVKLTYNKLTANLGRDLQFENGAIAVTLTAELPLQY |
| | | | TNKLQLNIGAGLRYNGASRKLDVNINQNKGLTWDNDAVIP |
| | | | KLGSGLQFDPNGNIAVIPETVKPQTLWTTADPSPNCSVYQ |
| | | | NLDARLWLALVKSGDMVHGSIALKALKGTLLNPTASYISI |
| | | | VIYFYSNGVRRTNYPTFDNEGTLANSATWGYREGQSANTN |
| | | | VTNATEFMPSSTRYPVNKGDNIQNQSFSYTCIKGDFAMPV |
| | | | PFRVTYNHALEGYSLKFTWRVVANQAFDIPCCSFSYITE |
| 194 | RhAd59 | fiber-1 | MYLLITTHDFHLSQMKRTRVDEDFNPVYPYDAEQSSSVPF |
| | | protein | VTPPFTSSNGLTESPSGVLTLNYSQPLTTINGKLTLNVDG |
| | | | GLNINNDKKLQVRTNENEGVTLQNNAVSVKIGNDLRFDSR |
| | | | GQITLNPVSLWTTPSESANCTLYNSSANFFLCLTRNDAHV |
| | | | VGTVNLTGINGKFSSMTENSVIVQLAFDENGTLQSSPLAQ |
| | | | NVWGIRQTPNSPNAVNLLTFMPSSLVYPREGGGKPKNNLY |
| | | | FNTFLRGNLSRPITLTLTLNSLPTGYTLTFKWGTYLNEPF |
| | | | SVPTTCFCYVAEQ |
| 195 | RhAd59 | fiber-2 | MKRARVDESFNPVYPYDSTSTPAIPFVTPPFVSNDGLQEN |
| | | protein | PPGILSLQIAKPLFFNTQRKIALSLGRGLAITSAGELENT |
| | | | QRIQSTPPLLLNSNTLSMNYSSPLTVLDNKLALNYLSPLR |
| | | | WNDSLTFNFTSPLHVADGNLTFSYTSPLHVTNNSLALNYS |
| | | | APLRLLNNSLTFTYANPFKIANSSLSLKLDYPSALRLNNN |
| | | | GSLQVIAGWGLGIAGNNDLYVKRQAPLAVYESGSNAGTLY |
| | | | LRLGPNSALRTVNDNLTLNTGQGLQITNNTLSVKLGNGLR |
| | | | FNNTGAIELAQ |
| 196 | RhAd59 | long fiber | MKRARVDEDFNPVYPYDPPYAPIMPFITPPFTSSDGLQEK |
| | | protein | PFGVLSINYKDPITTQNGSLTLKVGNGLSINNQGQLTSTQ |
| | | | TTVTPPLTNESDTLGLAYSDPLTVKNNSLTLSHSAPFAVS |
| | | | NHSLSLKTTTPFFINQQNELALSTDAPLQVTAGSLRLQSA |
| | | | APLGIAEQTLRLLFSNPLFLQDNFLTLAIERPLAITSSGN |
| | | | LTVQLNPPFQSTSTGLALSLSNPLTISNGTLAMSIKRPFI |
| | | | IDDNKLYMDFRPPLRLFNSEPQLGVHFNPPITVQQGGLAL |
| | | | NTGDGLQVYLNKLTLNIGRDLQYENGSLVVKLTPAPPLQY |
| | | | TTQLQLNMGAGLHLGATKQLTLDVDTNRGLTWNNNKLAVN |
| | | | LGDGLQFNSYGQIIPTVKKDTLWTTADPSPNCSIYKDLDA |
| | | | KLWLSLVKSGGMVHGSIALQALKDTLLSPTDSFITIILYF |
| | | | DENGVRAPYPTFDNEGTLAKDATWGYRQGPSADTNVTNAL |
| | | | EFMPSSARYPRGKHEQAQNQTFGYTCLQGNFSWPIPFRVQ |
| | | | YNYVRTGFSFKFTWQWVNRQKFDIPCCSFSYITEQ |
| 197 | RhAd60 | fiber-1 | MSTSLPLPTIFIFSEMKRTRVDEDFNPVYPYDTTSTPAIP |
| | | protein | FISPPFVNSDGFQESPPGILSLRLNKPLSFTNHAVSLNVG |
| | | | SGLTINSTGFLEATQQISTANPLLLQNNTLSLISSAPLYV |
| | | | NGSALALRLNSPFVVHNNGLAINLAEPLYVTNNSLNLRLA |
| | | | SPFYVNNNSLNLRLPNPSGLSIDGSGLKVKAGYGLRLHAN |
| | | | NDLYVYTDFPLDKYDNGAKQGQLYLRTGKGLQLSNNDLTV |
| | | | KLGAGLKFDNNGAITLAST |
| 198 | RhAd60 | fiber-2 | MKRARIDEDYNPVYPYNTSTPPFIAPPFVDNDGFQENPQG |
| | | protein | VLSLRISKPLYFDMNKRLSLSLGQGLSTTSAGELESAQNV |
| | | | RANPPLSINSNTVSLRYSSPLNLTENGLVLGYSDPLRWGG |
| | | | ALTFNYTSPLRYENDSLTFNYTSPLKLMNNSLAIEVNSSK |
| | | | GVTSKNGAIAVQLSPDLKFDSNGSIAFGIQTLWTFPTTTS |
| | | | NCTVFTSNDSLLSLCLTKCGAHVLGSVSLTGMAGALLNMT |
| | | | QTSVTIQFTFDNTGSLTSSPLINNAWGIRQNDSASPNPTF |
| | | | NALTFMPNSTVYSRGGSGEPRNNYYTQTYLRGNIRKPIVL |
| | | | TVTYNSANTGYSLSFKWDAIATEKFATPTSSFCYISEQ |
| 199 | RhAd60 | long fiber | MGCYCNRKICHSYIFILLHFRTIKSCYTPSRFFQMKRARV |
| | | protein | DEDFNPVYPYDSPNAPIMPFITPPFTSSDGLQEKPLGVLS |
| | | | LNYKDPVTTQNGSLTLKLGSGLNINDQGQLTSTAGEVEPP |
| | | | LTNNNNKLALAYSQPLAVENNRLIVSYNAPLAVTGNSLGL |
| | | | QVSEPIFINDNDKLALQTAAPLKTNAGTLRLQSAAPLGLA |
| | | | EETLKLLFSKPLYLQGDFLSLGIERPLAIAAAGTLALQLT |
| | | | PPLTTNDSGLTISTADPVTVSNGNLGLRISRPLVVQNNSL |
| | | | SLALTPPLRLFNSEPVLGLGFTFPITWVDNLLSLNIGNGL |
| | | | TLLYNKLTANLGRDLQFQNGAIAVSLTTEPPLQYTTKLQL |
| | | | NVGAGLRYNGASRKLDVDINQQKGLTWENNAVVPKLGQGL |
| | | | YFDPQGNIAMSPTNVKPDTLWTTADPSPNCSVYTDLDAKL |
| | | | WLSLVKCGGMVQGSIALQAQKGTLMSVTDPSVTIILHFND |
| | | | NGVRINYPTLDNQGTLGRDSTWGYRQGQSANTNVTNAVEF |
| | | | MPNSSRYPRGRGEQAQNQTVGYTCLQGNLSMPIFFQVQYN |
| | | | YVTTGYSFKFTWQAVQRQKFDIPCCSFSYITEE |
| 200 | RhAd61 | fiber-1 | MRWWAANFLQTLKEILLSTSLLFPTIFIFSEMKRTRVDED |
| | | protein | FNPVYPYNTEPTPSAPFVTPPFVSSNGLMESPSGVLSIRY |
| | | | TQPITVVNNSLSLKTGNGLSVSSDGKLQIAINTNKGLSNE |
| | | | DNKIAVKVGPELYFDADGAITMNSQTLWTNPNETANCSVF |
| | | | QSLDSLLTLCLTKNGAHVLGSVSLTGLSGPLLKMTTTSVT |
| | | | VQLIFDSNGVLTTSQLNTNSWGMRANTNLNAPVTNALPFM |
| | | | PNSTIYARGNAGEPRSNYYVQTYLRGNLNKQITLSISFNA |
| | | | SSSGYSLTFKWSAIATEKFATPTSSFCYIAEQ |
| 201 | RhAd61 | fiber-2 | MKRTRIEDDFNPVYPYDSVSAPAVPFVSPPFVNSDGLKES |
| | | protein | PPGVLSLQIAKPLLFDAQRKVTLSLGRGLAVNSAGQLEST |
| | | | QILQTNPPLLLSNNMLSLDCAFPLSVANNQLSLKYTDPLR |
| | | | LVNDSLTFNFTSPLHLANGNLTFSYTTPLNIINNSLALNY |
| | | | SPPLRLVNSNLTMGYADPFQVANSNLSLKIANPSGLRIRN |
| | | | GNLQIAAGYGLQIAGNNDLYVRRETPLDVYTSGAKEGILY |
| | | | LRLGPNSGLQVLSDNLSLKTGQGLQILSNTLSVKLGRGLR |
| | | | FDSSGAIELST |
| 202 | RhAd61 | long fiber | MKRARIEEDFNPVYPYDPPYAPIMPFITPPFTSSDGLQEK |
| | | protein | PFGVLSLNYKNPITTQNGSLTLKLGNGLNINNQGELTSTA |
| | | | GAVQPPLTNADNKLTLAYSEPLTVQNKRLTLSHSAPLAVA |
| | | | DNSLSLQVTDPIFINADNKLALQTDAPLNTTTGVLRLKSA |
| | | | APLGLVEQTLNLLFAKPLYLQGDFLALAIERPLAVTADGS |
| | | | LALALDPPLKTSNTGLGLLTAAPLTVTNGNLNLNVKRPFV |
| | | | IQDNSLYMDFRAPLRLFNSEPQLGVNFTSPLTVRADALTL |
| | | | DTGNGLQVLFNKLTLNLGRDLEYDNGAIAVRLNPAAPLQY |
| | | | TNQLQLNLGAGLRLGVSKKLTVDVDENKGLTWNGNQLVAS |
| | | | LGTGLAFDNYGRISWVSTAAGDDTLWTTADPSPNCTVYEQ |
| | | | LDARLWLSLVKCGGMVQGSVALKAEKGRLLNPTASFISIV |
| | | | MYFNRYGVWRTDYPTIGDDQGIIANSATWGYRRGESADTN |
| | | | VTNALKFMPSSRRYPINQGNNIQNQMITYTCIQGDVSMPV |
| | | | PLKITFNHAFEGYSFKFTWRWVANQRFDIPCCSFSYITEE |
| 203 | RhAd62 | fiber-1 | MKRTRVDEDFNPVYPYDSTSTPAVPFISPPFVNSDGLQEN |
| | | protein | PPGVLSLRIAKPLYFDMERKLALSLGRGLAITSTGQLEST |
| | | | QSVQTTPPLVVNNSNTLVLRYSSPLGLSGDNLILNCSDPL |
| | | | RVINNSLTFSYLSPLRFEGGSLTFNYTSPLKLLNSSLAIG |
| | | | INSNKGLGNDSDELSVKLTSDLKENNDGKIAFGIQSLCTT |
| | | | PTAASNCTVFTNGDSLLCLCLTKCGAHVLGSVSLTGMQGT |
| | | | ITAMTQNYISIQFLFDHNGALTSSPLLNNNTWGIRQSDTS |
| | | | SANPAYNALAFMPNSTVYVRGQSGEPRNNYYTQTYLRGNV |
| | | | KKPIILTVTYNSAASGYSLTSKWDAVTEKFATPTSSFCYI |
| | | | TEQ |
| 204 | RhAd62 | long fiber | MKRARVDEDFNPVYPYDSPNAPIMPFITPPFTSSDGLQEK |
| | | protein | PLGVLSLNYKDPVTTQNGSLTLKLGSGLNINDQGQLTSTA |
| | | | GEVEPPLTNNNNKLALAYSQPLAVENNRLIVSYNAPLAVT |
| | | | GNSLGLQVSEPIFINDNDKLALQTAAPLKTNAGTLRLQSA |
| | | | APLGLAEETLKLLFSKPLYLQGDFLSLGIERPLAIAAAGT |
| | | | LALQLTPPLTTNDSGLTISTADPVTVSNGNLGLRISRPLV |
| | | | VQNNSLSLALTPPLRLFNSEPVLGLGFTFPITWVDNLLSL |
| | | | NIGNGLTLLYNKLTANLGRDLQFQNGAIAVSLTTEPPLQY |
| | | | TTKLQLNVGAGLRYNGASRKLDVDINQQKGLTWENNAVVP |
| | | | KLGQGLYFDPQGNIAMSPTNVKPDTLWTTADPSPNCSVYT |
| | | | DLDAKLWLSLVKCGGMVQGSIALQAQKGTLMSVTDPSVTI |
| | | | ILHFNDNGVRINYPTLDNQGTLGRDSTWGYRQGQSANTNV |
| | | | TNAVEFMPNSSRYPRGRGEQAQNQTVGYTCLQGNLSMPIF |
| | | | FQVQYNYVTTGYSFKFTWQAVQRQKFDIPCCSFSYITEE |
| 205 | RhAd63 | fiber-1 | MSTSLPLPTIFIFSEMKRTRVDEDFNPVYPYDTTSTPAIP |
| | | protein | FISPPFVNSDGFQESPPGILSLRLNKPLSFTNHAVSLNVG |
| | | | SGLTINSTGFLEATQQISTANPLLLQNNTLSLISSAPLYV |
| | | | NGSALALRLNSPFVVHNNGLAINLAEPLYVTNNSLNLRLA |
| | | | SPFYVNNNSLNLRLPNPSGLSIDGSGLKVKAGYGLRLHAN |
| | | | NDLYVYTDFPLDKYDNGAKQGQLYLRTGKGLQLSNNDLTV |
| | | | KLGAGLKFDNNGAITLAST |
| 206 | RhAd63 | fiber-2 | MKRARIDEDYNPVYPYNTSTPPFIAPPFVDNDGFQENPQG |
| | | protein | VLSLRISKPLYFDMNKRLSLSLGQGLSTTSAGELESAQNV |
| | | | RANPPLSINSNTVSLRYSSPLNLTENGLVLGYSDPLRWGG |
| | | | ALTFNYTSPLRYENDSLTFNYTSPLKLMNNSLAIEVNSSK |
| | | | GVTSKNGAIAVQLSPDLKFDSNGSIAFGIQTLWTFPTTTS |
| | | | NCTVFTSNDSLLSLCLTKCGAHVLGSVSLTGMAGALLNMT |
| | | | QTSVTIQFTFDNTGSLTSSPLINNAWGIRQNDSASPNPTF |
| | | | NALTFMPNSTVYSRGGSGEPRNNYYTQTYLRGNIRKPIVL |
| | | | TVTYNSANTGYSLSFKWDAIATEKFATPTSSFCYISEQ |
| 207 | RhAd63 | long fiber | MGCYCNRKICHSYIFILLHFRTIKSCYTPSRFFQMKRARV |
| | | protein | DEDFNPVYPYDSPNAPIMPFITPPFTSSDGLQEKPLGVLS |
| | | | LNYKDPVTTQNGSLTLKLGSGLNINDQGQLTSTAGEVEPP |
| | | | LTNNNNKLALAYSQPLAVENNRLIVSYNAPLAVTGNSLGL |
| | | | QVSEPIFINDNDKLALQTAAPLKTNAGTLRLQSAAPLGLA |
| | | | EETLKLLFSKPLYLQGDFLSLGIERPLAIAAAGTLALQLT |
| | | | PPLTTNDSGLTISTADPVTVSNGNLGLRISRPLVVQNNSL |
| | | | SLALTPPLRLFNSEPVLGLGFTFPITWDNLLSLNIGNGLT |
| | | | LLYNKLTANLGRDLQFQNGAIAVSLTTEPPLQYTTKLQLN |
| | | | VGAGLRYNGASRKLDVDINQQKGLTWENNAVVPKLGQGLY |
| | | | FDPQGNIAMSPTNVKPDTLWTTADPSPNCSVYTDLDAKLW |
| | | | LSLVKCGGMVQGSIALQAQKGTLMSVTDPSVTIILHFNDN |
| | | | GVRINYPTLDNQGTLGRDYANFFPSPVQLCNHWIFLQIHL |
| | | | ASCTKAKV |
| 208 | RhAd64 | fiber-1 | MSTSLPLPTIFIFSEMKRTRVDEDFNPVYPYDTTSTPAIP |
| | | protein | FISPPFVNSDGFQESPPGILSLRLNKPLSFTNHAVSLNVG |
| | | | SGLTINSTGFLEATQQISTANPLLLQNNTLSLISSAPLYV |
| | | | NGSALALRLNSPFVVHNNGLAINLAEPLYVTNNSLNLRLA |
| | | | SPFYVNNNSLNLRLPNPSGLSIDGSGLKVKAGYGLRLHAN |
| | | | NDLYVYTDFPLDKYDNGAKQGQLYLRTGKGLQLSNNDLTV |
| | | | KLGAGLKFDNNGAITLAST |
| 209 | RhAd64 | fiber-2 | MKRARIDEDYNPVYPYNTSTPPFIAPPFVDNDGFQENPQG |
| | | protein | VLSLRISKPLYFDMNKRLSLSLGQGLSTTSAGELESAQNV |
| | | | RANPPLSINSNTVSLRYSSPLNLTENGLVLGYSDPLRVVG |
| | | | GALTFNYTSPLRYENDSLTFNYTSPLKLMNNSLAIEVNSS |
| | | | KGVTSKNGAIAVQLSPDLKFDSNGSIAFGIQTLWTFPTTT |
| | | | SNCTVFTSNDSLLSLCLTKCGAHVLGSVSLTGMAGALLNM |
| | | | TQTSVTIQFTFDNTGSLTSSPLINNAWGIRQNDSASPNPT |
| | | | FNALTFMPNSTVYSRGGSGEPRNNYYTQTYLRGNIRKPIV |
| | | | LTVTYNSANTGYSLSFKWDAIATEKFATPTSSFCYISEQ |
| 210 | RhAd64 | long fiber | MGCYCNRKICHAYIFILLHFRTIKSCYTPSRFFQMKRARV |
| | | protein | DEDFNPVYPYDSPNAPIMPFITPPFTSSDGLQEKPLGVLS |
| | | | LNYKDPVTTQNGSLTLKLGSGLNINDQGQLTSTAGEVEPP |
| | | | LTNNNNKLALAYSQPLAVENNRLIVSYNAPLAVTGNSLGL |
| | | | QVSEPIFINDNDKLALQTAAPLKTNAGTLRLQSAAPLGLA |
| | | | EETLKLLFSKPLYLQGDFLSLGIERPLAIAAAGTLALQLT |
| | | | PPLTTNDSGLTISTADPVTVSNGNLGLRISRPLVVQNNSL |
| | | | SLALTPPLRLFNSEPVLGLGFTFPITVVDNLLSLNIGNGL |
| | | | TLLYNKLTANLGRDLQFQNGAIAVSLTTEPPLQYTTKLQL |
| | | | NVGAGLRYNGASRKLDVDINQQKGLTWENNAVVPKLGQGL |
| | | | YFDPQGNIAMSPTNVKPDTLWTTADPSPNCSVYTDLDAKL |
| | | | WLSLVKCGGMVQGSIALQAQKGTLMSVTDPSVTIILHFND |
| | | | NGVRINYPTLDNQGTLGRDSTWGYRQGQSANTNVTNAVEF |
| | | | MPNSSRYPRGRGEQAQNQTVGYTCLQGNLSMPIFFQVQYN |
| | | | YVTTGYSFKFTWQAVQRQKFDIPCCSFSYITEE |
| 211 | RhAd65 | fiber-1 | MSTSLPLPTIFIFSEMKRTRVDEDFNPVYPYDTTSTPAIP |
| | | protein | FISPPFVNSDGFQESPPGILSLRLNKPLSFTNHAVSLNVG |
| | | | SGLTINSTGFLEATQQISTANPLLLQNNTLSLISSAPLYV |
| | | | NGSALALRLNSPFWHNNGLAINLAEPLYVTNNSLNLRLAS |
| | | | PFYVNNNSLNLRLPNPSGLSIDGSGLKVKAGYGLRLHANN |
| | | | DLYVYTDFPLDKYDNGAKQGQLYLRTGKGLQLSNNDLTVK |
| | | | LGAGLKFDNNGAITLAST |
| 212 | RhAd65 | fiber-2 | MKRARIDEDYNPVYPYNTSTPPFIAPPFVDNDGFQENPQG |
| | | protein | VLSLRISKPLYFDMNKRLSLSLGQGLSTTSAGELESAQNV |
| | | | RANPPLSINSNTVSLRYSSPLNLTENGLVLGYSDPLRWVG |
| | | | GALTFNYTSPLRYENDSLTFNYTSPLKLMNNSLAIEVNSS |
| | | | KGVTSKNGAIAVQLSPDLKFDSNGSIAFGIQTLWTFPTTT |
| | | | SNCTVFTSNDSLLSLCLTKCGAHVLGSVSLTGMAGALLNM |
| | | | TQTSVTIEFTFDNTGSLTSSPLINNAWGIRQNDSASPNPT |
| | | | FNALTFMPNSTVYSRGGSGEPRNNYYTQTYLRGNIRKPIV |
| | | | LTVTYNSANTGYSLSFKWDAIATEKFATPTSSFCYISEQ |
| 213 | RhAd65 | long fiber | MGCYCNRKICHSYIFILLHFRTIKSCYTPSRFFQMKRARV |
| | | protein | DEDFNPVYPYDSPNAPIMPFITPPFTSSDGLQEKPLGVLS |
| | | | LNYKDPVTTQNGSLTLKLGSGLNINDQGQLTSTAGEVEPP |
| | | | LTNNNNKLALAYSQPLAVENNRLIVSYNAPLAVTGNSLGL |
| | | | QVSEPIFINDNDKLALQTAAPLKTNAGTLRLQSAAPLGLA |
| | | | EETLKLLFSKPLYLQGDFLSLGIERPLAIAAAGTLALQLT |
| | | | PPLTTNDSGLTISTADPVTVSNGNLGLRISRPLVVQNNSL |
| | | | SLALTPPLRLFNSEPVLGLGFTFPITVVDNLLSLNIGNGL |
| | | | TLLYNKLTANLGRDLQFQNGAIAVSLTTEPPLQYTTKLQL |
| | | | NVGAGLRYNGASRKLDVDINQQKGLTWENNAVVPKLGQGL |
| | | | YFDPQGNIAMSPTNVKPDTLWTTADPSPNCSVYTDLDAKL |
| | | | WLSLVKCGGMVQGSIALQAQKGTLMSVTEPSVTIILHFND |
| | | | NGVRINYPTLDNQGTLGRDSTWGYRQGQSANTNVTNAVEF |
| | | | MPNSSRYPRGRGEQAQNQTVGYTCLQGNLSMPIFFQVQYN |
| | | | YVTTGYSFKFTWQAVQRQKFDIPCCSFSYITEE |
| 214 | RhAd66 | fiber-1 | MSTSLPLPTIFIFSEMKRTRVDEDFNPVYPYDTTSTPAIP |
| | | protein | FISPPFVNSDGFQESPPGILSLRLNKPLSFTNHAVSLNVG |
| | | | SGLTINSTGFLEATQQISAANPLLLQNNTLSLISSAPLYV |
| | | | NGSALALRLNSPFVVHNNGLAINLAEPLYVTNNSLNLRLA |
| | | | SPFYVNNNSLNLRLPNPSGLSIDGSGLKVKAGYGLRLHAN |
| | | | NDLYVYTDFPLDKYDNGAKQGQLYLRTGKGLQLSNNDLTV |
| | | | KLGAGLKFDNNGAITLAST |
| 215 | RhAd66 | fiber-2 | MKRARIDEDYNPVYPYNTSTPPFIAPPFVDNDGFQENPQG |
| | | protein | VLSLRISKPLYFDINKRLSLSLGQGLSTTSAGELESAQNV |
| | | | RANPPLSINSNTVSLRYSSPLNLTENGLVLGYSDPLRVGG |
| | | | ALTFNYTSPLRYENDSLTFNYTSPLKLMNNSLAIEVNSSK |
| | | | GVTSKNGAIAVQLSPDLKFDSNGSIAFGIQTLWTFPTTTS |
| | | | NCTVFTSNDSLLSLCLTKCGAHVLGSVSLTGMAGALLNMT |
| | | | QTSVTIQFTFDNTGSLTSSPLINNAWGIRQNDSASPNPTF |
| | | | NALTFMPNSTVYSRGGSGEPRNNYYTQTYLRGNIRKPIVL |
| | | | TVTYNSANTGYSLSFKWDAIATEKFATPTSSFCYISEQ |
| 216 | RhAd66 | long fiber | MGCYCNRKICHSYIFILLHFRTIKSCYTPSRFFQMKRARV |
| | | protein | DEDFNPVYPYDSPNAPIMPFITPPFTSSDGLQEKPLGVLS |
| | | | LNYKDPVTTQNGSLTLKLGSGLNINDQGQLTSTAGEVEPP |
| | | | LTNNNNKLALAYSQPLAVENNRLIVSYNAPLAVTGNSLGL |
| | | | QVSEPIFINDNDKLALQTAAPLKTNAGTLRLQSAAPLGLA |
| | | | EETLKLLFSKPLYLQGDFLSLGIERPLAIAAAGTLALQLT |
| | | | PPLTTNDSGLTISTADPVTVSNGNLGLRISRPLVVQNNSL |
| | | | SLALTPPLRLFNSEPVLGLGFTFPITVVDNLLSLNIGNGL |
| | | | TLLYNKLTANLGRDLQFQNGAIAVSLTTEPPLQYTTKLQL |
| | | | NVGAGLRYNGASRKLDVDINQQKGLTWENNAVVPKLGQGL |
| | | | YFDPQGNIAMSPTNVKPDTLWTTADPSPNCSVYTDLDAKL |
| | | | WLSLVKCGGMVQGSIALQAQKGTLMSVTDPSVTIILHFND |
| | | | NGVRINYPTLDNQGTLGRDSTWGYRQGQSANTNVTNAVEF |
| | | | MPNSSRYPRGRGEQAQNQTVGYTCLQGNLSMPIFFQVQYN |
| | | | YVTTGYSFKFTWQAVQRQKFDIPCCSFSYITEE |
| 217 | RhAd67 | fiber-1 | MYLLITTHDFHLSQMKRTRVDEDFNPVYPYDAEQSSSVPF |
| | | protein | VTPPFTSSNGLTESPSGVLTLNYSQPLTTINGKLTLNVDG |
| | | | GLNINNDKKLQVRTNENEGVTLQNNAVSVKIGNDLRFDSR |
| | | | GQITLNPVSLWTTPSESANCTLYNSSANFFLCLTRNDAHV |
| | | | VGTVNLTGINGKFSSMTENSVIVQLAFDENGTLQSSPLAQ |
| | | | NVWAYVKLLTLQTLLICLHLCPAA |
| 218 | RhAd67 | fiber-2 | MKRARVDESFNPVYPYDSTSTPAIPFVTPPFVSNDGLQEN |
| | | protein | PPGILSLQIAKPLFFNTQRKIALSLGRGLAITSAGELENT |
| | | | QRIQSTPPLLLNSNTLSMNYSSPLTVLDNKLALNYLSPLR |
| | | | WNDSLTFNFTSPLHVADGNLTFSYTSPLHVTNNSLALNYS |
| | | | APLRLLNNSLTFTYANPFKIANSSLSLKLDYPSALRLNNN |
| | | | GSLQVIAGWGLGIAGNNDLYVKRQAPLAVYESGSNAGTLY |
| | | | LRLGPNSALRTVNDNLTLNTGQGLQITNNTLSVKLGNGLR |
| | | | FNNTGAIELAQ |
| 219 | RhAd67 | long fiber | MKRARVDEDFNPVYPYDPPYAPIMPFITPPFTSSDGLQEK |
| | | protein | PFGVLSINYKDPITTQNGSLTLKVGNGLSINNQGQLTSTQ |
| | | | TTVTPPLTNESDTLGLAYSDPLTVKNNSLTLSHSAPFAVS |
| | | | NHSLSLKTTTPFFINQQNELALSTDAPLQVTAGSLRLQSA |
| | | | APLGIAEQTLRLLFSNPLFLQDNFLTLAIERPLAITSSGN |
| | | | LTVQLNPPFQSTSTGLALSLSNPLTISNGTLAMSIKRPFI |
| | | | IDDNKLYMDFRPPLRLFNSEPQLGVHFNPPITVQQGGLAL |
| | | | NTGDGLQVYLNKLTLNIGRDLQYENGSLVVKLTPAPPLQY |
| | | | TTQLQLNMGAGLHLGATKQLTLDVDTNRGLTWNNNKLAVN |
| | | | LGDGLQFNSYGQIIPTVKKDTLWTTADPSPNCSIYKDLDA |
| | | | KLWLSLVKSGGMVHGSIALQALKDTLLSPTDSFITIILYF |
| | | | DENGVRAPYPTFDNEGTLAKDATWGYRQGPSADTNVTNAL |
| | | | EFMPSSARYPRGKHEQAQNQTFGYTCLQGNFSWPIPFRVQ |
| | | | YNYVRTGFSFKFTWQVVNRQKFDIPCCSFSYITEQ |
| 220 | RhAd51 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTVVQNNDFTPTEAGTQTINFDERSRWGGQLK |
| | | | TILHTNMPNINEFMSTNKFRAKLMVEKSNAETRQPRYEWF |
| | | | EFTIPEGNYSETMTIDLMNNAIVDNYLQVGRQNGVLESDI |
| | | | GVKFDTRNFRLGWDPVTKLVMPGVYTNEAFHPDIVLLPGC |
| | | | GVDFTQSRLSNLLGIRKRRPFQEGFQIMYEDLEGGNIPAL |
| | | | LDVSKYEASIQRAKAEGREIRGDTFAVAPQDLEIVPLTKD |
| | | | SKDRSYNIINNTTDTLYRSWFLAYNYGDPEKGVRSWTILT |
| | | | TTDVTCGSQQVYWSLPDMMQDPVTFRPSTQVSNFPVVGTE |
| | | | LLPVHAKSFYNEQAVYSQLIRQSTALTHVFNRFPENQILV |
| | | | RPPAPTITTVSENVPALTDHGTLPLRSSISGVQRVTITDA |
| | | | RRRTCPYVYKALGWVAPKVLSSRTF |
| 221 | RhAd52 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTWVQNNDFTPTEAGTQTINFDERSRWGGQLK |
| | | | TILHTNMPNINEFMSTNKFRARLMVKKVENQPPEYEWFEF |
| | | | TIPEGNYSETMTIDLMNNAIVDNYLQVGRQNGVLESDIGV |
| | | | KFDTRNFRLGWDPVTKLVMPGVYTNEAFHPDIVLLPGCGV |
| | | | DFTQSRLSNLLGIRKRRPFQEGFQIMYEDLEGGNIPALLD |
| | | | VTKYEQSVQRAKAEGREIRGDTFAVSPQDLVIEPLEHDSK |
| | | | NRSYNLLPNKTDTAYRSWFLAYNYGDPEKGVRSWTILTTT |
| | | | DVTCGSQQVYWSLPDMMQDPVTFRPSTQVSNFPVVGTELL |
| | | | PVHAKSFYNEQAVYSQLIRQSTALTHVFNRFPENQILVRP |
| | | | PAPTITTVSENVPALTDHGTLPLRSSISGVQRVTITDARR |
| | | | RTCPYVYKALGVVAPKVLSSRTF |
| 222 | RhAd53 | penton | MRRAVRVTPAVYAEGPPPSYESVMGSANVPATLEAPYVPP |
| | | protein | RYLGPTEGRNSIRYSELAPLYDTTKVYLVDNKSADIASLN |
| | | | YQNDHSNFLTTVVQNNDFTPTEAGTQTINFDERSRWGGQL |
| | | | KTILHTNMPNINEFMSTNKFRARLMVEKTSGQPPKYEWFE |
| | | | FTIPEGNYSETMTIDLMNNAIVDNYLQVGRQNGVLESDIG |
| | | | VKFDTRNFRLGWDPVTKLVMPGVYTNEAFHPDIVLLPGCG |
| | | | VDFTQSRLSNLLGIRKRRPFQEGFQIMYEDLEGGNIPGLL |
| | | | DVPAYEQSLQQAQEEGRVTRGDTFATAPNEVVIKPLLKDS |
| | | | KDRSYNIITDTTDTLYRSWFLAYNYGDPENGVRSWTILTT |
| | | | TDVTCGSQQVYWSLPDMMQDPVTFRPSTQVSNFPVVGTEL |
| | | | LPVHAKSFYNEQAVYSQLIRQSTALTHVFNRFPENQILVR |
| | | | PPAPTITTVSENVPALTDHGTLPLRSSISGVQRVTITDAR |
| | | | RRTCPYVYKALGVVAPKVLSSRTF |
| 223 | RhAd54 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTWVQNNDFTPTEAGTQTINFDERSRWGGQLK |
| | | | TILHTNMPNINEFMSTNKFRARLMVKKAENQPPEYEWFEF |
| | | | TIPEGNYSETMTIDLMNNAIVDNYLQVGRQNGVLESDIGV |
| | | | KFDTRNFRLGWDPVTKLVMPGVYTNEAFHPDIVLLPGCGV |
| | | | DFTQSRLSNLLGIRKRRPFQEGFQIMYEDLEGGNIPGLLD |
| | | | VPAYEESVKQAEAQGREIRGDTFATEPHELVIKPLEQDSK |
| | | | KRSYNIISGTMNTLYRSWFLAYNYGDPEKGVRSWTILTTT |
| | | | DVTCGSQQVYWSLPDMMQDPVTFRPSTQVSNFPVVGTELL |
| | | | PVHAKSFYNEQAVYSQLIRQSTALTHVFNRFPENQILVRP |
| | | | PAPTITTVSENVPALTDHGTLPLRSSISGVQRVTITDARR |
| | | | RTCPYVYKALGWVAPKVLSSRTF |
| 224 | RhAd55 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTWVQNNDFTPTEAGTQTINFDERSRWGGQLK |
| | | | TILHTNMPNINEFMSTNKFRAKLMVEKSNAETRQPRYEWF |
| | | | EFTIPEGNYSETMTIDLMNNAIVDNYLQVGRQNGVLESDI |
| | | | GVKFDTRNFRLGWDPVTKLVMPGVYTNEAFHPDIVLLPGC |
| | | | GVDFTQSRLSNLLGIRKRRPFQEGFQIMYEDLEGGNIPAL |
| | | | LDVSKYEASIQRAKAEGREIRGDTFAVAPQDLEIVPLTKD |
| | | | SKDRSYNIINNTTDTLYRSWFLAYNYGDPEKGVRSWTILT |
| | | | TTDVTCGSQQVYWSLPDMMQDPVTFRPSTQVSNFPVVGTE |
| | | | LLPVHAKSFYNEQAVYSQLIRQSTALTHVFNRFPENQILV |
| | | | RPPAPTITTVSENVPALTDHGTLPLRSSISGVQRVTITDA |
| | | | RRRTCPYVYKALGWVAPKVLSSRTF |
| 225 | RhAd56 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTWVQNNDFTPTEAGTQTINFDERSRWGGQLK |
| | | | TILHTNMPNINEFMSTNKFRARLMVKKVENQPPEYEWFEF |
| | | | TIPEGNYSETMTIDLMNNAIVDNYLQVGRQNGVLESDIGV |
| | | | KFDTRNFRLGWDPVTKLVMPGVYTNEAFHPDIVLLPGCGV |
| | | | DFTQSRLSNLLGIRKRRPFQEGFQIMYEDLEGGNIPGLLD |
| | | | VPAYEDSVKKAEAQGREIRGDTFATEPHALVIKPLTEDSK |
| | | | KRSYNIITGTMDTEYRSWFLAYNYGDPEKGVRSWTILTTT |
| | | | DVTCGSQQVYWSLPDMMQDPVTFRPSTQVSNFPVVGTELL |
| | | | PVHAKSFYNEQAVYSQLIRQSTALTHVFNRFPENQILVRP |
| | | | PAPTITTVSENVPALTDHGTLPLRSSISGVQRVTITDARR |
| | | | RTCPYVYKALGVVAPKVLSSRTF |
| 226 | RhAd57 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTVVQNNDFTPTEAGTQTINFDERSRWGGQLK |
| | | | TILHTNMPNINEFMSTNKFRARLMVKKVENQPPEYEWFEF |
| | | | TIPEGNYSETMTIDLMNNAIVDNYLQVGRQNGVLESDIGV |
| | | | KFDTRNFRLGWDPVTKLVMPGVYTNEAFHPDIVLLPGCGV |
| | | | DFTQSRLSNLLGIRKRRPFQEGFQIMYEDLEGGNIPALLD |
| | | | VSKYEDSVKKAEAQGREIRGDTFAVAPQDLVIEPLTKDSK |
| | | | NRSYNIITGTMDTKYRSWFLAYNYGDPEKGVRSWTILTTT |
| | | | DVTCGSQQVYWSLPDMMQDPVTFRPSTQVSNFPVVGTELL |
| | | | PVHAKSFYNEQAVYSQLIRQSTALTHVFNRFPENQILVRP |
| | | | PAPTITTVSENVPALTDHGTLPLRSSISGVQRVTITDARR |
| | | | RTCPYVYKALGWVAPKVLSSRTF |
| 227 | RhAd58 | penton | MRRAVRVTPAVYAEGPPPSYESVMGSANVPATLEAPYVPP |
| | | protein | RYLGPTEGRNSIRYSELAPLYDTTKVYLVDNKSADIASLN |
| | | | YQNDHSNFLTTVVQNNDFTPTEAGTQTINFDERSRWGGQL |
| | | | KTILHTNMPNINEFMSTNKFRARLMVEKTNAETQQPRYEW |
| | | | FEFTIPEGNYSETMTIDLMNNAIVDNYLQVGRQNGVLESD |
| | | | IGVKFDTRNFRLGWDPVTKLVMPGVYTNEAFHPDIVLLPG |
| | | | CGVDFTQSRLSNLLGIRKRRPFQEGFQIMYEDLEGGNIPA |
| | | | LLDVSKYEASVQRAEAEGREIRGDTFAVAPQDLVIEPLTK |
| | | | DSKNRSYNIITGTTDTQYRSWFLAYNYGDPDKGVRSWTIL |
| | | | TTTDVTCGSQQVYWSLPDMMQDPVTFRPSTQVSNFPVVGT |
| | | | ELLPVHAKSFYNEQAVYSQLIRQSTALTHVFNRFPENQIL |
| | | | VRPPAPTITTVSENVPALTDHGTLPLRSSISGVQRVTITD |
| | | | ARRRTCPYVYKALGWVAPKVLSSRTF |
| 228 | RhAd59 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTWVQNNDFTPTEAGTQTINFDERSRWGGQLK |
| | | | TILHTNMPNINEFMSTNKFRARLMVKKAENQPPEYEWFEF |
| | | | TIPEGNYSETMTIDLMNNAIVDNYLQVGRQNGVLESDIGV |
| | | | KFDTRNFRLGWDPVTKLVMPGVYTNEAFHPDIVLLPGCGV |
| | | | DFTQSRLSNLLGIRKRRPFQEGFQIMYEDLEGGNIPGLLD |
| | | | VPAYEDSVKKAEAQGREIRGDTFATEPHALVIKPLTEDSK |
| | | | KRSYNIITGTMDTEYRSWFLAYNYGDPEKGVRSWTILTTT |
| | | | DVTCGSQQVYWSLPDMMQDPVTFRPSTQVSNFPVVGTELL |
| | | | PVHAKSFYNEQAVYSQLIRQSTALTHVFNRFPENQILVRP |
| | | | PAPTITTVSENVPALTDHGTLPLRSSISGVQRVTITDARR |
| | | | RTCPYVYKALGWVAPKVLSSRTF |
| 229 | RhAd60 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTWVQNNDFTPTEAGTQTINFDERSRWGGQLK |
| | | | TILHTNMPNINEFMSTNKFRAKLMVEKSNAETRQPRYEWF |
| | | | EFTIPEGNYSETMTIDLMNNAIVDNYLQVGRQNGVLESDI |
| | | | GVKFDTRNFRLGWDPVTKLVMPGVYTNEAFHPDIVLLPGC |
| | | | GVDFTQSRLSNLLGIRKRRPFQEGFQIMYEDLEGGNIPAL |
| | | | LDVSKYEASIQRAKAEGREIRGDTFAVAPQDLEIVPLTKD |
| | | | SKDRSYNIINNTTDTLYRSWFLAYNYGDPEKGVRSWTILT |
| | | | TTDVTCGSQQVYWSLPDMMQDPVTFRPSTQVSNFPVVGTE |
| | | | LLPVHAKSFYNEQAVYSQLIRQSTALTHVFNRFPENQILV |
| | | | RPPAPTITTVSENVPALTDHGTLPLRSSISGVQRVTITDA |
| | | | RRRTCPYVYKALGWVAPKVLSSRTF |
| 230 | RhAd61 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTVVQNNDFTPTEAGTQTINFDERSRWGGQLK |
| | | | TILHTNMPNINEFMSTNKFRAKLMVEKSNAETQQPRYEWF |
| | | | EFTIPEGNYSETMTIDLMNNAIVDNYLQVGRQNGVLESDI |
| | | | GVKFDTRNFRLGWDPVTKLVMPGVYTNEAFHPDIVLLPGC |
| | | | GVDFTQSRLSNLLGIRKRRPFQEGFQIMYEDLEGGNIPGL |
| | | | LDVPAYEQSLQQAQEEGRVTRGDTFATAPHELVIQPLTKD |
| | | | SKNRSYNLIPNSTDTLYRSWFLAYNYGDPEKGVRSWTILT |
| | | | TTDVTCGSQQVYWSLPDMMQDPVTFRPSTQVSNFPVVGTE |
| | | | LLPVHAKSFYNEQAVYSQLIRQSTALTHVFNRFPENQILV |
| | | | RPPAPTITTVSENVPALTDHGTLPLRSSISGVQRVTITDA |
| | | | RRRTCPYVYKALGWAPKVLSSRTF |
| 231 | RhAd62 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTWVQNNDFTPTEAGTQTINFDERSRWGGQLK |
| | | | TILHTNMPNINEFMSTNKFRARLMVKKVENQPPEYEWFEF |
| | | | TIPEGNYSETMTIDLMNNAIVDNYLQVGRQNGVLESDIGV |
| | | | KFDTRNFRLGWDPVTKLVMPGVYTNEAFHPDIVLLPGCGV |
| | | | DFTQSRLSNLLGIRKRRPFQEGFQIMYEDLEGGNIPALLD |
| | | | VSKYEDSVKKAEAQGREIRGDTFAVAPQDLVIEPLTKDSK |
| | | | NRSYNIITGTMDTKYRSWFLAYNYGDPEKGVRSWTILTTT |
| | | | DVTCGSQQVYWSLPDMMQDPVTFRPSTQVSNFPVVGTELL |
| | | | PVHAKSFYNEQAVYSQLIRQSTALTHVFNRFPENQILVRP |
| | | | PAPTITTVSENVPALTDHGTLPLRSSISGVQRVTITDARR |
| | | | RTCPYVYKALGWAPKVLSSRTF |
| 232 | RhAd63 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTVVQNNDFTPTEAGTQTINFDERSRWGGQLK |
| | | | TILHTNMPNINEFMSTNKFRAKLMVEKSNAETRQPRYEWF |
| | | | EFTIPEGNYSETMTIDLMNNAIVDNYLQVGRQNGVLESDI |
| | | | GVKFDTRNFRLGWDPVTKLVMPGVYTNEAFHPDIVLLPGC |
| | | | GVDFTQSRLSNLLGIRKRRPFQEGFQIMYEDLEGGNIPAL |
| | | | LDVSKYEASIQRAKAEGREIRGDTFAVAPQDLEIVPLTKD |
| | | | SKDRSYNIINNTTDTLYRSWFLAYNYGDPEKGVRSWTILT |
| | | | TTDVTCGSQQVYWSLPDMMQDPVTFRPSTQVSNFPVVGTE |
| | | | LLPVHAKSFYNEQAVYSQLIRQSTALTHVFNRFPENQILV |
| | | | RPPAPTITTVSENVPALTDHGTLPLRSSISGVQRVTITDA |
| | | | RRRTCPYVYKALGWAPKVLSSRTF |
| 233 | RhAd64 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTVVQNNDFTPTEAGTQTINFDERSRWGGQLK |
| | | | TILHTNMPNINEFMSTNKFRAKLMVEKSNAETQQPRYEWF |
| | | | EFTIPEGNYSETMTIDLMNNAIVDNYLQVGRQNGVLESDI |
| | | | GVKFDTRNFRLGWDPVTKLVMPGVYTNEAFHPDIVLLPGC |
| | | | GVDFTQSRLSNLLGIRKRRPFQEGFQIMYEDLEGGNIPGL |
| | | | LDVPAYEQSLQQAQEEGRVTRGDTFATAPHELVIQPLTKD |
| | | | SKNRSYNLIPNSTDTLYRSWFLAYNYGDPEKGVRSWTILT |
| | | | TTDVTCGSQQVYWSLPDMMQDPVTFRPSTQVSNFPVVGTE |
| | | | LLPVHAKSFYNEQAVYSQLIRQSTALTHVFNRFPENQILV |
| | | | RPPAPTITTVSENVPALTDHGTLPLRSSISGVQRVTITDA |
| | | | RRRTCPYVYKALGVVAPKVLSSRTF |
| 234 | RhAd65 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTVVQNNDFTPTEAGTQTINFDERSRWGGQLK |
| | | | TILHTNMPNINEFMSTNKFRARLMVKKVENQPPEYEWFEF |
| | | | TIPEGNYSETMTIDLMNNAIVDNYLQVGRQHGVLESDIGV |
| | | | KFDTRNFRLGWDPVTKLVMPGVYTNEAFHPDIVLLPGCGV |
| | | | DFTQSRLSNLLGIRKRRPFQEGFQIMYEDLEGGNIPALLD |
| | | | VSKYEDSVKKAEAQGREIRGDTFAVAPQDLVIEPLTKDSK |
| | | | NRSYNIITGTMDTEYRSWFLAYNYGDPEKGVRSWTILTTT |
| | | | DVTCGSQQVYWSLPDMMQDPVTFRPSTQVSNFPVVGTELL |
| | | | PVHAKSFYNEQAVYSQLIRQSTALTHVFNRFPENQILVRP |
| | | | PAPTITTVSENVPALTDHGTLPLRSSISGVQRVTITDARR |
| | | | RTCPYVYKALGWVAPKVLSSRTF |
| 235 | RhAd66 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTWVQNNDFTPTEAGTQTINFDERSRWGGQLK |
| | | | TILHTNMPNINEFMSTNKFRAKLMVEKISGQPPKYEWFEF |
| | | | TIPEGNYSETMTIDLMNNAIVDNYLQVGRQNGVLESDIGV |
| | | | KFDTRNFRLGWDPVTKLVMPGVYTNEAFHPDIVLLPGCGV |
| | | | DFTQSRLSNLLGIRKRRPFQEGFQIMYEDLEGGNIPGLLD |
| | | | VPAYEDSVKKAEAQGREIRGDTFATEPHALVIKPLTEDSK |
| | | | KRSYNIITGTMDTEYRSWFLAYNYGDPEKGVRSWTILTTT |
| | | | DVTCGSQQVYWSLPDMMQDPVTFRPSTQVGNFPVVGTELL |
| | | | PVHAKSFYNEQAVYSQLIRQSTALTHVFNRFPENQILVRP |
| | | | PAPTITTVSENVPALTDHGTLPLRSSISGVQRVTITDARR |
| | | | RTCPYVYKALGWVAPKVLSSRTF |
| 236 | RhAd67 | penton | MRRAVRVTPAAYEGPPPSYESVMGSANVPATLEAPYVPPR |
| | | protein | YLGPTEGRNSIRYSELAPLYDTTKVYLVDNKSADIASLNY |
| | | | QNDHSNFLTTVVQNNDFTPTEAGTQTINFDERSRWGGQLK |
| | | | TILHTNMPNINEFMSTNKFRAKLMVEKSNAETQQPRYEWF |
| | | | EFTIPEGNYSETMTIDLMNNAIVDNYLQVGRQNGVLESDI |
| | | | GVKFDTRNFRLGWDPVTKLVMPGVYTNEAFHPDIVLLPGC |
| | | | GVDFTQSRLSNLLGIRKRRPFQEGFQIMYEDLEGGNIPGL |
| | | | LDVPAYEQSLQQAQEEGRVTRGDTFATAPHELVIQPLTKD |
| | | | SKNRSYNLIPNSTDTLYRSWFLAYNYGDPEKGVRSWTILT |
| | | | TTDVTCGSQQVYWSLPDMMQDPVTFRPSTQVSNFPWVGTE |
| | | | LLPVHAKSFYNEQAVYSQLIRQSTALTHVFNRFPENQILV |
| | | | RPPAPTITTVSENVPALTDHGTLPLRSSISGVQRVTITDA |
| | | | RRRTCPYVYKALGWVAPKVLSSRTF |
| 237 | WO202219 | SARS-- | MFVFLVLLPLVSSQCVNLTTRTQLPPAYTNSFTRGVYYPD |
| | 7720A2 | CoV-2 | KVFRSSVLHSTQDLFLPFFSNVTWFHAIHVSGTNGTKRFD |
| | SEQ ID | spike with | NPVLPFNDGVYFASTEKSNIIRGWIFGTTLDSKTQSLLIV |
| | NO: 1 | signal | NNATNWVIKVCEFQFCNDPFLGVYYHKNNKSWMESEFRVY |
| | | sequence, | SSANNCTFEYVSQPFLMDLEGKQGNFKNLREFVFKNIDGY |
| | | mutated | FKIYSKHTPINLVRDLPQGFSALEPLVDLPIGINITRFQT |
| | | furin | LLALHRSYLTPGDSSSGWTAGAAAYYVGYLQPRTFLLKYN |
| | | cleavage | ENGTITDAVDCALDPLSETKCTLKSFTVEKGIYQTSNFRV |
| | | site, and | QPTESIVRFPNITNLCPFGEVFNATRFASVYAWNRKRISN |
| | | proline | CVADYSVLYNSASFSTFKCYGVSPTKLNDLCFTNVYADSF |
| | | stabilizing | VIRGDEVRQIAPGQTGKIADYNYKLPDDFTGCVIAWNSNN |
| | | mutations | LDSKVGGNYNYLYRLFRKSNLKPFERDISTEIYQAGSTPC |
| | | | NGVEGFNCYFPLQSYGFQPTNGVGYQPYRWWVLSFELLHA |
| | | | PATVCGPKKSTNLVKNKCVNFNFNGLTGTGVLTESNKKFL |
| | | | PFQQFGRDIADTTDAVRDPQTLEILDITPCSFGGVSVITP |
| | | | GTNTSNQVAVLYQDVNCTEVPVAIHADQLTPTWRVYSTGS |
| | | | NVFQTRAGCLIGAEHVNNSYECDIPIGAGICASYQTQTNS |
| | | | PGGSGSVASQSIIAYTMSLGAENSVAYSNNSIAIPTNFTI |
| | | | SVTTEILPVSMTKTSVDCTMYICGDSTECSNLLLQYGSFC |
| | | | TQLNRALTGIAVEQDKNTQEVFAQVKQIYKTPPIKDFGGF |
| | | | NFSQILPDPSKPSKRSFIEDLLFNKVTLADAGFIKQYGDC |
| | | | LGDIAARDLICAQKFNGLTVLPPLLTDEMIAQYTSALLAG |
| | | | TITSGWTFGAGAALQIPFAMQMAYRFNGIGVTQNVLYENQ |
| | | | KLIANQFNSAIGKIQDSLSSTASALGKLQDVVNQNAQALN |
| | | | TLVKQLSSNFGAISSVLNDILSRLDPPEAEVQIDRLITGR |
| | | | LQSLQTYVTQQLIRAAEIRASANLAATKMSECVLGQSKRV |
| | | | DFCGKGYHLMSFPQSAPHGVVFLHVTYVPAQEKNFTTAPA |
| | | | ICHDGKAHFPREGVFVSNGTHWFVTQRNFYEPQIITTDNT |
| | | | FVSGNCDWVIGIVNNTVYDPLQPELDSFKEELDKYFKNHT |
| | | | SPDVDLGDISGINASVVNIQKEIDRLNEVAKNLNESLIDL |
| | | | QELGKYEQYIKWPWYIWLGFIAGLIAIVMVTIMLCCMTSC |
| | | | CSCLKGCCSCGSCCKFDEDDSEPVLKGVKLHYT |
| 238 | WO202219 | Signature- | MFVFLVLLPLVSSQCVNFTNRTQLPSAYTNSFTRGVYYPD |
| | 7720A2 | based | KVFRSSVLHSTQDLFLPFFSNVTWFHAIHVSGTNGTKRFA |
| | SEQ ID | Epitope | NPVLPFNDGVYFASTEKSNIIRGWIFGTTLDSKTQSLLIV |
| | NO: 2 | Targeted | NNATNVVIKVCEFQFCNDPFLGVYYHKNNKSWTESEFRVY |
| | | 1 (SET 1) | SSANNCTFEYVSQPFLMDLEGKQGNFKNLREFVFKNIDGY |
| | | | FKIYSKHTPINLVRDLPQGFSALEPLVDLPIGINITRFQT |
| | | | LHRSYLTPGDSSSGWTAGAAAYYVGYLQPRTFLLKYNENG |
| | | | TITDAVDCALDPLSETKCTLKSFTVEKGIYQTSNFRVQPT |
| | | | ESIVRFPNITNLCPFGEVFNATRFASVYAWNRKRISNCVA |
| | | | DYSVLYNSASFSTFKCYGVSPTKLNDLCFTNVYADSFVIR |
| | | | GDEVRQIAPGQTGNIADYNYKLPDDFTGCVIAWNSNNLDS |
| | | | KVGGNYNYLFRLFRKSNLKPFERDISTEIYQAGSTPCNGV |
| | | | KGFNCYFPLQSYGFQPTYGVGYQPYRVVVLSFELLHAPAT |
| | | | VCGPKKSTNLVKNKCVNFNFNGLTGTGVLTESNKKFLPFQ |
| | | | QFGRDIADTTDAVRDPQTLEILDITPCSFGGVSVITPGTN |
| | | | TSNQVAVLYQGVNCTEVPVAIHADQLTPTWRVYSTGSNVF |
| | | | QTRAGCLIGAEHVNNSYECDIPIGAGICASYQTQTNSPGG |
| | | | SGSVASQSIIAYTMSLGAENSVAYSNNSIAIPTNFTISVT |
| | | | TEILPVSMTKTSVDCTMYICGDSTECSNLLLQYGSFCTQL |
| | | | NRALTGIAVEQDKNTQEVFAQVKQIYKTPPIKDFGGFNFS |
| | | | QILPDPSKPSKRSFIEDLLFNKVTLADAGFIKQYGDCLGD |
| | | | IAARDLICAQKFNGLTVLPPLLTDEMIAQYTSALLAGTIT |
| | | | SGWTFGAGAALQIPFAMQMAYRFNGIGVTQNVLYENQKLI |
| | | | ANQFNSAIGKIQDSLSSTASALGKLQDVVNQNAQALNTLV |
| | | | KQLSSNFGAISSVLNDILSRLDPPEAEVQIDRLITGRLQS |
| | | | LQTYVTQQLIRAAEIRASANLAATKMSECVLGQSKRVDFC |
| | | | GKGYHLMSFPQSAPHGVVFLHVTYVPAQEKNFTTAPAICH |
| | | | DGKAHFPREGVFVSNGTHWFVTQRNFYEPQIITTDNTFVS |
| | | | GNCDWVIGIVNNTVYDPLQPELDSFKEELDKYFKNHTSPD |
| | | | VDLGDISGINASVVNIQKEIDRLNEVAKNLNESLIDLQEL |
| | | | GKYEQYIKWPWYIWLGFIAGLIAIVMVTIMLCCMTSCCSC |
| | | | LKGCCSCGSCCKFDEDDSEPVLKGVKLHYT |
| 239 | WO202219 | Signature- | MFVFLVLLPLVSIQCVNLTTRTQLPPAYTNSFTRGVYYPD |
| | 7720A2 | based | KVFRSSVLHSTQDLFLPFFSNVTWFHAISGTNGTKRFDNP |
| | SEQ ID | Epitope | VLPFNDGVYFASTEKSNIIRGWIFGTTLDSKTQSLLIVNN |
| | NO: 3 | Targeted | ATNWVIKVCEFQFCNDPFLGVYHKNNKSCMESEFRVYSSA |
| | | 2 (SET 2) | NNCTFEYVSQPFLMDLEGKQGNFKNLREFVFKNIDGYFKI |
| | | | YSKHTPINLVRDLPQGFSALEPLVDLPIGINITRFQTLLA |
| | | | LHRSYLTPGGSSSGWTAGSAAYYVGYLQPRTFLLKYNENG |
| | | | TITDAVDCALDPLSETKCTLKSFTVEKGIYQTSNFRVQPT |
| | | | ESIVRFPNITNLCPFGEVFNATRFASVYAWNRKRISNCVA |
| | | | DYSVLYNSASFSTFKCYGVSPTKLNDLCFTNVYADSFVIR |
| | | | GDEVRQIAPGQTGKIADYNYKLPDDFTGCVIAWNSNNLDS |
| | | | KVGGNYNYRYRLFRKSNLKPFERDISTEIYQAGNTPCNGV |
| | | | EGENCYFPLQSYGFQPTNGVGYQPYRWVLSFELLHAPATV |
| | | | CGPKKSTNLVKNKCVNFNFNGLTGTGVLTESNKKFLPFQQ |
| | | | FGRDIADTTDAVRDPQTLEILDITPCSFGGVSVITPGTNT |
| | | | SNQVAVLYQGVNCTEVPVAIHADQLTPTWRVYSTGSNVFQ |
| | | | TRAGCLIGAEHVNNSYECDIPIGAGICASYQTQTNSPGGS |
| | | | GSVASQSIIAYTMSLGAENSVAYSNNSIAIPTNFTISVTT |
| | | | EILPVSMTKTSVDCTMYICGDSTECSNLLLQYGSFCTQLN |
| | | | RALTGIAVEQDKNTQEVFAQVKQIYKTPPIKDFGGFNFSQ |
| | | | ILPDPSKPSKRSFIEDLLFNKVTLADAGFIKQYGDCLGDI |
| | | | AARDLICAQKFNGLTVLPPLLTDEMIAQYTSALLAGTITS |
| | | | GWTFGAGAALQIPFAMQMAYRFNGIGVTQNVLYENQKLIA |
| | | | NQFNSAIGKIQDSLSSTASALGKLQDVVNQNAQALNTLVK |
| | | | QLSSNFGAISSVLNDILSRLDPPEAEVQIDRLITGRLQSL |
| | | | QTYVTQQLIRAAEIRASANLAATKMSECVLGQSKRVDFCG |
| | | | KGYHLMSFPQSAPHGVVFLHVTYVPAQEKNFTTAPAICHD |
| | | | GKAHFPREGVFVSNGTHWFVTQRNFYEPQIITTDNTFVSG |
| | | | NCDWVIGIVNNTVYDPLQPELDSFKEELDKYFKNHTSPDV |
| | | | DLGDISGINASVVNIQKEIDRLNEVAKNLNESLIDLQELG |
| | | | KYEQYIKWPWYIWLGFIAGLIAIVMVTIMLCCMTSCCSCL |
| | | | KGCCSCGSCCKFDEDDSEPVLKGVKLHYT |
| 240 | WO202219 | Signature- | MFVFLVLLPLVSSQCVNLTTRTQLPPAYTNSFTRGVYYPD |
| | 7720A2 | based | KVFRSSVLHSTQDLFLPFFSNVTWFHAIHVSGTNGTKRFY |
| | SEQ ID | Epitope | NPVLPFNDGVYFASTEKSNIIRGWIFGTTLDSKTQSLLIV |
| | NO: 4 | Targeted | NNATNVVIKVCEFQFCNDPFFGVYYHKNNKSWMESELRVY |
| | | 3 (SET 3) | SSANNCTFEYVSQPFLMDLEGKQGNFKNLREFVFKNIDGY |
| | | | FKIYSKHTPINLVRDLPQGFSALEPLVDLPIGINITRFQT |
| | | | LLALHRSYLTPGDSFSGWTAGAAAYYVGYLQPRTFLLKYN |
| | | | ENGTITDAVDCALDPLSETKCTLKSFTVEKGIYQTSNFRV |
| | | | QPTESIVRFPNITNLCPFGEVFNATRFASVYAWNRKRISN |
| | | | CVADYSFLYNSASFSTFKCYGVSPTKLNDLCFTNVYADSF |
| | | | VIRGDEVRQIAPGQTGTIADYNYKLPDDFTGCVIAWNSKN |
| | | | LDSKVGGNYNYLYRLFRKSNLKPFERDISTEIYQAGRTPC |
| | | | NGVEGENCYFPLQPYGFQPTTGVGYQPYRWWVLSFELLHA |
| | | | PATVCGPKKSTNLVKNKCVNFNFNGLTGTGVLTESNKKFL |
| | | | PFQQFGRDIADTTDAVRDPQTLEILDITPCSFGGVSVITP |
| | | | GTNTSNQVAVLYHDVNCTEVPVAIHADQLTPTWRVYSTGS |
| | | | NVFQTRAGCLIGAEHVNNSYECDIPIGAGICASYQTQTNS |
| | | | RGGSGSVASQSIIAYTMSLGAENSVAYSNNSIAIPTNFTI |
| | | | SVTTEILPVSMTKTSVDCTMYICGDSTECSNLLLQYGSFC |
| | | | TQLNRALTGIAVEQDKNTQEVFAQVKQIYKTPPIKDFGGF |
| | | | NFSQILPDPSKPSKRSFIEDLLFNKVTLADAGFIKQYGDC |
| | | | LGDIAARDLICAQKFNGLTVLPPLLTDEMIAQYTSALLAG |
| | | | TITSGWTFGAGAALQIPFAMQMAYRFNGIGVTQNVLYENQ |
| | | | KLIANQFNSAIGKIQDSLSSTASALGKLQDVVNQNAQALN |
| | | | TLVKQLSSNFGAISSVLNDILSRLDPPEAEVQIDRLITGR |
| | | | LQSLQTYVTQQLIRAAEIRASANLAATKMSECVLGQSKRV |
| | | | DFCGKGYHLMSFPQSAPHGVFLHVTYVPAQEKNFTTAPAI |
| | | | CHDGKAHFPREGVFVSNGTHWFVTQRNFYEPQIITTDNTF |
| | | | VSGNCDWVIGIVNNTVYDPLQPELDSFKEELDKYFKNHTS |
| | | | PDVDLGDISGINASVVNIQKEIDRLNEVAKNLNESLIDLQ |
| | | | ELGKYEQYIKWPWYIWLGFIAGLIAIVMVTIMLCCMTSCC |
| | | | SCLKGCCSCGSCCKFDEDDSEPVLKGVKLHYT |
| 241 | WO202219 | SARS-- | SEPVLKGVKLHYT |
| | 7720A2 | CoV-2 | |
| | SEQ ID | spike | |
| | NO: 13 | cytoplasmic | |
| | | region | |
| 242 | WO202219 | SARS-- | YIKWPWYIWLGFIAGLIAIVMVTIMLCCMTSCCSCLKGCC |
| | 7720A2 | CoV-2 | SCGSCCKFDEDD |
| | SEQ ID | spike | |
| | NO: 14 | trans- | |
| | | membrane | |
| | | domain | |
| 243 | WO202219 | T4-fibritin | YIPEAPRDGQAYVRKDGEWVLLSTFL |
| | 7720A2 | foldon | |
| | SEQ ID | trimerization | |
| | NO: 15 | domain | |
| 244 | WO202219 | Linker | GSGG |
| | 7720A2 | sequence | |
| | SEQ ID | | |
| | NO: 16 | | |
| 245 | WO202219 | Linker | GGSGG |
| | 7720A2 | sequence | |
| | SEQ ID | | |
| | NO: 17 | | |
| 246 | WO202219 | Furin | RRAR |
| | 7720A2 | cleavage | |
| | SEQ ID | site | |
| | NO: 18 | | |
| 247 | WO202219 | Furin | GGSG |
| | 7720A2 | cleavage | |
| | SEQ ID | site | |
| | NO: 19 | | |
| 248 | WO202219 | SARS-- | MFVFLVLLPLVSSQCVN |
| | 7720A2 | CoV-2 | |
| | SEQ ID | spike | |
| | NO: 20 | signal | |
| | | sequence | |
| 249 | WO202219 | SARS-- | LTTRTQLPPAYTNSFTRGVYYPDKVFRSSVLHSTQDLFLP |
| | 7720A2 | CoV-2 | FFSNVTWFHAIHVSGTNGTKRFDNPVLPFNDGVYFASTEK |
| | SEQ ID | spike with | SNIIRGWIFGTTLDSKTQSLLIVNNATNVVIKVCEFQFCN |
| | NO: 34 | proline | DPFLGVYYHKNNKSWMESEFRVYSSANNCTFEYVSQPFLM |
| | | stabilizing | DLEGKQGNFKNLREFVFKNIDGYFKIYSKHTPINLVRDLP |
| | | mutations | QGFSALEPLVDLPIGINITRFQTLLALHRSYLTPGDSSSG |
| | | | WTAGAAAYYVGYLQPRTFLLKYNENGTITDAVDCALDPLS |
| | | | ETKCTLKSFTVEKGIYQTSNFRVQPTESIVRFPNITNLCP |
| | | | FGEVFNATRFASVYAWNRKRISNCVADYSVLYNSASFSTF |
| | | | KCYGVSPTKLNDLCFTNVYADSFVIRGDEVRQIAPGQTGK |
| | | | IADYNYKLPDDFTGCVIAWNSNNLDSKVGGNYNYLYRLFR |
| | | | KSNLKPFERDISTEIYQAGSTPCNGVEGFNCYFPLQSYGF |
| | | | QPTNGVGYQPYRVVVLSFELLHAPATVCGPKKSTNLVKNK |
| | | | CVNFNFNGLTGTGVLTESNKKFLPFQQFGRDIADTTDAVR |
| | | | DPQTLEILDITPCSFGGVSVITPGTNTSNQVAVLYQDVNC |
| | | | TEVPVAIHADQLTPTWRVYSTGSNVFQTRAGCLIGAEHVN |
| | | | NSYECDIPIGAGICASYQTQTNSPRRARSVASQSIIAYTM |
| | | | SLGAENSVAYSNNSIAIPTNFTISVTTEILPVSMTKTSVD |
| | | | CTMYICGDSTECSNLLLQYGSFCTQLNRALTGIAVEQDKN |
| | | | TQEVFAQVKQIYKTPPIKDFGGFNFSQILPDPSKPSKRSF |
| | | | IEDLLFNKVTLADAGFIKQYGDCLGDIAARDLICAQKFNG |
| | | | LTVLPPLLTDEMIAQYTSALLAGTITSGWTFGAGAALQIP |
| | | | FAMQMAYRFNGIGVTQNVLYENQKLIANQFNSAIGKIQDS |
| | | | LSSTASALGKLQDVVNQNAQALNTLVKQLSSNFGAISSVL |
| | | | NDILSRLDKVEAEVQIDRLITGRLQSLQTYVTQQLIRAAE |
| | | | IRASANLAATKMSECVLGQSKRVDFCGKGYHLMSFPQSAP |
| | | | HGVVFLHVTYVPAQEKNFTTAPAICHDGKAHFPREGVFVS |
| | | | NGTHWFVTQRNFYEPQIITTDNTFVSGNCDVVIGIVNNTV |
| | | | YDPLQPELDSFKEELDKYFKNHTSPDVDLGDISGINASWV |
| | | | NIQKEIDRLNEVAKNLNESLIDLQELGKYEQYIKWPWYIW |
| | | | LGFIAGLIAIVMVTIMLCCMTSCCSCLKGCCSCGSCCKFD |
| | | | EDDSEPVLKGVKLHYT |
| 250 | WO202219 | SARS-- | MFVFLVLLPLVSSQCVNLTTRTQLPPAYTNSFTRGVYYPD |
| | 7720A2 | CoV-2 | KVFRSSVLHSTQDLFLPFFSNVTWFHAIHVSGTNGTKRFD |
| | SEQ ID | spike with | NPVLPFNDGVYFASTEKSNIIRGWIFGTTLDSKTQSLLIV |
| | NO: 35 | signal | NNATNVVIKVCEFQFCNDPFLGVYYHKNNKSWMESEFRVY |
| | | sequence | SSANNCTFEYVSQPFLMDLEGKQGNFKNLREFVFKNIDGY |
| | | | FKIYSKHTPINLVRDLPQGFSALEPLVDLPIGINITRFQT |
| | | | LLALHRSYLTPGDSSSGWTAGAAAYYVGYLQPRTFLLKYN |
| | | | ENGTITDAVDCALDPLSETKCTLKSFTVEKGIYQTSNFRV |
| | | | QPTESIVRFPNITNLCPFGEVFNATRFASVYAWNRKRISN |
| | | | CVADYSVLYNSASFSTFKCYGVSPTKLNDLCFTNVYADSF |
| | | | VIRGDEVRQIAPGQTGKIADYNYKLPDDFTGCVIAWNSNN |
| | | | LDSKVGGNYNYLYRLFRKSNLKPFERDISTEIYQAGSTPC |
| | | | NGVEGFNCYFPLQSYGFQPTNGVGYQPYRWWVLSFELLHA |
| | | | PATVCGPKKSTNLVKNKCVNFNFNGLTGTGVLTESNKKFL |
| | | | PFQQFGRDIADTTDAVRDPQTLEILDITPCSFGGVSVITP |
| | | | GTNTSNQVAVLYQDVNCTEVPVAIHADQLTPTWRVYSTGS |
| | | | NVFQTRAGCLIGAEHVNNSYECDIPIGAGICASYQTQTNS |
| | | | PRRARSVASQSIIAYTMSLGAENSVAYSNNSIAIPTNFTI |
| | | | SVTTEILPVSMTKTSVDCTMYICGDSTECSNLLLQYGSFC |
| | | | TQLNRALTGIAVEQDKNTQEVFAQVKQIYKTPPIKDFGGF |
| | | | NFSQILPDPSKPSKRSFIEDLLFNKVTLADAGFIKQYGDC |
| | | | LGDIAARDLICAQKFNGLTVLPPLLTDEMIAQYTSALLAG |
| | | | TITSGWTFGAGAALQIPFAMQMAYRFNGIGVTQNVLYENQ |
| | | | KLIANQFNSAIGKIQDSLSSTASALGKLQDVVNQNAQALN |
| | | | TLVKQLSSNFGAISSVLNDILSRLDKVEAEVQIDRLITGR |
| | | | LQSLQTYVTQQLIRAAEIRASANLAATKMSECVLGQSKRV |
| | | | DFCGKGYHLMSFPQSAPHGVVFLHVTYVPAQEKNFTTAPA |
| | | | ICHDGKAHFPREGVFVSNGTHWFVTQRNFYEPQIITTDNT |
| | | | FVSGNCDWVIGIVNNTVYDPLQPELDSFKEELDKYFKNHT |
| | | | SPDVDLGDISGINASWVNIQKEIDRLNEVAKNLNESLIDL |
| | | | QELGKYEQYIKWPWYIWLGFIAGLIAIVMVTIMLCCMTSC |
| | | | CSCLKGCCSCGSCCKFDEDDSEPVLKGVKLHYT |
| 251 | WO202219 | SARS-- | MFVFLVLLPLVSSQCVNLTTRTQLPPAYTNSFTRGVYYPD |
| | 7720A2 | CoV-2 | KVFRSSVLHSTQDLFLPFFSNVTWFHAIHVSGTNGTKRFD |
| | SEQ ID | spike with | NPVLPFNDGVYFASTEKSNIIRGWIFGTTLDSKTQSLLIV |
| | NO: 36 | signal | NNATNVVIKVCEFQFCNDPFLGVYYHKNNKSWMESEFRVY |
| | | sequence | SSANNCTFEYVSQPFLMDLEGKQGNFKNLREFVFKNIDGY |
| | | and not | FKIYSKHTPINLVRDLPQGFSALEPLVDLPIGINITRFQT |
| | | containing | LLALHRSYLTPGDSSSGWTAGAAAYYVGYLQPRTFLLKYN |
| | | a | ENGTITDAVDCALDPLSETKCTLKSFTVEKGIYQTSNFRV |
| | | cytoplasmic | QPTESIVRFPNITNLCPFGEVFNATRFASVYAWNRKRISN |
| | | region | CVADYSVLYNSASFSTFKCYGVSPTKLNDLCFTNVYADSF |
| | | | VIRGDEVRQIAPGQTGKIADYNYKLPDDFTGCVIAWNSNN |
| | | | LDSKVGGNYNYLYRLFRKSNLKPFERDISTEIYQAGSTPC |
| | | | NGVEGFNCYFPLQSYGFQPTNGVGYQPYRWWVLSFELLHA |
| | | | PATVCGPKKSTNLVKNKCVNFNFNGLTGTGVLTESNKKFL |
| | | | PFQQFGRDIADTTDAVRDPQTLEILDITPCSFGGVSVITP |
| | | | GTNTSNQVAVLYQDVNCTEVPVAIHADQLTPTWRVYSTGS |
| | | | NVFQTRAGCLIGAEHVNNSYECDIPIGAGICASYQTQTNS |
| | | | PRRARSVASQSIIAYTMSLGAENSVAYSNNSIAIPTNFTI |
| | | | SVTTEILPVSMTKTSVDCTMYICGDSTECSNLLLQYGSFC |
| | | | TQLNRALTGIAVEQDKNTQEVFAQVKQIYKTPPIKDFGGF |
| | | | NFSQILPDPSKPSKRSFIEDLLFNKVTLADAGFIKQYGDC |
| | | | LGDIAARDLICAQKFNGLTVLPPLLTDEMIAQYTSALLAG |
| | | | TITSGWTFGAGAALQIPFAMQMAYRFNGIGVTQNVLYENQ |
| | | | KLIANQFNSAIGKIQDSLSSTASALGKLQDVVNQNAQALN |
| | | | TLVKQLSSNFGAISSVLNDILSRLDKVEAEVQIDRLITGR |
| | | | LQSLQTYVTQQLIRAAEIRASANLAATKMSECVLGQSKRV |
| | | | DFCGKGYHLMSFPQSAPHGVVFLHVTYVPAQEKNFTTAPA |
| | | | ICHDGKAHFPREGVFVSNGTHWFVTQRNFYEPQIITTDNT |
| | | | FVSGNCDWVIGIVNNTVYDPLQPELDSFKEELDKYFKNHT |
| | | | SPDVDLGDISGINASVVNIQKEIDRLNEVAKNLNESLIDL |
| | | | QELGKYEQYIKWPWYIWLGFIAGLIAIVMVTIMLCCMTSC |
| | | | CSCLKGCCSCGSCCKFDEDD |
| 252 | WO202219 | SARS-- | LTTRTQLPPAYTNSFTRGVYYPDKVFRSSVLHSTQDLFLP |
| | 7720A2 | CoV-2 | FFSNVTWFHAIHVSGTNGTKRFDNPVLPFNDGVYFASTEK |
| | SEQ ID | spike | SNIIRGWIFGTTLDSKTQSLLIVNNATNVVIKVCEFQFCN |
| | NO: 38 | | DPFLGVYYHKNNKSWMESEFRVYSSANNCTFEYVSQPFLM |
| | | | DLEGKQGNFKNLREFVFKNIDGYFKIYSKHTPINLVRDLP |
| | | | QGFSALEPLVDLPIGINITRFQTLLALHRSYLTPGDSSSG |
| | | | WTAGAAAYYVGYLQPRTFLLKYNENGTITDAVDCALDPLS |
| | | | ETKCTLKSFTVEKGIYQTSNFRVQPTESIVRFPNITNLCP |
| | | | FGEVFNATRFASVYAWNRKRISNCVADYSVLYNSASFSTF |
| | | | KCYGVSPTKLNDLCFTNVYADSFVIRGDEVRQIAPGQTGK |
| | | | IADYNYKLPDDFTGCVIAWNSNNLDSKVGGNYNYLYRLFR |
| | | | KSNLKPFERDISTEIYQAGSTPCNGVEGFNCYFPLQSYGF |
| | | | QPTNGVGYQPYRVVVLSFELLHAPATVCGPKKSTNLVKNK |
| | | | CVNFNFNGLTGTGVLTESNKKFLPFQQFGRDIADTTDAVR |
| | | | DPQTLEILDITPCSFGGVSVITPGTNTSNQVAVLYQDVNC |
| | | | TEVPVAIHADQLTPTWRVYSTGSNVFQTRAGCLIGAEHVN |
| | | | NSYECDIPIGAGICASYQTQTNSPGGSGSVASQSIIAYTM |
| | | | SLGAENSVAYSNNSIAIPTNFTISVTTEILPVSMTKTSVD |
| | | | CTMYICGDSTECSNLLLQYGSFCTQLNRALTGIAVEQDKN |
| | | | TQEVFAQVKQIYKTPPIKDFGGFNFSQILPDPSKPSKRSF |
| | | | IEDLLFNKVTLADAGFIKQYGDCLGDIAARDLICAQKFNG |
| | | | LTVLPPLLTDEMIAQYTSALLAGTITSGWTFGAGAALQIP |
| | | | FAMQMAYRFNGIGVTQNVLYENQKLIANQFNSAIGKIQDS |
| | | | LSSTASALGKLQDVVNQNAQALNTLVKQLSSNFGAISSVL |
| | | | NDILSRLDPPEAEVQIDRLITGRLQSLQTYVTQQLIRAAE |
| | | | IRASANLAATKMSECVLGQSKRVDFCGKGYHLMSFPQSAP |
| | | | HGVVFLHVTYVPAQEKNFTTAPAICHDGKAHFPREGVFVS |
| | | | NGTHWFVTQRNFYEPQIITTDNTFVSGNCDVVIGIVNNTV |
| | | | YDPLQPELDSFKEELDKYFKNHTSPDVDLGDISGINASVV |
| | | | NIQKEIDRLNEVAKNLNESLIDLQELGKYEQYIKWPWYIW |
| | | | LGFIAGLIAIVMVTIMLCCMTSCCSCLKGCCSCGSCCKFD |
| | | | EDDSEPVLKGVKLHYT |
| 253 | WO202219 | SARS- | MFVFLVLLPLVSSQCVNLTTRTQLPPAYTNSFTRGVYYPD |
| | 7720A2 | CoV-2 | KVFRSSVLHSTQDLFLPFFSNVTWFHAISGTNGTKRFDNP |
| | SEQ ID | spike | VLPFNDGVYFASTEKSNIIRGWIFGTTLDSKTQSLLIVNN |
| | NO: 40 | protein | ATNVVIKVCEFQFCNDPFLGVYHKNNKSWMESEFRVYSSA |
| | | EG1 | NNCTFEYVSQPFLMDLEGKQGNFKNLREFVFKNIDGYFKI |
| | | | YSKHTPINLVRDLPQGFSALEPLVDLPIGINITRFQTLLA |
| | | | LHRSYLTPGDSSSGWTAGAAAYYVGYLQPRTFLLKYNENG |
| | | | TITDAVDCALDPLSETKCTLKSFTVEKGIYQTSNFRVQPT |
| | | | ESIVRFPNITNLCPFGEVFNATRFASVYAWNRKRISNCVA |
| | | | DYSVLYNSASFSTFKCYGVSPTKLNDLCFTNVYADSFVIR |
| | | | GDEVRQIAPGQTGKIADYNYKLPDDFTGCVIAWNSNNLDS |
| | | | KVGGNYNYLYRLFRKSNLKPFERDISTEIYQAGSTPCNGV |
| | | | EGFNCYFPLQSYGFQPTYGVGYQPYRVWVLSFELLHAPAT |
| | | | VCGPKKSTNLVKNKCVNFNFNGLTGTGVLTESNKKFLPFQ |
| | | | QFGRDIDDTTDAVRDPQTLEILDITPCSFGGVSVITPGTN |
| | | | TSNQVAVLYQGVNCTEVPVAIHADQLTPTWRVYSTGSNVF |
| | | | QTRAGCLIGAEHVNNSYECDIPIGAGICASYQTQTNSHRR |
| | | | ARSVASQSIIAYTMSLGAENSVAYSNNSIAIPINFTISVT |
| | | | TEILPVSMTKTSVDCTMYICGDSTECSNLLLQYGSFCTQL |
| | | | NRALTGIAVEQDKNTQEVFAQVKQIYKTPPIKDFGGFNFS |
| | | | QILPDPSKPSKRSFIEDLLFNKVTLADAGFIKQYGDCLGD |
| | | | IAARDLICAQKFNGLTVLPPLLTDEMIAQYTSALLAGTIT |
| | | | SGWTFGAGAALQIPFAMQMAYRFNGIGVTQNVLYENQKLI |
| | | | ANQFNSAIGKIQDSLSSTASALGKLQDVVNQNAQALNTLV |
| | | | KQLSSNFGAISSVLNDILARLDKVEAEVQIDRLITGRLQS |
| | | | LQTYVTQQLIRAAEIRASANLAATKMSECVLGQSKRVDFC |
| | | | GKGYHLMSFPQSAPHGVFLHVTYVPAQEKNFTTAPAICHD |
| | | | GKAHFPREGVFVSNGTHWFVTQRNFYEPQIITTHNTFVSG |
| | | | NCDWVIGIVNNTVYDPLQPELDSFKEELDKYFKNHTSPDV |
| | | | DLGDISGINASVVNIQKEIDRLNEVAKNLNESLIDLQELG |
| | | | KYEQYIKWPWYIWLGFIAGLIAIVMVTIMLCCMTSCCSCL |
| | | | KGCCSCGSCCKFDEDDSEPVLKGVKLHYT |
| 254 | WO202219 | SARS- | MFVFFVLLPLVSSQCVNFTNRTQLPSAYTNSFTRGFYYPD |
| | 7720A2 | CoV-2 | KVFRSSVLHSTRDLFLPFFSNVTWFHAIHVSGTNGTKRFG |
| | SEQ ID | spike | NPVLPFNDGVYFASIEKSNIIRGWVFGTTLDSKTQSLFIV |
| | NO: 41 | protein | NNATNVFIKVCEFQFCNYPFLGVYYHKNNKSWMESGVYSS |
| | | EG2 | ANNCSFEYVSQPFLMHLEGKQGNFKNLSEFVFKNIDGYFK |
| | | | VYSKHTPINLVRGLPQGFSVLEPLVDLPIGVNITRFQTLH |
| | | | RSYLTPGGSSSGWTAGSAAYYVGYLQLRTFLLKYNENGII |
| | | | TDAVDCALDPLSEIKCTLKSFTLEKGIYQTSNSRVQPTES |
| | | | IIRFPNITNLCSFGEVFNATSFASVYAWNRRRISNCVADY |
| | | | SLLYNSASFSTFKCYGVSLTKLNDLCFTSVYADSFVIRGD |
| | | | EVIQIAPGQTGTIADYNYKLPNDFTGCVIAWNSKNLDSKV |
| | | | GGNYNYRYRLFRKSNLKPFERDVSTEIYQAGSKPCNGVKG |
| | | | FNCYFPLQSYGFQPTNGVGYQPYRVVVFSFELLHAPSTVC |
| | | | GPKKSSNLVKNKCVSFNFNGLTGIGVLTESNKNFLPFQQF |
| | | | GRDIADTTDAVRDPQTLDILDITPCSFGSVSVITPGTNAS |
| | | | NQVAVLYHGVNCTEVPFAIHADQLTSTWRVYSTGFNVFQT |
| | | | RAGCLIGAEYVNNSYECDIPIGAGVCASYQTQTNSPRRAR |
| | | | SVASQPIIAYTMSLGVENSVAYSNNSIAIPTNFTISVTTE |
| | | | ILPVSMAKTSVDCTMYICGDSIECSNLLLQYGGFCTQLNR |
| | | | ALTVIAVEQDKNTKEVFAQVKQIYKTPPIKHFGGFNFSQI |
| | | | LPDSSKPSKRSFIEDLFFNKVTLADVGFIKQYGDCLGDIS |
| | | | ARDLICAQKFNGLNVLPPLLTDEMVAQYTSALLSGTITSG |
| | | | WTLGAGAALQIPFSMQMAYRFNGVGVTQNVLYENQKFIAN |
| | | | QFNSAIGKIQDSLFSTASALGKLQNVVNQNAQALNMLVKQ |
| | | | LSSNFGAISSVLNDILSRLDKVEAEVQIDRLVTGRLQSLQ |
| | | | IYVTQQLIRAVEIRASANLAAIKMSECVLGQSKRFDFCGK |
| | | | GYHIMSFPQSAPYGVVFLHVTYVPAHEKNFTTAPAICHYG |
| | | | KAHFPREGFFVSNGTHWFLTQRNFYEPQIITTDNTFVSGN |
| | | | CDVVVGIVNNTVYHPLQPELDSFKEELYKYFKNHTSSDVD |
| | | | LGDISGINASFVNIQKEIDRLNEVANNLNESLIDLQELGK |
| | | | YEHYIKWPWYIWLVFIAGLIAILMVTIMLCCITSCCSCLK |
| | | | GCCSCGFCCKFDEDDSEPLLKGVKLHYI |
| 255 | WO202219 | SARS- | MFVFLVLLLLVSSQCVNLRTRTQLPPAYTNSFIRGVYYPD |
| | 7720A2 | CoV-2 | KVFRSSVLYSTQDLFLPFFSNVTWFHVISGTNGTKRFANP |
| | SEQ ID | spike | VLPFNDGVYFASTEKFNIIRGWIFGTTLDLKTQSLLIVNN |
| | NO: 42 | protein | ATNAVIKVCEFQFCNDPFLDVYYHKNNKSRMESEFRVYSI |
| | | EG3 | ANNCTFEYVSQPFFMDLEGKQGNFKNFREFVFKNIYGYFK |
| | | | IYSKHTPTNLVRDLPQGFSVLEPLVHLPIGINITRFQTLL |
| | | | ALYRSYLTPGDSSSGLTAGAAAYYLGYLQPRTFLLKYNQN |
| | | | GTITDAVDCSLDPLSETKCTLKSFIVEKGIYQTSNFRVQP |
| | | | IESIVRFPNITNFCPFGEVFNATKFASVYAWNRKKISNCV |
| | | | ADYSFLYNSASFSIFKCYGVSPNKLNDLCFTNIYADSFVI |
| | | | RGDQVRQIAPGQTGNIADYNYKLPYDFTGCVIAWNSNKLD |
| | | | SKVGGNYNYQYRLFRKSNLTPFERDISTQIYQAGSTPCNG |
| | | | VKGFNCYSPLQSYGFQPTTGVGYQPYRLVLSFELLHSPAT |
| | | | VCGPKKSTNLIKNKCVNFNFNGLIGTGVLTESNKKFLSFQ |
| | | | QFGRDIDDITDAVRDPQTLQILDITPCSFGGISVITPGTN |
| | | | ISNQVAVLYQDVNCTEVPVSIHADQLTPSWRVYSTGSSVF |
| | | | QTRAGCLIGVEHVNNSYECDIPIGAGICVSYQTQTNSRRR |
| | | | ARSVASQSIIVYTMSLGAENLVAYSNNSIAIPVNFTISVT |
| | | | TEILPVSITKTSVDCTVYICGDSTECSDLLLQYGSFCIQL |
| | | | NRALTGISVEQDKNTQDVFAQVKQIYKIPPIKDFGGFNFA |
| | | | QILPDPSKSSKRSFIEDLLFNKVILADAGFIKQYGDCLGN |
| | | | IAARDLICAQNFNGLTVLPPLLTDEMIVQYTSALLAGTII |
| | | | SGWTFGAGVALQIPFAMQIAYRFNGIGITQNVLYENQKLI |
| | | | SNQFNSAIGKIQYSLSSTASALRKLQDVVNQNARALNTLV |
| | | | KQLNSNFGAISSFLNDILSRLDNVEAEVQIDRLIIGRLQS |
| | | | LQTYVIQQLIRAAEIRSSANLAATKMSECALGQSKRVDFC |
| | | | GNGYHLMSFPQSVPHGVVFLHVNYVPAQEKNFTTSPAICH |
| | | | DGKSHFPREGVFVSNGTDWFVTQRNFYKPQIITTDNTFVS |
| | | | VNCDWVIGIFNNTVYDPLQPELYSFKEELDKYFKNHTSLD |
| | | | VDLGDISGINVSWNIQKEIDHLNEVAKNLNQSLIDLQELG |
| | | | NYEQYIKWPWYIWLCFIAGLIAIVIVTIMLCCMISCCSCL |
| | | | KGFCSCGSCCKFDEDNSEPVLKGVKLYYT |
| 256 | WO202219 | SARS- | MFGFLVLLPLVSIQCVNFTTRTQLPPSYTNSFTRGIYYPD |
| | 7720A2 | CoV-2 | KVFRFSVLHSTQDFFLPFFSNVTRFHAIHVSGTNVIKRFD |
| | SEQ ID | spike | NPVLSFNDGVYFAFTEKSNIIRGWIFGTTLNSKTQSLLIL |
| | NO: 43 | protein | NNATNVVIKVCQFQFCNDPFLGVYYHKTNKSWMESESRVY |
| | | EG4 | SSANNCTFEYVAQPFLMDLEVKQGNFKNLREFLFKNIDGY |
| | | | FKIHSKHTPINLVRDFPQGFSALEPLADLPIGINITKFQT |
| | | | LLALHNSSSGWTAGAPAYYVGYLQPSTFLLKYNENGTITD |
| | | | ALDCALDPLSETMCTLKSFTVQKGIYQTSNFRFQPTESIV |
| | | | RFSNITNLCPFSEVFNATRFSSVYAWNRKRISNCFADYSV |
| | | | LYNTASFSTFKCYGLSPTKLNDLCFTHVYADSFVIKGDEV |
| | | | RQIAPGKTGKIADYNYKLPDDFIGCVIAWNSNSLDSKVGG |
| | | | NYNYMYRLFRKSNFKPFERDISIEIYQAGSKPCNGVEGFN |
| | | | CYFPLQPYGFQPTYGVGHQPYRVVVLSFELLHAPVTVCGP |
| | | | KKSTNLFKNKCVNFNLNGLTGTGVLTDSNKKFLPFQQLGR |
| | | | DIDDTTDSVRDPQTLEIFDITPCSFGGVSVVTPGTNTSNK |
| | | | VAVLYQGVSCTEVPVAIHAGQLTPTWRVYSIGSNVFQTRS |
| | | | GCLIGAEHVNNSFECDIPIGAGICASYQTHTNSPRRARSV |
| | | | VSQSIIAYTISLGAENSWVYSNNSIAIPINFIISVTTEIL |
| | | | PVSMIKTSVDCTMYICGDSNECSNLLLQYGSFCRQLNRAL |
| | | | TGIAIEQDKNTQEVFAQIKQIYKTPPIRDFGGFNFSQILP |
| | | | DPSKLSKRSFIEDLLFNKLTLADAGFIRQYGDCLGDIVAR |
| | | | DLICAQKFNGLIVLPPLLTDEIIAQYTSALLVGTITSGWT |
| | | | FGVGAALQIPFAMQMSYRFNGIGVTQNILYENQKLIANQF |
| | | | NIAIGKIQDSLSFTASALGKLQHVVNQNAQALNTLAKQLS |
| | | | SNFGAISSFLNDILARLDRVEAEVQIDGLITGRLQSLHTY |
| | | | VTQQLIKAAEIRASANLGATKMSECVLGQSQRVDFCGKGY |
| | | | HLISFPQSAPHGVVFFHVTYVPAQENNFTTAPAICHEGKA |
| | | | HFPREGVFVSNGTYWFVTQRNFYEPQIITTYNTFVSGNCG |
| | | | VVIGIVNNTVYDPWQPELDSFKDELDKYFKNHTSPYVDLG |
| | | | DISGISASVVNIQKDIDRLNEVAKSLNESLIDLQQLGKYE |
| | | | QYIKWPWYTWLGFIAGLIAIVMLTIMLCCMTSCCSFLKGC |
| | | | CSCGPCCKFDEDDSELVLKGVKLHYA |
| 257 | WO202219 | SARS-- | MFVFLVLLPLVSSQCVNLTTRTQLPPAYTNSFTRGVYYPD |
| | 7720A2 | CoV-2 | KVFRSSVLHSTQDLFLPFFSNVTWFHAISGTNGTKRFDNP |
| | SEQ ID | spike | VLPFNDGVYFASTEKSNIIRGWIFGTTLDSKTQSLLIVNN |
| | NO: 65 | | ATNVVIKVCEFQFCNDPFLGVYHKNNKSWMESEFRVYSSA |
| | | | NNCTFEYVSQPFLMDLEGKQGNFKNLREFVFKNIDGYFKI |
| | | | YSKHTPINLVRDLPQGFSALEPLVDLPIGINITRFQTLLA |
| | | | LHRSYLTPGDSSSGWTAGAAAYYVGYLQPRTFLLKYNENG |
| | | | TITDAVDCALDPLSETKCTLKSFTVEKGIYQTSNFRVQPT |
| | | | ESIVRFPNITNLCPFGEVFNATRFASVYAWNRKRISNCVA |
| | | | DYSVLYNSASFSTFKCYGVSPTKLNDLCFTNVYADSFVIR |
| | | | GDEVRQIAPGQTGKIADYNYKLPDDFTGCVIAWNSNNLDS |
| | | | KVGGNYNYLYRLFRKSNLKPFERDISTEIYQAGSTPCNGV |
| | | | EGFNCYFPLQSYGFQPTYGVGYQPYRVWVLSFELLHAPAT |
| | | | VCGPKKSTNLVKNKCVNFNFNGLTGTGVLTESNKKFLPFQ |
| | | | QFGRDIDDTTDAVRDPQTLEILDITPCSFGGVSVITPGTN |
| | | | TSNQVAVLYQGVNCTEVPVAIHADQLTPTWRVYSTGSNVF |
| | | | QTRAGCLIGAEHVNNSYECDIPIGAGICASYQTQTNSHGG |
| | | | SGSVASQSIIAYTMSLGAENSVAYSNNSIAIPINFTISVT |
| | | | TEILPVSMTKTSVDCTMYICGDSTECSNLLLQYGSFCTQL |
| | | | NRALTGIAVEQDKNTQEVFAQVKQIYKTPPIKDFGGFNFS |
| | | | QILPDPSKPSKRSFIEDLLFNKVTLADAGFIKQYGDCLGD |
| | | | IAARDLICAQKFNGLTVLPPLLTDEMIAQYTSALLAGTIT |
| | | | SGWTFGAGAALQIPFAMQMAYRFNGIGVTQNVLYENQKLI |
| | | | ANQFNSAIGKIQDSLSSTASALGKLQDVVNQNAQALNTLV |
| | | | KQLSSNFGAISSVLNDILARLDPPEAEVQIDRLITGRLQS |
| | | | LQTYVTQQLIRAAEIRASANLAATKMSECVLGQSKRVDFC |
| | | | GKGYHLMSFPQSAPHGVFLHVTYVPAQEKNFTTAPAICHD |
| | | | GKAHFPREGVFVSNGTHWFVTQRNFYEPQIITTHNTFVSG |
| | | | NCDWVIGIVNNTVYDPLQPELDSFKEELDKYFKNHTSPDV |
| | | | DLGDISGINASVVNIQKEIDRLNEVAKNLNESLIDLQELG |
| | | | KYEQYIKWPWYIWLGFIAGLIAIVMVTIMLCCMTSCCSCL |
| | | | KGCCSCGSCCKFDEDDSEPVLKGVKLHYT |
| 258 | WO202219 | SARS-- | MFVFFVLLPLVSSQCVNFTNRTQLPSAYTNSFTRGFYYPD |
| | 7720A2 | CoV-2 | KVFRSSVLHSTRDLFLPFFSNVTWFHAIHVSGTNGTKRFG |
| | SEQ ID | spike | NPVLPFNDGVYFASIEKSNIIRGWVFGTTLDSKTQSLFIV |
| | NO: 66 | | NNATNVFIKVCEFQFCNYPFLGVYYHKNNKSWMESGVYSS |
| | | | ANNCSFEYVSQPFLMHLEGKQGNFKNLSEFVFKNIDGYFK |
| | | | VYSKHTPINLVRGLPQGFSVLEPLVDLPIGVNITRFQTLH |
| | | | RSYLTPGGSSSGWTAGSAAYYVGYLQLRTFLLKYNENGII |
| | | | TDAVDCALDPLSEIKCTLKSFTLEKGIYQTSNSRVQPTES |
| | | | IIRFPNITNLCSFGEVFNATSFASVYAWNRRRISNCVADY |
| | | | SLLYNSASFSTFKCYGVSLTKLNDLCFTSVYADSFVIRGD |
| | | | EVIQIAPGQTGTIADYNYKLPNDFTGCVIAWNSKNLDSKV |
| | | | GGNYNYRYRLFRKSNLKPFERDVSTEIYQAGSKPCNGVKG |
| | | | FNCYFPLQSYGFQPTNGVGYQPYRVVVFSFELLHAPSTVC |
| | | | GPKKSSNLVKNKCVSFNFNGLTGIGVLTESNKNFLPFQQF |
| | | | GRDIADTTDAVRDPQTLDILDITPCSFGSVSVITPGTNAS |
| | | | NQVAVLYHGVNCTEVPFAIHADQLTSTWRVYSTGFNVFQT |
| | | | RAGCLIGAEYVNNSYECDIPIGAGVCASYQTQTNSPGGSG |
| | | | SVASQPIIAYTMSLGVENSVAYSNNSIAIPTNFTISVTTE |
| | | | ILPVSMAKTSVDCTMYICGDSIECSNLLLQYGGFCTQLNR |
| | | | ALTVIAVEQDKNTKEVFAQVKQIYKTPPIKHFGGFNFSQI |
| | | | LPDSSKPSKRSFIEDLFFNKVTLADVGFIKQYGDCLGDIS |
| | | | ARDLICAQKFNGLNVLPPLLTDEMVAQYTSALLSGTITSG |
| | | | WTLGAGAALQIPFSMQMAYRFNGVGVTQNVLYENQKFIAN |
| | | | QFNSAIGKIQDSLFSTASALGKLQNVVNQNAQALNMLVKQ |
| | | | LSSNFGAISSVLNDILSRLDPPEAEVQIDRLVTGRLQSLQ |
| | | | IYVTQQLIRAVEIRASANLAAIKMSECVLGQSKRFDFCGK |
| | | | GYHIMSFPQSAPYGVVFLHVTYVPAHEKNFTTAPAICHYG |
| | | | KAHFPREGFFVSNGTHWFLTQRNFYEPQIITTDNTFVSGN |
| | | | CDVVVGIVNNTVYHPLQPELDSFKEELYKYFKNHTSSDVD |
| | | | LGDISGINASFVNIQKEIDRLNEVANNLNESLIDLQELGK |
| | | | YEHYIKWPWYIWLVFIAGLIAILMVTIMLCCITSCCSCLK |
| | | | GCCSCGFCCKFDEDDSEPLLKGVKLHYI |
| 259 | WO202219 | | MFVFLVLLLLVSSQCVNLRTRTQLPPAYTNSFIRGVYYPD |
| | 7720A2 | | KVFRSSVLYSTQDLFLPFFSNVTWFHVISGTNGTKRFANP |
| | SEQ ID | | VLPFNDGVYFASTEKFNIIRGWIFGTTLDLKTQSLLIVNN |
| | NO: 67 | | ATNAVIKVCEFQFCNDPFLDVYYHKNNKSRMESEFRVYSI |
| | | | ANNCTFEYVSQPFFMDLEGKQGNFKNFREFVFKNIYGYFK |
| | | | IYSKHTPTNLVRDLPQGFSVLEPLVHLPIGINITRFQTLL |
| | | | ALYRSYLTPGDSSSGLTAGAAAYYLGYLQPRTFLLKYNQN |
| | | | GTITDAVDCSLDPLSETKCTLKSFIVEKGIYQTSNFRVQP |
| | | | IESIVRFPNITNFCPFGEVFNATKFASVYAWNRKKISNCV |
| | | | ADYSFLYNSASFSIFKCYGVSPNKLNDLCFTNIYADSFVI |
| | | | RGDQVRQIAPGQTGNIADYNYKLPYDFTGCVIAWNSNKLD |
| | | | SKVGGNYNYQYRLFRKSNLTPFERDISTQIYQAGSTPCNG |
| | | | VKGFNCYSPLQSYGFQPTTGVGYQPYRLVLSFELLHSPAT |
| | | | VCGPKKSTNLIKNKCVNFNFNGLIGTGVLTESNKKFLSFQ |
| | | | QFGRDIDDITDAVRDPQTLQILDITPCSFGGISVITPGTN |
| | | | ISNQVAVLYQDVNCTEVPVSIHADQLTPSWRVYSTGSSVF |
| | | | QTRAGCLIGVEHVNNSYECDIPIGAGICVSYQTQTNSRGG |
| | | | SGSVASQSIIVYTMSLGAENLVAYSNNSIAIPVNFTISVT |
| | | | TEILPVSITKTSVDCTVYICGDSTECSDLLLQYGSFCIQL |
| | | | NRALTGISVEQDKNTQDVFAQVKQIYKIPPIKDFGGFNFA |
| | | | QILPDPSKSSKRSFIEDLLFNKVILADAGFIKQYGDCLGN |
| | | | IAARDLICAQNFNGLTVLPPLLTDEMIVQYTSALLAGTII |
| | | | SGWTFGAGVALQIPFAMQIAYRFNGIGITQNVLYENQKLI |
| | | | SNQFNSAIGKIQYSLSSTASALRKLQDVVNQNARALNTLV |
| | | | KQLNSNFGAISSFLNDILSRLDPPEAEVQIDRLIIGRLQS |
| | | | LQTYVIQQLIRAAEIRSSANLAATKMSECALGQSKRVDFC |
| | | | GNGYHLMSFPQSVPHGVVFLHVNYVPAQEKNFTTSPAICH |
| | | | DGKSHFPREGVFVSNGTDWFVTQRNFYKPQIITTDNTFVS |
| | | | VNCDWVIGIFNNTVYDPLQPELYSFKEELDKYFKNHTSLD |
| | | | VDLGDISGINVSWVNIQKEIDHLNEVAKNLNQSLIDLQEL |
| | | | GNYEQYIKWPWYIWLCFIAGLIAIVIVTIMLCCMISCCSC |
| | | | LKGFCSCGSCCKFDEDNSEPVLKGVKLYYT |
| 260 | WO202219 | SARS-- | MFGFLVLLPLVSIQCVNFTTRTQLPPSYTNSFTRGIYYPD |
| | 7720A2 | CoV-2 | KVFRFSVLHSTQDFFLPFFSNVTRFHAIHVSGTNVIKRFD |
| | SEQ ID | spike | NPVLSFNDGVYFAFTEKSNIIRGWIFGTTLNSKTQSLLIL |
| | NO: 68 | SARS-- | NNATNVVIKVCQFQFCNDPFLGVYYHKTNKSWMESESRVY |
| | | CoV-2 | SSANNCTFEYVAQPFLMDLEVKQGNFKNLREFLFKNIDGY |
| | | spike | FKIHSKHTPINLVRDFPQGFSALEPLADLPIGINITKFQT |
| | | | LLALHNSSSGWTAGAPAYYVGYLQPSTFLLKYNENGTITD |
| | | | ALDCALDPLSETMCTLKSFTVQKGIYQTSNFRFQPTESIV |
| | | | RFSNITNLCPFSEVFNATRFSSVYAWNRKRISNCFADYSV |
| | | | LYNTASFSTFKCYGLSPTKLNDLCFTHVYADSFVIKGDEV |
| | | | RQIAPGKTGKIADYNYKLPDDFIGCVIAWNSNSLDSKVGG |
| | | | NYNYMYRLFRKSNFKPFERDISIEIYQAGSKPCNGVEGFN |
| | | | CYFPLQPYGFQPTYGVGHQPYRVVVLSFELLHAPVTVCGP |
| | | | KKSTNLFKNKCVNFNLNGLTGTGVLTDSNKKFLPFQQLGR |
| | | | DIDDTTDSVRDPQTLEIFDITPCSFGGVSWTPGTNTSNKV |
| | | | AVLYQGVSCTEVPVAIHAGQLTPTWRVYSIGSNVFQTRSG |
| | | | CLIGAEHVNNSFECDIPIGAGICASYQTHTNSPGGSGSVV |
| | | | SQSIIAYTISLGAENSWVYSNNSIAIPINFIISVTTEILP |
| | | | VSMIKTSVDCTMYICGDSNECSNLLLQYGSFCRQLNRALT |
| | | | GIAIEQDKNTQEVFAQIKQIYKTPPIRDFGGFNFSQILPD |
| | | | PSKLSKRSFIEDLLFNKLTLADAGFIRQYGDCLGDIVARD |
| | | | LICAQKFNGLIVLPPLLTDEIIAQYTSALLVGTITSGWTF |
| | | | GVGAALQIPFAMQMSYRFNGIGVTQNILYENQKLIANQFN |
| | | | IAIGKIQDSLSFTASALGKLQHVVNQNAQALNTLAKQLSS |
| | | | NFGAISSFLNDILARLDPPEAEVQIDGLITGRLQSLHTYV |
| | | | TQQLIKAAEIRASANLGATKMSECVLGQSQRVDFCGKGYH |
| | | | LISFPQSAPHGVVFFHVTYVPAQENNFTTAPAICHEGKAH |
| | | | FPREGVFVSNGTYWFVTQRNFYEPQIITTYNTFVSGNCGV |
| | | | VIGIVNNTVYDPWQPELDSFKDELDKYFKNHTSPYVDLGD |
| | | | ISGISASVVNIQKDIDRLNEVAKSLNESLIDLQQLGKYEQ |
| | | | YIKWPWYTWLGFIAGLIAIVMLTIMLCCMTSCCSFLKGCC |
| | | | SCGPCCKFDEDDSELVLKGVKLHYA |
| 261 | WO202219 | SARS- | MADSNGTITVEELKKLLEQWNLVIGFLFLTWICLLQFAYA |
| | 7720A2 | CoV-2 | NRNRFLYIIKLIFLWLLWPVTLACFVLAAVYRINWITGGI |
| | SEQ ID | membrane | AIAMACLVGLMWLSYFIASFRLFARTRSMWSFNPETNILL |
| | NO: 75 | polypeptide | NVPLHGTILTRPLLESELVIGAVILRGHLRIAGHHLGRCD |
| | | | IKDLPKEITVATSRTLSYYKLGASQRVAGDSGFAAYSRYR |
| | | | IGNYKLNTDHSSSSDNIALLVQ |
| 262 | WO202219 | SARS- | MAGSNGTITVEELKKLIEQWNLVIGFLLLTWICLLQFSYA |
| | 7720A2 | CoV-2 | NRNRFLYVIKLIFLWLLWPLTLACFVLAALYRINWITGGI |
| | SEQ ID | membrane | ATAMACLVGLMWLIYFIASFRLFVRTRSMWSFNSETNILL |
| | NO: 76 | polypeptide | NVPLYGTILTRPLLESELVIGWVILRGHLRIAGHYLGRCD |
| | | | IKDLPREITVATSRMLSYYKLGASQRVSGDSGFAAYTRYR |
| | | | IGNYKLNTYHSSSSDNIALLLQ |
| 263 | WO202219 | SARS- | MSDSNGTITVEELKKLFEQWNLVIGFLFLTWICFLQFAYA |
| | 7720A2 | CoV-2 | NRNSFLYIIKLIFLWLLWPITLACFVLAAFYRINWITGGI |
| | SEQ ID | membrane | AIAMSCLVGLMWLSYFISSFRLFARTRSIWSFNPETNILL |
| | NO: 77 | polypeptide | NVPFHGTILTRPLLESEIVIGAVILRGQLRIAGHHLGLCD |
| | | | IKDLPKELTVATSRTLSYYRLGASQRVACDSGFAAYSRHR |
| | | | IGNYKLNIDHSSSSDNVALLVQ |
| 264 | WO202219 | SARS- | MSDNGPQNQRNAPRITFGGPSDSTGSNQNGERSGARSKQR |
| | 7720A2 | CoV-2 | RPQGLPNNTASWFTALTQHGKEDLKFPRGQGVPINTNSSP |
| | SEQ ID | nucleocapsid | DDQIGYYRRATRRIRGGDGKMKDLSPRWYFYYLGTGPEAG |
| | NO: 78 | polypeptide | LPYGANKDGIIWVATEGALNTPKDHIGTRNPANNAAIVLQ |
| | | | LPQGTTLPKGFYAEGSRGGSQASSRSSSRSRNSSRNSTPG |
| | | | SSRGTSPARMAGNGGDAALALLLLDRLNQLESKMSGKGQQ |
| | | | QQGQTVTKKSAAEASKKPRQKRTATKAYNVTQAFGRRGPE |
| | | | QTQGNFGDQELIRQGTDYKHWPQIAQFAPSASAFFGMSRI |
| | | | GMEVTPSGTWLTYTGAIKLDDKDPNFKDQVILLNKHIDAY |
| | | | KTFPPTEPKKDKKKKADETQALPQRQKKQQTVTLLPAADL |
| | | | DDFSKQLQQSMSSADSTQA |
| 265 | WO202219 | SARS- | MSLNGPQNQRNALRITFGGPSGSTGSNQNGEHSGARSKQR |
| | 7720A2 | CoV-2 | RPQGLSNNTASWFTSLTQHGKEDLKFSRGQGVPINTNSSR |
| | SEQ ID | nucleocapsid | DDQIGYYRRTTRRIRGGDGKMKYLSPRWYFYYLGTGPESG |
| | NO: 79 | polypeptide | LPYGANKYGIIWVATEGAFNTPKDHIGTRNLANNAAIVLQ |
| | | | LPQGTTLSKGFYAEGSRGGSQAYSRSSSRSRNSLRNSTPG |
| | | | SSKRTSPARMAGNGGDAALVLLLLDRLNQLESKMFGKGQQ |
| | | | QQGQTVTKKSASEASKKPRQKRTATKAYNLTQAFGRRGQE |
| | | | QTQGNFGDQELTRQGTDYKHWPQIAQFSPSASAFFGMSRI |
| | | | GMEVIPSGTWLTYIGAIKLDDKDSNFKDQVILLSKHIDAY |
| | | | KTFPSTEPKKDKKKKTDETQALPQKQKKQQIVTLLPAVDL |
| | | | DDFSKQLQQSMSIADSTQA |
| 266 | WO202219 | SARS- | MSDNGPQNHRNAPRITFVGPSDSTGSNQNRERSGARSKQC |
| | 7720A2 | CoV-2 | RPQGLPNNIASWFTALTQHGKENLKFPRGQGIPINTNSSP |
| | SEQ ID | nucleocapsid | YDQIGYYRRSTRRIRGGDGKMKGLSPRWYFYYLGTGPEAV |
| | NO: 80 | polypeptide | LPYGANKDGIIWVAIEGALNTPKDYIGTRNPANNASIVLQ |
| | | | LPQGTILPKGFYAEGSRGGIQASSRSSSRIRNSSRNSTLG |
| | | | SSRGTSPARIAGNGGDAAIALLLLDRLNQLESKISGKGQQ |
| | | | QQCQTVTKKSAADASKKPRQKRTATKVYNVTQAFGRRGSE |
| | | | QTQGNFGDHELIRQGTDYKYWPQIAQFAPSASSFFGMSRI |
| | | | GMEVTPLGTWLTYTGAIKLDNKDPNFKDQFILLNKHIDAY |
| | | | KTFPPIEPKKDKKKKAYETQALPQRQKKQQIVTLLPAADL |
| | | | YDFSKQLQQSMSSSDSTQA |

1. A method of treating a subject at risk of thrombosis with thrombocytopenia syndrome (“TTS”) associated with adenoviral vector therapy, comprising administering to a subject at risk of TTS low negative charge adenoviral vector therapy.

2. A method of reducing the risk of thrombosis with thrombocytopenia syndrome (“TTS”) associated with adenoviral vector therapy, comprising administering to a subject at risk of TTS low negative charge adenoviral vector therapy.

3. The method of claim 2, wherein the risk of TTS is reduced as compared to administration of human or chimp adenoviral vector therapy, such as human Ad5 vector therapy, human Ad26 vector therapy, or chimp AdY25 (ChAdOx1) vector therapy.

4. The method of claim 2, wherein the low negative charge adenoviral vector comprises a hexon protein having hypervariable regions having a Z (charge) average of −2.5 or greater at pH 7.4, optionally wherein the hexon protein has hypervariable regions having a Z (charge) average of −2.3 or greater at pH 7.4.

5. The method of claim 2, wherein the low negative charge adenoviral vector comprises a hexon protein having hypervariable regions having a Z (charge) sum of −15 or greater at pH 7.4.

6-13. (canceled)

14. The method of claim 2, which further comprises classifying a subject's risk of TTS.

15. The method of claim 2, which further comprises identifying the subject at risk of TTS.

16. The method of claim 2, which further comprises selecting the subject at risk of TTS for rhesus adenoviral vector therapy.

17. The method of claim 2, wherein the subject is a human subject.

18. The method of claim 2, wherein the subject is positive for anti-PF4 antibodies.

19. The method of claim 18, which further comprises testing the subject for anti-PF4 antibodies prior to said administering step.

20. The method of claim 2, wherein the subject is a female.

21. The method of claim 2, wherein the subject is between the ages of 18 and 70.

22. The method of claim 2, wherein the subject is white, optionally further wherein the subject is non-Hispanic.

23. The method of claim 2, wherein the subject is obese, has hypertension, is diabetic, is on estrogen therapy (e.g., an estrogen-based contraceptive or an estrogen-based hormone replacement therapy), has a venous thrombosis risk factor (e.g., cirrhosis, malignancy, fertility treatment, or a venous catheter), has iron deficiency anemia, and/or has hypothyroidism.

24-26. (canceled)

27. The method of claim 2, wherein the low negative charge adenoviral vector therapy is administered mucosally, such as by buccal administration, e.g., by buccal swab or buccal spray, or by intranasal administration, e.g., by nasal spray.

28-29. (canceled)

30. The method of claim 2, which further comprises administering an additional dose of the low negative charge adenoviral vector therapy to the subject.

31. The method of claim 30, wherein the additional dose of the low negative charge adenoviral vector therapy is administered via the same route as the preceding dose.

32-36. (canceled)

37. The method of claim 2, wherein the low negative charge adenoviral vector therapy is an adenovirus-based vaccine.

38. The method of claim 37, wherein the adenovirus-based vaccine is a vaccine that elicits an immune response against a pathogenic virus, such as a respiratory virus, e.g., a coronavirus such as SARS-COV-2, optionally wherein the adenovirus-based vaccine comprises a transgene that encodes a coronavirus spike protein or a fragment thereof; an influenza virus; or a respiratory syncytial virus.

39-71. (canceled)


SEQUENCE LISTING (266 sequences)

SEQ ID NO: 1 - protein, 25 aa, synthetic construct
  VARIANT 17: note = Any amino acid
WETTDNKTKVRAQAPFXSTAITKEG

SEQ ID NO: 2 - protein, 49 aa, unidentified
WDEAATALEINLEEEDDDNEDEVDEQAEQQKTHVFGQAPYSGINITKEG

SEQ ID NO: 3 - protein, 31 aa, unidentified
WEQKKAGNGDTMETHTFGVAPMGGENITIDG

SEQ ID NO: 4 - protein, 37 aa, unidentified
WETKEKQGTTGGVQQEKDVTKTFGVAATGGINITNQG

SEQ ID NO: 5 - protein, 34 aa, unidentified
WEEKKNGGGSDANQMQTHTFGVAAMGGIEITAKG

SEQ ID NO: 6 - protein, 32 aa, synthetic construct
WADTNDSNKLKVRGQAPFVSTYGSATALTKDG

SEQ ID NO: 7 - protein, 28 aa, synthetic construct
WTTTNGGNKTNTFAQAPLIGTAIDGTNG

SEQ ID NO: 8 - protein, 27 aa, synthetic construct
WTTTNGGNKTNSFAQASYIGQSLSKDG

SEQ ID NO: 9 - protein, 29 aa, synthetic construct
WTNSDSKVKVRAQAPFVSSYGATAITKEG

SEQ ID NO: 10 - protein, 29 aa, synthetic construct
WADSDSKLKVRAQAPFVSSYGNNAITKEG

SEQ ID NO: 11 - protein, 29 aa, synthetic construct
WITNGGTAKTNSFAQASYIGMGANLTTNG

SEQ ID NO: 12 - protein, 25 aa, synthetic construct
WEGSDNKTHVRGQAPFISDEITKEG

SEQ ID NO: 13 - protein, 29 aa, synthetic construct
WTNTDSKVKVRAQAPFVSSYGATAITKEG

SEQ ID NO: 14 - protein, 25 aa, synthetic construct
WNGTDSKTNVRGQAPFISDAITKEG

SEQ ID NO: 15 - protein, 26 aa, synthetic construct
WTDTSDNKLKAYAQAPYQSQGLTKDG

SEQ ID NO: 16 - protein, 32 aa, synthetic construct
WEDTNDSNKLKVRGQAPYVSTFGAATALTKDG

SEQ ID NO: 17 - protein, 25 aa, synthetic construct
WNGTDSKTNVRGQAPFISDAITKEG

SEQ ID NO: 18 - protein, 29 aa, synthetic construct
WITNGGTAKTNSFAQASYIGMGANLTTNG

SEQ ID NO: 19 - protein, 29 aa, synthetic construct
WENSDNKLKVRAQAPFVSSYGATAITKDG

SEQ ID NO: 20 - protein, 29 aa, synthetic construct
WADSDSKLKVRAQAPFVSSYGNNAITKEG

SEQ ID NO: 21 - protein, 29 aa, synthetic construct
WITNGGTAKTNSFAQASYIGMGANLTTNG

SEQ ID NO: 22 - protein, 29 aa, synthetic construct
WENSDNKLKVRAQAPFVSSYGATAITKDG

SEQ ID NO: 23 - protein, 13 aa, synthetic construct
  VARIANT 9: note = Any amino acid
VGVDTTDSXTAQY

SEQ ID NO: 24 - protein, 10 aa, unidentified
IGVEGQTPKY

SEQ ID NO: 25 - protein, 14 aa, unidentified
IGTDATADQDKPIY

SEQ ID NO: 26 - protein, 15 aa, unidentified
LGTDETAENGKKDIY

SEQ ID NO: 27 - protein, 16 aa, unidentified
IGIDATKEEDNGKEIY

SEQ ID NO: 28 - protein, 13 aa, synthetic construct
VGVDTSEASQAVY

SEQ ID NO: 29 - protein, 10 aa, synthetic construct
IGQDNGQAVY

SEQ ID NO: 30 - protein, 13 aa, synthetic construct
VAVDTAAGGAAVY

SEQ ID NO: 31 - protein, 14 aa, synthetic construct
VGVTLTDSGSTPQY

SEQ ID NO: 32 - protein, 14 aa, synthetic construct
VGVILTDSGQTAQY

SEQ ID NO: 33 - protein, 13 aa, synthetic construct
VAVDTNNANAAVY

SEQ ID NO: 34 - protein, 13 aa, synthetic construct
VGTDTGNPGQAIY

SEQ ID NO: 35 - protein, 14 aa, synthetic construct
VGVVLTDSGSTPQY

SEQ ID NO: 36 - protein, 13 aa, synthetic construct
VGTDTGNHGQAIY

SEQ ID NO: 37 - protein, 14 aa, synthetic construct
VGLVVTESGQTPQY

SEQ ID NO: 38 - protein, 14 aa, synthetic construct
VGVTVSESARTPQY

SEQ ID NO: 39 - protein, 13 aa, synthetic construct
VGTDTGNPGQAIY

SEQ ID NO: 40 - protein, 13 aa, synthetic construct
VAVDTNNANAAVY

SEQ ID NO: 41 - protein, 14 aa, synthetic construct
VGVVLTDSTQTPQY

SEQ ID NO: 42 - protein, 14 aa, synthetic construct
VGVILTDSGQTAQY

SEQ ID NO: 43 - protein, 13 aa, synthetic construct
VAVDTNNANAAVY

SEQ ID NO: 44 - protein, 14 aa, synthetic construct
VGVVLTDSTQTPQY

SEQ ID NO: 45 - protein, 11 aa, synthetic construct
WNSEVGTDKAA

SEQ ID NO: 46 - protein, 10 aa, unidentified
WYETEINHAA

SEQ ID NO: 47 - protein, 9 aa, unidentified
WQETESFYG

SEQ ID NO: 48 - protein, 9 aa, unidentified
WQENEAFYG

SEQ ID NO: 49 - protein, 9 aa, unidentified
WQDSDNYYG

SEQ ID NO: 50 - protein, 12 aa, synthetic construct
WNSEVGNDDRVA

SEQ ID NO: 51 - protein, 10 aa, synthetic construct
WNTNPTTNAA

SEQ ID NO: 52 - protein, 10 aa, synthetic construct
WNENPTTNAA

SEQ ID NO: 53 - protein, 12 aa, synthetic construct
WNSDVGTDDKIA

SEQ ID NO: 54 - protein, 12 aa, synthetic construct
WNSEVGTNDKVA

SEQ ID NO: 55 - protein, 10 aa, synthetic construct
WNINPTQNAA

SEQ ID NO: 56 - protein, 12 aa, synthetic construct
WNSDVGTNDKAA

SEQ ID NO: 57 - protein, 12 aa, synthetic construct
WNSDVGTNDKIA

SEQ ID NO: 58 - protein, 12 aa, synthetic construct
WNSDVGATDKAA

SEQ ID NO: 59 - protein, 10 aa, synthetic construct
WNLEQEDKAA

SEQ ID NO: 60 - protein, 12 aa, synthetic construct
WNSEVGNDDKLA

SEQ ID NO: 61 - protein, 12 aa, synthetic construct
WNSDVGATDKAA

SEQ ID NO: 62 - protein, 10 aa, synthetic construct
WNINPTQNAA

SEQ ID NO: 63 - protein, 12 aa, synthetic construct
WNSEVGNDDKVA

SEQ ID NO: 64 - protein, 12 aa, synthetic construct
WNSEVGTNDKVA

SEQ ID NO: 65 - protein, 10 aa, synthetic construct
WNINPTQNAA

SEQ ID NO: 66 - protein, 12 aa, synthetic construct
WNSEVGNDDKVA

SEQ ID NO: 67 - protein, 16 aa, synthetic construct
  VARIANT 6: note = Any amino acid
  VARIANT 8: note = Any amino acid
  VARIANT 9: note = Any amino acid
ATQPAXDXXNNVELQF

SEQ ID NO: 68 - protein, 19 aa, unidentified
GILVKQQNGKLESQVEMQF

SEQ ID NO: 69 - protein, 21 aa, unidentified
AKLKVGADGVPTKEFDIDLAF

SEQ ID NO: 70 - protein, 21 aa, unidentified
AKFKPVNEGEQPKDLDIDFAY

SEQ ID NO: 71 - protein, 22 aa, unidentified
AKFKTPEKEGEEPKELDIDLNF

SEQ ID NO: 72 - protein, 18 aa, synthetic construct
AIQPTAGNGDNQAVELQF

SEQ ID NO: 73 - protein, 9 aa, synthetic construct
ASGDVTFQF

SEQ ID NO: 74 - protein, 20 aa, synthetic construct
VKITDPNNDKTGANNVSLNF

SEQ ID NO: 75 - protein, 16 aa, synthetic construct
ATPSASQDVQNPELQF

SEQ ID NO: 76 - protein, 16 aa, synthetic construct
ATQPASQETQNPELQF

SEQ ID NO: 77 - protein, 20 aa, synthetic construct
VKITDGSTDPTGANSVSMEF

SEQ ID NO: 78 - protein, 10 aa, synthetic construct
ATNQQVDLQF

SEQ ID NO: 79 - protein, 16 aa, synthetic construct
ATPSASQDVQNPELQF

SEQ ID NO: 80 - protein, 10 aa, synthetic construct
ATNQQVDMQF

SEQ ID NO: 81 - protein, 10 aa, synthetic construct
AKNQEVDLQF

SEQ ID NO: 82 - protein, 19 aa, synthetic construct
AIQPAGSSGDTNPAVELQF

SEQ ID NO: 83 - protein, 10 aa, synthetic construct
ATNQQVDMQF

SEQ ID NO: 84 - protein, 20 aa, synthetic construct
VKITDGSTDPTGANSVSMEF

SEQ ID NO: 85 - protein, 18 aa, synthetic construct
ATQPAAGQDAQNAVELQF

SEQ ID NO: 86 - protein, 16 aa, synthetic construct
ATQPASQETQNPELQF

SEQ ID NO: 87 - protein, 20 aa, synthetic construct
VKITDGTTDPTGANSVSMEF

SEQ ID NO: 88 - protein, 18 aa, synthetic construct
ATQPAAGQDAQNAVELQF

SEQ ID NO: 89 - protein, 13 aa, synthetic construct
FATTSNNNTPKVV

SEQ ID NO: 90 - protein, 19 aa, unidentified
FSTTEAAAGNGDNLTPKVV

SEQ ID NO: 91 - protein, 19 aa, unidentified
FDTPGGTVNGQDEYKADIV

SEQ ID NO: 92 - protein, 22 aa, unidentified
FDVPGGSPPAGGSGEEYKADII

SEQ ID NO: 93 - protein, 22 aa, unidentified
FDIPSTGTGGNGTNVNFKPDMI

SEQ ID NO: 94 - protein, 13 aa, synthetic construct
FATTSTPTAPKAV

SEQ ID NO: 95 - protein, 14 aa, synthetic construct
FDTASDNGNNPKVV

SEQ ID NO: 96 - protein, 15 aa, synthetic construct
FNTAADNGNNNPKVV

SEQ ID NO: 97 - protein, 13 aa, synthetic construct
FASTNVANTPKAV

SEQ ID NO: 98 - protein, 14 aa, synthetic construct
FAASSTTVNTPKAV

SEQ ID NO: 99 - protein, 14 aa, synthetic construct
FTTTADNQNQAKVV

SEQ ID NO: 100 - protein, 13 aa, synthetic construct
FSTGSSNTTPKVV

SEQ ID NO: 101 - protein, 13 aa, synthetic construct
FASTNVANTPKAV

SEQ ID NO: 102 - protein, 13 aa, synthetic construct
FATGSSNTTPKVV

SEQ ID NO: 103 - protein, 14 aa, synthetic construct
FATPGDTQNTAKVV

SEQ ID NO: 104 - protein, 13 aa, synthetic construct
FATTSATTTPKAV

SEQ ID NO: 105 - protein, 13 aa, synthetic construct
FATGSSNTTPKVV

SEQ ID NO: 106 - protein, 14 aa, synthetic construct
FTTTADNQNQAKVV

SEQ ID NO: 107 - protein, 13 aa, synthetic construct
FATTSATTTPKAV

SEQ ID NO: 108 - protein, 13 aa, synthetic construct
FAASSTVNTPKAV

SEQ ID NO: 109 - protein, 14 aa, synthetic construct
FTTTADNQNQAKVV

SEQ ID NO: 110 - protein, 13 aa, synthetic construct
FATTSATTTPKAV

SEQ ID NO: 111 - protein, 16 aa, synthetic construct
PTVTAGTTSSEALLGQ

SEQ ID NO: 112 - protein, 14 aa, unidentified
PTIKEGNSRELMGQ

SEQ ID NO: 113 - protein, 14 aa, unidentified
PGKDDASSEINLVQ

SEQ ID NO: 114 - protein, 14 aa, unidentified
PGTSDNSSEINLVQ

SEQ ID NO: 115 - protein, 14 aa, unidentified
PGKEDASSESNLTQ

SEQ ID NO: 116 - protein, 16 aa, synthetic construct
PTVVAGTTSSEALLTQ

SEQ ID NO: 117 - protein, 14 aa, synthetic construct
PTADNTNSENLLGQ

SEQ ID NO: 118 - protein, 16 aa, synthetic construct
PDVTGDATSAETLLGQ

SEQ ID NO: 119 - protein, 16 aa, synthetic construct
PTVTEGITSSEALLTQ

SEQ ID NO: 120 - protein, 16 aa, synthetic construct
PSVTEGTTSSEALLTQ

SEQ ID NO: 121 - protein, 16 aa, synthetic construct
PDVADNAKAAETLLGQ

SEQ ID NO: 122 - protein, 16 aa, synthetic construct
PTVTAGVTNAEALLAQ

SEQ ID NO: 123 - protein, 16 aa, synthetic construct
PTVTEGITSSEALLTQ

SEQ ID NO: 124 - protein, 16 aa, synthetic construct
PIVTAGTTNAEALLAQ

SEQ ID NO: 125 - protein, 14 aa, synthetic construct
PDDDSTSSKLLLGQ

SEQ ID NO: 126 - protein, 16 aa, synthetic construct
PTVTAGTTSSEALLTQ

SEQ ID NO: 127 - protein, 16 aa, synthetic construct
PIVTAGTTNAEALLAQ

SEQ ID NO: 128 - protein, 16 aa, synthetic construct
PDVADNAKAAETLLGQ

SEQ ID NO: 129 - protein, 16 aa, synthetic construct
PTVTAGTTSSEALLSQ

SEQ ID NO: 130 - protein, 16 aa, synthetic construct
PSVTEGTTSSEALLAQ

SEQ ID NO: 131 - protein, 16 aa, synthetic construct
PDVADNAKAAETLLGQ

SEQ ID NO: 132 - protein, 16 aa, synthetic construct
PTVTAGTTSSEALLSQ

SEQ ID NO: 133 - protein, 34 aa, synthetic construct
  VARIANT 13: note = Any amino acid
  VARIANT 30: note = Any amino acid
  VARIANT 33: note = Any amino acid
GMGVTDTYTGIKXNNGGGNTWEANBDVFAXRGXE

SEQ ID NO: 134 - protein, 33 aa, unidentified
GVINTETLTKVKPKTGQENGWEKDATEFSDKNE

SEQ ID NO: 135 - protein, 36 aa, unidentified
GSGTNAAYQGVKVKNGNDGDVESEWENDDTVAARNQ

SEQ ID NO: 136 - protein, 36 aa, unidentified
GTGTNSTYQGVKITNGNDGAEESEWEKDDAISRQNQ

SEQ ID NO: 137 - protein, 32 aa, unidentified
GAGTNAVYQGVKVKTTNNTEWEKDTAVSEHNQ

SEQ ID NO: 138 - protein, 30 aa, synthetic construct
GMVVTDTYKAIKVNGSGWTANTDVFSERVE

SEQ ID NO: 139 - protein, 29 aa, synthetic construct
AQGIANTYKGVKKNNGNWAKDDAVVETNE

SEQ ID NO: 140 - protein, 29 aa, synthetic construct
GQGISNTYKGVKYNTNTWTQDTDVVTTNE

SEQ ID NO: 141 - protein, 33 aa, synthetic construct
GMAVTDTYSPIKVNGGGNGWEANNGVFTERGVE

SEQ ID NO: 142 - protein, 33 aa, synthetic construct
GMAVTDTYSPIKLNGGQNGWEANNDVYAERGVE

SEQ ID NO: 143 - protein, 31 aa, synthetic construct
GQGISNTYRGLSVQNGQNTWQANNNAAENNE

SEQ ID NO: 144 - protein, 31 aa, synthetic construct
GMAVTDTYSGIKPSDNGGTFQADNTVADRVE

SEQ ID NO: 145 - protein, 33 aa, synthetic construct
GMAVTDTYSPIKVNTAGNGWEANNDVFTDRGVE

SEQ ID NO: 146 - protein, 32 aa, synthetic construct
GMAVTDTYTGIKPDNGGTTFSNDDTFATRGIE

SEQ ID NO: 147 - protein, 35 aa, synthetic construct
GMVVTDNYNSVTPQNGGSGNTWQADNTTFSQRGAQ

SEQ ID NO: 148 - protein, 33 aa, synthetic construct
GMVVTDTYTPIKPNGSGSVTWTANTDVFSNRVE

SEQ ID NO: 149 - protein, 32 aa, synthetic construct; identical to SEQ ID NO: 146

SEQ ID NO: 150 - protein, 31 aa, synthetic construct; identical to SEQ ID NO: 143

SEQ ID NO: 151 - protein, 34 aa, synthetic construct
GTVVTDTYTPIQPNSNGGGTWVAKDNVFTDRGVE

SEQ ID NO: 152 - protein, 33 aa, synthetic construct; identical to SEQ ID NO: 142

SEQ ID NO: 153 - protein, 31 aa, synthetic construct
GQGISNTYKGLSVQNGQNTWQANNNAAENNE

SEQ ID NO: 154 - protein, 34 aa, synthetic construct; identical to SEQ ID NO: 151

SEQ ID NO: 155 - protein, 952 aa, unidentified
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 156 - protein, 942 aa, unidentified
MATPSMLPQWAYMHIAGQDASEYLSPGLVQFARATDTYFSLGNKFRNPTVAPTHDVTTDRSQRLTLRFVPVDREDNTYSYKVRYTLAVGDNRVLDMASTYFDIRGVLDRGPSFKPYSGTAYNSLAPKGAPNSSQWEQKKAGNGDTMETHTFGVAPMGGENITIDGLQIGTDATADQDKPIYADKTFQPEPQVGEENWQETESFYGGRALKKDTSMKPCYGSYARPTNVKGGQAKLKVGADGVPTKEFDIDLAFFDTPGGTVNGQDEYKADIVMYTENTYLETPDTHVVYKPGKDDASSEINLVQQSMPNRPNYIGFRDNFIGLMYYNSTGNMGVLAGQASQLNAVVDLQDRNTELSYQLLLDSLGDRTRYFSMWNQAVDSYDPDVRIIENHGVEDELPNYCFPLDGSGTNAAYQGVKVKNGNDGDVESEWENDDTVAARNQLCKGNIFAMEINLQANLWRSFLYSNVALYLPDSYKYTPANITLPTNTNTYDYMNGRVVPPSLVDAYINIGARWSLDPMDNVNPFNHHRNAGLRYRSMLLGNGRYVPFHIQVPQKFFAIKSLLLLPGSYTYEWNFRKDVNMILQSSLGNDLRTDGASISFTSINLYATFFPMAHNTASTLEAMLRNDTNDQSFNDYLSAANMLYPIPANATNVPISIPSRNWAAFRGWSFTRLKTKETPSLGSGFDPYFVYSGSIPYLDGTFYLNHTFKKVSITFDSSVSWPGNDRLLTPNEFEIKRTVDGEGYNVAQCNMTKDWFLVQMLAHYNIGYQGFYVPEGYKDRMYSFFRNFQPMSRQVVDEVNYKDYQAVTLAYQHNNSGFVGYLAPTMRQGQPYPANYPYPLIGKSAVTSVTQKKFLCDRVMWRIPFSSNFMSMGALTDLGQNMLYANSAHALDMNFEVDPMDESTLLYVVFEVFDVVRVHQPHRGVIEAVYLRTPFSAGNATT

SEQ ID NO: 157 - protein, 962 aa, unidentified
MCLTARERAKMATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNPSQWETKEKQGTTGGVQQEKDVTKTFGVAATGGINITNQGLLLGTDETAENGKKDIYADKTFQPEPQVGEENWQENEAFYGGRALKKDTKMKPCYGSFARPTNEKGGQAKFKPVNEGEQPKDLDIDFAYFDVPGGSPPAGGSGEEYKADIILYTENVNLETPDTHVVYKPGTSDNSSEINLVQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLNGTGTNSTYQGVKITNGNDGAEESEWEKDDAISRQNQICKGNVYAMEINLQANLWKSFLYSNVALYLPDSYKYTPANVKLPANTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATT

SEQ ID NO: 158 - protein, 947 aa, unidentified
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNPSQWEEKKNGGGSDANQMQTHTFGVAAMGGIEITAKGLQIGIDATKEEDNGKEIYADKTFQPEPQIGEENWQDSDNYYGGRAIKKETKMKPCYGSFARPTNEKGGQAKFKTPEKEGEEPKELDIDLNFFDIPSTGTGGNGTNVNFKPDMIMYAENVNLETPDTHIVYKPGKEDASSESNLTQQSMPNRPNYIGFRDNFVGLMYYNSTGNMGVLAGQASQLNAVVDLQDRNTELSYQLLLDSLGDRTRYFSMWNSAVDSYDPDVRIIENHGVEDELPNYCFPLDGAGTNAVYQGVKVKTTNNTEWEKDTAVSEHNQICKGNVYAMEINLQANLWKSFLYSNVALYLPDSYKYTPANVTLPTNTNTYEYMNGRVVAPSLVDAYINIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATFFPMAHNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFHVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATT

SEQ ID NO: 159 - protein, 933 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPAEWADTNDSNKLKVRGQAPFVSTYGSATALTKDGIQVGVDTSEASQAVYADRSYQPEPQIGETEWNSEVGNDDRVAGRVLKKTTPMFPCYGSYAKPTNEKGGQAIQPTAGNGDNQAVELQFFATTSTPTAPKAVLYAEDVAIEAPDTHLVFKPTVVAGTTSSEALLTQQAAPNRPNYIAFRDNFIGLMYYNSTGNMGVLAGQASQLNAVVDLQDRNTELSYQLMLDALGDRSRYFSMWNQAVDSYDPDVRIVENHGVEDELPNYCFPLGGMVVTDTYKAIKVNGSGWTANTDVFSERVEIGSGNLFAMEINLQANLWRSFLYSNIGLYLPDSLKLTPDNITLPENKNTYQYMNGRVTPPGLVDTYVNVGARWSPDVMDSINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLVQMLSHYNIGYQGFYVPENYKDRMYSFFRNFQPMSRQVVDSVAYRDYYQDVKLPYQHNNSGFVGYMGPTMREGQAYPANYPYPLIGETAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 160 - protein, 912 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYTGTAYNALAPKGAPNACQWTTTNGGNKTNTFAQAPLIGTAIDGTNGLQIGQDNGQAVYADKTFQPEPQVGESQWNTNPTTNAAGRVLKTTTRMLPCYGSFARPTNEKGGQASGDVTFQFFDTASDNGNNPKVVLYGEDVNIESPDTHLIYKPTADNTNSENLLGQQAAPNRANYIAFRDNFIGLMYYNSTGNMGVLAGQASQLNAVVDLQDRNTELSYQLMLDAIGDRSRYFSMWNQAVDSYDPDVRIIENHGVEDELPNYCFPLNAQGIANTYKGVKKNNGNWAKDDAVVETNEIGIGNVFAMEINLTANLWRNFLYSNIALYLPDSYKYSPGNITLPENKNSYNYINGRVTAPGLVDTFVNIGARWSPDPMDNVNPFNHHRNAGLRYRSMLLGNGRYVPFHIQVPQKFFAIKNLLLLPGSYTYEWNFRKDVNMILQSTLGNDLRVDGASVRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPEAYKDRMYSFFRNFQPMSRQVVDADRYEQYKKVTVEYQHNNSGFVGYMGPTMREGQAYPANYPYPLIGDTAVPSLTQKKFLCDRTMWRIPFSSNFMSMGALTDLGQNMLYANSAHALDMTFEVDPMDEPTLLYVLFEVFDVVRIHQPHRGVIEAVYLRTPFSAGNATT

SEQ ID NO: 161 - protein, 928 aa, synthetic construct
MATPSMMPQWSYMHIAGQDASEYLSPGLVQFARATESYFSLGNKFRNPTVAPTHDVTTDRSQRLTLRFVPVDREDTAYSYKVRFTLAVGDNRVLDMASTYFDIRGMLDRGPSFKPYSGTAYNSLAPKGAPNPSQWTTTNGGNKTNSFAQASYIGQSLSKDGVQVAVDTAAGGAAVYADKTFQPEPQVGISQWNENPTTNAAGRILKPTTAMRPCYGSYAYPTNEKGGQVKITDPNNDKTGANNVSLNFFNTAADNGNNNPKVVLYSEDVNLEGPDTHLVFKPDVTGDATSAETLLGQQAAPNRPNYIGFRDNFIGLMYYNSTGNMGVLAGQASQLNAVVDLQDRNTELSYQLMLDALGDRSRYFSMWNQAVDSYDPDVRIIENHGVEDELPNYCFPLNGQGISNTYKGVKYNTNTWTQDTDVVTTNEISIGNIFAMEINLAANLWRSFLYSNVALYLPDSYKYTPDNIELPTNKNSYGYINGRVTAPTAIDTYVNIGARWSPDPMDNVNPFNHHRNAGLRYRSMLLGNGRYVPFHIQVPQKFFAIKNLLLLPGSYTYEWNFRKDVNMILQSTLGNDLRVDGASVRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPEAYKDRMYSFFRNFQPMSRQVVDTVNYANYKEVTMPFQHNNSGFVGYMGPTMREGQAYPANYPYPLIGATAVPSLTQKKFLCDRTMWRIPFSSNFMSMGALTDLGQNMLYANSAHALDMTFEVDPMDEPTLLYVLFEVFDVVRIHQPHRGVIEAVYLRTPFSAGNATT

SEQ ID NO: 162 - protein, 931 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPAEWTNSDSKVKVRAQAPFVSSYGATAITKEGIQVGVTLTDSGSTPQYADKTYQPEPQIGELQWNSDVGTDDKIAGRVLKKTTPMFPCYGSYARPTNEKGGQATPSASQDVQNPELQFFASTNVANTPKAVLYAEDVSIEAPDTHLVFKPTVTEGITSSEALLTQQAAPNRPNYIAFRDNFIGLMYYNSTGNMGVLAGQASQLNAVVDLQDRNTELSYQLMLDALGDRSRYFSMWNQAVDSYDPDVRIIENHGVEDELPNYCFPLGGMAVTDTYSPIKVNGGGNGWEANNGVFTERGVEIGSGNMFAMEINLQANLWRSFLYSNIGLYLPDSLKITPDNITLPENKTTYQYMNGRVTPPGLVDTYVNVGARWSPDVMDSINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLVQMLSHYNIGYQGFYVPENYKDRMYSFFRNFQPMSRQIVDSTAYTNYQDVKLPYQHNNSGFVGYMGPTMREGQAYPANYPYPLIGATAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 163 - protein, 932 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPAEWADSDSKLKVRAQAPFVSSYGNNAITKEGIQVGVILTDSGQTAQYADKTYQPEPQIGELQWNSEVGTNDKVAGRVLKKTTPMFPCYGSYAKPTNAKGGQATQPASQETQNPELQFFAASSTTVNTPKAVLYAEDVSIEAPDTHLVFKPSVTEGTTSSEALLTQQAAPNRPNYIAFRDNFIGLMYYNSTGNMGVLAGQASQLNAVVDLQDRNTELSYQLMLDALGDRSRYFSMWNQAVDSYDPDVRIVENHGVEDELPNYCFPLGGMAVTDTYSPIKLNGGQNGWEANNDVYAERGVEIGSGNLFAMEINLQANLWRSFLYSNIGLYLPDSLKITPDNITLPENKNTYQYMNGRVTPPGLIDTYVNVGARWSPDVMDSINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQIVDSTAYTNYQDVKLPYQHNNSGFVGYMGPTMREGQAYPANYPYPLIGATAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 164 - protein, 931 aa, synthetic construct
MATPSMMPQWSYMHIAGQDASEYLSPGLVQFARATDTYFSLGNKFRNPTVAPTHDVTTDRSQRLTLRFVPVDREDTAYSYKVRFTLAVGDNRVLDMASTYFDIRGMLDRGPSFKPYSGTAYNSLAPKGAPNPSQWITNGGTAKTNSFAQASYIGMGANLTTNGIQVAVDTNNANAAVYANKIYQPEPQVGTSRWNINPTQNAAGRILKPTTPMQPCYGSYAYPTNENGGQVKITDGSTDPTGANSVSMEFFTTTADNQNQAKVVLYSEDVNLEAPDTHLVFKPDVADNAKAAETLLGQQSAPNRPNYIGFRDNFIGLMYYNSTGNMGVLAGQASQLNAVVDLQDRNTELSYQLMLDALGDRSRYFSMWNQAVDSYDPDVRIIENHGVEDELPNYCFPLNGQGISNTYRGLSVQNGQNTWQANNNAAENNEISIGNIFAMEINLAANLWRSFLYSNVALYLPDSYKYTPDNIELPTNQNSYGYINGRVTAPNAIDTYVNIGARWSPDPMDNVNPFNHHRNAGLRYRSMLLGNGRYVPFHIQVPQKFFAIKNLLLLPGSYTYEWNFRKDVNMILQSTLGNDLRVDGASVRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQVVDSVNYANYKEVKMPFQHNNSGFVGYMGPTMREGQAYPANYPYPLIGATAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 165 - protein, 918 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPSEWEGSDNKTHVRGQAPFISDEITKEGIKVGTDTGNPGQAIYAKKAYQPEPQVGETQWNSDVGTNDKAAGRILKKTTPMVPCYGSYAKPTNEEGGQATNQQVDLQFFSTGSSNTTPKVVLYSEDVSLETPDTHLVFKPTVTAGVTNAEALLAQQAAPNRPNYIAFRDNFIGLMYYNSTGNMGVLAGQASQLNAVVDLQDRNTELSYQLMLDALGDRSRYFSMWNQAVDSYDPDVRIVENHGVEDELPNYCFPLGGMAVTDTYSGIKPSDNGGTFQADNTVADRVEIGSGNMFAMEINLQANLWRGFLYSNIGLYLPDTLKLTPDNITLPENKNTYQYMNGRVTPPGLVDTYVNVGARWSPDVMDSINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQIVDSTAYTNYQDVKLPYQHNNSGFVGYMGPTMREGQAYPANYPYPLIGATAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 166 - protein, 931 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPAEWTNTDSKVKVRAQAPFVSSYGATAITKEGVQVGVVLTDSGSTPQYADKTYQPEPQIGEQQWNSDVGTNDKIAGRVLKKTTPMFPCYGSYARPTNEKGGQATPSASQDVQNPELQFFASTNVANTPKAVLYAEDVAIEAPDTHLVFKPTVTEGITSSEALLTQQAAPNRPNYIAFRDNFIGLMYYNSTGNMGVLAGQASQLNAVVDLQDRNTELSYQLMLDALGDRSRYFSMWNQAVDSYDPDVRIIENHGVEDELPNYCFPLGGMAVTDTYSPIKVNTAGNGWEANNDVFTDRGVEIGSGNMFAMEINLQANLWRSFLYSNIGLYLPDSLKITPDNITLPENKNTYQYMNGRVTPPGLVDTYVNVGARWSPDVMDSINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQIVDSTSYTNYQDVKLPYQHNNSGFVGYMGPTMREGQAYPANYPYPLIGATAVPSLTQKKFLCDRVMWRIPFSSNFMSMGALTDLGQNMLYANSAHALDMTFELDPMDEPTLLYVLFEVFDVVRIHQPHRGVIEAVYLRTPFSAGNATT

SEQ ID NO: 167 - protein, 919 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPSEWNGTDSKTNVRGQAPFISDAITKEGIKVGTDTGNHGQAIYAKKEYQPEPQVGETQWNSDVGATDKAAGRILKKTTPMVPCYGSYAKPTNEEGGQATNQQVDMQFFATGSSNTTPKVVLYSEDVTIETPDTHLVFKPIVTAGTTNAEALLAQQAAPNRPNYIGFRDNFIGLMYYNSTGNMGVLAGQASQLNAVVDLQDRNTELSYQLMLDALGDRSRYFSMWNQAVDSYDPDVRIVENHGVEDELPNYCFPLGGMAVTDTYTGIKPDNGGTTFSNDDTFATRGIEIGSGNMFAMEINLQANLWRSFLYSNIGLYLPDTLKITPDNITLPDNKNTYQYMNGRVTPPGLVDTYVNVGARWSPDVMDGINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQVVDTVTYTDYKDVKLPYQHNNSGFVGYMGPTMREGQAYPANYPYPLIGETAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 168 - protein, 921 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPSEWTDTSDNKLKAYAQAPYQSQGLTKDGIQVGLVVTESGQTPQYANKVYQPEPQIGENQWNLEQEDKAAGRVLKKDTPMFPCYGSYARPTNEQGGQAKNQEVDLQFFATPGDTQNTAKVVLYAENVNLETPDTHLVFKPDDDSTSSKLLLGQQAAPNRPNYIGFRDNFIGLMYYNSTGNMGVLAGQASQLNAVVDLQDRNTELSYQLMLDALGDRSRYFSMWNQAVDSYDPDVRIIENHGVEDELPNYCFPLGGMVVTDNYNSVTPQNGGSGNTWQADNTTFSQRGAQIGSGNMFALEINLQANLWRGFLYSNIGLYLPDSLKITPDNITLPENKNTYQYMNGRVTPPGLIDTYVNVGARWSPDVMDSINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQVVDTVTYTDYKDVKLPYQHNNSGFVGYMGPTMREGQAYPANYPYPLIGETAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 169 - protein, 937 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPAEWEDTNDSNKLKVRGQAPYVSTFGAATALTKDGIQVGVTVSESARTPQYADKSYQPEPQIGETEWNSEVGNDDKLAGRVLKKSTPMFPCYGSYAKPTNEKGGQAIQPAGSSGDTNPAVELQFFATTSATTTPKAVLYAEDVAIEAPDTHLVFKPTVTAGTTSSEALLTQQAAPNRPNYIAFRDNFIGLMYYNSTGNMGVLAGQASQLNAVVDLQDRNTELSYQLMLDALGDRSRYFSMWNQAVDSYDPDVRIVENHGVEDELPNYCFPLGGMVVTDTYTPIKPNGSGSVTWTANTDVFSNRVEIGSGNLFAMEINLQANLWRSFLYSNIGLYLPDSLKITPDNITLPENKNTYQYMNGRVTPPGLIDTYVNVGARWSPDVMDSINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQIVDSTAYTNYQDVKLPYQHNNSGFVGYMGPTMREGQAYPANYPYPLIGATAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 170 - protein, 919 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPSEWNGTDSKTNVRGQAPFISDAITKEGIKVGTDTGNPGQAIYAKKEYQPEPQVGETQWNSDVGATDKAAGRILKKTTPMVPCYGSYAKPTNEEGGQATNQQVDMQFFATGSSNTTPKVVLYSEDVTIETPDTHLVFKPIVTAGTTNAEALLAQQAAPNRPNYIGFRDNFIGLMYYNSTGNMGVLAGQASQLNAVVDLQDRNTELSYQLMLDALGDRSRYFSMWNQAVDSYDPDVRIVENHGVEDELPNYCFPLGGMAVTDTYTGIKPDNGGTTFSNDDTFATRGIEIGSGNMFAMEINLQANLWRSFLYSNIGLYLPDTLKITPDNITLPDNKNTYQYMNGRVTPPGLVDTYVNVGARWSPDVMDGINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQVVDTVTYTDYKDVKLPYQHNNSGFVGYMGPTMREGQAYPANYPYPLIGATAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 171 - protein, 931 aa, synthetic construct
MATPSMMPQWSYMHIAGQDASEYLSPGLVQFARATDTYFSLGNKFRNPTVAPTHDVTTDRSQRLTLRFVPVDREDTAYSYKVRFTLAVGDNRVLDMASTYFDIRGMLDRGPSFKPYSGTAYNSLAPKGAPNPSQWITNGGTAKTNSFAQASYIGMGANLTTNGIQVAVDTNNANAAVYANKIYQPEPQVGTSQWNINPTQNAAGRILKPTTPMQPCYGSYAYPTNENGGQVKITDGSTDPTGANSVSMEFFTTTADNQNQAKVVLYSEDVNLEAPDTHLVFKPDVADNAKAAETLLGQQSAPNRPNYIGFRDNFIGLMYYNSTGNMGVLAGQASQLNAVVDLQDRNTELSYQLMLDALGDRSRYFSMWNQAVDSYDPDVRIIENHGVEDELPNYCFPLNGQGISNTYRGLSVQNGQNTWQANNNAAENNEISIGNIFAMEINLAANLWRSFLYSNVALYLPDSYKYTPDNIELPTNQNSYGYINGRVTAPNAIDTYVNIGARWSPDPMDNVNPFNHHRNAGLRYRSMLLGNGRYVPFHIQVPQKFFAIKNLLLLPGSYTYEWNFRKDVNMILQSTLGNDLRVDGASVRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQVVDSVNYANYKEVKMPFQHNNSGFVGYMGPTMREGQAYPANYPYPLIGATAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 172 - protein, 935 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPAEWENSDNKLKVRAQAPFVSSYGATAITKDGIQVGVVLTDSTQTPQYADKTYQPEPQIGELQWNSEVGNDDKVAGRVLKKTTPMFPCYGSYAKPTNDKGGQATQPAAGQDAQNAVELQFFATTSATTTPKAVLYAEDVSIETPDTHLVFKPTVTAGTTSSEALLSQQAAPNRPNYIAFRDNFIGLMYYNSTGNMGVLAGQASQLNAVVDLQDRNTELSYQLMLDALGDRSRYFSMWNQAVDSYDPDVRIVENHGVEDELPNYCFPLGGTVVTDTYTPIQPNSNGGGTWVAKDNVFTDRGVEISSGNMFAMEINLQANLWRSFLYSNIGLYLPDTLKITPDNITLPENKNTYQYMNGRVTPPGLVDTYVNVGARWSPDVMDSINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQVVDTVAYKDYYQDVKLPYQHNNSGFVGYMGPTMREGQAYPANYPYPLIGATAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 173 - protein, 931 aa, synthetic construct
MATPSMMPQWSYMHIAGQDASEYLSPGLVQFARATDTYFSLGNKFRNPTVAPTHDVTTDRSQRLTLRFVPVDREDTAYSYKVRYTLAVGDNRVLDMASTYFDIRGVLDRGPSFKPYSGTAYNSLAPKGAPNPAEWADSDSKLKVRAQAPFVSSYGNNAITKEGIQVGVILTDSGQTAQYADKTYQPEPQIGELQWNSEVGTNDKVAGRVLKKTTPMFPCYGSYAKPTNAKGGQATQPASQETQNPELQFFAASSTVNTPKAVLYAEDVSIEAPDTHLVFKPSVTEGTTSSEALLAQQAAPNRPNYIAFRDNFIGLMYYNSTGNMGVLAGQASQLNAVVDLQDRNTELSYQLMLDALGDRSRYFSMWNQAVDSYDPDVRIVENHGVEDELPNYCFPLGGMAVTDTYSPIKLNGGQNGWEANNDVYAERGVEIGSGNLFAMEINLQANLWRSFLYSNIGLYLPDTLKITPDNITLPENKNTYQYMNGRVTPPGLVDTYVNVGARWSPDVMDSINPFNHHRNAGLRYRSMLLGNGRYVPFHIQVPQKFFAIKNLLLLPGSYTYEWNFRKDVNMILQSSLGNDLRVDGASIRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQIVDSTAYTNYQDVKLPYQHNNSGFVGYMGPTMREGQAYPANYPYPLIGATAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 174 - protein, 931 aa, synthetic construct
MATPSMMPQWSYMHIAGQDASEYLSPGLVQFARATDTYFSLGNKFRNPTVAPTHDVTTDRSQRLTLRFVPVDREDTAYSYKVRFTLAVGDNRVLDMASTYFDIRGMLDRGPSFKPYSGTAYNSLAPKGAPNPSQWITNGGTAKTNSFAQASYIGMGANLTTNGIQVAVDTNNANAAVYANKIYQPEPQVGTSQWNINPTQNAAGRILKPTTPMQPCYGSYAYPTNENGGQVKITDGTTDPTGANSVSMEFFTTTADNQNQAKVVLYSEDVNLEAPDTHLVFKPDVADNAKAAETLLGQQSAPNRPNYIGFRDNFIGLMYYNSTGNMGVLAGQASQLNAVVDLQDRNTELSYQLMLDALGDRSRYFSMWNQAVDSYDPDVRIIENHGVEDELPNYCFPLNGQGISNTYKGLSVQNGQNTWQANNNAAENNEISIGNIFAMEINLAANLWRSFLYSNVALYLPDSYKYTPDNIELPTNQNSYGYINGRVTAPNAIDTYVNIGARWSPDPMDNVNPFNHHRNAGLRYRSMLLGNGRYVPFHIQVPQKFFAIKNLLLLPGSYTYEWNFRKDVNMILQSTLGNDLRVDGASVRFDSINLYANFFPMAHNTASTLEAMLRNDTNDQSFNDYLCAANMLYPIPANATSVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQSNMTKDWFLIQMLSHYNIGYQGFYVPENYKDRMYSFFRNFQPMSRQVVDSVNYANYKEVKMPFQHNNSGFVGYMGPTMREGQAYPANYPYPLIGATAVPSLTQKKFLCDRVMWRIPFSSNFMSMGSLTDLGQNMLYANSAHALDMTFEVDPMDEPTLLYVLFEVFDVVRIHQPHRGVIEAVYLRTPFSAGNATT

SEQ ID NO: 175 - protein, 935 aa, synthetic construct; identical to SEQ ID NO: 172

SEQ ID NO: 176 - protein, 364 aa, synthetic construct
MKRTRVDEDFNPVYPYDSTSTPAVPFISPPFVNSDGLQENPPGVLSLRIAKPLYFDMERKLALSLGRGLAITSTGQLESTQSVQTTPPLVVNNSNTLVLRYSSPLGLSGDNLILNCSDPLRVVNNSLTFSYLSPLRFEGGSLTFNYTSPLKLLNSSLAIGINSNKGLGNDSDELSVKLTSDLKFNNDGKIAFGIQSLCTTPTAASNCTVFTNGDSLLCLCLTKCGAHVLGSVSLTGMQGTITAMTQNYISIQFLFDNNGALTSSPLLNNNTWGIRQNDTSSANPAYNALAFMPNSTVYVRGQSGEPRNNYYTQTYLRGNVKKPIILTVTYNSAASGYSLTFKWDAVVTEKFATPTSSFCYITEQ

SEQ ID NO: 177 - protein, 363 aa, synthetic construct
MKRTRVDEDFNPVYPYDTTTTPAVPFISPPFVNSDGLQENPPGVLSLRIAKPLYFDMERKLALSLGRGLTITAAGQLESTQSVQTNPPLIITNNNTLTLRHSPPLNLTDNSLVLGYSSPLRVTDNKLTFNFTSPLRYENENLTFNYTEPLKLINNSLAIDINSSKGLSSVGGSLAVNLSSDLKFDSNGSIAFGIQTLWTAPTSTGNCTVYSEGDSLLSLCLTKCGAHVLGSVSLTGLTGTITQMTDISVTIQFTFDNNGKLLSSPLINNAFSIRQNDSTASNPTYNALAFMPNSTIYARGGGGEPRNNYYVQTYLRGNVQKPIILTVTYNSAATGYSLSFKWTALAREKFATPTTSFCYITEQ

SEQ ID NO: 178 - protein, 360 aa, synthetic construct
MKRTRVDEDFNPVYPYDTTTTPAVPFISPPFVNSDGLQENPPGVLSLRIAKPLYFDMERKLALSLGRGLTITANGQLESTQSVQTNPPLTVTNNNTLILRHSSPLILTDNNLTVGFSSPLRVIDNKLTFTFTSPLRYENETLTFNYTEPLTLMNSNLALNVNSSKGLRVDGGSLGTNLSPDLRFNSSGAIAFGIQTLWTPPTSNPNCTVYTESDSLLSLCLTKCGAHVLGSVSLTGVAGTMINMAETSLAIEFTFDDTGKLLHSPLVNTTFSIRQGDSPASNPTYNALAFMPNSTLYARGGSGEPRNNYYVQTYLRGNVQRPITLTVTFNSAATGYSLSFKWTAVAREKFAAPATSFCYI

SEQ ID NO: 179 - protein, 559 aa, synthetic construct
MKRARVDEDFNPVYPYDPPYAPVMPFITPPFTSSDGLQEKPLGVLSLNYKDPITTQNGSLTLKIGNGLTLDNQGQLTSTAGEVEPPLTNANNKLALAYSEPLAVKSNRLTLSHTAPLVVANNSLALQVSEPIFINDDDKLALQTAAPLVTNAGTLRLQSAAPLGLVENTLRLLFSKPLYLQNDFLALGIERPLAIAAAGTLALQLTPPLKTNDDGLTLSTVEPLTVKNGNLGLQISRPLVVQNSSLSLAITPPLRLFNSDPVLGLGFTFPLAVTDNLLSLNMGDGVKLTYNKLTANLGRDLQFENGAIAVTLTAESPLQYTNKLQLNIGAGLRYNGASRKLDVNINQNKGLTWDNDAVIPKLGSGLQFDPNGNIAVIPETVKPQTLWTTADPSPNCSVYQDLDARLWLALVKSGDMVHGSIALKALKGTLLNPTASYISIVIYFYSNGVRRTNYPTFDNEGTLANSATWGYREGQSANTNVTNATEFMPSSTRYPVNKGDNIQNQSFSYTCIKGDFAMPVPFRVTYNHALEGYSLKFTWRVVANQAFDIPCCSFSYITE

SEQ ID NO: 180 - protein, 560 aa, synthetic construct
MKRARVDEDFNPVYPYDPPHAPVMPFITPPFTSSDGLQEKPLGVLSLNYRDPITTQNGSLTVKLGNGLTLDNQGQLTSTAGEVEPPLTNANNKLALVYSDPLAVKRNSLTLSHTAPLVIADNSLALQVSEPIFINDKDKLALQTAAPLVTNAGTLRLQSAAPLGIADQTLKLLFTNPLYLQNNFLTLAIERPLAITNSGKLALQLSPPLQTADTGLTLQTNVPLTVSNGTLGLAIKRPLIVQDNNLFLDFRAPLRLFNSDPVLGLNFYTPLAVRDEALTVNTGRGLTVSYDGLILNLGKDLRFDNNTVSVALSAALPLQYTDQLRLNVGAGLRYNPVSKKLDVNPNQNKGLTWENDYLIVKLGNGLGFDGNGNIAVSPQVTSPDTLWTTADPSPNCSIYTDLDAKMWLSLVKQGGVVHGSVALKALKGTLLSPTESAIVIILHFDNYGVRILNYPTLGTQGTLGNNATWGYRQGESADTNVLNALAFMPSSKRYPRGRGSEVQNQTVGYTCIQGDLSMPVPYQIQYNYGPTGYSFKFIWRTVSRQPFDIPCCFFSYITEE

SEQ ID NO: 181 - protein, 560 aa, synthetic construct
MKRARVDEDFNPVYPYDPPYAPIMPFITPPFTSSDGLQEKPLGVLSLKYKDPITTQNGSLTLKLGNGLNINNQGQLTSSAGEVEPPLTNADNKLALAYSDPLTLKNSRLTLSHNAPLAINNNSLSLEVSEPIFINNDNKLSLKADAPLTTSAGTLRLQSAAPLGLAEQTLKLLFSNPLYLRGDFLTLAIERPLAVTADGLLSLALNPPLTTTNTGLALSTAVPLTVTNGNLSLNVKRPFIIQDGSLYMDFRPPLYLFNSEPQLGVNFNAPLTVRDNGLAINTGDGLTVTYNKLTLNLGRDLQYENGAAAVKLSTAPPLQYTTQLQLNLGAGLRLGPTRNLDVAINHNKGLAWENNEVVTKLGQGLYFDSSGSIALSPTNPRPDTLWTTADPSPNCTVYESLDSRLWLALVKCGGMVHGSIALQAEKGQLLRPTASFISIVIYFYSDGVRRTNYPTIGNDEGTLANSATWGYRQGQSADTNVTNAVEFMPSLHRYPINQGDNIKNQMITYTCIQGNVNMPVPLKITFNHALEGYSLKFTWRVVANEKFDIPCCSFSYITEQ

SEQ ID NO: 182 - protein, 363 aa, synthetic construct
MKRTRVDEDFNPVYPYDTTTTPAVPFISPPFVNSDGLQENPPGVLSLRIAKPLYFDMERKLALSLGRGLTITAAGQLESTQSVQTNPPLIITNNNTLTLRHSPPLNLTDNSLVLGYSSPLRVTDNKLTFNFTSPLRYENENLTFKYTEPLKLINNSLAIDINSSKGLSSVGGSLAVNLSSDLKFDSNGSIAFGIQTLWTAPTSTGNCTVYSEGDSLLSLCLTKCGAHVLGSVSLTGLTGTITQMTDISVTIQFTFDNNGKLLSSPLINNAFSIRQNDSTASNPTYNALAFMPNSTIYARGGGGEPRNNYYVQTYLRGNVQKPIILTVTYNSAATGYSLSFKWTALAREKFATPTTSFCYITEQ

SEQ ID NO: 183 - protein, 559 aa, synthetic construct
MKRVRIDEDFNPVYPYDPPYAPVMPFITPPFTSSDGLQEKPLGVLSLNYKDPITTQNGSLTLKIGNGLTLDNQGQLTSTAGEVEPPLTNANNKLALAYSEPLAVKSNRITLSHTAPLVVADDSLALQVSEPIFIDDDDKLALQTAAPLVTNAGTLRLQSAAPLGLVENTLRLLFSKPLYLQNDFLALAIERPLAIAAAGTLALQLTPPLKTNDDGLTLSTVEPLTVKNGNLGLQISRPLVVQNSSLSLAITPPLRLFNSDPVLGLGFTFPLAVTNNLLSLNMGDGVKLTYNKLTANLGRDLQFENGAIAVTLTAELPLQYTNKLQLNIGAGLRYNGASRKLDVNINQNKGLTWDNDAVIPKLGSGLQFDPNGNIAVIPETVKPQTLWTTADPSPNCSVYQNLDARLWLALVKSGDMVHGSIALKALKGTLLNPTASYISIVIYFYSNGVRRTNYPTFDNEGTLANSATWGYREGQSANTNVTNATEFMPSSTRYPVNKGDNIQNQSFSYTCIKGDFAMPVPFRVTYNHALEGYSLKFTWRVVANQAFDIPCCSFSYITE

SEQ ID NO: 184 - protein, 293 aa, synthetic construct
MYLLITTHDFHLSQMKRTRVDEDFNPVYPYDAEQSSSVPFVTPPFTSSNGLTESPSGVLTLNYSQPLTTINGKLTLNVDGGLNINNDKKLQVRTNENEGVTLQNNAVSVKIGNDLRFDSRGQITLNPVSLWTTPSESANCTLYNSSANFFLCLTRNDAHVVGTVNLTGINGKFSSMTENSVIVQLAFDENGTLQSSPLAQNVWGIRQTPNSPNAVNLLTFMPSSLVYPREGGGKPKNNLYFNTFLRGNLSRPITLTLTLNSLPTGYTLTFKWGTYLNEPFSVPTTCFCYVAEQ

SEQ ID NO: 185 - protein, 292 aa, synthetic construct
MKRARVDESFNPVYPYDSTSTPAIPFVTPPFVSNDGLQENPPGILSLQIAKPLFFNTQRKIALSLGRGLAITSAGELENTQRIQSTPPLLLNSNTLSMNYSSPLTVLDNKLALNYLSPLRVVNDSLTFNFTSPLHVADGNLTFSYTSPLQVTNNSLALNYSAPLRLLNNSLTFTYANPFKIANSSLSLKLDYPSALRLNNNGSLQVIAGWGLGIAGNNDLYVKRQAPLAVYESGSNAGTLYLRLGPNSALRTVNDNLTLNTGQGLQITNNTLSVKLGNGLRFNNTGAIELAQ

SEQ ID NO: 186 - protein, 555 aa, synthetic construct
MKRARVDEDFNPVYPYDPPYAPIMPFITPPFTSSDGLQEKPFGVLSINYKDPITTQNGSLTLKVGNGLSINNQGQLTSTQTTVTPPLTNESDTLGLAYSDPLTVKNNSLTLSHSAPFAVSNHSLSLKTTTPFFINQQNELALSTDAPLQVTAGSLRLQSAAPLGIAEQTLRLLFSNPLFLQDNFLTLAIERPLAITSSGNLTVQLNPPFQSTSTGLALSLSNPLTISNGTLAMSIKRPFIIDDNKLYMDFRPPLRLFNSEPQLGVHFNPPITVQQGGLALNTGDGLQVYLNKLTLNIGRDLQYENGSLVVKLTPAPPLQYTTQLQLNMGAGLHLGATKQLTLDVDTNRGLTWNNNKLAVNLGDGLQFNSYGQIIPTVKKDTLWTTADPSPNCSIYKDLDAKLWLSLVKSGGMVHGSIALQALKDTLLSPTDSFITIILYFDENGVRAPYPTFDNEGTLAKDATWGYRQGPSADTNVTNALEFMPSSARYPRGKHEQAQNQTFGYTCLQGNFSWPIPFRVQYNYVRTGFSFKFTWQVVNRQKFDIPCCSFSYITEQ

SEQ ID NO: 187 - protein, 363 aa, synthetic construct; identical to SEQ ID NO: 177

SEQ ID NO: 188 - protein, 560 aa, synthetic construct; identical to SEQ ID NO: 180

SEQ ID NO: 189 - protein, 293 aa, synthetic construct; identical to SEQ ID NO: 184

SEQ ID NO: 190 - protein, 292 aa, synthetic construct; identical to SEQ ID NO: 185

SEQ ID NO: 191 - protein, 555 aa, synthetic construct
MKRARVDEDFNPVYPYDPPYAPIMPFITPPFTSSDGLQEKPFGVLSINYKDPITTQNGSLTLKVGNGLSINNQGQLTSTQTTVTPPLTNESDTLGLAYSDPLTVKNNSLTLSHSAPFAVSNHSLSLKTTTPFFINQQNELALSTDAPLQVTAGSLRLQSAAPLGIAEQTLRLLFSNPLFLQDNFLTLAIERPLAITSSGNLTVQLNPPFQSTSTGLALSLSNPLTISNGTLAMSIKRPFIIDDNKLYMDFRPPLRLFDSEPQLGVHFNPPITVQQGGLALNTGDGLQVYLNKLTLNIGRDLQYENGSLVVKLTPAPPLQYTTQLQLNMGAGLHLGATKQLTLDVDTNRGLTWNNNKLAVNLGDGLQFNSYGQIIPTVKKDTLWTTADPSPNCSIYKDLDAKLWLSLVKSGGMVHGSIALQALKDTLLSPTDSFITIILYFDENGVRAPYPTFDNEGTLAKDATWGYRQGPSADTNVTNALEFMPSSARYPRGKHEQAQNQTFGYTCLQGNFSWPIPFRVQYNYVRTGFSFKFTWQVVNRQKFDIPCCSFSYITEQ

SEQ ID NO: 192 - protein, 348 aa, synthetic construct
MKRTRVDEDFNPVYPYDTTSTPAVPFISPPFVNSDGLQENPPGILSLRIDRPLYFNTQRQLALSLGNGLAITAGGELESTLSVRPNPPLVINGNSLSLRYSDPLRVVNDSLTFNFTSPLRFESGSLTFHYASPLKLMNDSLALDVEPSKGLSTDGNKLAVKLSSDLKFDNNGAIAFGIQSLWTAPTTTPNCMTYAENDSLLSLCLTKCGAHVLGSVSLTGFTGDLLNMKQTTVSIEFRFDNNGILTTSPLVNSSSWGIRQNNGIYPNPVYNALAFMPNSTVYSRGGGGEPRNNYYTQTYLRGNTQIPITLTVTYNSANTGYSLTFKWTALTNQKFAAPTASFCYISEQ

SEQ ID NO: 193 - protein, 559 aa, synthetic construct; identical to SEQ ID NO: 183

SEQ ID NO: 194 - protein, 293 aa, synthetic construct; identical to SEQ ID NO: 184

SEQ ID NO: 195 - protein, 292 aa, synthetic construct
MKRARVDESFNPVYPYDSTSTPAIPFVTPPFVSNDGLQENPPGILSLQIAKPLFFNTQRKIALSLGRGLAITSAGELENTQRIQSTPPLLLNSNTLSMNYSSPLTVLDNKLALNYLSPLRVVNDSLTFNFTSPLHVADGNLTFSYTSPLHVTNNSLALNYSAPLRLLNNSLTFTYANPFKIANSSLSLKLDYPSALRLNNNGSLQVIAGWGLGIAGNNDLYVKRQAPLAVYESGSNAGTLYLRLGPNSALRTVNDNLTLNTGQGLQITNNTLSVKLGNGLRFNNTGAIELAQ

SEQ ID NO: 196 - protein, 555 aa, synthetic construct; identical to SEQ ID NO: 186

SEQ ID NO: 197 - protein, 259 aa, synthetic construct
MSTSLPLPTIFIFSEMKRTRVDEDFNPVYPYDTTSTPAIPFISPPFVNSDGFQESPPGILSLRLNKPLSFTNHAVSLNVGSGLTINSTGFLEATQQISTANPLLLQNNTLSLISSAPLYVNGSALALRLNSPFVVHNNGLAINLAEPLYVTNNSLNLRLASPFYVNNNSLNLRLPNPSGLSIDGSGLKVKAGYGLRLHANNDLYVYTDFPLDKYDNGAKQGQLYLRTGKGLQLSNNDLTVKLGAGLKFDNNGAITLAST

SEQ ID NO: 198 - protein, 359 aa, synthetic construct
MKRARIDEDYNPVYPYNTSTPPFIAPPFVDNDGFQENPQGVLSLRISKPLYFDMNKRLSLSLGQGLSTTSAGELESAQNVRANPPLSINSNTVSLRYSSPLNLTENGLVLGYSDPLRVVGGALTFNYTSPLRYENDSLTFNYTSPLKLMNNSLAIEVNSSKGVTSKNGAIAVQLSPDLKFDSNGSIAFGIQTLWTFPTTTSNCTVFTSNDSLLSLCLTKCGAHVLGSVSLTGMAGALLNMTQTSVTIQFTFDNTGSLTSSPLINNAWGIRQNDSASPNPTFNALTFMPNSTVYSRGGSGEPRNNYYTQTYLRGNIRKPIVLTVTYNSANTGYSLSFKWDAIATEKFATPTSSFCYISEQ

SEQ ID NO: 199 - protein, 593 aa, synthetic construct
MGCYCNRKICHSYIFILLHFRTIKSCYTPSRFFQMKRARVDEDFNPVYPYDSPNAPIMPFITPPFTSSDGLQEKPLGVLSLNYKDPVTTQNGSLTLKLGSGLNINDQGQLTSTAGEVEPPLTNNNNKLALAYSQPLAVENNRLIVSYNAPLAVTGNSLGLQVSEPIFINDNDKLALQTAAPLKTNAGTLRLQSAAPLGLAEETLKLLFSKPLYLQGDFLSLGIERPLAIAAAGTLALQLTPPLTTNDSGLTISTADPVTVSNGNLGLRISRPLVVQNNSLSLALTPPLRLFNSEPVLGLGFTFPITVVDNLLSLNIGNGLTLLYNKLTANLGRDLQFQNGAIAVSLTTEPPLQYTTKLQLNVGAGLRYNGASRKLDVDINQQKGLTWENNAVVPKLGQGLYFDPQGNIAMSPTNVKPDTLWTTADPSPNCSVYTDLDAKLWLSLVKCGGMVQGSIALQAQKGTLMSVTDPSVTIILHFNDNGVRINYPTLDNQGTLGRDSTWGYRQGQSANTNVTNAVEFMPNSSRYPRGRGEQAQNQTVGYTCLQGNLSMPIFFQVQYNYVTTGYSFKFTWQAVQRQKFDIPCCSFSYITEE

SEQ ID NO: 200 - protein, 312 aa, synthetic construct
MRWWAANFLQTLKEILLSTSLLFPTIFIFSEMKRTRVDEDFNPVYPYNTEPTPSAPFVTPPFVSSNGLMESPSGVLSIRYTQPITVVNNSLSLKTGNGLSVSSDGKLQIAINTNKGLSNEDNKIAVKVGPELYFDADGAITMNSQTLWTNPNETANCSVFQSLDSLLTLCLTKNGAHVLGSVSLTGLSGPLLKMTTTSVTVQLIFDSNGVLTTSQLNTNSWGMRANTNLNAPVTNALPFMPNSTIYARGNAGEPRSNYYVQTYLRGNLNKQITLSISFNASSSGYSLTFKWSAIATEKFATPTSSFCYIAEQ

SEQ ID NO: 201 - protein, 291 aa, synthetic construct
MKRTRIEDDFNPVYPYDSVSAPAVPFVSPPFVNSDGLKESPPGVLSLQIAKPLLFDAQRKVTLSLGRGLAVNSAGQLESTQILQTNPPLLLSNNMLSLDCAFPLSVANNQLSLKYTDPLRLVNDSLTFNFTSPLHLANGNLTFSYTTPLNIINNSLALNYSPPLRLVNSNLTMGYADPFQVANSNLSLKIANPSGLRIRNGNLQIAAGYGLQIAGNNDLYVRRETPLDVYTSGAKEGILYLRLGPNSGLQVLSDNLSLKTGQGLQILSNTLSVKLGRGLRFDSSGAIELST

SEQ ID NO: 202 - protein, 560 aa, synthetic construct
MKRARIEEDFNPVYPYDPPYAPIMPFITPPFTSSDGLQEKPFGVLSLNYKNPITTQNGSLTLKLGNGLNINNQGELTSTAGAVQPPLTNADNKLTLAYSEPLTVQNKRLTLSHSAPLAVADNSLSLQVTDPIFINADNKLALQTDAPLNTTTGVLRLKSAAPLGLVEQTLNLLFAKPLYLQGDFLALAIERPLAVTADGSLALALDPPLKTSNTGLGLLTAAPLTVTNGNLNLNVKRPFVIQDNSLYMDFRAPLRLFNSEPQLGVNFTSPLTVRADALTLDTGNGLQVLFNKLTLNLGRDLEYDNGAIAVRLNPAAPLQYTNQLQLNLGAGLRLGVSKKLTVDVDENKGLTWNGNQLVASLGTGLAFDNYGRISVVSTAAGDDTLWTTADPSPNCTVYEQLDARLWLSLVKCGGMVQGSVALKAEKGRLLNPTASFISIVMYFNRYGVWRTDYPTIGDDQGIIANSATWGYRRGESADTNVTNALKFMPSSRRYPINQGNNIQNQMITYTCIQGDVSMPVPLKITFNHAFEGYSFKFTWRVVANQRFDIPCCSFSYITEE

SEQ ID NO: 203 - protein, 364 aa, synthetic construct
MKRTRVDEDFNPVYPYDSTSTPAVPFISPPFVNSDGLQENPPGVLSLRIAKPLYFDMERKLALSLGRGLAITSTGQLESTQSVQTTPPLVVNNSNTLVLRYSSPLGLSGDNLILNCSDPLRVINNSLTFSYLSPLRFEGGSLTFNYTSPLKLLNSSLAIGINSNKGLGNDSDELSVKLTSDLKFNNDGKIAFGIQSLCTTPTAASNCTVFTNGDSLLCLCLTKCGAHVLGSVSLTGMQGTITAMTQNYISIQFLFDHNGALTSSPLLNNNTWGIRQSDTSSANPAYNALAFMPNSTVYVRGQSGEPRNNYYTQTYLRGNVKKPIILTVTYNSAASGYSLTSKWDAVVTEKFATPTSSFCYITEQ

SEQ ID NO: 204 - protein, 559 aa, synthetic construct
MKRARVDEDFNPVYPYDSPNAPIMPFITPPFTSSDGLQEKPLGVLSLNYKDPVTTQNGSLTLKLGSGLNINDQGQLTSTAGEVEPPLTNNNNKLALAYSQPLAVENNRLIVSYNAPLAVTGNSLGLQVSEPIFINDNDKLALQTAAPLKTNAGTLRLQSAAPLGLAEETLKLLFSKPLYLQGDFLSLGIERPLAIAAAGTLALQLTPPLTTNDSGLTISTADPVTVSNGNLGLRISRPLVVQNNSLSLALTPPLRLFNSEPVLGLGFTFPITVVDNLLSLNIGNGLTLLYNKLTANLGRDLQFQNGAIAVSLTTEPPLQYTTKLQLNVGAGLRYNGASRKLDVDINQQKGLTWENNAVVPKLGQGLYFDPQGNIAMSPTNVKPDTLWTTADPSPNCSVYTDLDAKLWLSLVKCGGMVQGSIALQAQKGTLMSVTDPSVTIILHFNDNGVRINYPTLDNQGTLGRDSTWGYRQGQSANTNVTNAVEFMPNSSRYPRGRGEQAQNQTVGYTCLQGNLSMPIFFQVQYNYVTTGYSFKFTWQAVQRQKFDIPCCSFSYITEE

SEQ ID NO: 205 - protein, 259 aa, synthetic construct; identical to SEQ ID NO: 197

SEQ ID NO: 206 - protein, 359 aa, synthetic construct; identical to SEQ ID NO: 198

SEQ ID NO: 207 - protein, 529 aa, synthetic construct
MGCYCNRKICHSYIFILLHFRTIKSCYTPSRFFQMKRARVDEDFNPVYPYDSPNAPIMPFITPPFTSSDGLQEKPLGVLSLNYKDPVTTQNGSLTLKLGSGLNINDQGQLTSTAGEVEPPLTNNNNKLALAYSQPLAVENNRLIVSYNAPLAVTGNSLGLQVSEPIFINDNDKLALQTAAPLKTNAGTLRLQSAAPLGLAEETLKLLFSKPLYLQGDFLSLGIERPLAIAAAGTLALQLTPPLTTNDSGLTISTADPVTVSNGNLGLRISRPLVVQNNSLSLALTPPLRLFNSEPVLGLGFTFPITVVDNLLSLNIGNGLTLLYNKLTANLGRDLQFQNGAIAVSLTTEPPLQYTTKLQLNVGAGLRYNGASRKLDVDINQQKGLTWENNAVVPKLGQGLYFDPQGNIAMSPTNVKPDTLWTTADPSPNCSVYTDLDAKLWLSLVKCGGMVQGSIALQAQKGTLMSVTDPSVTIILHFNDNGVRINYPTLDNQGTLGRDYANFFPSPVQLCNHWIFLQIHLASCTKAKV

SEQ ID NO: 208 - protein, 259 aa, synthetic construct; identical to SEQ ID NO: 197

SEQ ID NO: 209 - protein, 359 aa, synthetic construct; identical to SEQ ID NO: 198

SEQ ID NO: 210 - protein, 593 aa, synthetic construct
MGCYCNRKICHAYIFILLHFRTIKSCYTPSRFFQMKRARVDEDFNPVYPYDSPNAPIMPFITPPFTSSDGLQEKPLGVLSLNYKDPVTTQNGSLTLKLGSGLNINDQGQLTSTAGEVEPPLTNNNNKLALAYSQPLAVENNRLIVSYNAPLAVTGNSLGLQVSEPIFINDNDKLALQTAAPLKTNAGTLRLQSAAPLGLAEETLKLLFSKPLYLQGDFLSLGIERPLAIAAAGTLALQLTPPLTTNDSGLTISTADPVTVSNGNLGLRISRPLVVQNNSLSLALTPPLRLFNSEPVLGLGFTFPITVVDNLLSLNIGNGLTLLYNKLTANLGRDLQFQNGAIAVSLTTEPPLQYTTKLQLNVGAGLRYNGASRKLDVDINQQKGLTWENNAVVPKLGQGLYFDPQGNIAMSPTNVKPDTLWTTADPSPNCSVYTDLDAKLWLSLVKCGGMVQGSIALQAQKGTLMSVTDPSVTIILHFNDNGVRINYPTLDNQGTLGRDSTWGYRQGQSANTNVTNAVEFMPNSSRYPRGRGEQAQNQTVGYTCLQGNLSMPIFFQVQYNYVTTGYSFKFTWQAVQRQKFDIPCCSFSYITEE

SEQ ID NO: 211 - protein, 259 aa, synthetic construct; identical to SEQ ID NO: 197

SEQ ID NO: 212 - protein, 359 aa, synthetic construct
MKRARIDEDYNPVYPYNTSTPPFIAPPFVDNDGFQENPQGVLSLRISKPLYFDMNKRLSLSLGQGLSTTSAGELESAQNVRANPPLSINSNTVSLRYSSPLNLTENGLVLGYSDPLRVVGGALTFNYTSPLRYENDSLTFNYTSPLKLMNNSLAIEVNSSKGVTSKNGAIAVQLSPDLKFDSNGSIAFGIQTLWTFPTTTSNCTVFTSNDSLLSLCLTKCGAHVLGSVSLTGMAGALLNMTQTSVTIEFTFDNTGSLTSSPLINNAWGIRQNDSASPNPTFNALTFMPNSTVYSRGGSGEPRNNYYTQTYLRGNIRKPIVLTVTYNSANTGYSLSFKWDAIATEKFATPTSSFCYISEQ

SEQ ID NO: 213 - protein, 593 aa, synthetic construct
MGCYCNRKICHSYIFILLHFRTIKSCYTPSRFFQMKRARVDEDFNPVYPYDSPNAPIMPFITPPFTSSDGLQEKPLGVLSLNYKDPVTTQNGSLTLKLGSGLNINDQGQLTSTAGEVEPPLTNNNNKLALAYSQPLAVENNRLIVSYNAPLAVTGNSLGLQVSEPIFINDNDKLALQTAAPLKTNAGTLRLQSAAPLGLAEETLKLLFSKPLYLQGDFLSLGIERPLAIAAAGTLALQLTPPLTTNDSGLTISTADPVTVSNGNLGLRISRPLVVQNNSLSLALTPPLRLFNSEPVLGLGFTFPITVVDNLLSLNIGNGLTLLYNKLTANLGRDLQFQNGAIAVSLTTEPPLQYTTKLQLNVGAGLRYNGASRKLDVDINQQKGLTWENNAVVPKLGQGLYFDPQGNIAMSPTNVKPDTLWTTADPSPNCSVYTDLDAKLWLSLVKCGGMVQGSIALQAQKGTLMSVTEPSVTIILHFNDNGVRINYPTLDNQGTLGRDSTWGYRQGQSANTNVTNAVEFMPNSSRYPRGRGEQAQNQTVGYTCLQGNLSMPIFFQVQYNYVTTGYSFKFTWQAVQRQKFDIPCCSFSYITEE

SEQ ID NO: 214 - protein, 259 aa, synthetic construct
MSTSLPLPTIFIFSEMKRTRVDEDFNPVYPYDTTSTPAIPFISPPFVNSDGFQESPPGILSLRLNKPLSFTNHAVSLNVGSGLTINSTGFLEATQQISAANPLLLQNNTLSLISSAPLYVNGSALALRLNSPFVVHNNGLAINLAEPLYVTNNSLNLRLASPFYVNNNSLNLRLPNPSGLSIDGSGLKVKAGYGLRLHANNDLYVYTDFPLDKYDNGAKQGQLYLRTGKGLQLSNNDLTVKLGAGLKFDNNGAITLAST

SEQ ID NO: 215 - protein, 359 aa, synthetic construct
MKRARIDEDYNPVYPYNTSTPPFIAPPFVDNDGFQENPQGVLSLRISKPLYFDINKRLSLSLGQGLSTTSAGELESAQNVRANPPLSINSNTVSLRYSSPLNLTENGLVLGYSDPLRVVGGALTFNYTSPLRYENDSLTFNYTSPLKLMNNSLAIEVNSSKGVTSKNGAIAVQLSPDLKFDSNGSIAFGIQTLWTFPTTTSNCTVFTSNDSLLSLCLTKCGAHVLGSVSLTGMAGALLNMTQTSVTIQFTFDNTGSLTSSPLINNAWGIRQNDSASPNPTFNALTFMPNSTVYSRGGSGEPRNNYYTQTYLRGNIRKPIVLTVTYNSANTGYSLSFKWDAIATEKFATPTSSFCYISEQ

SEQ ID NO: 216 - protein, 593 aa, synthetic construct; identical to SEQ ID NO: 199

SEQ ID NO: 217 - protein, 224 aa, synthetic construct
MYLLITTHDFHLSQMKRTRVDEDFNPVYPYDAEQSSSVPFVTPPFTSSNGLTESPSGVLTLNYSQPLTTINGKLTLNVDGGLNINNDKKLQVRTNENEGVTLQNNAVSVKIGNDLRFDSRGQITLNPVSLWTTPSESANCTLYNSSANFFLCLTRNDAHVVGTVNLTGINGKFSSMTENSVIVQLAFDENGTLQSSPLAQNVWAYVKLLTLQTLLICLHLCPAA

SEQ ID NO: 218 - protein, 292 aa, synthetic construct; identical to SEQ ID NO: 195

SEQ ID NO: 219 - protein, 555 aa, synthetic construct; identical to SEQ ID NO: 186

SEQ ID NO: 220 - protein, 505 aa, synthetic construct
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRAKLMVEKSNAETRQPRYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPALLDVSKYEASIQRAKAEGREIRGDTFAVAPQDLEIVPLTKDSKDRSYNIINNTTDTLYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 221 - protein, 503 aa, synthetic construct
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVKKVENQPPEYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPALLDVTKYEQSVQRAKAEGREIRGDTFAVSPQDLVIEPLEHDSKNRSYNLLPNKTDTAYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 222 - protein, 504 aa, synthetic construct
MRRAVRVTPAVYAEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVEKTSGQPPKYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPGLLDVPAYEQSLQQAQEEGRVTRGDTFATAPNEVVIKPLLKDSKDRSYNIITDTTDTLYRSWFLAYNYGDPENGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 223 - protein, 503 aa, synthetic construct
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVKKAENQPPEYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPGLLDVPAYEESVKQAEAQGREIRGDTFATEPHELVIKPLEQDSKKRSYNIISGTMNTLYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 224 - protein, 505 aa, synthetic construct; identical to SEQ ID NO: 220

SEQ ID NO: 225 - protein, 503 aa, synthetic construct
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVKKVENQPPEYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPGLLDVPAYEDSVKKAEAQGREIRGDTFATEPHALVIKPLTEDSKKRSYNIITGTMDTEYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 226 - protein, 503 aa, synthetic construct
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVKKVENQPPEYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPALLDVSKYEDSVKKAEAQGREIRGDTFAVAPQDLVIEPLTKDSKNRSYNIITGTMDTKYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 227 - protein, 506 aa, synthetic construct
MRRAVRVTPAVYAEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVEKTNAETQQPRYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPALLDVSKYEASVQRAEAEGREIRGDTFAVAPQDLVIEPLTKDSKNRSYNIITGTTDTQYRSWFLAYNYGDPDKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 228 - protein, 503 aa, synthetic construct
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVKKAENQPPEYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPGLLDVPAYEDSVKKAEAQGREIRGDTFATEPHALVIKPLTEDSKKRSYNIITGTMDTEYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 229 - protein, 505 aa, synthetic construct; identical to SEQ ID NO: 220

SEQ ID NO: 230 - protein, 505 aa, synthetic construct
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRAKLMVEKSNAETQQPRYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPGLLDVPAYEQSLQQAQEEGRVTRGDTFATAPHELVIQPLTKDSKNRSYNLIPNSTDTLYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 231 - protein, 503 aa, synthetic construct; identical to SEQ ID NO: 226

SEQ ID NO: 232 - protein, 505 aa, synthetic construct; identical to SEQ ID NO: 220

SEQ ID NO: 233 - protein, 505 aa, synthetic construct; identical to SEQ ID NO: 230

SEQ ID NO: 234 - protein, 503 aa, synthetic construct
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVKKVENQPPEYEWFEFTIPEGNYSETMTIDLMNNAIVDNYLQVGRQHGVLESDIGVKFDTRNFRLGWDPVTKLVMPGVYTNEAFHPDIVLLPGCGVDFTQSRLSNLLGIRKRRPFQEGFQIMYEDLEGGNIPALLDVSKYEDSVKKAEAQGREIRGDTFAVAPQDLVIEPLTKDSKNRSYNIITGTMDTEYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 235 - protein, 503 aa, synthetic construct
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRAKLMVEKISGQPPKYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPGLLDVPAYEDSVKKAEAQGREIRGDTFATEPHALVIKPLTEDSKKRSYNIITGTMDTEYRSWFLAYNYGDPEKGVRSWTILTTTDVTCGSQQVYWSLPDMMQDPVTFRPSTQVGNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 236 - protein, 505 aa, synthetic construct; identical to SEQ ID NO: 230

SEQ ID NO: 237 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 238 - protein, 1270 aa, synthetic construct
MFVFLVLLPLVSSQCVNFTNRTQLPSAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWTESEFRVYSSANNCTFEYVSQPFLMDLEGKQGNFKNLREFVFKNIDGYFKIYSKHTPINLVRDLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLFRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 239 - protein, 1270 aa, synthetic construct
MFVFLVLLPLVSIQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGGSSSGWTAGSAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGNTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 240 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFYNPVLPFNDGVYFASTEKSNIIRGWIFGTTLDSKTQSLLIVNNATNVVIKVCEFQFCNDPFFGVYYHKNNKSWMESELRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSFSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSFLYNSASFSTFKCYGVSPTKLNDLCFTNVYADSFVIRGDEVRQIAPGQTGTIADYNYKLPDDFTGCVIAWNSKNLDSKVGGNYNYLYRLFRKSNLKPFERDISTEIYQAGRTPCNGVEGFNCYFPLQPYGFQPTTGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYHDVNCTEVPVAIHADQLTPTWRVYSTGSNVFQTRAGCLIGAEHVNNSYECDIPIGAGICASYQTQTNSRGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 241 - protein, 13 aa, Severe acute respiratory syndrome coronavirus 2
SEPVLKGVKLHYT

SEQ ID NO: 242 - protein, 52 aa, Severe acute respiratory syndrome coronavirus 2
YIKWPWYIWLGFIAGLIAIVMVTIMLCCMTSCCSCLKGCCSCGSCCKFDEDD

SEQ ID NO: 243 - protein, 26 aa, unidentified
YIPEAPRDGQAYVRKDGEWVLLSTFL

SEQ ID NO: 244 - protein, 4 aa, synthetic construct
GSGG

SEQ ID NO: 245 - protein, 5 aa, synthetic construct
GGSGG

SEQ ID NO: 246 - protein, 4 aa, synthetic construct
RRAR

SEQ ID NO: 247 - protein, 4 aa, synthetic construct
GGSG

SEQ ID NO: 248 - protein, 17 aa, Severe acute respiratory syndrome coronavirus 2
MFVFLVLLPLVSSQCVN

SEQ ID NO: 249 - protein, 1256 aa, synthetic construct
LTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 250 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 251 - protein, 1260 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDD

SEQ ID NO: 252 - protein, 1256 aa, Severe acute respiratory syndrome coronavirus 2
LTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 253 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 254 - protein, 1268 aa, Severe acute respiratory syndrome coronavirus 2
MFVFFVLLPLVSSQCVNFTNRTQLPSAYTNSFTRGFYYPDKVFRSSVLHSTRDLFLPFFSNVTWFHAIHVSGTNGTKRFGNPVLPFNDGVYFASIEKSNIIRGWVFGTTLDSKTQSLFIVNNATNVFIKVCEFQFCNYPFLGVYYHKNNKSWMESGVYSSANNCSFEYVSQPFLMHLEGKQGNFKNLSEFVFKNIDGYFKVYSKHTPINLVRGLPQGFSVLEPLVDLPIGVNITRFQTLHRSYLTPGGSSSGWTAGSAAYYVGYLQLRTFLLKYNENGIITDAVDCALDPLSEIKCTLKSFTLEKGIYQTSNSRVQPTESIIRFPNITNLCSFGEVFNATSFASVYAWNRRRISNCVADYSLLYNSASFSTFKCYGVSLTKLNDLCFTSVYADSFVIRGDEVIQIAPGQTGTIADYNYKLPNDFTGCVIAWNSKNLDSKVGGNYNYRYRLFRKSNLKPFERDVSTEIYQAGSKPCNGVKGFNCYFPLQSYGFQPTNGVGYQPYRVVVFSFELLHAPSTVCGPKKSSNLVKNKCVSFNFNGLTGIGVLTESNKNFLPFQQFGRDIADTTDAVRDPQTLDILDITPCSFGSVSVITPGTNASNQVAVLYHGVNCTEVPFAIHADQLTSTWRVYSTGFNVFQTRAGCLIGAEYVNNSYECDIPIGAGVCASYQTQTNSPRRARSVASQPIIAYTMSLGVENSVAYSNNSIAIPTNFTISVTTEILPVSMAKTSVDCTMYICGDSIECSNLLLQYGGFCTQLNRALTVIAVEQDKNTKEVFAQVKQIYKTPPIKHFGGFNFSQILPDSSKPSKRSFIEDLFFNKVTLADVGFIKQYGDCLGDISARDLICAQKFNGLNVLPPLLTDEMVAQYTSALLSGTITSGWTLGAGAALQIPFSMQMAYRFNGVGVTQNVLYENQKFIANQFNSAIGKIQDSLFSTASALGKLQNVVNQNAQALNMLVKQLSSNFGAISSVLNDILSRLDKVEAEVQIDRLVTGRLQSLQIYVTQQLIRAVEIRASANLAAIKMSECVLGQSKRFDFCGKGYHIMSFPQSAPYGVVFLHVTYVPAHEKNFTTAPAICHYGKAHFPREGFFVSNGTHWFLTQRNFYEPQIITTDNTFVSGNCDVVVGIVNNTVYHPLQPELDSFKEELYKYFKNHTSSDVDLGDISGINASFVNIQKEIDRLNEVANNLNESLIDLQELGKYEHYIKWPWYIWLVFIAGLIAILMVTIMLCCITSCCSCLKGCCSCGFCCKFDEDDSEPLLKGVKLHYI

SEQ ID NO: 255 - protein, 1271 aa, Severe acute respiratory syndrome coronavirus 2
MFVFLVLLLLVSSQCVNLRTRTQLPPAYTNSFIRGVYYPDKVFRSSVLYSTQDLFLPFFSNVTWFHVISGTNGTKRFANPVLPFNDGVYFASTEKFNIIRGWIFGTTLDLKTQSLLIVNNATNAVIKVCEFQFCNDPFLDVYYHKNNKSRMESEFRVYSIANNCTFEYVSQPFFMDLEGKQGNFKNFREFVFKNIYGYFKIYSKHTPTNLVRDLPQGFSVLEPLVHLPIGINITRFQTLLALYRSYLTPGDSSSGLTAGAAAYYLGYLQPRTFLLKYNQNGTITDAVDCSLDPLSETKCTLKSFIVEKGIYQTSNFRVQPIESIVRFPNITNFCPFGEVFNATKFASVYAWNRKKISNCVADYSFLYNSASFSIFKCYGVSPNKLNDLCFTNIYADSFVIRGDQVRQIAPGQTGNIADYNYKLPYDFTGCVIAWNSNKLDSKVGGNYNYQYRLFRKSNLTPFERDISTQIYQAGSTPCNGVKGFNCYSPLQSYGFQPTTGVGYQPYRLVVLSFELLHSPATVCGPKKSTNLIKNKCVNFNFNGLIGTGVLTESNKKFLSFQQFGRDIDDITDAVRDPQTLQILDITPCSFGGISVITPGTNISNQVAVLYQDVNCTEVPVSIHADQLTPSWRVYSTGSSVFQTRAGCLIGVEHVNNSYECDIPIGAGICVSYQTQTNSRRRARSVASQSIIVYTMSLGAENLVAYSNNSIAIPVNFTISVTTEILPVSITKTSVDCTVYICGDSTECSDLLLQYGSFCIQLNRALTGISVEQDKNTQDVFAQVKQIYKIPPIKDFGGFNFAQILPDPSKSSKRSFIEDLLFNKVILADAGFIKQYGDCLGNIAARDLICAQNFNGLTVLPPLLTDEMIVQYTSALLAGTIISGWTFGAGVALQIPFAMQIAYRFNGIGITQNVLYENQKLISNQFNSAIGKIQYSLSSTASALRKLQDVVNQNARALNTLVKQLNSNFGAISSFLNDILSRLDNVEAEVQIDRLIIGRLQSLQTYVIQQLIRAAEIRSSANLAATKMSECALGQSKRVDFCGNGYHLMSFPQSVPHGVVFLHVNYVPAQEKNFTTSPAICHDGKSHFPREGVFVSNGTDWFVTQRNFYKPQIITTDNTFVSVNCDVVIGIFNNTVYDPLQPELYSFKEELDKYFKNHTSLDVDLGDISGINVSVVNIQKEIDHLNEVAKNLNQSLIDLQELGNYEQYIKWPWYIWLCFIAGLIAIVIVTIMLCCMISCCSCLKGFCSCGSCCKFDEDNSEPVLKGVKLYYT

SEQ ID NO: 256 - protein, 1266 aa, Severe acute respiratory syndrome coronavirus 2
MFGFLVLLPLVSIQCVNFTTRTQLPPSYTNSFTRGIYYPDKVFRFSVLHSTQDFFLPFFSNVTRFHAIHVSGTNVIKRFDNPVLSFNDGVYFAFTEKSNIIRGWIFGTTLNSKTQSLLILNNATNVVIKVCQFQFCNDPFLGVYYHKTNKSWMESESRVYSSANNCTFEYVAQPFLMDLEVKQGNFKNLREFLFKNIDGYFKIHSKHTPINLVRDFPQGFSALEPLADLPIGINITKFQTLLALHNSSSGWTAGAPAYYVGYLQPSTFLLKYNENGTITDALDCALDPLSETMCTLKSFTVQKGIYQTSNFRFQPTESIVRFSNITNLCPFSEVFNATRFSSVYAWNRKRISNCFADYSVLYNTASFSTFKCYGLSPTKLNDLCFTHVYADSFVIKGDEVRQIAPGKTGKIADYNYKLPDDFIGCVIAWNSNSLDSKVGGNYNYMYRLFRKSNFKPFERDISIEIYQAGSKPCNGVEGFNCYFPLQPYGFQPTYGVGHQPYRVVVLSFELLHAPVTVCGPKKSTNLFKNKCVNFNLNGLTGTGVLTDSNKKFLPFQQLGRDIDDTTDSVRDPQTLEIFDITPCSFGGVSVVTPGTNTSNKVAVLYQGVSCTEVPVAIHAGQLTPTWRVYSIGSNVFQTRSGCLIGAEHVNNSFECDIPIGAGICASYQTHTNSPRRARSVVSQSIIAYTISLGAENSVVYSNNSIAIPINFIISVTTEILPVSMIKTSVDCTMYICGDSNECSNLLLQYGSFCRQLNRALTGIAIEQDKNTQEVFAQIKQIYKTPPIRDFGGFNFSQILPDPSKLSKRSFIEDLLFNKLTLADAGFIRQYGDCLGDIVARDLICAQKFNGLIVLPPLLTDEIIAQYTSALLVGTITSGWTFGVGAALQIPFAMQMSYRFNGIGVTQNILYENQKLIANQFNIAIGKIQDSLSFTASALGKLQHVVNQNAQALNTLAKQLSSNFGAISSFLNDILARLDRVEAEVQIDGLITGRLQSLHTYVTQQLIKAAEIRASANLGATKMSECVLGQSQRVDFCGKGYHLISFPQSAPHGVVFFHVTYVPAQENNFTTAPAICHEGKAHFPREGVFVSNGTYWFVTQRNFYEPQIITTYNTFVSGNCGVVIGIVNNTVYDPWQPELDSFKDELDKYFKNHTSPYVDLGDISGISASVVNIQKDIDRLNEVAKSLNESLIDLQQLGKYEQYIKWPWYTWLGFIAGLIAIVMLTIMLCCMTSCCSFLKGCCSCGPCCKFDEDDSELVLKGVKLHYA

SEQ ID NO: 257 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHGGSGSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 258 - protein, 1268 aa, Severe acute respiratory syndrome coronavirus 2
MFVFFVLLPLVSSQCVNFTNRTQLPSAYTNSFTRGFYYPDKVFRSSVLHSTRDLFLPFFSNVTWFHAIHVSGTNGTKRFGNPVLPFNDGVYFASIEKSNIIRGWVFGTTLDSKTQSLFIVNNATNVFIKVCEFQFCNYPFLGVYYHKNNKSWMESGVYSSANNCSFEYVSQPFLMHLEGKQGNFKNLSEFVFKNIDGYFKVYSKHTPINLVRGLPQGFSVLEPLVDLPIGVNITRFQTLHRSYLTPGGSSSGWTAGSAAYYVGYLQLRTFLLKYNENGIITDAVDCALDPLSEIKCTLKSFTLEKGIYQTSNSRVQPTESIIRFPNITNLCSFGEVFNATSFASVYAWNRRRISNCVADYSLLYNSASFSTFKCYGVSLTKLNDLCFTSVYADSFVIRGDEVIQIAPGQTGTIADYNYKLPNDFTGCVIAWNSKNLDSKVGGNYNYRYRLFRKSNLKPFERDVSTEIYQAGSKPCNGVKGFNCYFPLQSYGFQPTNGVGYQPYRVVVFSFELLHAPSTVCGPKKSSNLVKNKCVSFNFNGLTGIGVLTESNKNFLPFQQFGRDIADTTDAVRDPQTLDILDITPCSFGSVSVITPGTNASNQVAVLYHGVNCTEVPFAIHADQLTSTWRVYSTGFNVFQTRAGCLIGAEYVNNSYECDIPIGAGVCASYQTQTNSPGGSGSVASQPIIAYTMSLGVENSVAYSNNSIAIPTNFTISVTTEILPVSMAKTSVDCTMYICGDSIECSNLLLQYGGFCTQLNRALTVIAVEQDKNTKEVFAQVKQIYKTPPIKHFGGFNFSQILPDSSKPSKRSFIEDLFFNKVTLADVGFIKQYGDCLGDISARDLICAQKFNGLNVLPPLLTDEMVAQYTSALLSGTITSGWTLGAGAALQIPFSMQMAYRFNGVGVTQNVLYENQKFIANQFNSAIGKIQDSLFSTASALGKLQNVVNQNAQALNMLVKQLSSNFGAISSVLNDILSRLDPPEAEVQIDRLVTGRLQSLQIYVTQQLIRAVEIRASANLAAIKMSECVLGQSKRFDFCGKGYHIMSFPQSAPYGVVFLHVTYVPAHEKNFTTAPAICHYGKAHFPREGFFVSNGTHWFLTQRNFYEPQIITTDNTFVSGNCDVVVGIVNNTVYHPLQPELDSFKEELYKYFKNHTSSDVDLGDISGINASFVNIQKEIDRLNEVANNLNESLIDLQELGKYEHYIKWPWYIWLVFIAGLIAILMVTIMLCCITSCCSCLKGCCSCGFCCKFDEDDSEPLLKGVKLHYI

SEQ ID NO: 259 - protein, 1271 aa, Severe acute respiratory syndrome coronavirus 2
MFVFLVLLLLVSSQCVNLRTRTQLPPAYTNSFIRGVYYPDKVFRSSVLYSTQDLFLPFFSNVTWFHVISGTNGTKRFANPVLPFNDGVYFASTEKFNIIRGWIFGTTLDLKTQSLLIVNNATNAVIKVCEFQFCNDPFLDVYYHKNNKSRMESEFRVYSIANNCTFEYVSQPFFMDLEGKQGNFKNFREFVFKNIYGYFKIYSKHTPTNLVRDLPQGFSVLEPLVHLPIGINITRFQTLLALYRSYLTPGDSSSGLTAGAAAYYLGYLQPRTFLLKYNQNGTITDAVDCSLDPLSETKCTLKSFIVEKGIYQTSNFRVQPIESIVRFPNITNFCPFGEVFNATKFASVYAWNRKKISNCVADYSFLYNSASFSIFKCYGVSPNKLNDLCFTNIYADSFVIRGDQVRQIAPGQTGNIADYNYKLPYDFTGCVIAWNSNKLDSKVGGNYNYQYRLFRKSNLTPFERDISTQIYQAGSTPCNGVKGFNCYSPLQSYGFQPTTGVGYQPYRLVVLSFELLHSPATVCGPKKSTNLIKNKCVNFNFNGLIGTGVLTESNKKFLSFQQFGRDIDDITDAVRDPQTLQILDITPCSFGGISVITPGTNISNQVAVLYQDVNCTEVPVSIHADQLTPSWRVYSTGSSVFQTRAGCLIGVEHVNNSYECDIPIGAGICVSYQTQTNSRGGSGSVASQSIIVYTMSLGAENLVAYSNNSIAIPVNFTISVTTEILPVSITKTSVDCTVYICGDSTECSDLLLQYGSFCIQLNRALTGISVEQDKNTQDVFAQVKQIYKIPPIKDFGGFNFAQILPDPSKSSKRSFIEDLLFNKVILADAGFIKQYGDCLGNIAARDLICAQNFNGLTVLPPLLTDEMIVQYTSALLAGTIISGWTFGAGVALQIPFAMQIAYRFNGIGITQNVLYENQKLISNQFNSAIGKIQYSLSSTASALRKLQDVVNQNARALNTLVKQLNSNFGAISSFLNDILSRLDPPEAEVQIDRLIIGRLQSLQTYVIQQLIRAAEIRSSANLAATKMSECALGQSKRVDFCGNGYHLMSFPQSVPHGVVFLHVNYVPAQEKNFTTSPAICHDGKSHFPREGVFVSNGTDWFVTQRNFYKPQIITTDNTFVSVNCDVVIGIFNNTVYDPLQPELYSFKEELDKYFKNHTSLDVDLGDISGINVSVVNIQKEIDHLNEVAKNLNQSLIDLQELGNYEQYIKWPWYIWLCFIAGLIAIVIVTIMLCCMISCCSCLKGFCSCGSCCKFDEDNSEPVLKGVKLYYT

SEQ ID NO: 260 - protein, 1266 aa, Severe acute respiratory syndrome coronavirus 2
MFGFLVLLPLVSIQCVNFTTRTQLPPSYTNSFTRGIYYPDKVFRFSVLHSTQDFFLPFFSNVTRFHAIHVSGTNVIKRFDNPVLSFNDGVYFAFTEKSNIIRGWIFGTTLNSKTQSLLILNNATNVVIKVCQFQFCNDPFLGVYYHKTNKSWMESESRVYSSANNCTFEYVAQPFLMDLEVKQGNFKNLREFLFKNIDGYFKIHSKHTPINLVRDFPQGFSALEPLADLPIGINITKFQTLLALHNSSSGWTAGAPAYYVGYLQPSTFLLKYNENGTITDALDCALDPLSETMCTLKSFTVQKGIYQTSNFRFQPTESIVRFSNITNLCPFSEVFNATRFSSVYAWNRKRISNCFADYSVLYNTASFSTFKCYGLSPTKLNDLCFTHVYADSFVIKGDEVRQIAPGKTGKIADYNYKLPDDFIGCVIAWNSNSLDSKVGGNYNYMYRLFRKSNFKPFERDISIEIYQAGSKPCNGVEGFNCYFPLQPYGFQPTYGVGHQPYRVVVLSFELLHAPVTVCGPKKSTNLFKNKCVNFNLNGLTGTGVLTDSNKKFLPFQQLGRDIDDTTDSVRDPQTLEIFDITPCSFGGVSVVTPGTNTSNKVAVLYQGVSCTEVPVAIHAGQLTPTWRVYSIGSNVFQTRSGCLIGAEHVNNSFECDIPIGAGICASYQTHTNSPGGSGSVVSQSIIAYTISLGAENSVVYSNNSIAIPINFIISVTTEILPVSMIKTSVDCTMYICGDSNECSNLLLQYGSFCRQLNRALTGIAIEQDKNTQEVFAQIKQIYKTPPIRDFGGFNFSQILPDPSKLSKRSFIEDLLFNKLTLADAGFIRQYGDCLGDIVARDLICAQKFNGLIVLPPLLTDEIIAQYTSALLVGTITSGWTFGVGAALQIPFAMQMSYRFNGIGVTQNILYENQKLIANQFNIAIGKIQDSLSFTASALGKLQHVVNQNAQALNTLAKQLSSNFGAISSFLNDILARLDPPEAEVQIDGLITGRLQSLHTYVTQQLIKAAEIRASANLGATKMSECVLGQSQRVDFCGKGYHLISFPQSAPHGVVFFHVTYVPAQENNFTTAPAICHEGKAHFPREGVFVSNGTYWFVTQRNFYEPQIITTYNTFVSGNCGVVIGIVNNTVYDPWQPELDSFKDELDKYFKNHTSPYVDLGDISGISASVVNIQKDIDRLNEVAKSLNESLIDLQQLGKYEQYIKWPWYTWLGFIAGLIAIVMLTIMLCCMTSCCSFLKGCCSCGPCCKFDEDDSELVLKGVKLHYA

SEQ ID NO: 261 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 262 - protein, 222 aa, Severe acute respiratory syndrome coronavirus 2
MAGSNGTITVEELKKLIEQWNLVIGFLLLTWICLLQFSYANRNRFLYVIKLIFLWLLWPLTLACFVLAALYRINWITGGIATAMACLVGLMWLIYFIASFRLFVRTRSMWSFNSETNILLNVPLYGTILTRPLLESELVIGVVILRGHLRIAGHYLGRCDIKDLPREITVATSRMLSYYKLGASQRVSGDSGFAAYTRYRIGNYKLNTYHSSSSDNIALLLQ

SEQ ID NO: 263 - protein, 222 aa, Severe acute respiratory syndrome coronavirus 2
MSDSNGTITVEELKKLFEQWNLVIGFLFLTWICFLQFAYANRNSFLYIIKLIFLWLLWPITLACFVLAAFYRINWITGGIAIAMSCLVGLMWLSYFISSFRLFARTRSIWSFNPETNILLNVPFHGTILTRPLLESEIVIGAVILRGQLRIAGHHLGLCDIKDLPKELTVATSRTLSYYRLGASQRVACDSGFAAYSRHRIGNYKLNIDHSSSSDNVALLVQ

SEQ ID NO: 264 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 265 - protein, 419 aa, Severe acute respiratory syndrome coronavirus 2
MSLNGPQNQRNALRITFGGPSGSTGSNQNGEHSGARSKQRRPQGLSNNTASWFTSLTQHGKEDLKFSRGQGVPINTNSSRDDQIGYYRRTTRRIRGGDGKMKYLSPRWYFYYLGTGPESGLPYGANKYGIIWVATEGAFNTPKDHIGTRNLANNAAIVLQLPQGTTLSKGFYAEGSRGGSQAYSRSSSRSRNSLRNSTPGSSKRTSPARMAGNGGDAALVLLLLDRLNQLESKMFGKGQQQQGQTVTKKSASEASKKPRQKRTATKAYNLTQAFGRRGQEQTQGNFGDQELTRQGTDYKHWPQIAQFSPSASAFFGMSRIGMEVIPSGTWLTYIGAIKLDDKDSNFKDQVILLSKHIDAYKTFPSTEPKKDKKKKTDETQALPQKQKKQQIVTLLPAVDLDDFSKQLQQSMSIADSTQA

SEQ ID NO: 266 - protein, 419 aa, Severe acute respiratory syndrome coronavirus 2
MSDNGPQNHRNAPRITFVGPSDSTGSNQNRERSGARSKQCRPQGLPNNIASWFTALTQHGKENLKFPRGQGIPINTNSSPYDQIGYYRRSTRRIRGGDGKMKGLSPRWYFYYLGTGPEAVLPYGANKDGIIWVAIEGALNTPKDYIGTRNPANNASIVLQLPQGTILPKGFYAEGSRGGIQASSRSSSRIRNSSRNSTLGSSRGTSPARIAGNGGDAAIALLLLDRLNQLESKISGKGQQQQCQTVTKKSAADASKKPRQKRTATKVYNVTQAFGRRGSEQTQGNFGDHELIRQGTDYKYWPQIAQFAPSASSFFGMSRIGMEVTPLGTWLTYTGAIKLDNKDPNFKDQFILLNKHIDAYKTFPPIEPKKDKKKKAYETQALPQRQKKQQIVTLLPAADLYDFSKQLQQSMSSSDSTQA